(19) United States
(12) Patent Application Publication
(10) Pub. No.: US 2016/0329505 A1
(43) Pub. Date: Nov. 10, 2016

(54) COMPOUND, MATERIAL FOR ORGANIC ELECTROLUMINESCENT ELEMENTS, INK COMPOSITION, ORGANIC ELECTROLUMINESCENT ELEMENT AND ELECTRONIC DEVICE

(71) Applicant: IDEMITSU KOSAN CO., LTD., Tokyo (JP)

(72) Inventors: Kiyoshi IKEDA, Sodegaura-shi (JP); Hironori KAWAKAMI, Tokyo (JP); Taro YAMAKI, Chiba-shi (JP)

(73) Assignee: IDEMITSU KOSAN CO., LTD., Tokyo (JP)

(21) Appl. No.: 15/104,709

(22) PCT Filed: Dec. 16, 2014
(86) PCT No.: PCT/JP2014/083314

§ 371 Date: Jun. 15, 2016

(30) Foreign Application Priority Data
Dec. 18, 2013 (JP) 2013-261792

(51) Int. Cl. H01L 51/00; C07D 403/14; C09D 11/52; C09K 11/02; C09K 11/06

(52) U.S. Cl. CPC H01L 51/0072; C09K 11/025; C09K 11/06; H01L 51/0085; C09D 11/52; C07D 403/14; H01L 51/0067; H01L 51/5016

(57) ABSTRACT

Provided are an organic electroluminescence device having improved performance and an electronic equipment containing the device, and also provided are a compound, a material for organic electroluminescence devices and an ink composition that enable the device and the equipment. The organic electroluminescence device contains a cathode, an anode, and one or more organic tin film layers containing a light emitting layer between the cathode and the anode, wherein at least one layer of the one or more organic thin film layers contains a compound represented by the formula (1) wherein Cz¹ is a group represented by the formula (Cz-1), Cz² is a group represented by the formula (Cz-2), A is a residue of a substituted or unsubstituted, nitrogen-containing aromatic hetero ring having 6 to 30 ring atoms, L¹ and L² each independently represent a substituted or unsubstituted arylene group having 6 to 60 ring carbon atoms, and n1 and n2 each are independently an integer of 0 to 4.

### TECHNICAL FIELD

The present invention relates to a compound, a material for organic electroluminescence devices, an ink composition, an organic electroluminescence device, and an electronic equipment.

### BACKGROUND ART

There is known an organic electroluminescence device which has organic thin film layers containing a light emitting layer between an anode and a cathode, and which emits light from excitons formed through recombination of holes and electrons injected into the light emitting layer.

An organic electroluminescent device is expected as a light emitting device excellent in light emission efficiency, imaging quality, energy saving performance and flat-panel design, taking advantage of self-emitting devices. Using various light emitting materials in the light emitting layer therein, an organic electroluminescent device can give a variety of emission colors and a lot of studies thereof in practical use for a display or the like are being made much. In particular, studies of light emitting materials of three primary colors of red, green and blue are most active, and earnest studies for improving the characteristics thereof are being made.

PTL 1 has an object of providing compounds having both hole transporting capability and electron transporting capability and excellent in carrier balance, and describes a compound containing a biscarbazole structure and a nitrogen-containing aromatic heterocyclic structure in one and the same molecule, and a compound containing a tricarbazole structure and a nitrogen-containing aromatic heterocyclic structure in one and the same molecule. However, the latter tricarbazole structure-containing compound is not applied to organic EL devices, and the reference says nothing relating to the performance of the compound as an organic EL device material. As other references, PTLs 2 to 6 describe organic EL device materials.

In the field of organic EL devices, it is desired to develop materials useful for the devices for the purpose of further improving the performance of the devices.

### CITATION LIST

### Patent Literature

PTL 1: WO2012/086170

PTL 2: JP 2012-149257 A

PTL 3: WO 2010/085676

PTL 4: WO2012/069121

PTL 5: WO2012/077902

PTL 6: WO2013/081088

### SUMMARY OF INVENTION

### Technical Problem

One object of the present invention is to provide an organic EL device having excellent characteristics and an electronic equipment containing it. Another object is to provide a compound, a material for organic electroluminescent devices and an ink composition that enable an organic EL device having excellent characteristics and an electronic equipment containing it.

### Solution to Problem

According to one aspect of the present invention, there is provided an organic electroluminescence device including a cathode, an anode and one or more organic thin film layers including a light emitting layer between the cathode and the anode, wherein at least one layer of the one or more organic thin film layers contains a compound represented by the formula (1):

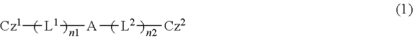

wherein Cz¹ represents a group represented by the formula (Cz-1),
Cz² represents a group represented by the formula (Cz-2),
A represents a residue of a substituted or unsubstituted nitrogen-containing aromatic hetero ring having 6 to 30 ring atoms,
L¹ and L² each independently represent a substituted or unsubstituted arylene group having 6 to 60 ring carbon atoms,
n1 and n2 each independently indicate an integer of 0 to 4,
when n1=0, A and Cz¹ bond via a single bond,
when n2=0, A and Cz² bond via a single bond,
when n1 is an integer of 2 to 4, L¹'s may be the same as or different from each other, and L¹'s may form a ring,
   when n2 is an integer of 2 to 4, L²'s may be the same as or different from each other, and L²'s may form a ring;

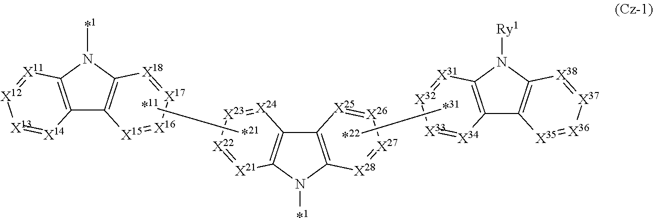

wherein X¹¹ to X¹⁴ each independently represent N or C-*²,
one of X¹⁵ to X¹⁸ is a carbon atom bonding to *¹¹, and the other three each are independently N or C-*²,
one of X²¹ to X²⁴ is a carbon atom bonding to *²¹, and the other three each are independently N or C-*²,
one of X²⁵ to X²⁸ is a carbon atom bonding to *²², and the other three each are independently N or C-*²,
one of X³¹ to X³⁴ is a carbon atom bonding to *³¹, and the other three are independently N or CRx¹,
X³⁵ to X³⁸ each independently represent N or CRx¹,
one of *¹'s or one of *²'s bonds to L¹ in the formula (1),
*¹ not bonding to L¹ bonds to Ry¹,
*² not bonding to L¹ bonds to Rx¹,
Ry¹ each independently represents a hydrogen atom or a substituent,
Rx¹ each independently represents a hydrogen atom or a substituent,
Rx¹'s may form a ring;

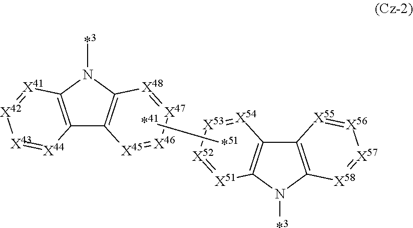

wherein X⁴¹ to X⁴⁴ each independently represent N or C-*⁴,
one of X⁴⁵ to X⁴⁸ is a carbon atom bonding to *⁴¹, and the other three each are independently N or C-*⁴,
one of X⁵¹ to X⁵⁴ is a carbon atom bonding to *⁵¹, and the other three each are independently N or C-*⁴,
X⁵⁵ to X⁵⁸ each independently represent N or C-*⁴,
one of *³'s or one of *⁴'s bonds to L² in the formula (1),
*³ not bonding to L² bonds to Ry²,
*⁴ not bonding to L² bonds to Rx²,
Ry² each independently represents a hydrogen atom or a substituent,
Rx² each independently represents a hydrogen atom or a substituent,
Rx²'s may form a ring.

According to one aspect of the present invention, there is provided an electronic equipment containing the above-mentioned organic electroluminescence device.

According to one aspect of the present invention, there is provided a compound represented by the formula (1):

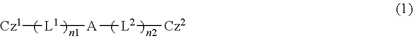

wherein Cz¹ represents a group represented by the formula (Cz-1),
Cz² represents a group represented by the formula (Cz-2),
A represents a residue of a substituted or unsubstituted nitrogen-containing aromatic hetero ring having 6 to 30 ring atoms,
L¹ and L² each independently represent a substituted or unsubstituted arylene group having 6 to 60 ring carbon atoms,
n1 and n2 each independently indicate an integer of 0 to 4,
provided that the structure represented by -(L¹)ₙ₁-Cz¹ differs from the structure represented by -(L²)ₙ₂-Cz²,
when n1=0, A and Cz¹ bond via a single bond,
when n2=0, A and Cz² bond via a single bond,
when n1 is an integer of 2 to 4, L¹'s may be the same as or different from each other, and L¹'s may form a ring,
when n2 is an integer of 2 to 4, L²'s may be the same as or different from each other, and L²'s may form a ring;

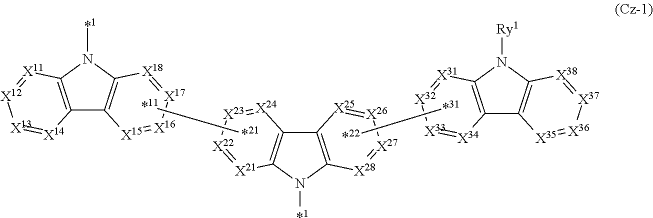

wherein X¹¹ to X¹⁴ each independently represent N or C-*²,
one of X¹⁵ to X¹⁸ is a carbon atom bonding to *¹¹, and the other three each are independently N or C-*²,
one of X²¹ to X²⁴ is a carbon atom bonding to *²¹, and the other three each are independently N or C-*²,
one of X²⁵ to X²⁸ is a carbon atom bonding to *²², and the other three each are independently N or C-*²,
one of X³¹ to X³⁴ is a carbon atom bonding to *³¹, and the other three are independently N or CRx¹,
X³⁵ to X³⁸ each independently represent N or CRx¹,
one of *¹'s or one of *²'s bonds to L¹ in the formula (1),
*¹ not bonding to L¹ bonds to Ry¹,
*² not bonding to L¹ bonds to Rx¹,
Ry¹ each independently represents a hydrogen atom or a substituent, and
Rx¹ each independently represents a hydrogen atom or a substituent,
Rx¹'s may form a ring;

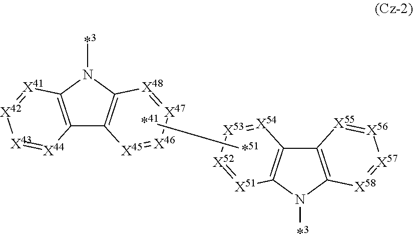

wherein X⁴¹ to X⁴⁴ each independently represent N or C-*⁴,
one of X⁴⁵ to X⁴⁸ is a carbon atom bonding to *⁴¹, and the other three each are independently N or C-*⁴,
one of X⁵¹ to X⁵⁴ is a carbon atom bonding to *⁵¹, and the other three each are independently N or C-*⁴,
X⁵⁵ to X⁵⁸ each independently represent N or C-*⁴,
one of *³'s or one of *⁴'s bonds to L² in the formula (1),
*³ not bonding to L² bonds to Ry²,
*⁴ not bonding to L² bonds to Rx²,
Ry² each independently represents a hydrogen atom or a substituent,
Rx² each independently represents a hydrogen atom or a substituent,
Rx²'s may form a ring.

According to one aspect of the present invention, there is provided a material for organic electroluminescence devices including the above-mentioned compound.

According to one aspect of the present invention, there is provided an ink composition containing a solvent and the above-mentioned compound dissolved in the solvent.

### Advantageous Effects of Invention

The present invention provides an organic EL device having improved performance and an electronic equipment containing it. The present invention also provides a compound, a material for organic electroluminescence devices and an ink composition that enable them.

### BRIEF DESCRIPTION OF DRAWING

FIG. 1 is a view showing a schematic configuration of one example of an organic EL device according to one aspect of the present invention.

### DESCRIPTION OF EMBODIMENTS

In this description, the “number of ring carbon atoms” means the number of the carbon atoms among the atoms constituting the ring itself of a compound having a structure where atoms bond to form a ring (for example, a monocyclic compound, a condensed cyclic compound, a crosslinked compound, a carbon cyclic compound, a heterocyclic compound). In the case where the ring is substituted with a substituent, the carbon atoms contained in the substituent are not contained in the number of ring carbon atoms. Unless otherwise specifically indicated, the same shall apply to the “number of ring carbon atoms” to be described hereinunder. For example, a benzene ring has 6 ring carbon atoms, a naphthalene ring has 10 ring carbon atoms, a pyridinyl group has 5 ring carbon atoms, and a furanyl group has 4 ring carbon atoms. In the case where a benzene ring or a naphthalen ring is substituted with, for example, an alkyl group as a substituent, the number of the carbon atoms of the alkyl group is not contained in the number of the ring carbon atoms. In the case where, for example, a fluorene ring bonds to a fluorene ring as a substituent (including a spirofluorene ring), the carbon number of the fluorene ring as the substituent is not contained in the number of the ring carbon atoms.

In this description, the “number of ring atoms” means the number of the atoms constituting the ring itself of a compound (for example, a monocyclic compound, a condensed cyclic compound, a crosslinked compound, a carbon cyclic compound, a heterocyclic compound) having a structure where atoms bond to form a ring (for example, a single ring, a condensed ring, an aggregated ring). The atoms not constituting a ring (for example, a hydrogen atom bonding to the bond of the atom constituting a ring), or in the case where the ring is substituted with a substituent, the atom contained in substituent is not contained in the number of ring atoms. Regarding the “number of ring atoms” to be described hereinunder, the same shall apply unless otherwise specifically indicated. For example, a pyridine ring has 6 ring atoms, a quinazoline ring has 10 ring atoms, and a furan ring has 5 ring atoms. The hydrogen atom bonding to the carbon atom of the pyridine ring or the quinazoline ring as well as the atom constituting the substituent is not contained in the number of the ring atoms. In the case where, for example, a fluorene ring bonds to a fluorene ring as a substituent (including a spirofluorene ring), the atom number of the fluorene ring as the substituent is not contained in the number of the ring atoms.

In this description, “XX to YY carbon atoms” in the expression of “a substituted or unsubstituted group ZZ having XX to YY carbon atoms” indicates the carbon number in the case where the group ZZ is unsubstituted, and the carbon number of the substituent in the case where the group is substituted is not contained. Here, “YY” is larger than “XX”, and “XX” and “YY” each indicate an integer of 1 or more.

In this description, “hydrogen atom” includes isotopes different in the neutron numbers, i.e., light hydrogen (protium), heavy hydrogen (deuterium), and tritiated hydrogen (tritium).

### [Compound]

The compound of one aspect of the present invention is described.

The compound of one aspect of the present invention is a compound represented by the formula (1) (hereinafter the “compound represented by the formula (1)” may be referred to as the “compound (1)”):

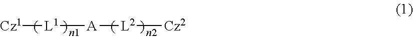

wherein Cz¹ represents a group represented by the formula (Cz-1),
Cz² represents a group represented by the formula (Cz-2),
A represents a residue of a substituted or unsubstituted nitrogen-containing aromatic hetero ring having 6 to 30 ring atoms,
L¹ and L² each independently represent a substituted or unsubstituted arylene group having 6 to 60 ring carbon atoms,
n1 and n2 each independently indicate an integer of 0 to 4,
when n1=0, A and Cz¹ bond via a single bond,
when n2=0, A and Cz² bond via a single bond,
when n1 is an integer of 2 to 4, L¹'s may be the same as or different from each other, and L¹'s may form a ring,
when n2 is an integer of 2 to 4, L²'s may be the same as or different from each other, and L²'s may form a ring;

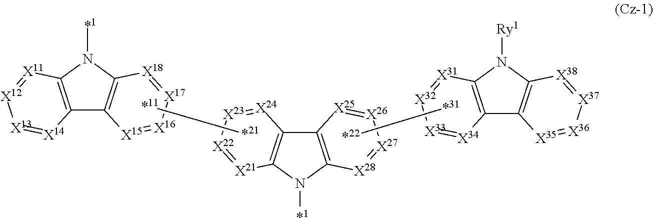

wherein X¹¹ to X¹⁴ each independently represent N or C-*²,
one of X¹⁵ to X¹⁸ is a carbon atom bonding to *¹¹, and the other three each are independently N or C-*²,
one of X²¹ to X²⁴ is a carbon atom bonding to *²¹, and the other three each are independently N or C-*²,
one of X²⁵ to X²⁸ is a carbon atom bonding to *²², and the other three each are independently N or C-*²,
one of X³¹ to X³⁴ is a carbon atom bonding to *³¹, and the other three are independently N or CRx¹,
X³⁵ to X³⁸ each independently represent N or CRx¹,
one of *¹'s or one of *²'s bonds to L¹ in the formula (1),
*¹ not bonding to L¹ bonds to Ry¹,
*² not bonding to L¹ bonds to Rx¹,
Ry¹ each independently represents a hydrogen atom or a substituent,
Rx¹ each independently represents a hydrogen atom or a substituent, and
Rx¹'s may form a ring;

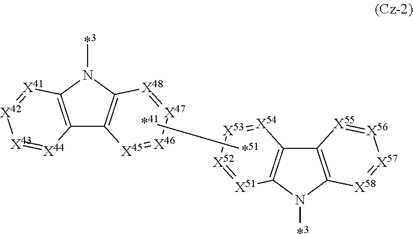

wherein X⁴¹ to X⁴⁴ each independently represent N or C-*⁴,
one of X⁴⁵ to X⁴⁸ is a carbon atom bonding to *⁴¹, and the other three each are independently N or C-*⁴,
one of X⁵¹ to X⁵⁴ is a carbon atom bonding to *⁵¹, and the other three each are independently N or C-*⁴,
X⁵⁵ to X⁵⁸ each independently represent N or C-*⁴,
one of *³'s or one of *⁴'s bonds to L² in the formula (1),
*³ not bonding to L² bonds to Ry²,
*⁴ not bonding to L² bonds to Rx²,
Ry² each independently represents a hydrogen atom or a substituent,
Rx² each independently represents a hydrogen atom or a substituent, and
Rx²'s may form a ring.

The compound (1) has the above-mentioned specific structure, and therefore has a low ionization potential and is excellent in hole injecting capability. Accordingly, using the compound (1) in an organic EL device improves the emission efficiency of the device.

Cz¹ in the formula (1) is, as described above, a group represented by the formula (Cz-1). Preferred embodiments of the formula (Cz-1) are described below.

Preferably, X¹¹ to X¹⁴ each are independently C-*².

Preferably, one of X¹⁶ and X¹⁷ is a carbon atom bonding to *¹¹, and more preferably, X¹⁶ is a carbon atom bonding to *¹¹. Preferably, three of X¹⁵ to X¹⁸ not bonding to *¹¹ each are independently C-*².

Preferably, one of X²² and X²³ is a carbon atom bonding to *²¹, and more preferably, X²³ is a carbon atom bonding to *²¹. Preferably, three of X²¹ to X²⁴ not bonding to *²¹ each are independently C-*².

Preferably, one of X² and X²⁷ is a carbon atom bonding to *²², and more preferably, X²⁶ is a carbon atom bonding to *²². Preferably, three of X²⁵ to X²⁶ not bonding to *²² each are independently C-*².

Preferably, one of X³² and X³³ is a carbon atom bonding to *³¹, and more preferably, X³³ is a carbon atom bonding to *³¹. Preferably, three of X³¹ to X³⁴ not bonding to *³¹ each are independently CRx¹.

Preferably, X³⁵ to X³⁸ each are independently CRx¹.

Preferably, one of *¹'s bonds to L¹ in the formula (1), and the others each bond to Ry¹, and every *² bonds to Rx¹.

Cz¹ in the formula (1) is preferably a group represented by the formula (Cz-11):

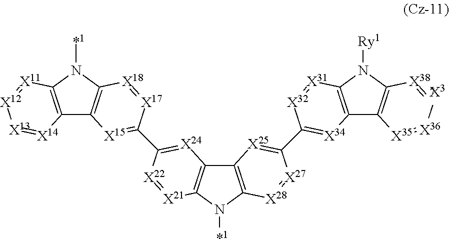

wherein X¹¹ to X¹⁵, X¹⁷ to X¹⁸, X²¹ to X²², X²⁴ to X²⁵ and X²⁷ to X²⁸ each independently represent N or C-*²,
X³¹ to X³² and X³⁴ to X³⁸ each independently represent N or CRx¹,
one of *¹'s or one of *²'s bonds to L¹ in the formula (1),
*¹ not bonding to L¹ bonds to Ry¹,
*² not bonding to L¹ bonds to Rx¹,
Ry¹ each independently represents a hydrogen atom or a substituent,
Rx¹ each independently represents a hydrogen atom or a substituent, and
Rx¹'s may form a ring.

Preferably, X¹¹ to X¹⁵, X¹⁷ to X¹⁸, X²¹ to X²², X²⁴ to X²⁵ and X²⁷ to X²⁸ each are independently C-*².

Preferably, X³¹ to X³² and X³⁴ to X³⁸ each are independently CRx¹.

Preferably, one of *¹'s bonds to L¹ in the formula (1), the others each bond to Ry¹, and every *² bonds to Rx¹.

Cz¹ in the formula (1) is preferably a group represented by the formula (Cz-12):

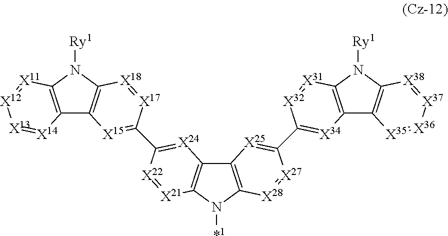

wherein X¹¹ to X¹⁵, X¹⁷ to X¹⁸, X²¹ to X²², X²⁴ to X²⁵, X²⁷ to X²⁸, X³¹ to X³² and X³⁴ to X³⁸ each are independently N or CRx¹,
*¹ bonds to L¹ in the formula (1),
Ry¹ each independently represents a hydrogen atom or a substituent,
Rx¹ each independently represents a hydrogen atom or a substituent, and
Rx¹'s may form a ring.

Preferably, X¹¹ to X¹⁵, X¹⁷ to X¹⁸, X²¹ to X²², X²⁴ to X²⁵, X²⁷ to X²⁸, X³¹ to X³² and X³⁴ to X³⁸ each are independently CRx¹.

Cz¹ in the formula (1) is preferably a group represented by the formula (Cz-13):

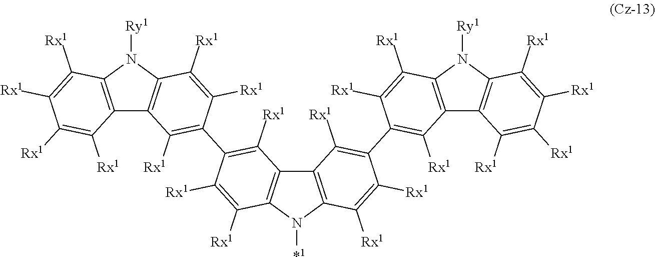

wherein *¹ bonds to L¹ in the formula (1),
Ry¹ each independently represents a hydrogen atom or a substituent,
Rx¹ each independently represents a hydrogen atom or a substituent, and
Rx¹'s may form a ring.

Cz¹ in the formula (1) is preferably a group represented by the formula (Cz-14):

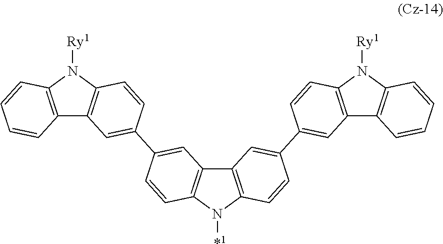

wherein *¹ bonds to L¹ in the formula (1), and Ry¹ each independently represents a hydrogen atom or a substituent.

Ry¹ in the formulae (Cz-1), (Cz-11), (Cz-12), (Cz-13) and (Cz-14) is described below.

Ry¹ each independently represents a hydrogen atom or a substituent. Examples of the substituent include those listed in the following <Group α>, to which, however, the substituent is not limited.

### <Group α>

A substituted or unsubstituted alkyl group having 1 to 50 (more preferably 1 to 18, even more preferably 1 to 8) carbon atoms, a substituted or unsubstituted cycloalkyl group having 3 to 50 (more preferably 3 to 10, even more preferably 3 to 8) ring carbon atoms, a substituted or unsubstituted aryl group having 6 to 60 (more preferably 6 to 25, even more preferably 6 to 18) ring carbon atoms, a substituted or unsubstituted aralkyl group having 7 to 61 (more preferably 7 to 25, even more preferably 7 to 18) carbon atoms, an amino group, a mono-substituted or di-substituted amino group having a substituent selected from a substituted or unsubstituted alkyl group having 1 to 50 (more preferably 1 to 18, even more preferably 1 to 8) carbon atoms and a substituted or unsubstituted aryl group having 6 to 60 (more preferably 6 to 25, even more preferably 6 to 18) ring carbon atoms, a substituted or unsubstituted alkoxy group having 1 to 50 (more preferably 1 to 18, even more preferably 1 to 8) carbon atoms, a substituted or unsubstituted cycloalkoxy group having 3 to 50 (more preferably 3 to 10, even more preferably 3 to 8) ring carbon atoms, a substituted or unsubstituted aryloxy group having 6 to 60 (more preferably 6 to 25, even more preferably 6 to 18) ring carbon atoms, a substituted or unsubstituted alkylthio group having 1 to 50 (more preferably 1 to 18, even more preferably 1 to 8) carbon atoms, a substituted or unsubstituted arylthio group having 6 to 60 (more preferably 6 to 25, even more preferably 6 to 18) ring carbon atoms, a mono-substituted, di-substituted or tri-substituted silyl group having a substituent selected from a substituted or unsubstituted alkyl group having 1 to 50 (more preferably 1 to 18, even more preferably 1 to 8) carbon atoms and a substituted or unsubstituted aryl group having 6 to 60 (more preferably 6 to 25, even more preferably 6 to 18) ring carbon atoms, a substituted or unsubstituted heteroaryl group having 5 to 60 (more preferably 5 to 30, even more preferably 5 to 26) ring atoms, a substituted or unsubstituted haloalkyl group having 1 to 50 (more preferably 1 to 18, even more preferably 1 to 8) carbon atoms, a halogen atom, a cyano group, a nitro group, a sulfonyl group having a substituent selected from a substituted or unsubstituted alkyl group having 1 to 50 (more preferably 1 to 18, even more preferably 1 to 8) carbon atoms and a substituted or unsubstituted aryl group having 6 to 60 (more preferably 6 to 25, even more preferably 6 to 18) ring carbon atoms, a di-substituted phosphoryl group having a substituent selected from a substituted or unsubstituted alkyl group having 1 to 50 (more preferably 1 to 18, even more preferably 1 to 8) carbon atoms and a substituted or unsubstituted aryl group having 6 to 60 (more preferably 6 to 25, even more preferably 6 to 18) ring carbon atoms, an alkylsulfonyloxy group, an arylsulfonyloxy group, an alkylcarbonyloxy group, an arylcarbonyloxy group, a boron-containing group, a zinc-containing group, a tin-containing group, a silicon-containing group, a magnesium-containing group, a lithium-containing group, a hydroxy group, an alkyl-substituted or aryl-substituted carbonyl group, a carboxyl group, a vinyl group, a (meth)acryloyl group, an epoxy group, and an oxetanyl group.

The substituents listed in the group α may be further substituted with a substituent in the group α. A plurality of these substituents in the group α may bond to each other to form a ring.

Examples of the alkyl group in the group α include a methyl group, an ethyl group, an n-propyl group, an isopropyl group, an n-butyl group, an isobutyl group, an s-butyl group, a t-butyl group, a pentyl group (including isomer groups), a hexyl group (including isomer groups), a heptyl group (including isomer groups), an octyl group (including isomer groups), a nonyl group (including isomer groups), a decyl group (including isomer groups), an undecyl group (including isomer groups), a dodecyl group (including isomer groups), a tridecyl group, a tetradecyl group, an octadecyl group, a tetracosanyl group, a tetracontanyl group, etc. These groups may be substituted.

The cycloalkyl group in the group α includes a cyclopropyl group, a cyclobutyl group, a cyclopentyl group, a cyclohexyl group, a cycloheptyl group, a cyclooctyl group, an adamantyl group, etc. These groups may be substituted.

Examples of the aryl group in the group α include a phenyl group, a naphthyl group, a naphthylphenyl group, a biphenylyl group, a terphenylyl group, a quaterphenyl group, a quinquephenylyl group, an acenaphthylenyl group, an anthryl group, a benzanthryl group, an aceanthryl group, a phenanthryl group, a benzophenanthryl group, a phenalenyl group, a fluorenyl group, a 9,9′-spirobifluorenyl group, a benzofluorenyl group, a dibenzofluorenyl group, a picenyl group, a pentaphenyl group, a pentacenyl group, a pyrenyl group, a chrysenyl group, a benzochrysenyl group, an s-indanyl group, an as-indanyl group, a fluoranthenyl group, a benzofluoranthenyl group, a tetracenyl group, a triphenylenyl group, a benzotriphenylenyl group, a perylenyl group, a coronyl group, a dibenzoanthryl group, a 9,9-dimethylfluorenyl group, a 9,9-diphenylfluorenyl group, etc. These groups may be substituted.

The heteroaryl group in the group α contains at least one, preferably 1 to 5 (more preferably 1 to 3, even more preferably 1 or 2) hetero atoms. Examples of the hetero atom include a nitrogen atom, a sulfur atom, an oxygen atom and a phosphorus atom. Examples of the heteroaryl group in the group α include a pyrrolyl group, a furyl group, a thienyl group, a pyridyl group, a pyridazinyl group, a pyrimidinyl group, a pyrazinyl group, a triazinyl group, an imidazolyl group, an oxazolyl group, a thiazolyl group, a pyrazolyl group, an isoxazolyl group, an isothiazolyl group, an oxadiazolyl group, a thiadiazolyl group, a triazolyl group, a tetrazolyl group, an indolyl group, an isoindolyl group, a benzofuranyl group, an isobenzofuranyl group, a benzothiophenyl group, an isobenzothiophenyl group, an indolidinyl group, a quinolidinyl group, a quinolyl group, an isoquinolyl group, a cinnolyl group, a phthalazinyl group, a quinazolinyl group, a quinoxalinyl group, a benzimidazolyl group, a benzoxazolyl group, a benzothiazolyl group, an indazolyl group, a benzisoxazolyl group, a benzisothiazolyl group, a dibenzofuranyl group, a dibenzothiophenyl group, a carbazolyl group, a bicarbazolyl group, a phenanthridinyl group, an acridinyl group, a phenanthrolinyl group, a phenazinyl group, a phenothiazinyl group, a phenoxazinyl group. an azatriphenylenyl group, a diazatriphenylenyl group, a xanthenyl group, an azacarbazolyl group, an azadibenzofuranyl group, an azadibenzothiophenyl group, a benzofuranobenzothiophenyl group, a benzothienobenzothiophenyl group, a dibenzofuranonaphthyl group, a dibenzothienonaphthyl group, a dinaphthothienothiophenyl group, a dinaphtho-<2′,3′:2,3:2′,3′:6,7>-carbazoyl group, etc. These groups may be substituted.

In this description, the substituted or unsubstituted carbazolyl group includes, in addition to the following carbazolyl groups:

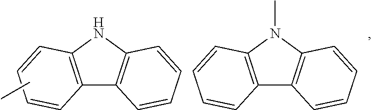

and a substituted carbazolyl group having any arbitrary substituent, for example, the following substituted carbazolyl groups.

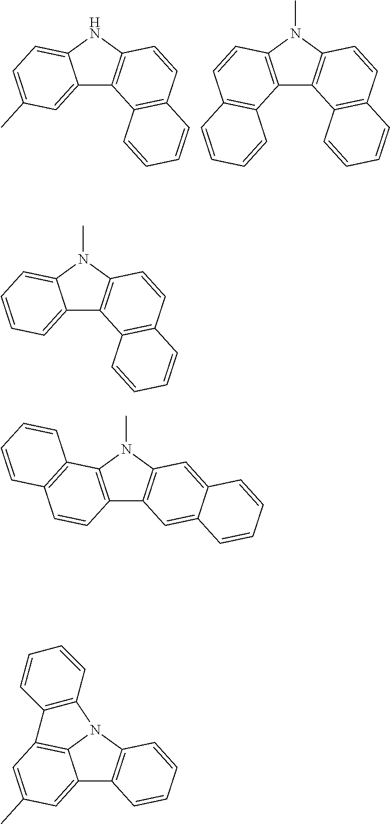

In this description, the substituted or unsubstituted dibenzofuranyl group and the substituted or unsubstituted dibenzothiophenyl group include, in addition to the following dibenzofuranyl group and dibenzothiophenyl group,

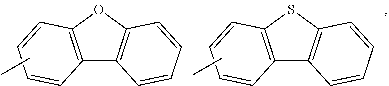

and a substituted dibenzofuranyl group and a substituted dibenzothiophenyl group having any arbitrary substituent, for example, the following substituted dibenzofuranyl groups and substituted dibenzothiophenyl groups:

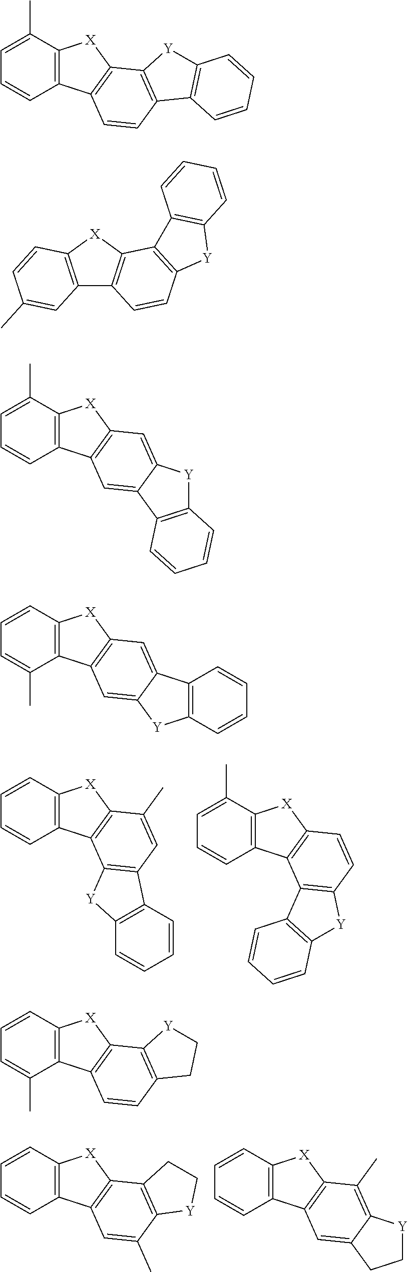

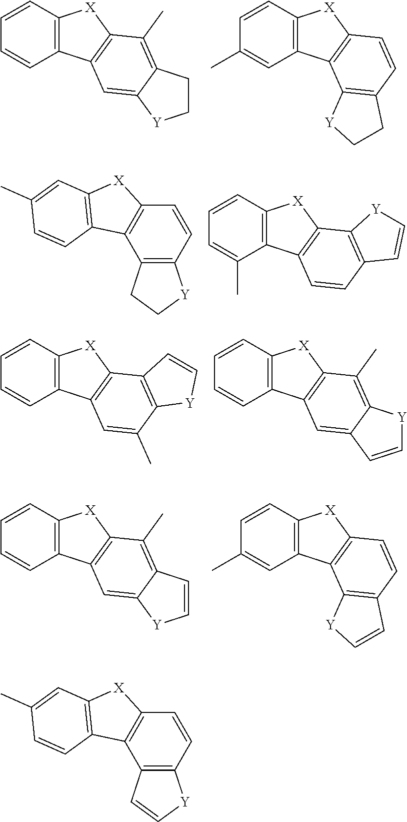

wherein X represents an oxygen atom or a sulfur atom, Y represents an oxygen atom, a sulfur atom, NH, NR^{a} (R^{a} represents an alkyl group or an aryl group), CH₂ or CR^{b}₂ (R^{b} represents an alkyl group or an aryl group).

The aralkyl group in the group α includes an aralkyl group having the above-mentioned aryl group with 6 to 60 ring carbon atoms, more concretely a benzyl group, a phenethyl group, a phenylpropyl group, etc. These may be substituted.

The mono-substituted or di-substituted amino group in the group α includes a mono-substituted or di-substituted amino group having a substituent selected from a group consisting of the above-mentioned alkyl group and the above-mentioned aryl group. These may be further substituted.

The alkoxy group in the group α includes an alkoxy group having the above-mentioned alkyl group with 1 to 50 carbon atoms, more concretely a methoxy group, an ethoxy group, a methoxy group, an i-propoxy group, an n-propoxy group, an n-butoxy group, an s-butoxy group, a t-butoxy group, etc., and these may be further substituted.

The cycloalkoxy group in this embodiment includes a cycloalkoxy group having the above-mentioned cycloalkyl group with 3 to 50 carbon atoms. These may be further substituted.

The aryloxy group in the group α includes an aryloxy group having the above-mentioned aryl group with 6 to 60 ring carbon atoms, more concretely a phenoxy group, etc. These may be further substituted.

The alkylthio group in the group α includes an alkylthio group having the above-mentioned alkyl group with 1 to 50 carbon atoms. These may be further substituted.

The arylthio group in the group α includes an arylthio group having the above-mentioned aryl group with 6 to 60 ring carbon atoms. These may be further substituted.

The mono-substituted, di-substituted or tri-substituted silyl group in the group α includes a mono-substituted, di-substituted or tri-substituted silyl group having a substituent selected from a group consisting of the above-mentioned alkyl group with 1 to 50 carbon atoms and the above-mentioned aryl group with 6 to 60 ring carbon atoms, more concretely a trimethylsilyl group, a triethylsilyl group, a t-butyldimethylsilyl group, a vinyldimethylsilyl group, a propyldimethylsilyl group, an isopropyldimethylsilyl group, a triphenylsilyl group, a phenyldimethylsilyl group, a t-butyldiphenylsilyl group, a tritolylsilyl group, etc. These may be further substituted.

The haloalkyl group in the group α includes the above-mentioned alkyl group with 1 to 50 carbon atoms in which one or more hydrogen atoms are substituted with a halogen atom (fluorine atom, chlorine atom, bromine atom, iodine atom), concretely a trifluoromethyl group, a pentafluoroethyl group, a heptafluoropropyl group, etc. These may be further substituted.

The sulfonyl group in the group α includes a sulfonyl group having a substituent selected from a group consisting of the above-mentioned alkyl group with 1 to 50 carbon atoms and the above-mentioned aryl group with 6 to 60 ring carbon atoms. These may be further substituted.

The di-substituted phosphoryl group in the group α includes a di-substituted phosphoryl group having a substituent selected from a group consisting of the above-mentioned alkyl group with 1 to 50 carbon atoms and the above-mentioned aryl group with 6 to 60 ring carbon atoms. These may be further substituted.

The alkylsulfonyloxy group, the arylsulfonyloxy group, the alkylcarbonyloxy group, the arylcarbonyloxy group, and the alkyl-substituted or aryl-substituted carbonyl group in the group α include those having a substituent selected from the above-mentioned alkyl group and the above-mentioned aryl group.

Ry¹ is preferably a substituted or unsubstituted aryl group having 6 to 60 ring carbon atoms, more preferably a phenyl group.

Rx¹ in the formulae (Cz-1), (Cz-11), (Cz-12) and (Cz-13) is described below.

Rx¹ is each independently a hydrogen atom or a substituent. Examples of the substituent include those listed in the above-mentioned <Group α>, but are not limited thereto.

Rx¹ is preferably a hydrogen atom, or a substituted or unsubstituted aryl group having 6 to 60 ring carbon atoms, more preferably a hydrogen atom.

Rx¹'s may form a ring. The ring includes, though not specifically limited thereto, a substituted or unsubstituted aromatic hydrocarbon ring having 6 to 30 ring carbon atoms and a substituted or unsubstituted aromatic hetero ring having 5 to 30 ring atoms, and preferably a benzene ring, a naphthalene ring, a pyridine ring, a quinoline ring and an isoquinoline ring.

Cz² in the formula (1) is, as described above, a group represented by the formula (Cz-2). Preferred embodiments of the formula (Cz-2) are described below.

Preferably, X⁴¹ to X⁴⁴ each are independently C-*⁴.

Preferably, one of X⁴⁶ and X⁴⁷ is a carbon atom bonding to *⁴¹, and more preferably X⁴⁶ is a carbon atom bonding to *¹¹. Preferably, three of X⁴⁵ to X⁴⁸ not bonding to *⁴¹ each are independently C-*⁴.

Preferably, one of X⁵² and X⁵³ is a carbon atom bonding to *⁵¹, more preferably X⁵³ is a carbon atom bonding to *⁵¹. Preferably, three of X⁵¹ to X⁵⁴ not bonding to *⁵¹ each are independently C-*⁴.

Preferably, X⁵⁵ to X⁵⁸ each are independently represent C-*⁴.

Preferably, one of *³'s bonds to L² in the formula (1), the others each bond to Rye, and every *⁴ bonds to Rx².

Cz² in the formula (1) is preferably a group represented by the formula (Cz-2a):

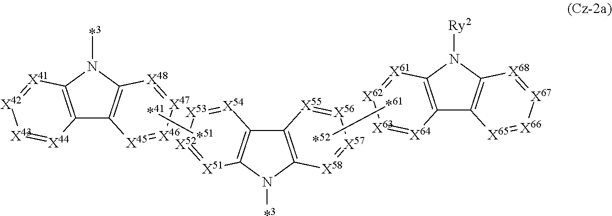

wherein X⁴¹ to X⁴⁴ each independently represent N or C-*⁴,
one of X⁴⁵ to X⁴⁸ is a carbon atom bonding to *⁴¹, and the other three each are independently N or C-*⁴,
one of X⁵¹ to X⁵⁴ is a carbon atom bonding to *⁵¹, and the other three each are independently N or C-*⁴,
one of X⁵⁵ to X⁵⁸ is a carbon atom bonding to *⁵², and the other three each are independently N or C-*⁴,
one of X⁶¹ to X⁶⁴ is a carbon atom bonding to *⁶¹, and the other three each are independently N or CRx²,
X⁶⁵ to X⁶⁸ each independently represent N or CRx²,
one of *³'s or one of *⁴'s bonds to L² in the formula (1),
*³ not bonding to L² bonds to Ry²,
*⁴ not bonding to L² bonds to Rx²,
Ry² each independently represents a hydrogen atom or a substituent,
Rx² each independently represents a hydrogen atom or a substituent, and
Rx²'s may form a ring.

Preferably, X⁴¹ to X⁴⁴ each are independently C-*⁴.

Preferably one of X⁴⁶ and X⁴⁷ is a carbon atom bonding to *⁴¹, more preferably X⁴⁶ is a carbon atom bonding to *⁴¹. Preferably, three of X⁴⁵ to X⁴⁸ not bonding to *⁴¹ each are independently C-*⁴.

Preferably, one of X⁵² and X⁵³ is a carbon atom bonding to *⁵¹, more preferably X⁵³ is a carbon atom bonding to *⁵¹. Preferably, three of X⁵¹ to X⁵⁴ not bonding to *⁵¹ each are independently C-*⁴.

Preferably, one of X⁵⁶ and X⁵⁷ is a carbon atom bonding to *⁵², more preferably X⁵⁶ is a carbon atom bonding to *⁵². Preferably, three of X⁵⁵ to X⁵⁸ not bonding to *⁵² each are independently C-*⁴.

Preferably one of X⁶² and X⁶³ is a carbon atom bonding to *⁶¹, more preferably X⁶³ is a carbon atom bonding to *⁶¹. Preferably, three of X⁶¹ to X⁶⁴ not bonding to *⁶¹ each are independently CRx².

Preferably, X⁶⁵ to X⁶⁸ each are independently CRx².

Preferably, one of *³'s bonds to L² in the formula (1) and the other bonds to Ry², and every *⁴ bonds to Rx².

Cz² in the formula (1) is preferably a group represented by the formula (CZ-21a):

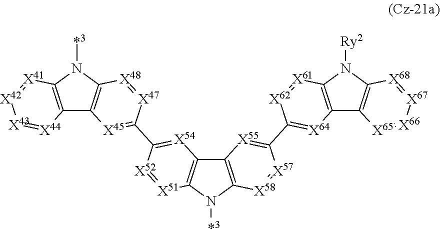

wherein X⁴¹ to X⁴⁵, X⁴⁷ to X⁴⁸, X⁵¹ to X⁵², X⁵⁴ to X⁵⁵ and X⁵⁷ to X⁵⁸ each independently represent N or C-*⁴,
X⁶¹ to X⁶² and X⁶⁴ to X⁶⁸ each independently represent N or CRx²,
one of *³'s or one of *⁴'s bonds to L² in the formula (1),
*³ not bonding to L² bonds to Ry²,
*⁴ not bonding to L² bonds to Rx²,
Ry² each independently represents a hydrogen atom or a substituent,
Rx² each independently represents a hydrogen atom or a substituent, and
Rx²'s may form a ring.

Preferably, X⁴¹ to X⁴⁵, X⁴⁷ to X⁴⁸, X⁵¹ to X⁵², X⁵⁴ to X⁵⁵ and X⁵⁷ to X⁵⁸ each are independently C-*⁴.

Preferably, X⁶¹ to X⁶² and X⁶⁴ to X⁶⁸ each are independently CRx².

Preferably one of *³'s bonds to L² in the formula (1), the others each bond to Ry², and every *⁴ bonds to Rx².

Cz² in the formula (1) is preferably a group represented by the formula (Cz-22a):

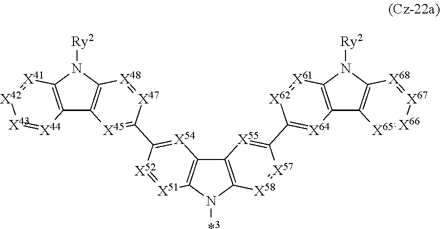

wherein X⁴¹ to X⁴⁵, X⁴⁷ to X⁴⁸, X⁵¹ to X⁵², X⁵⁴ to X⁵⁵, X⁵⁷ to X⁵⁸, X⁶¹ to X⁶² and X⁶⁴ to X⁶⁸ each independently represent N or CRx²,
*³ bonds to L² in the formula (1),
Ry² each independently represents a hydrogen atom or a substituent,
Rx² each independently represents a hydrogen atom or a substituent, and
Rx²'s may form a ring.

Preferably, X⁴¹ to X⁴⁵, X⁴⁷ to X⁴⁸, X⁵¹ to X⁵², X⁵⁴ to X⁵⁵, X⁵⁷ to X⁵⁸, X⁶¹ to X⁶² and X⁶⁴ to X⁶⁸ each are independently CRx².

Cz² in the formula (1) is preferably a group represented by the formula (Cz-23a):

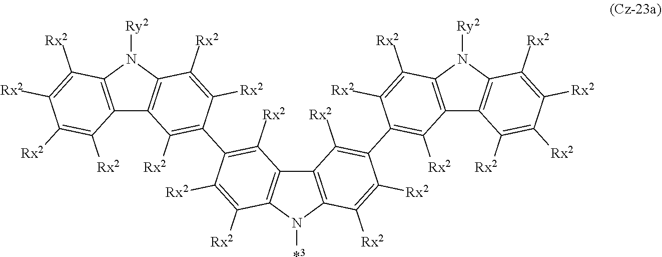

wherein *³ bonds to L² in the formula (1),
Ry² each independently represents a hydrogen atom or a substituent,
Rx² each independently represents a hydrogen atom or a substituent, and
Rx²'s may form a ring.

Cz² in the formula (1) is preferably a group represented by the formula (Cz-24a):

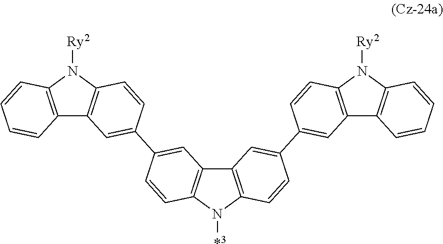

wherein *³ bonds to L² in the formula (1), and
Ry² each independently represents a hydrogen atom or a substituent.

Cz² in the formula (1) is preferably a group represented by the formula (Cz-21 b):

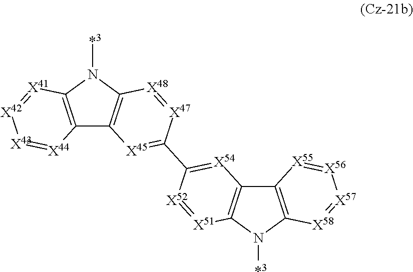

wherein X⁴¹ to X⁴⁵, X⁴⁷ to X⁴⁸, X⁵¹ to X⁵² and X⁵⁴ to X⁵⁸ each independently represent N or C-*⁴,
one of *³'s or one of *⁴'s bonds to L² in the formula (1),
*³ not bonding to L² bonds to Ry²,
*⁴ not bonding to L² bonds to Rx²,
Ry² each independently represents a hydrogen atom or a substituent,
Rx² each independently represents a hydrogen atom or a substituent, and
Rx²'s may form a ring.

Preferably, X⁴¹ to X⁴⁵, X⁴⁷ to X⁴⁸, X⁵¹ to X⁵² and X⁵⁴ to X⁵⁸ each are independently C-*⁴.

Preferably one of *³'s bonds to L² in the formula (1), the others each bond to Ry², and every *⁴ bonds to Rx².

Cz² in the formula (1) is preferably a group represented by the formula (Cz-22b):

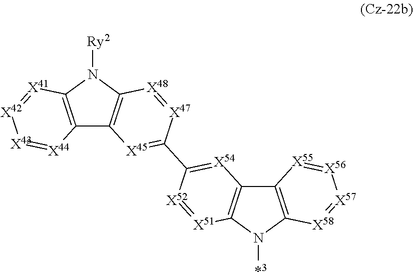

wherein X⁴¹ to X⁴⁵, X⁴⁷ to X⁴⁸, X⁵¹ to X⁵² and X⁵⁴ to X⁵⁸ each independently represent N or CRx²,
*³ bonds to L² in the formula (1),
Ry² represents a hydrogen atom or a substituent,
Rx² each independently represents a hydrogen atom or a substituent, and
Rx²'s may form a ring.

Preferably, X⁴¹ to X⁴⁵, X⁴⁷ to X⁴⁸, X⁵¹ to X⁵² and X⁵⁴ to X⁵⁸ each are independently CRx².

Cz² in the formula (1) is preferably a group represented by the formula (Cz-23 b):

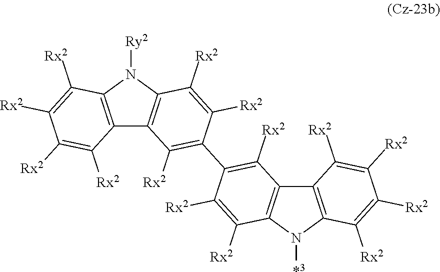

wherein *³ bonds to L² in the formula (1),
Ry² represents a hydrogen atom or a substituent,
Rx² each independently represents a hydrogen atom or a substituent, and
Rx²'s may form a ring.

Cz² in the formula (1) is preferably a group represented by the formula (Cz-24b):

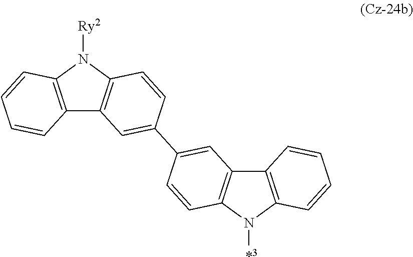

wherein *³ bonds to L² in the formula (1), and Ry² represents a hydrogen atom or a substituent.

Ry² in the formulae (Cz-2), (Cz-2a), (Cz-21a), (Cz-22a), (Cz-23a), (Cz-24a), (Cz-21 b), (Cz-22 b), (Cz-23b) and (Cz-24b) is described below.

Ry² each independently represents a hydrogen atom or a substituent. Examples of the substituent include, though not limited thereto, those listed in the above-mentioned <Group α>.

Ry² is preferably a substituted or unsubstituted aryl group having 6 to 60 ring carbon atoms, more preferably a phenyl group.

Rx² in the formulae (Cz-2), (Cz-2a), (Cz-21a), (Cz-22a), (Cz-23a), (Cz-21 b), (Cz-22b) and (Cz-23b) is described below.

Rx² each independently represents a hydrogen atom or a substituent. Examples of the substituent include, though not limited thereto, those listed in the above-mentioned <Group α>.

Rx² is preferably a hydrogen atom or a substituted or unsubstituted aryl group having 6 to 60 ring carbon atoms, more preferably a hydrogen atom.

Rx²'s may form a ring. The ring includes, though not limited thereto, a substituted or unsubstituted aromatic hydrocarbon ring having 6 to 30 ring carbon atoms and a substituted or unsubstituted aromatic hetero ring having 5 to 30 ring atoms, preferably a benzene ring, a naphthalene ring, a pyridine ring, a quinoline ring and an isoquinoline ring.

A in the formula (1) is, as described above, a residue of a substituted or unsubstituted nitrogen-containing aromatic hetero ring having 6 to 30 ring atoms. Here, the “residue of a nitrogen-containing aromatic hetero ring” means a divalent ring derived from a nitrogen-containing aromatic hetero ring by removing two hydrogen atoms from the ring. For example, in the case where the nitrogen-containing aromatic hetero ring is a pyridine ring, the residue of the nitrogen-containing aromatic hetero ring is a pyridylene group (or also referred to as a pyridine-diyl group). In the case where the nitrogen-containing aromatic hetero ring is substituted, the two hydrogen atoms to be removed from the ring are selected from the hydrogen atoms bonding to the atoms that constitute the ring itself of the nitrogen-containing aromatic hetero ring, and are not selected from the hydrogen atoms bonding to the substituent that substitutes on the nitrogen-containing aromatic hetero ring. For example, a group represented by the following (i) may be said to be a residue of a pyridine ring substituted with an alkyl group, but a group represented by the following (ii) cannot be said to be a residue of a pyridine ring substituted with an alkyl group.

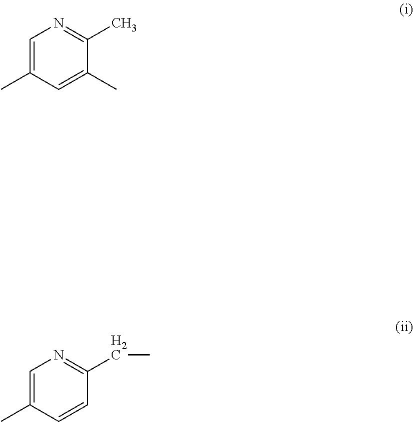

A is preferably a residue of a nitrogen-containing aromatic hetero ring selected from a group consisting of a substituted or unsubstituted pyridine ring, a substituted or unsubstituted pyrazine ring, a substituted or unsubstituted pyrimidine ring, a substituted or unsubstituted pyridazine ring, a substituted or unsubstituted triazine ring, a substituted or unsubstituted quinoline ring, a substituted or unsubstituted isoquinoline ring, a substituted or unsubstituted quinoxaline ring, a substituted or unsubstituted quinazoline ring, a substituted or unsubstituted cinnoline ring, a substituted or unsubstituted benzoquinazoline ring, and a substituted or unsubstituted azafluoranthene ring, more preferably a residue of a nitrogen-containing aromatic hetero ring selected from a group consisting of a substituted or unsubstituted pyrimidine ring, a substituted or unsubstituted triazine ring, a substituted or unsubstituted quinazoline ring, a substituted or unsubstituted benzoquinazoline ring, and a substituted or unsubstituted azafluoranthene ring, even more preferably a residue of a nitrogen-containing aromatic hetero ring selected from a group consisting of a substituted or unsubstituted pyrimidine ring, and a substituted or unsubstituted quinazoline ring.

A in the formula (1) is preferably a group represented by the formula (A-1) or (A-2):

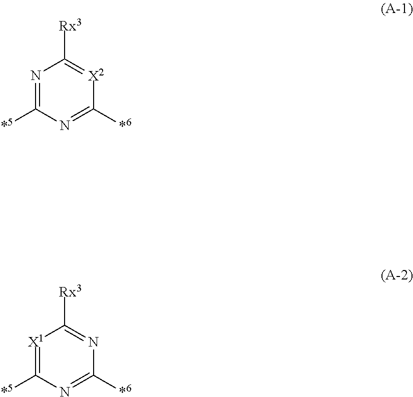

wherein X¹ and X² each independently represent N or CRx³,
Rx³ each independently represents a hydrogen atom or a substituent,
*⁵ bonds to L¹ in the formula (1),
*⁶ bonds to L² in the formula (1), and
Rx³'s may form a ring.

Preferably, X¹ and X² each are independently CRx³.

A in the formula (1) is preferably a group represented by the formula (A-3) or (A-4):

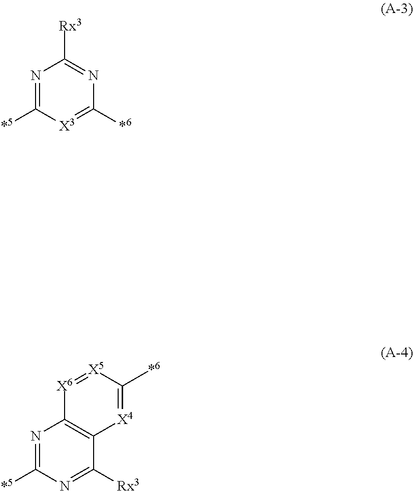

wherein X³ to X⁶ each independently represent N or CRx³,
Rx³ each independently represents a hydrogen atom or a substituent,
*⁵ bonds to L¹ in the formula (1),
*⁶ bonds to L² in the formula (1), and
Rx³'s may form a ring.

Preferably, X³ to X⁶ each are independently CRx³.

Rx³ each independently represents a hydrogen atom or a substituent. Examples of the substituent include, though not limited thereto, those listed in the above-mentioned <Group α>.

Rx³ is preferably a substituted or unsubstituted aryl group having 6 to 60 ring carbon atoms, or a substituted or unsubstituted heteroaryl group having 5 to 60 ring atoms, more preferably a substituted or unsubstituted aryl group having 6 to 60 ring carbon atoms, even more preferably a phenyl group.

Rx³'s may form a ring. The ring includes, though not limited thereto, a substituted or unsubstituted aromatic hydrocarbon ring having 6 to 30 ring carbon atoms, and a substituted or unsubstituted aromatic hetero ring having 5 to 30 ring atoms, preferably a benzene ring, a naphthalene ring, a pyridine ring, a quinoline ring and an isoquinoline ring.

L¹ and L² in the formula (1) each are, as described above, independently a substituted or unsubstituted arylene group having 6 to 60 ring carbon atoms.

Preferably, L¹ and L² each are independently a phenylene group or a naphthylene group.

n1 and n2 in the formula (1) each are, as described above, independently an integer of 0 to 4.

Preferably, n1 and n2 each are independently an integer of 0 to 2, more preferably 0 or 1.

When n1=0, this means that A and Cz¹ bond via a single bond.

When n2=0, this means that A and Cz² bond via a single bond.

When n1 is an integer of 2 to 4, L¹'s may be the same as or different from each other. L¹'s may form a ring.

When n2 is an integer of 2 to 4, L²'s may be the same as or different from each other. L²'s may form a ring.

In the formula (1), preferably, the structure represented by -(L¹)ₙ₁-Cz¹ differs from the structure represented by -(L²)ₙ₂-Cz². Having the configuration, the compound (1) can readily dissolve in solvent.

In order that the structure represented by -(L¹)ₙ₁-Cz¹ differs from the structure represented by -(L²)ₙ₂-Cz², for example, the following (a) to (e) are preferred.

(a): In the formula (1), n1≠n2.

(b): In the formula (1), any one of n1 and n2 is 0, and the other is an integer of 1 to 4.

(c): In the formula (1), n1=n2, and L¹ and L² differ.

(d): The compound (1) is represented by the formula (1-x) or (1-y):

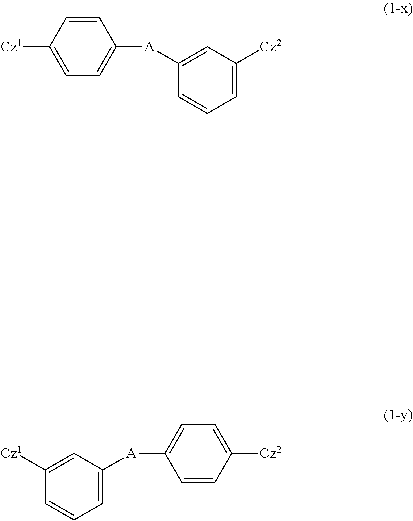

wherein Cz¹, Cz² and A are the same as Cz¹, Cz² and A in the formula (1).

(e): In the formula (1), Cz² is represented by the formula (Cz-24 b).

The compound (1) is preferably represented by the formula (1-2):

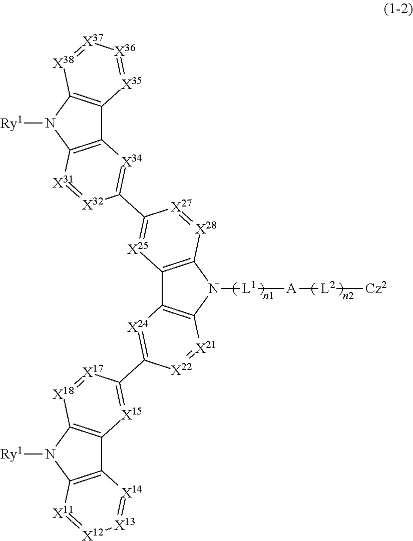

In the formula (1-2), X¹¹ to X¹⁵, X¹⁷ to X¹⁸, X²¹ to X²², X²⁴ to X²⁵, X²⁷ to X²⁸, X³¹ to X³² and X³⁴ to X³⁸ each independently represent N or CRx¹,

Ry¹ each independently represents a hydrogen atom or a substituent,
Rx¹ each independently represents a hydrogen atom or a substituent, and
Rx¹'s may form a ring.

In the formula (1-2), Cz², A, L¹, L², n1 and n2 are the same as Cz², A, L¹, L², n1 and n2 in the formula (1).

Preferably, X¹¹ to X¹⁵, X¹⁷ to X¹⁸, X²¹ to X²², X²⁴ to X²⁵, X²⁷ to X²⁸, X³¹ to X³² and X³⁴ to X³⁸ each are independently CRx¹.

In the formula (1-2), preferably, the structure represented by the formula (1-2-L) differs from the structure represented by -(L²)ₙ₂-Cz²:

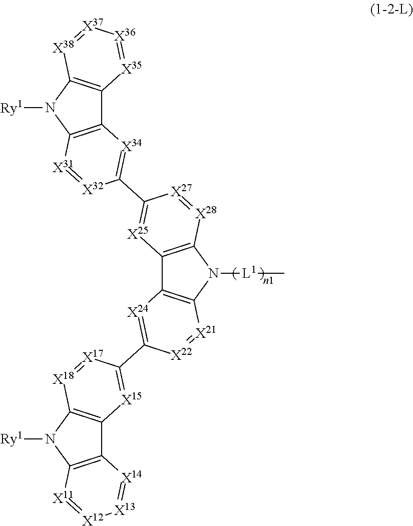

The compound (1) is preferably represented by the formula (1-2a):

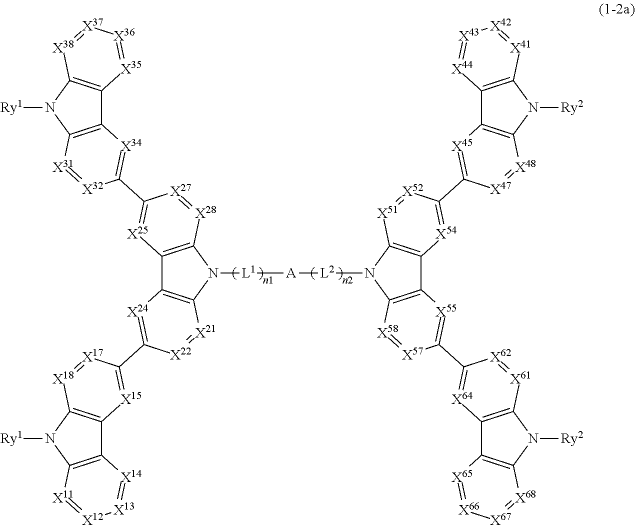

In the formula (1-2a), X¹¹ to X¹⁵, X¹⁷ to X¹⁸, X²¹ to X²², X²⁴ to X²⁵, X²⁷ to X²⁸, X³¹ to X³² and X³⁴ to X³⁸ each independently N or CRx¹,

Ry¹ each independently represents a hydrogen atom or a substituent,
Rx¹ each independently represents a hydrogen atom or a substituent,
X⁴¹ to X⁴⁵, X⁴⁷ to X⁴⁸, X⁵¹ to X⁵², X⁵⁴ to X⁵⁵, X⁵⁷ to X⁵⁸, X⁶¹ to X⁶² and X⁶⁴ to X⁶⁸ each independently represent N or CRx²,
Ry² each independently represents a hydrogen atom or a substituent,
Rx² each independently represents a hydrogen atom or a substituent,
Rx¹'s may form a ring, and
Rx²'s may form a ring.

In the formula (1-2a), A, L¹, L², n1 and n2 are the same as A, L¹, L², n1 and n2 in the formula (1).

Preferably, X¹¹ to X¹⁵, X¹⁷ to X¹⁸, X²¹ to X²², X²⁴ to X²⁵, X²⁷ to X²⁸, X³¹ to X³² and X³⁴ to X³⁸ each are independently CRx¹.

Preferably, X⁴¹ to X⁴⁵, X⁴⁷ to X⁴⁸, X⁵¹ to X⁵², X⁵⁴ to X⁵⁵, X⁵⁷ to X⁵⁸, X⁶¹ to X⁶² and X⁶⁴ to X⁶⁸ each are independently CRx².

In the formula (1-2a), preferably, the structure represented by the formula (1-2a-L) differs from the structure represented by the formula (1-2a-R).

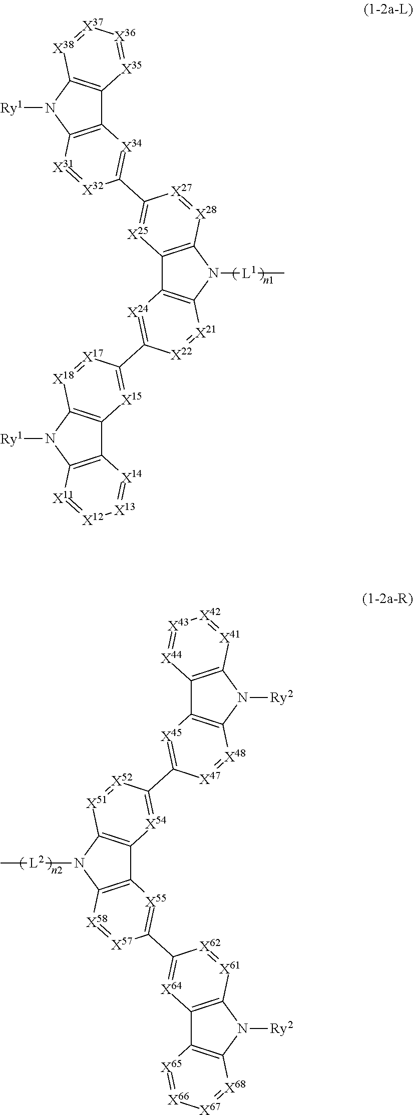

Preferably, the compound (1) is represented by the formula (1-2 b):

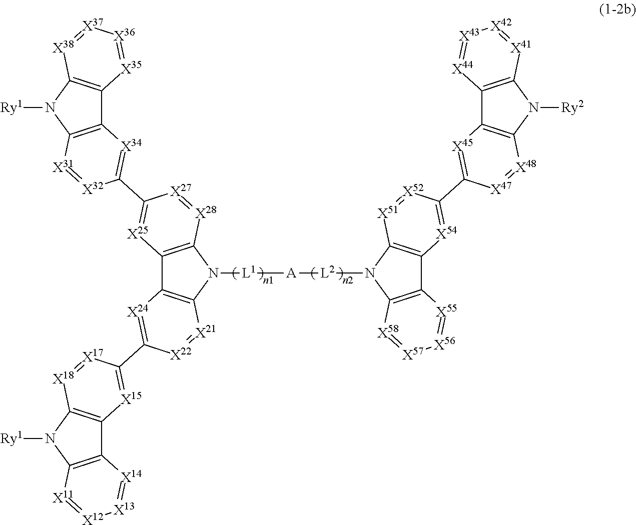

In the formula (1-2b), X¹¹ to X¹⁵, X¹⁷ to X¹⁸, X²¹ to X²², X²⁴ to X²⁵, X²⁷ to X²⁸, X³¹ to X³² and X³⁴ to X³⁸ each independently represent N or CRx¹,

Ry¹ each independently represents a hydrogen atom or a substituent,
Rx¹ each independently represents a hydrogen atom or a substituent,
X⁴¹ to X⁴⁵, X⁴⁷ to X⁴⁸, X⁵¹ to X⁵² and X⁵⁴ to X⁵⁸ each independently represent N or CRx²,
Ry² represents a hydrogen atom or a substituent,
Rx² each independently represents a hydrogen atom or a substituent,
Rx¹'s may form a ring, and
Rx²'s may form a ring.

In the formula (1-2b), A, L¹, L², n1 and n2 are the same as A, L¹, L², n1 and n2 in the formula (1).

Preferably, X¹¹ to X¹⁵, X¹⁷ to X¹⁸, X²¹ to X²², X²⁴ to X²⁵, X²⁷ to X²⁸, X³¹ to X³² and X³⁴ to X³⁸ each are independently CRx¹.

Preferably, X⁴¹ to X⁴⁵, X⁴⁷ to X⁴⁸, X⁵¹ to X⁵² and X⁵⁴ to X⁵⁸ each are independently CRx².

In the formula (1-2b), preferably, the structure represented by the formula (1-2b-L) differs from the structure represented by the formula (1-2b-R).

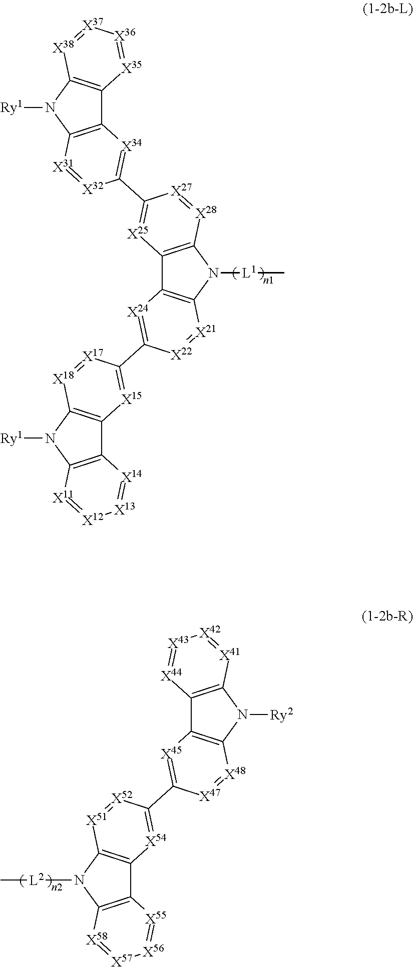

Preferably, the compound (1) is represented by the formula (1-3):

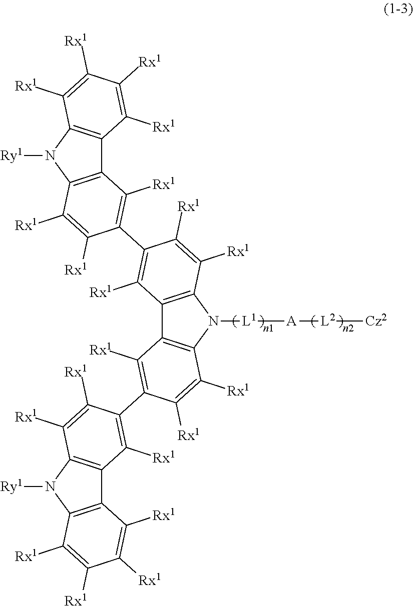

In the formula (1-3), Ry¹ each independently represents a hydrogen atom or a substituent, Rx¹ each independently represents a hydrogen atom or a substituent, and Rx¹'s may form a ring.

In the formula (1-3), Cz², A, L¹, L², n1 and n2 are the same as Cz², A, L¹, L², n1 and n2 in the formula (1).

In the formula (1-3), preferably the structure represented by the formula (1-3-L) differs from the structure represented by -(L²)ₙ₂-Cz².

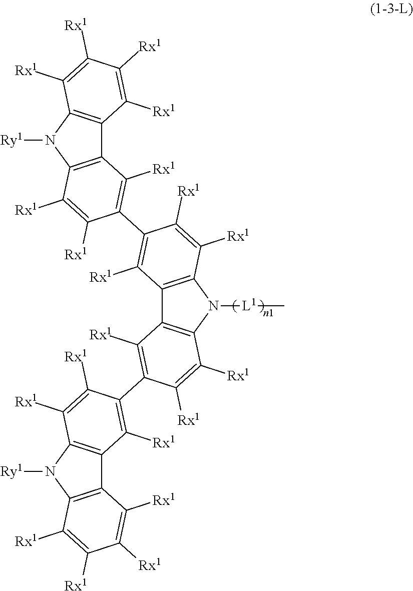

Preferably, the compound (1) is represented by the formula (1-3a):

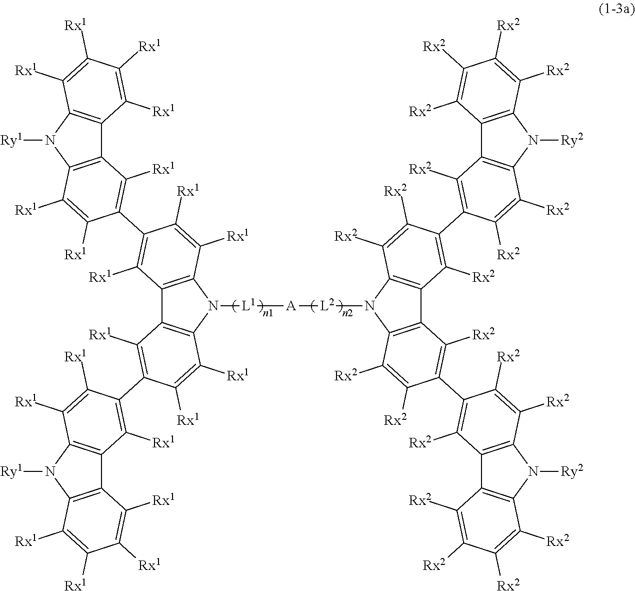

In the formula (1-3a), Ry¹ each independently represents a hydrogen atom or a substituent,

Rx¹ each independently represents a hydrogen atom or a substituent,
Ry² each independently represents a hydrogen atom or a substituent,
Rx² each independently represents a hydrogen atom or a substituent,
Rx¹'s may form a ring, and
Rx²'s may form a ring.

In the formula (1-3a), A, L¹, L², n1 and n2 are the same as A, L¹, L², n1 and n2 in the formula (1).

In the formula (1-3a), preferably, the structure represented by the formula (1-3a-L) differs from the structure represented by the formula (1-3a-R):

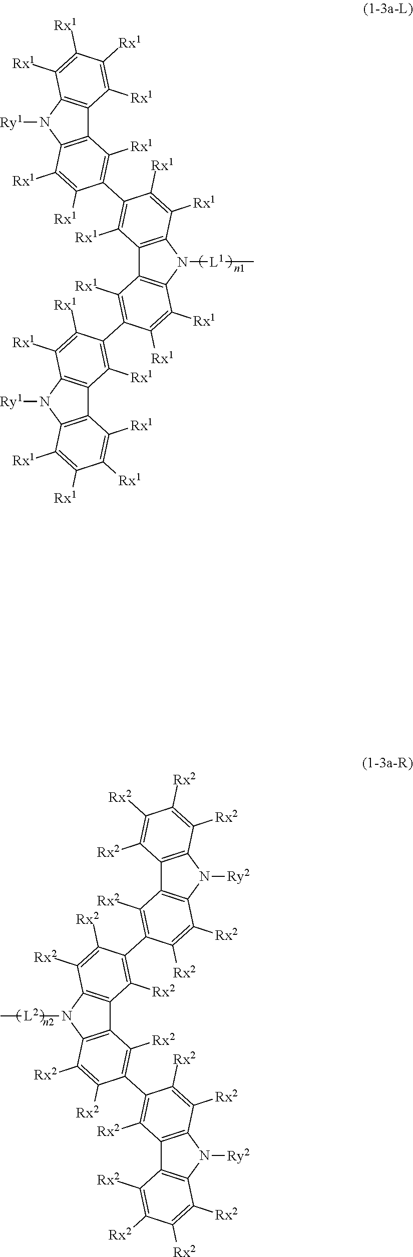

Preferably, the compound (1) is represented by the formula (1-3b).

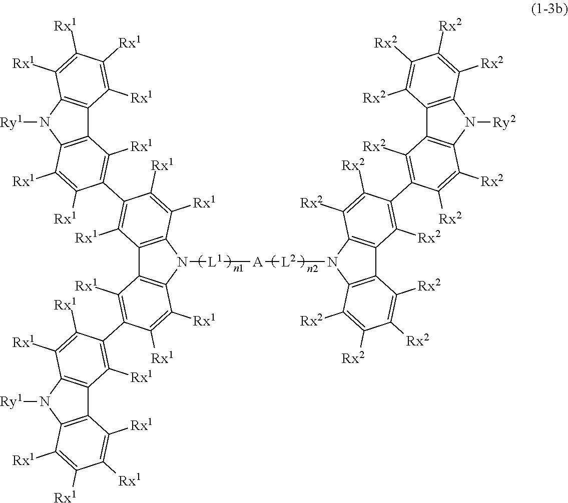

In the formula (1-3b), Ry¹ each independently represents a hydrogen atom or a substituent,

Rx¹ each independently represents a hydrogen atom or a substituent,
Ry² each independently represents a hydrogen atom or a substituent,
Rx² each independently represents a hydrogen atom or a substituent,
Rx¹'s may form a ring, and
Rx²'s may form a ring.

In the formula (1-3b), A, L¹, L², n1 and n2 are the same as A, L¹, L², n1 and n2 in the formula (1).

In the formula (1-3b), preferably, the structure represented by the formula (1-3b-L) differs from the structure represented by the formula (1-3 b-R).

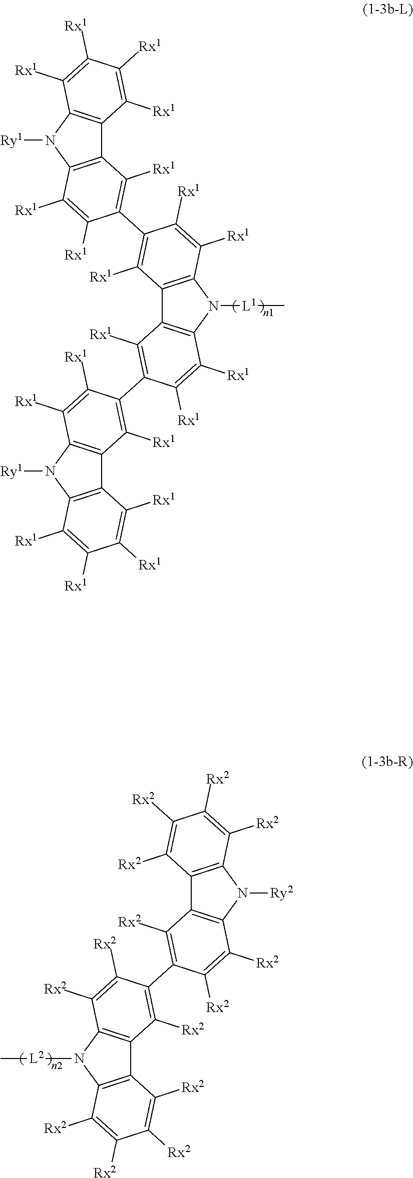

Preferably, the compound (1) is represented by the formula (1-4):

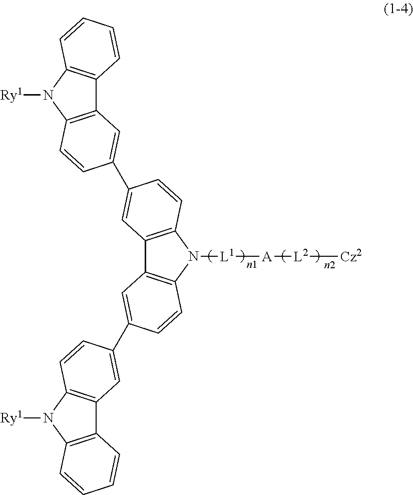

In the formula (1-4), Ry¹ each independently represents a hydrogen atom or a substituent.

In the formula (1-4), Cz², A, L¹, L², n1 and n2 are the same as Cz², A, L¹, L², n1 and n2 in the formula (1).

In the formula (1-4), preferably, the structure represented by the formula (1-4-L) differs from the structure represented by -(L²)ₙ₂-Cz².

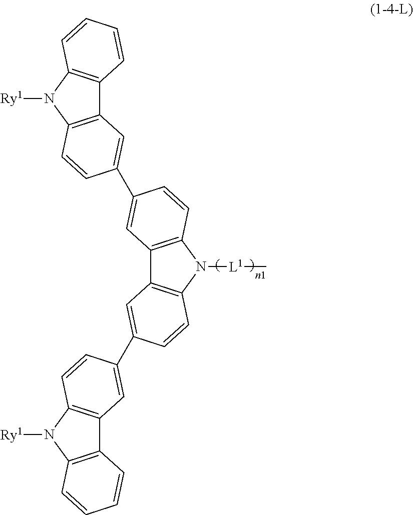

Preferably, the compound (1) is represented by the formula (1-4a).

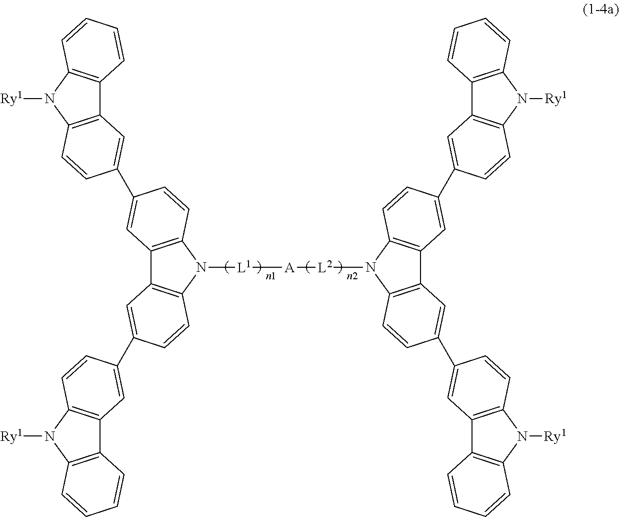

In the formula (1-4a), Ry¹ each independently represents a hydrogen atom or a substituent, Ry² each independently represents a hydrogen atom or a substituent.

In the formula (1-4a), A, L¹, L², n1 and n2 are the same as A, L¹, L², n1 and n2 in the formula (1).

In the formula (1-4a), preferably, the structure represented by the formula (1-4a-L) differs from the structure represented by the formula (1-4a-R).

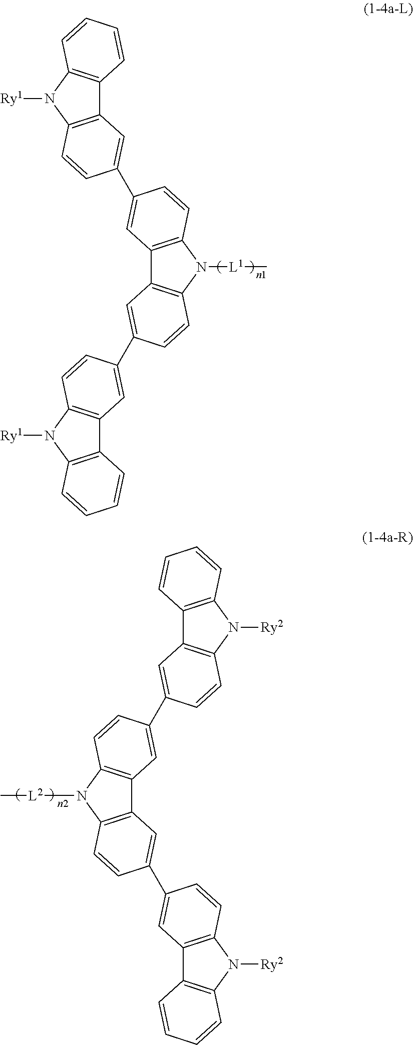

Preferably, the compound (1) is represented by the formula (1-4b):

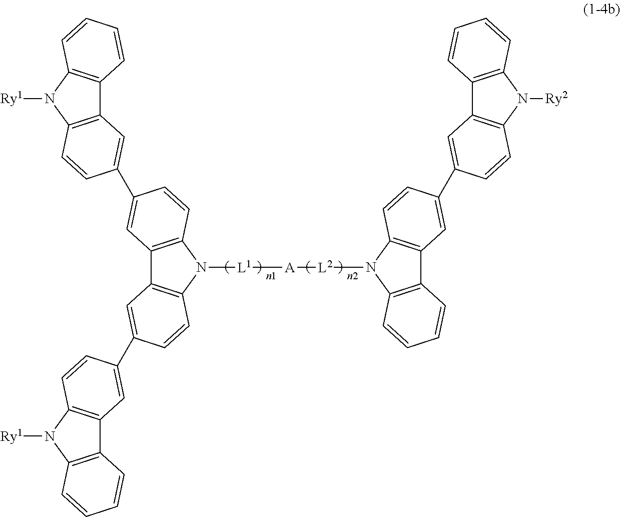

In the formula (1-4b), Ry¹ each independently represents a hydrogen atom or a substituent, Ry² represents a hydrogen atom or a substituent.

In the formula (1-4b), A, L¹, L², n1 and n2 are the same as A, L¹, L², n1 and n2 in the formula (1).

Preferred embodiments of Ry¹ in the formulae (1-2), (1-2a), (1-2b), (1-3), (1-3a), (1-3b), (1-4), (1-4a) and (1-4b) are the same as the preferred embodiments of Ry¹ in the formulae (Cz-1), (Cz-11), (Cz-12), (Cz-13) and (Cz-14).

Preferred embodiments of Rx¹ in the formulae (1-2), (1-2a), (1-2b), (1-3), (1-3a) and (1-3b) are the same as the preferred embodiments of Rx¹ in the formulae (Cz-1), (Cz-11), (Cz-12) and (Cz-13).

Preferred embodiments of Ry² in the formulae (1-2), (1-2a), (1-2b), (1-3), (1-3a), (1-3b), (1-4), (1-4a) and (1-4b) are the same as the preferred embodiments of Ry² in the formulae (Cz-2), (Cz-2a), (Cz-21a), (Cz-22a), (Cz-23a), (Cz-24a), (Cz-21 b), (Cz-22b), (Cz-23b) and (Cz-24 b).

Preferred embodiments of Rx² in the formulae (1-2), (1-2a), (1-2b), (1-3), (1-3a) and (1-3b) are the same as the preferred embodiments of Rx² in the formulae (Cz-2), (Cz-2a), (Cz-21a), (Cz-22a), (Cz-23a), (Cz-21b), (Cz-22b) and (Cz-23 b).

The optional substituent in the above-mentioned expression of “substituted or unsubstituted” is preferably selected from a group consisting of an alkyl group having 1 to 50 (preferably 1 to 18, more preferably 1 to 8) carbon atoms, a cycloalkyl group having 3 to 50 (preferably 3 to 10, more preferably 3 to 8, even more preferably 5 or 6) ring carbon atoms, an aryl group having 6 to 50 (preferably 6 to 25, more preferably 6 to 18) ring carbon atoms, an aralkyl group having 7 to 51 (preferably 7 to 30, more preferably 7 to 20) carbon atoms and having an aryl group with 6 to 50 (preferably 6 to 25, more preferably 6 to 18) ring carbon atoms, an amino group, a mono-substituted or di-substituted amino group having a substituent selected from an alkyl group having 1 to 50 (preferably 1 to 18, more preferably 1 to 8) carbon atoms and an aryl group having 6 to 50 (preferably 6 to 25, more preferably 6 to 18) ring carbon atoms, an alkoxy group having an alkyl group with 1 to 50 (preferably 1 to 18, more preferably 1 to 8) carbon atoms, an aryloxy group having an aryl group with 6 to 50 (preferably 6 to 25, more preferably 6 to 18) ring carbon atoms, a mon-substituted, di-substituted or tri-substituted silyl group having a substituent selected from an alkyl group with 1 to 50 (preferably 1 to 18, more preferably 1 to 8) carbon atoms and an aryl group with 6 to 50 (preferably 6 to 25, more preferably 6 to 18) ring carbon atoms, a heteroaryl group having 5 to 50 (preferably 5 to 24, more preferably 5 to 13) ring atoms, a haloalkyl group having 1 to 50 (preferably 1 to 18, more preferably 1 to 8) carbon atoms, a halogen atom (fluorine atom, chlorine atom, bromine atom, iodine atom), a cyano group, a nitro group, a sulfonyl group having a substituent selected from an alkyl group with 1 to 50 (preferably 1 to 18, more preferably 1 to 8) carbon atoms and an aryl group with 6 to 50 (preferably 6 to 25, more preferably 6 to 18) ring carbon atoms, a di-substituted phosphoryl group having a substituent selected from an alkyl group with 1 to 50 (preferably 1 to 18, more preferably 1 to 8) carbon atoms and an aryl group with 6 to 50 (preferably 6 to 25, more preferably 6 to 18) ring carbon atoms, an alkylsulfonyloxy group, an arylsulfonyloxy group, an alkylcarbonyloxy group, an arylcarbonyloxy group, a boron-containing group, a zinc-containing group, a tin-containing group, a silicon-containing group, a magnesium-containing group, a lithium-containing group, a hydroxy group, an alkyl-substituted or aryl-substituted carbonyl group, a carboxyl group, a vinyl group, a (meth)acryloyl group, an epoxy group, and an oxetanyl group.

These substituents may be further substituted with any of the above-mentioned optional substituents. A plurality of these substituents may bond to each other to form a ring.

“Unsubstituted” in the case of “substituted or unsubstituted” means that the group is not substituted with a substituent and a hydrogen atom bonds thereto.

Specific examples of the compound (1) are shown below. However, the compound (1) is not restricted to these specific examples.

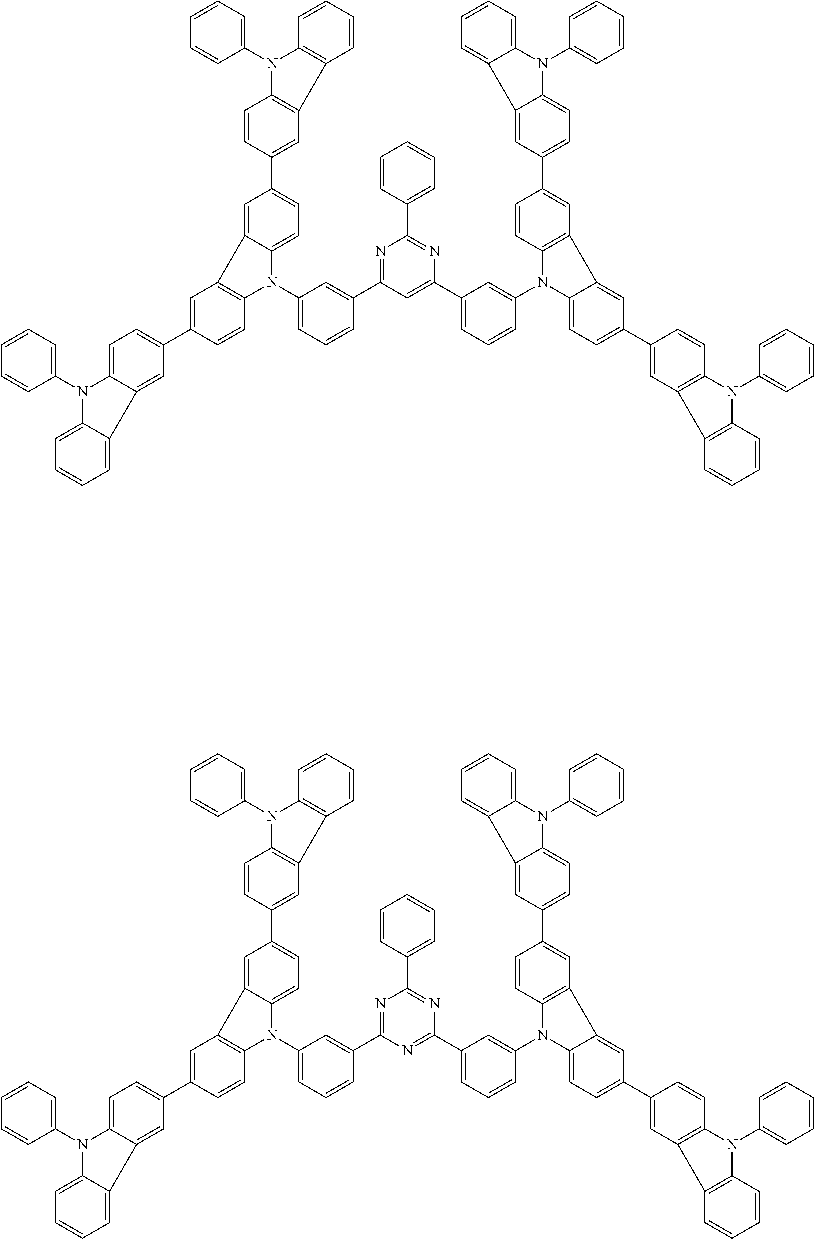 [SMILES: c1ccc(-c2nc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)cc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)n2)cc1 ; c1ccc(-c2nc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)nc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)n2)cc1]

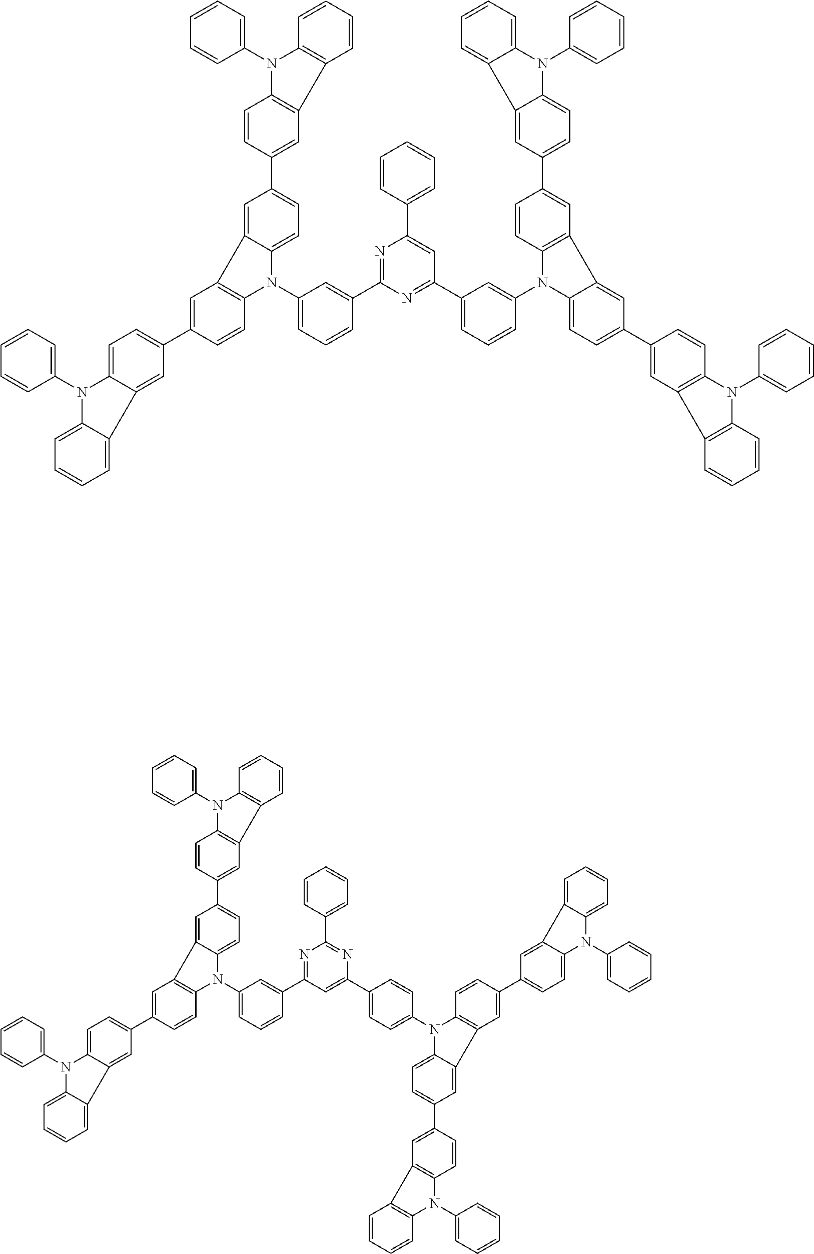 [SMILES: c1ccc(-c2cc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)nc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)n2)cc1 ; c1ccc(-c2nc(-c3ccc(-n4c5ccc(-c6ccc7c(c6)c6ccccc6n7-c6ccccc6)cc5c5cc(-c6ccc7c(c6)c6ccccc6n7-c6ccccc6)ccc54)cc3)cc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)n2)cc1]

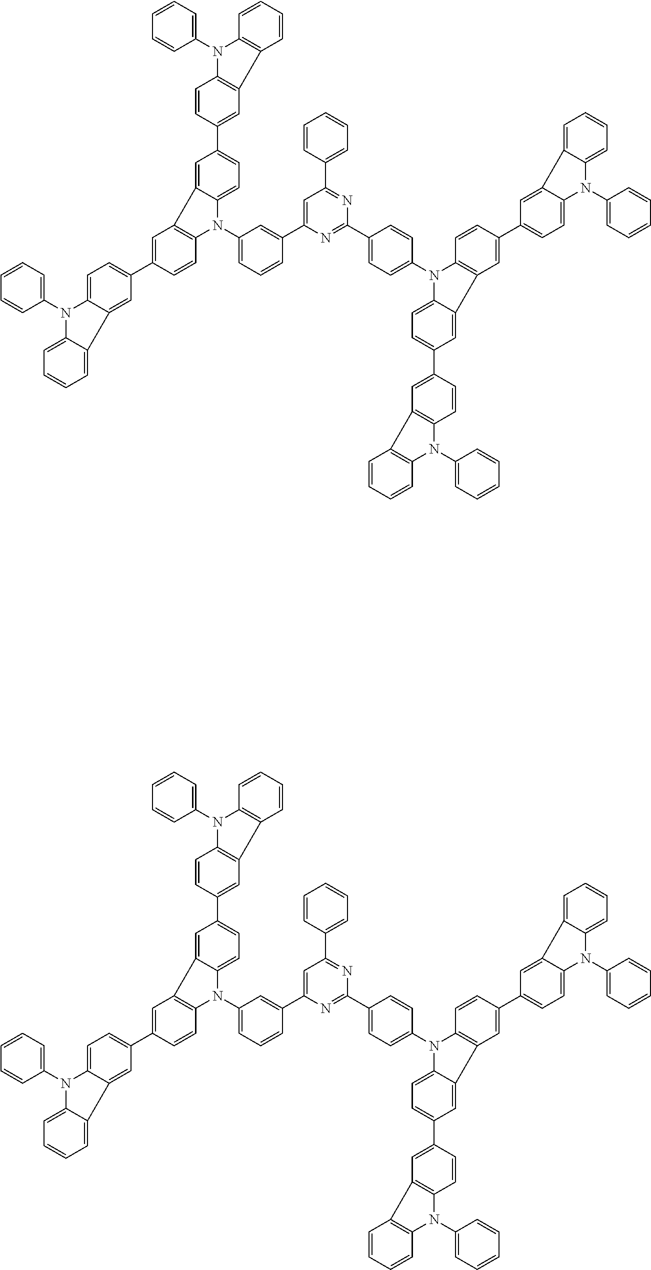 [SMILES: c1ccc(-c2cc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)nc(-c3ccc(-n4c5ccc(-c6ccc7c(c6)c6ccccc6n7-c6ccccc6)cc5c5cc(-c6ccc7c(c6)c6ccccc6n7-c6ccccc6)ccc54)cc3)n2)cc1 ; c1ccc(-c2cc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)nc(-c3ccc(-n4c5ccc(-c6ccc7c(c6)c6ccccc6n7-c6ccccc6)cc5c5cc(-c6ccc7c(c6)c6ccccc6n7-c6ccccc6)ccc54)cc3)n2)cc1]

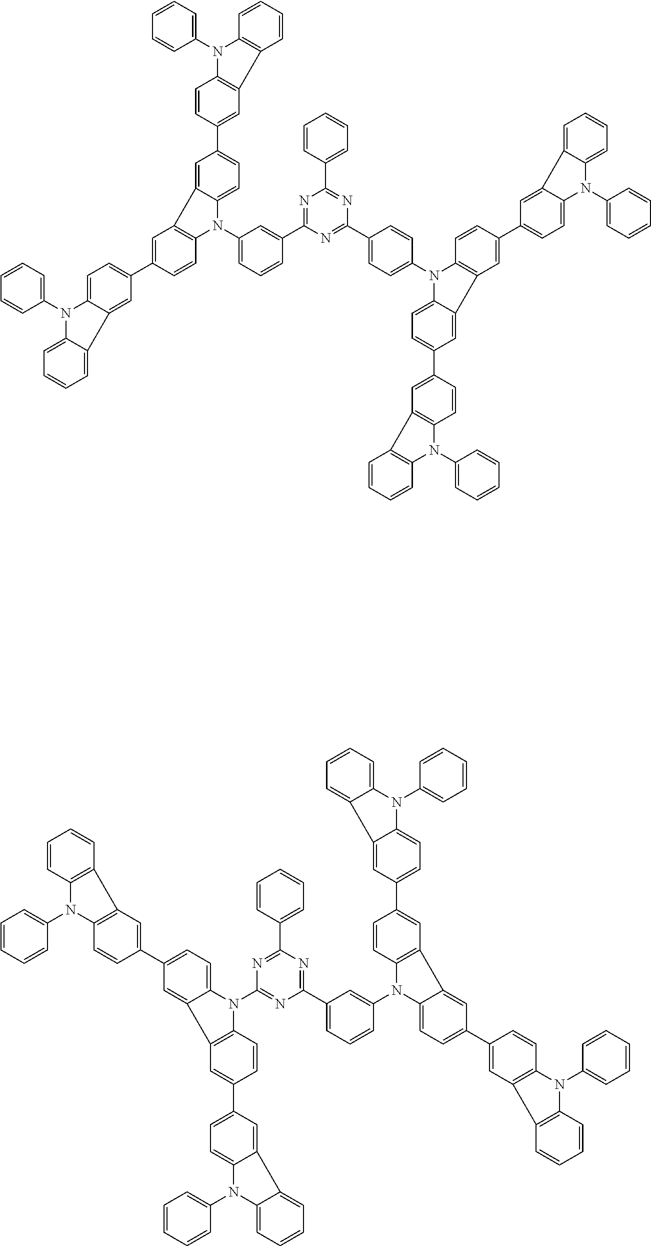 [SMILES: c1ccc(-c2nc(-c3ccc(-n4c5ccc(-c6ccc7c(c6)c6ccccc6n7-c6ccccc6)cc5c5cc(-c6ccc7c(c6)c6ccccc6n7-c6ccccc6)ccc54)cc3)nc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)n2)cc1 ; c1ccc(-c2nc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)nc(-n3c4ccc(-c5ccc6c(c5)c5ccccc5n6-c5ccccc5)cc4c4cc(-c5ccc6c(c5)c5ccccc5n6-c5ccccc5)ccc43)n2)cc1]

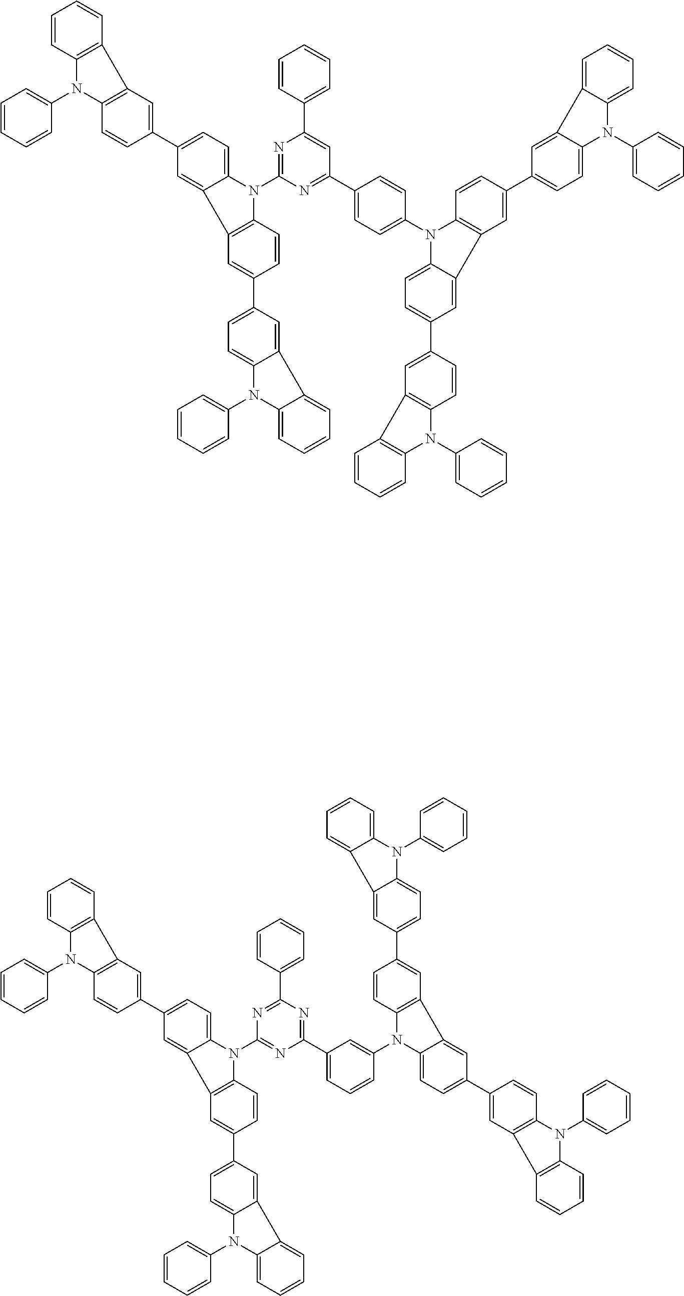 [SMILES: c1ccc(-c2cc(-c3ccc(-n4c5ccc(-c6ccc7c(c6)c6ccccc6n7-c6ccccc6)cc5c5cc(-c6ccc7c(c6)c6ccccc6n7-c6ccccc6)ccc54)cc3)nc(-n3c4ccc(-c5ccc6c(c5)c5ccccc5n6-c5ccccc5)cc4c4cc(-c5ccc6c(c5)c5ccccc5n6-c5ccccc5)ccc43)n2)cc1 ; c1ccc(-c2nc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)nc(-n3c4ccc(-c5ccc6c(c5)c5ccccc5n6-c5ccccc5)cc4c4cc(-c5ccc6c(c5)c5ccccc5n6-c5ccccc5)ccc43)n2)cc1]

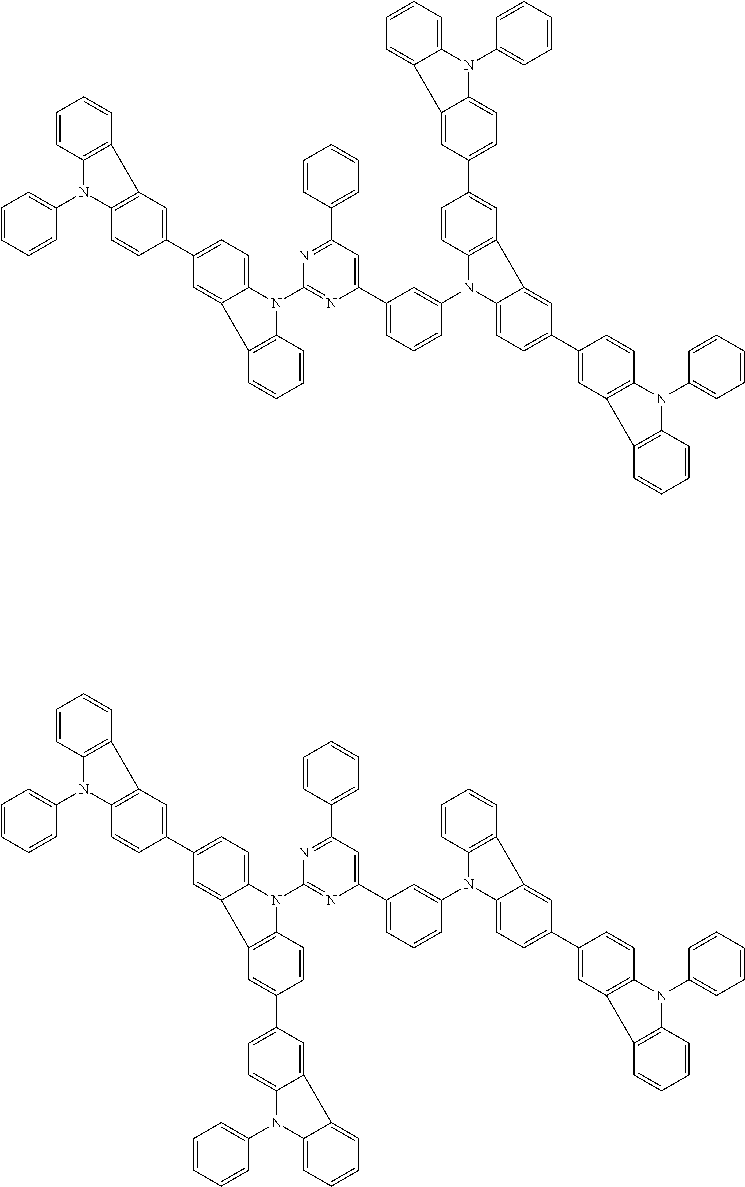 [SMILES: c1ccc(-c2cc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)nc(-n3c4ccccc4c4cc(-c5ccc6c(c5)c5ccccc5n6-c5ccccc5)ccc43)n2)cc1 ; c1ccc(-c2cc(-c3cccc(-n4c5ccccc5c5cc(-c6ccc7c(c6)c6ccccc6n7-c6ccccc6)ccc54)c3)nc(-n3c4ccc(-c5ccc6c(c5)c5ccccc5n6-c5ccccc5)cc4c4cc(-c5ccc6c(c5)c5ccccc5n6-c5ccccc5)ccc43)n2)cc1]

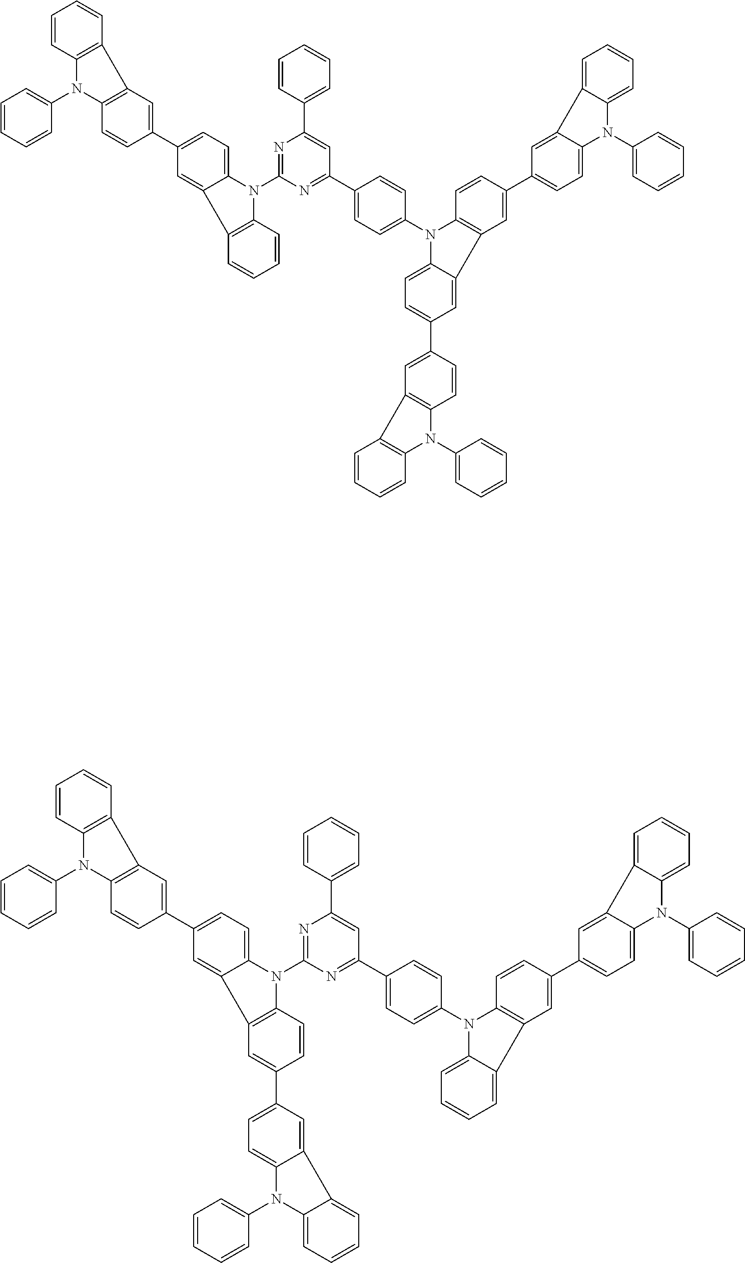 [SMILES: c1ccc(-c2cc(-c3ccc(-n4c5ccc(-c6ccc7c(c6)c6ccccc6n7-c6ccccc6)cc5c5cc(-c6ccc7c(c6)c6ccccc6n7-c6ccccc6)ccc54)cc3)nc(-n3c4ccccc4c4cc(-c5ccc6c(c5)c5ccccc5n6-c5ccccc5)ccc43)n2)cc1 ; c1ccc(-c2cc(-c3ccc(-n4c5ccccc5c5cc(-c6ccc7c(c6)c6ccccc6n7-c6ccccc6)ccc54)cc3)nc(-n3c4ccc(-c5ccc6c(c5)c5ccccc5n6-c5ccccc5)cc4c4cc(-c5ccc6c(c5)c5ccccc5n6-c5ccccc5)ccc43)n2)cc1]

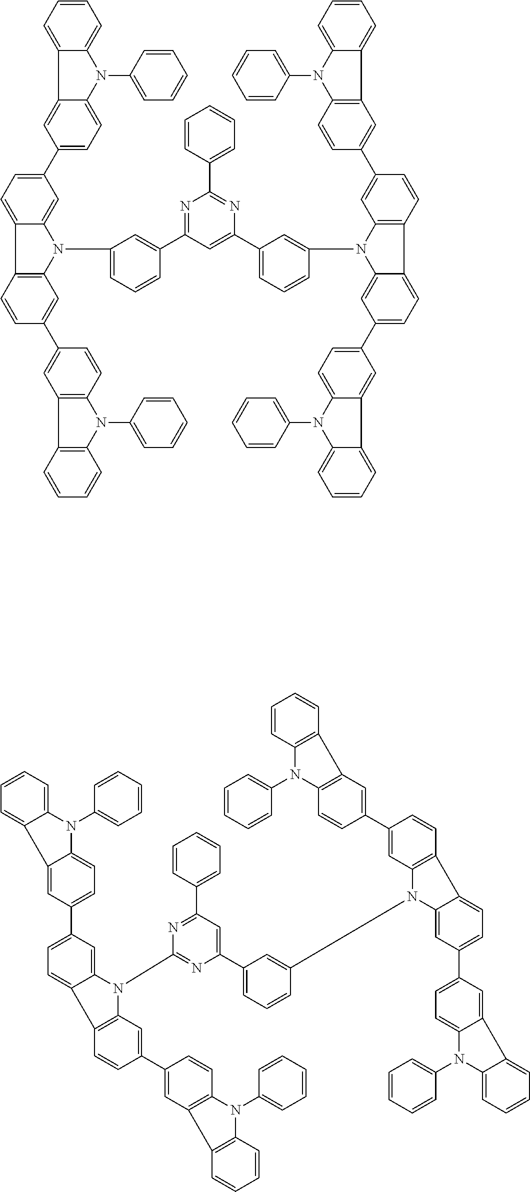 [SMILES: c1ccc(-c2nc(-c3cccc(-n4c5cc(-c6ccc7c(c6)c6ccccc6n7-c6ccccc6)ccc5c5ccc(-c6ccc7c(c6)c6ccccc6n7-c6ccccc6)cc54)c3)cc(-c3cccc(-n4c5cc(-c6ccc7c(c6)c6ccccc6n7-c6ccccc6)ccc5c5ccc(-c6ccc7c(c6)c6ccccc6n7-c6ccccc6)cc54)c3)n2)cc1 ; c1ccc(-c2cc(-c3cccc(-n4c5cc(-c6ccc7c(c6)c6ccccc6n7-c6ccccc6)ccc5c5ccc(-c6ccc7c(c6)c6ccccc6n7-c6ccccc6)cc54)c3)nc(-n3c4cc(-c5ccc6c(c5)c5ccccc5n6-c5ccccc5)ccc4c4ccc(-c5ccc6c(c5)c5ccccc5n6-c5ccccc5)cc43)n2)cc1]

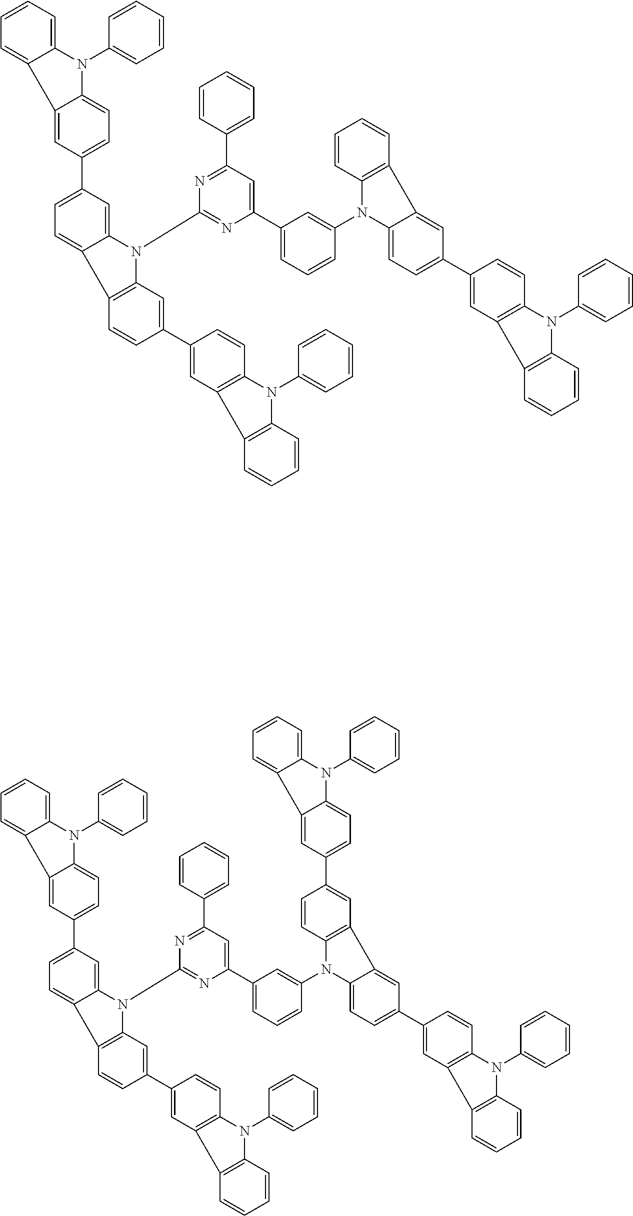 [SMILES: c1ccc(-c2cc(-c3cccc(-n4c5ccccc5c5cc(-c6ccc7c(c6)c6ccccc6n7-c6ccccc6)ccc54)c3)nc(-n3c4cc(-c5ccc6c(c5)c5ccccc5n6-c5ccccc5)ccc4c4ccc(-c5ccc6c(c5)c5ccccc5n6-c5ccccc5)cc43)n2)cc1 ; c1ccc(-c2cc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)nc(-n3c4cc(-c5ccc6c(c5)c5ccccc5n6-c5ccccc5)ccc4c4ccc(-c5ccc6c(c5)c5ccccc5n6-c5ccccc5)cc43)n2)cc1]

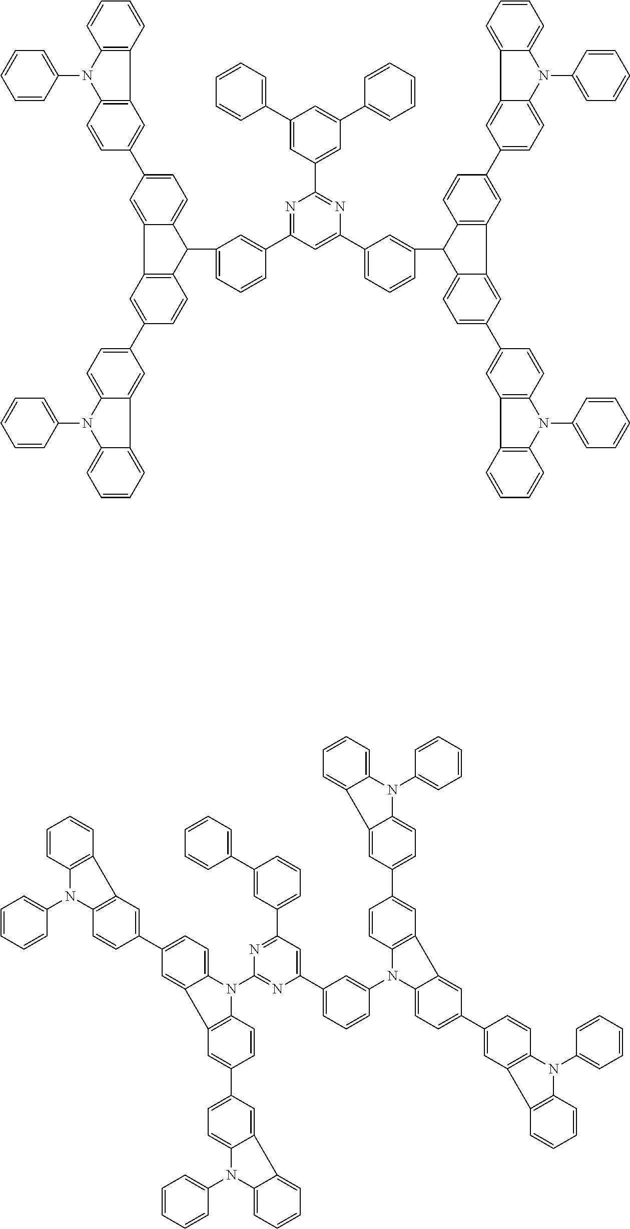 [SMILES: c1ccc(-c2cc(-c3ccccc3)cc(-c3nc(-c4cccc(C5c6ccc(-c7ccc8c(c7)c7ccccc7n8-c7ccccc7)cc6-c6cc(-c7ccc8c(c7)c7ccccc7n8-c7ccccc7)ccc65)c4)cc(-c4cccc(C5c6ccc(-c7ccc8c(c7)c7ccccc7n8-c7ccccc7)cc6-c6cc(-c7ccc8c(c7)c7ccccc7n8-c7ccccc7)ccc65)c4)n3)c2)cc1 ; c1ccc(-c2cccc(-c3cc(-c4cccc(-n5c6ccc(-c7ccc8c(c7)c7ccccc7n8-c7ccccc7)cc6c6cc(-c7ccc8c(c7)c7ccccc7n8-c7ccccc7)ccc65)c4)nc(-n4c5ccc(-c6ccc7c(c6)c6ccccc6n7-c6ccccc6)cc5c5cc(-c6ccc7c(c6)c6ccccc6n7-c6ccccc6)ccc54)n3)c2)cc1]

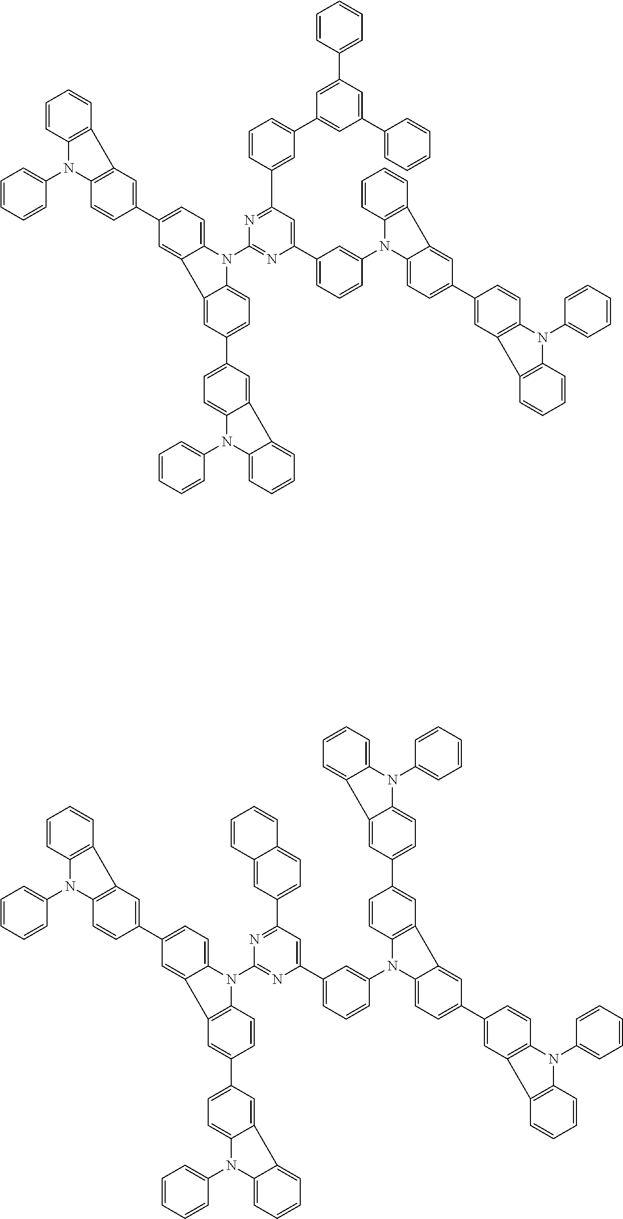 [SMILES: c1ccc(-c2cc(-c3ccccc3)cc(-c3cccc(-c4cc(-c5cccc(-n6c7ccccc7c7cc(-c8ccc9c(c8)c8ccccc8n9-c8ccccc8)ccc76)c5)nc(-n5c6ccc(-c7ccc8c(c7)c7ccccc7n8-c7ccccc7)cc6c6cc(-c7ccc8c(c7)c7ccccc7n8-c7ccccc7)ccc65)n4)c3)c2)cc1 ; c1ccc(-n2c3ccccc3c3cc(-c4ccc5c(c4)c4cc(-c6ccc7c(c6)c6ccccc6n7-c6ccccc6)ccc4n5-c4cccc(-c5cc(-c6ccc7ccccc7c6)nc(-n6c7ccc(-c8ccc9c(c8)c8ccccc8n9-c8ccccc8)cc7c7cc(-c8ccc9c(c8)c8ccccc8n9-c8ccccc8)ccc76)n5)c4)ccc32)cc1]

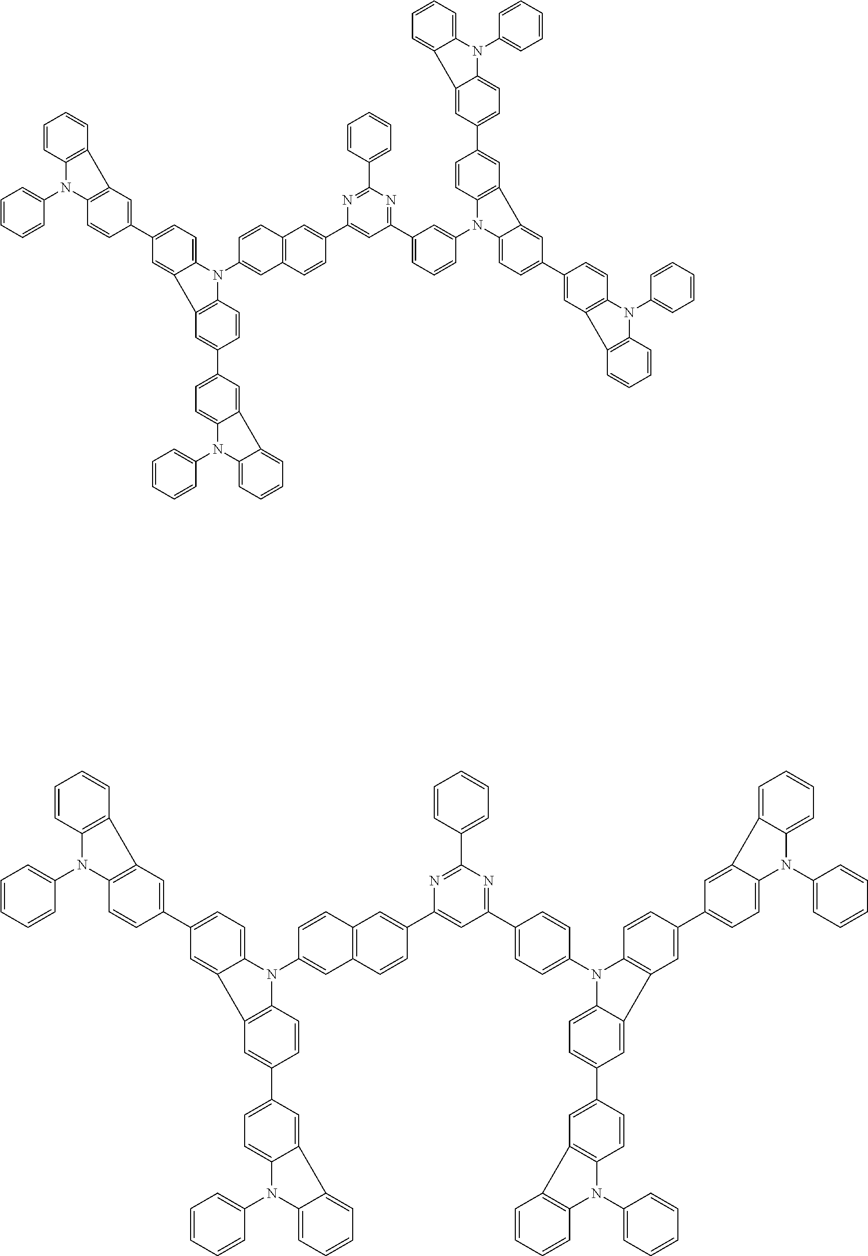 [SMILES: c1ccc(-c2nc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)cc(-c3ccc4cc(-n5c6ccc(-c7ccc8c(c7)c7ccccc7n8-c7ccccc7)cc6c6cc(-c7ccc8c(c7)c7ccccc7n8-c7ccccc7)ccc65)ccc4c3)n2)cc1 ; c1ccc(-c2nc(-c3ccc(-n4c5ccc(-c6ccc7c(c6)c6ccccc6n7-c6ccccc6)cc5c5cc(-c6ccc7c(c6)c6ccccc6n7-c6ccccc6)ccc54)cc3)cc(-c3ccc4cc(-n5c6ccc(-c7ccc8c(c7)c7ccccc7n8-c7ccccc7)cc6c6cc(-c7ccc8c(c7)c7ccccc7n8-c7ccccc7)ccc65)ccc4c3)n2)cc1]

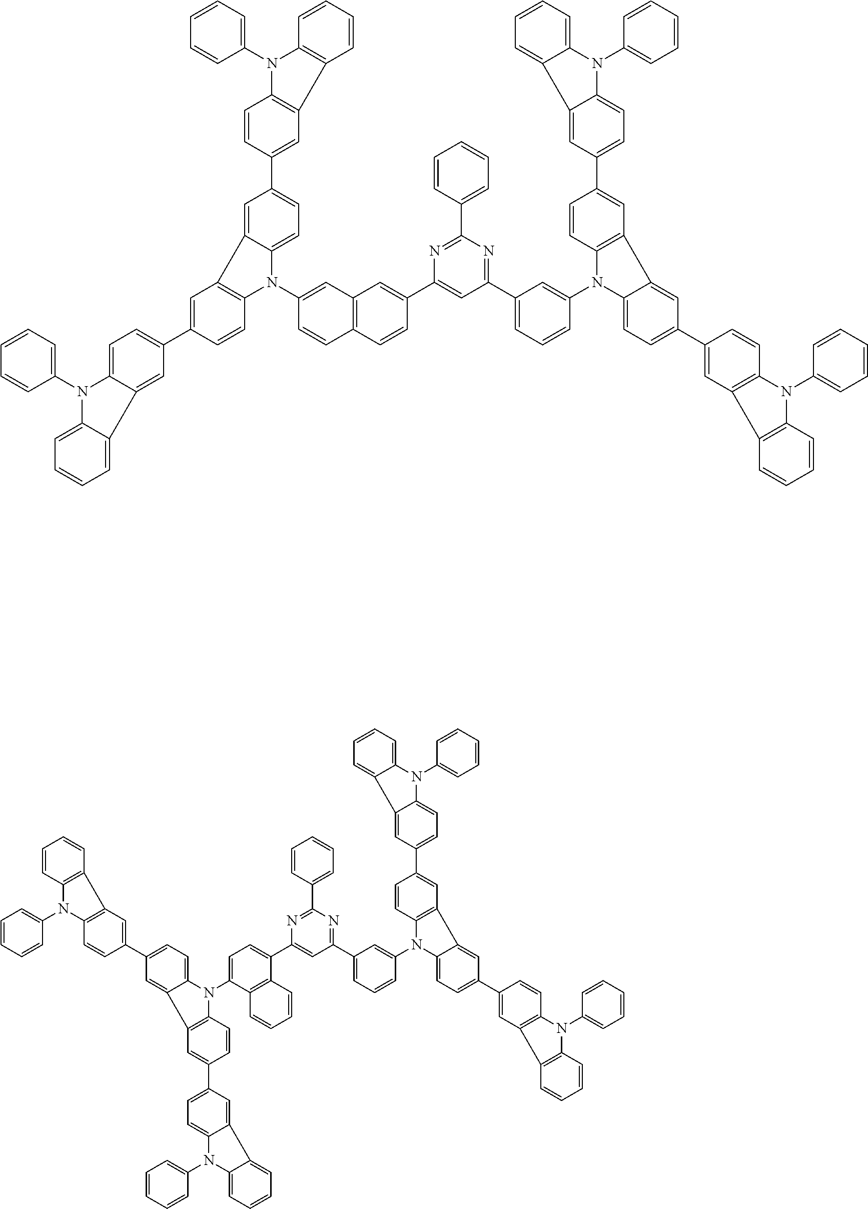 [SMILES: c1ccc(-c2nc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)cc(-c3ccc4ccc(-n5c6ccc(-c7ccc8c(c7)c7ccccc7n8-c7ccccc7)cc6c6cc(-c7ccc8c(c7)c7ccccc7n8-c7ccccc7)ccc65)cc4c3)n2)cc1 ; c1ccc(-c2nc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)cc(-c3ccc(-n4c5ccc(-c6ccc7c(c6)c6ccccc6n7-c6ccccc6)cc5c5cc(-c6ccc7c(c6)c6ccccc6n7-c6ccccc6)ccc54)c4ccccc34)n2)cc1]

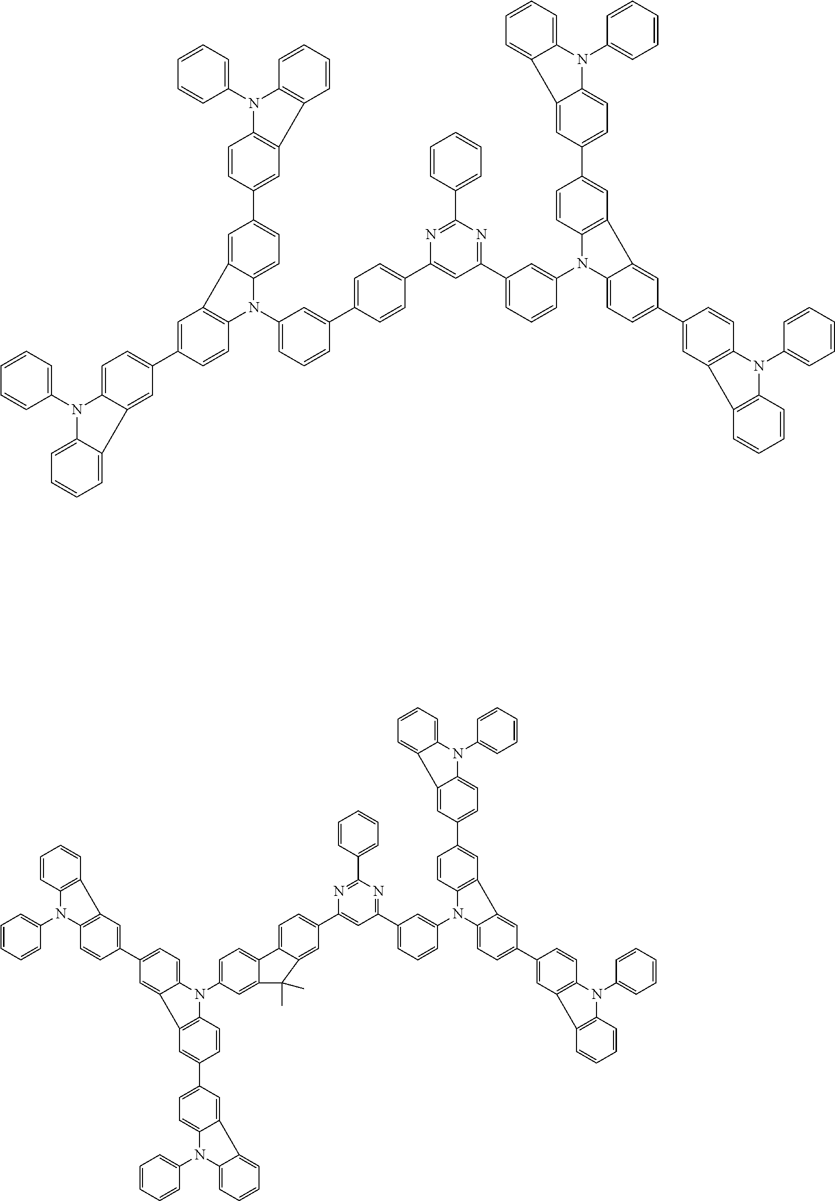 [SMILES: c1ccc(-c2nc(-c3ccc(-c4cccc(-n5c6ccc(-c7ccc8c(c7)c7ccccc7n8-c7ccccc7)cc6c6cc(-c7ccc8c(c7)c7ccccc7n8-c7ccccc7)ccc65)c4)cc3)cc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)n2)cc1 ; CC1(C)c2cc(-c3cc(-c4cccc(-n5c6ccc(-c7ccc8c(c7)c7ccccc7n8-c7ccccc7)cc6c6cc(-c7ccc8c(c7)c7ccccc7n8-c7ccccc7)ccc65)c4)nc(-c4ccccc4)n3)ccc2-c2ccc(-n3c4ccc(-c5ccc6c(c5)c5ccccc5n6-c5ccccc5)cc4c4cc(-c5ccc6c(c5)c5ccccc5n6-c5ccccc5)ccc43)cc21]

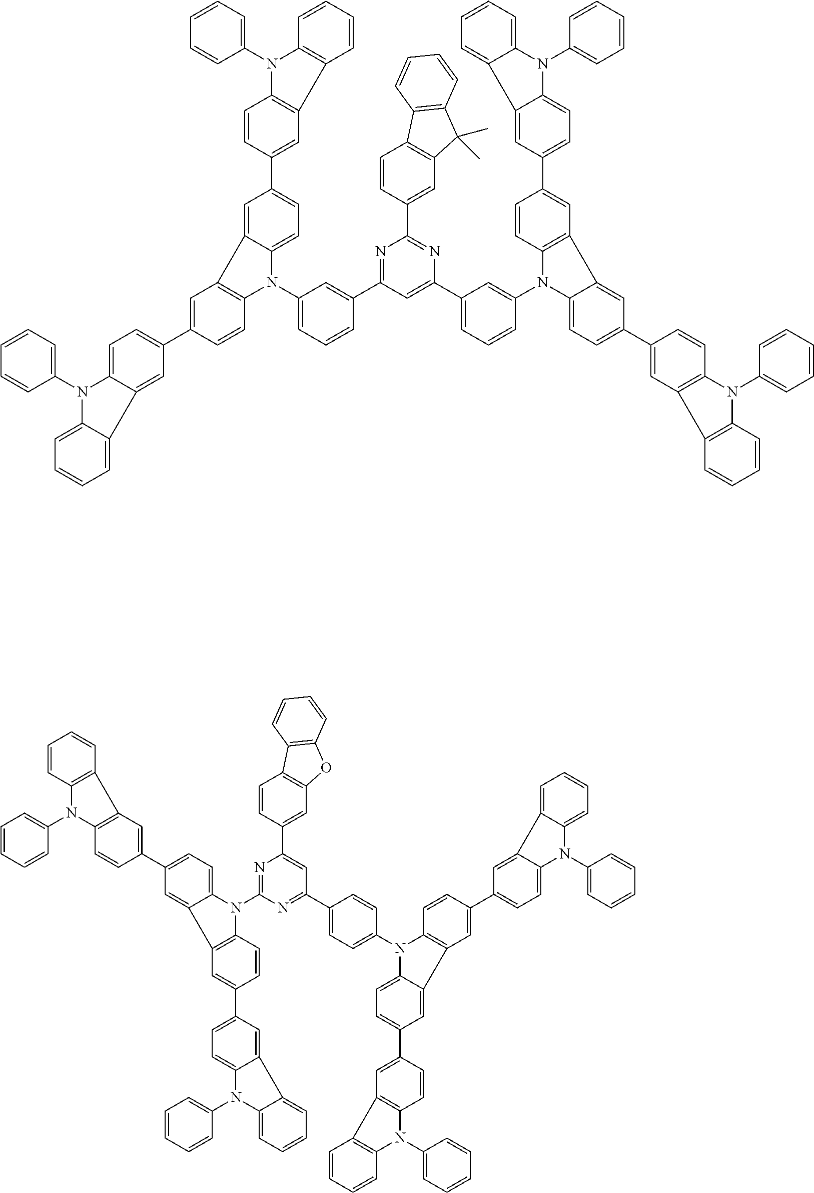 [SMILES: CC1(C)c2ccccc2-c2ccc(-c3nc(-c4cccc(-n5c6ccc(-c7ccc8c(c7)c7ccccc7n8-c7ccccc7)cc6c6cc(-c7ccc8c(c7)c7ccccc7n8-c7ccccc7)ccc65)c4)cc(-c4cccc(-n5c6ccc(-c7ccc8c(c7)c7ccccc7n8-c7ccccc7)cc6c6cc(-c7ccc8c(c7)c7ccccc7n8-c7ccccc7)ccc65)c4)n3)cc21 ; c1ccc(-n2c3ccccc3c3cc(-c4ccc5c(c4)c4cc(-c6ccc7c(c6)c6ccccc6n7-c6ccccc6)ccc4n5-c4ccc(-c5cc(-c6ccc7c(c6)oc6ccccc67)nc(-n6c7ccc(-c8ccc9c(c8)c8ccccc8n9-c8ccccc8)cc7c7cc(-c8ccc9c(c8)c8ccccc8n9-c8ccccc8)ccc76)n5)cc4)ccc32)cc1]

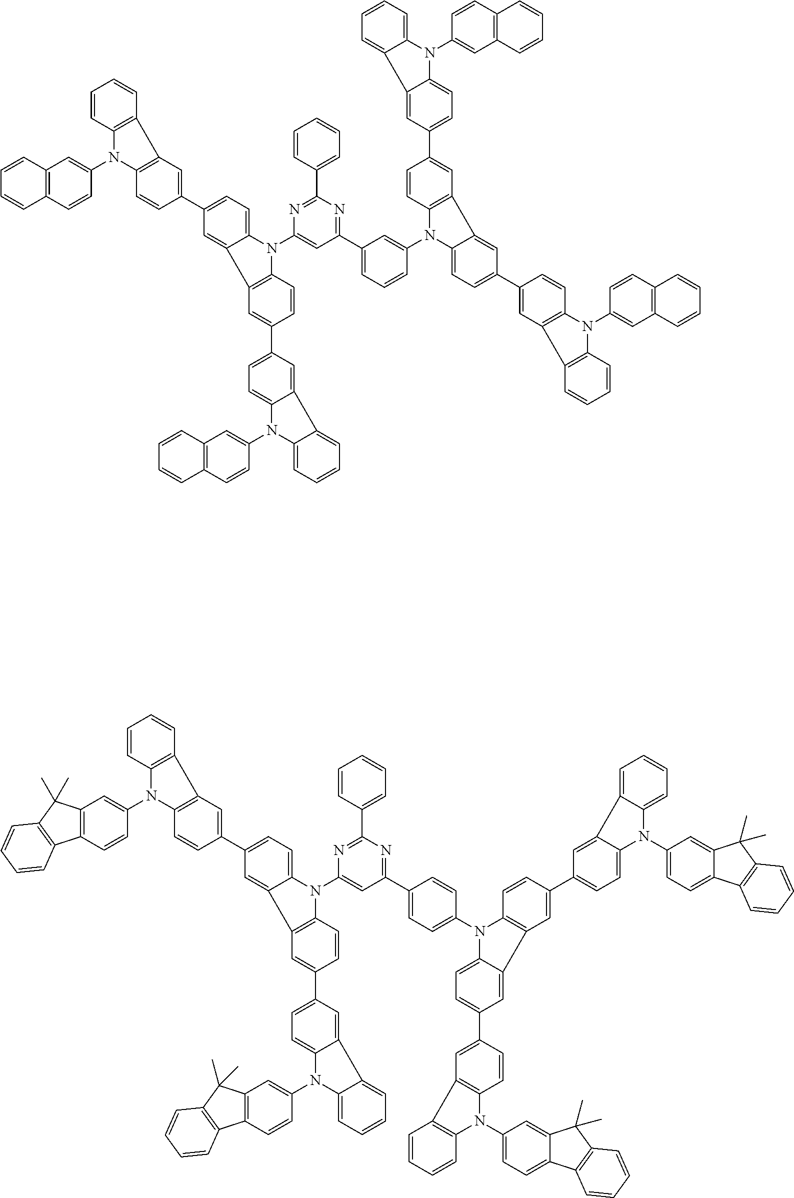

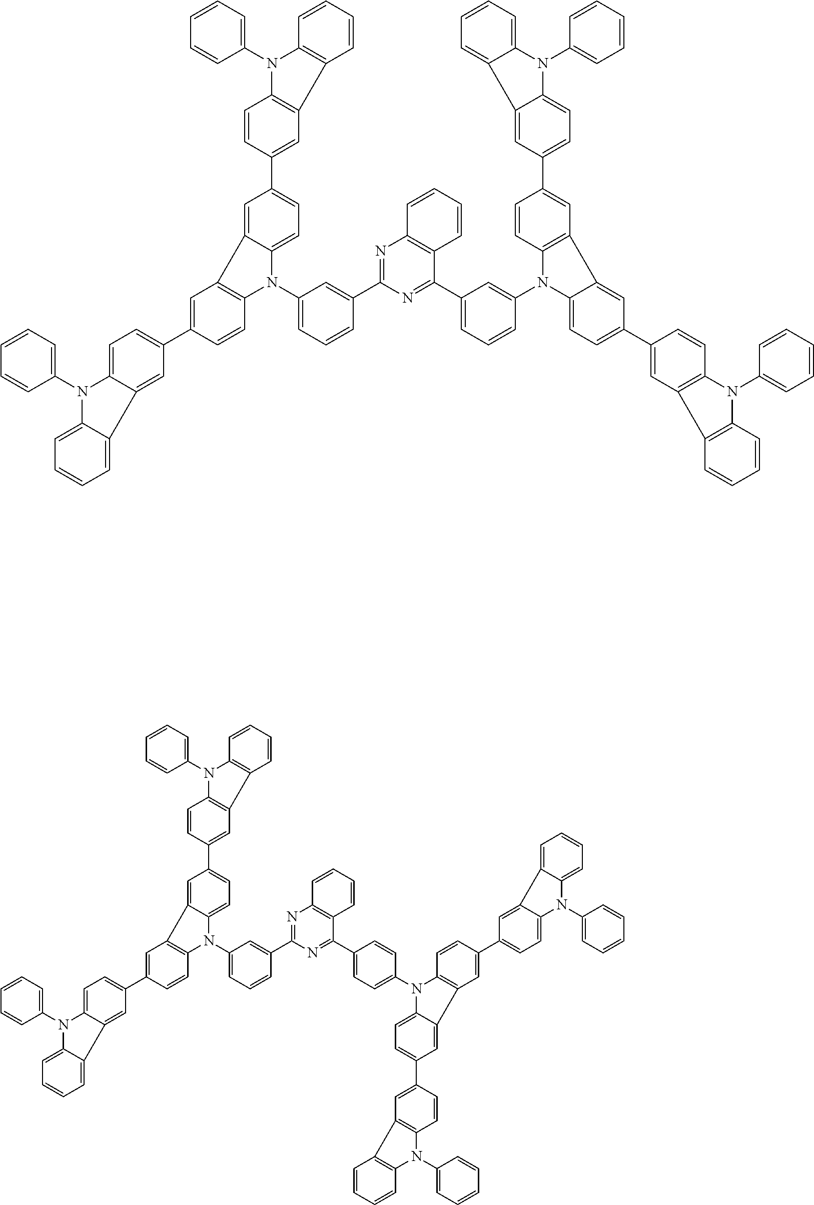 [SMILES: c1ccc(-n2c3ccccc3c3cc(-c4ccc5c(c4)c4cc(-c6ccc7c(c6)c6ccccc6n7-c6ccccc6)ccc4n5-c4cccc(-c5nc(-c6cccc(-n7c8ccc(-c9ccc%10c(c9)c9ccccc9n%10-c9ccccc9)cc8c8cc(-c9ccc%10c(c9)c9ccccc9n%10-c9ccccc9)ccc87)c6)c6ccccc6n5)c4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccc(-c5nc(-c6cccc(-n7c8ccc(-c9ccc%10c(c9)c9ccccc9n%10-c9ccccc9)cc8c8cc(-c9ccc%10c(c9)c9ccccc9n%10-c9ccccc9)ccc87)c6)nc6ccccc56)cc4)ccc32)cc1]

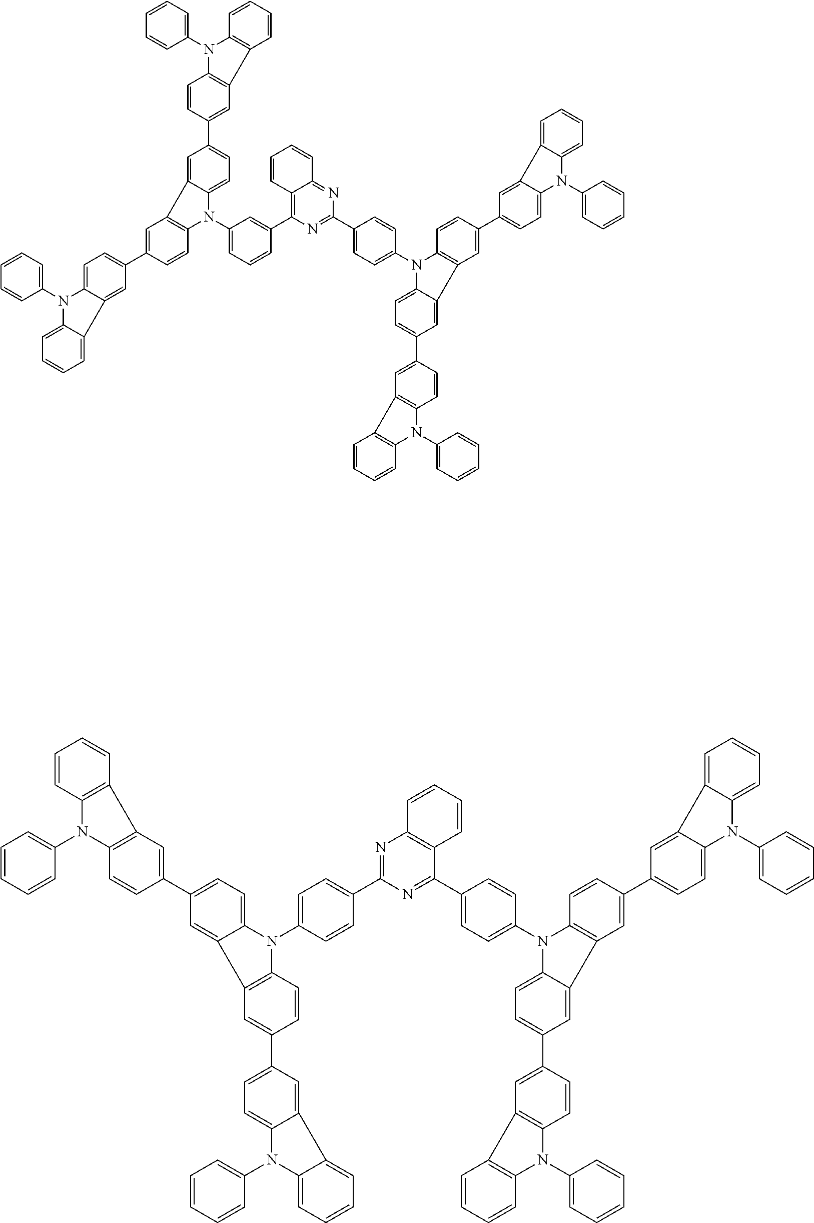 [SMILES: c1ccc(-n2c3ccccc3c3cc(-c4ccc5c(c4)c4cc(-c6ccc7c(c6)c6ccccc6n7-c6ccccc6)ccc4n5-c4ccc(-c5nc(-c6cccc(-n7c8ccc(-c9ccc%10c(c9)c9ccccc9n%10-c9ccccc9)cc8c8cc(-c9ccc%10c(c9)c9ccccc9n%10-c9ccccc9)ccc87)c6)c6ccccc6n5)cc4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccc(-c5nc(-c6ccc(-n7c8ccc(-c9ccc%10c(c9)c9ccccc9n%10-c9ccccc9)cc8c8cc(-c9ccc%10c(c9)c9ccccc9n%10-c9ccccc9)ccc87)cc6)c6ccccc6n5)cc4)ccc32)cc1]

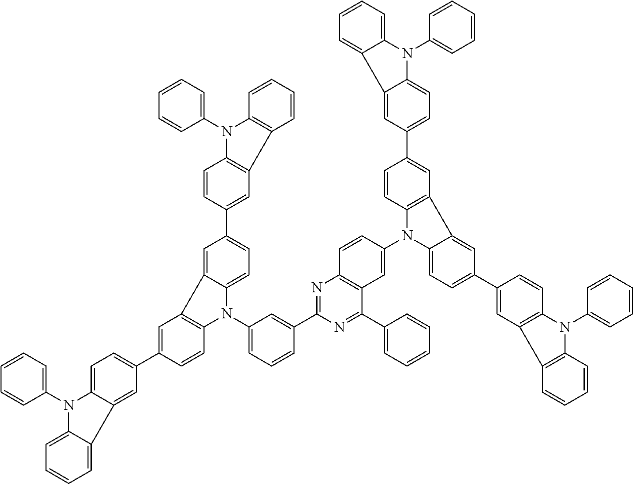 [SMILES: c1ccc(-c2nc(-c3cccc(-n4c5ccc(-c6ccc7c(c6)c6ccccc6n7-c6ccccc6)cc5c5cc(-c6ccc7c(c6)c6ccccc6n7-c6ccccc6)ccc54)c3)nc3ccc(-n4c5ccc(-c6ccc7c(c6)c6ccccc6n7-c6ccccc6)cc5c5cc(-c6ccc7c(c6)c6ccccc6n7-c6ccccc6)ccc54)cc23)cc1]

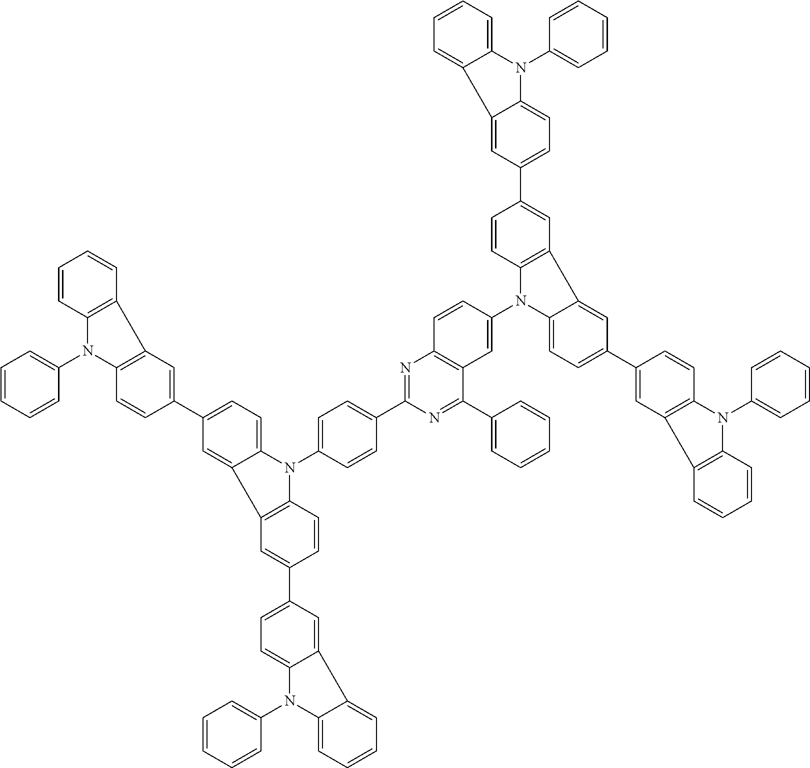 [SMILES: c1ccc(-c2nc(-c3ccc(-n4c5ccc(-c6ccc7c(c6)c6ccccc6n7-c6ccccc6)cc5c5cc(-c6ccc7c(c6)c6ccccc6n7-c6ccccc6)ccc54)cc3)nc3ccc(-n4c5ccc(-c6ccc7c(c6)c6ccccc6n7-c6ccccc6)cc5c5cc(-c6ccc7c(c6)c6ccccc6n7-c6ccccc6)ccc54)cc23)cc1]

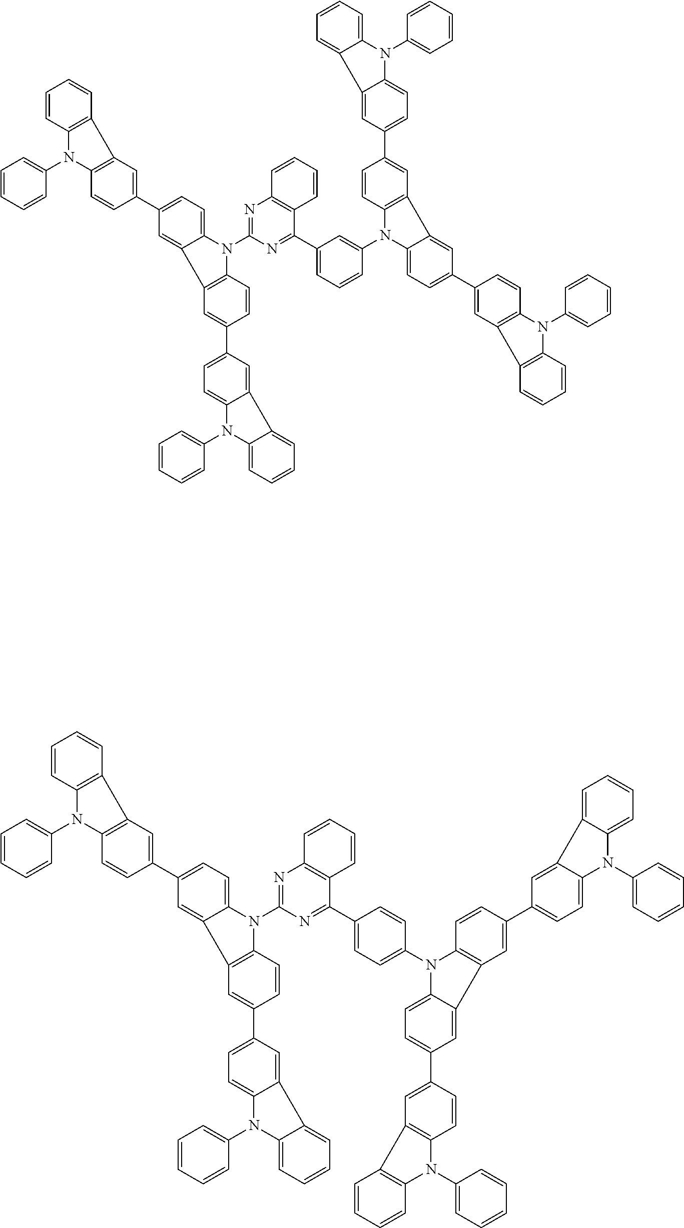 [SMILES: c1ccc(-n2c3ccccc3c3cc(-c4ccc5c(c4)c4cc(-c6ccc7c(c6)c6ccccc6n7-c6ccccc6)ccc4n5-c4cccc(-c5nc(-n6c7ccc(-c8ccc9c(c8)c8ccccc8n9-c8ccccc8)cc7c7cc(-c8ccc9c(c8)c8ccccc8n9-c8ccccc8)ccc76)nc6ccccc56)c4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccc(-c5nc(-n6c7ccc(-c8ccc9c(c8)c8ccccc8n9-c8ccccc8)cc7c7cc(-c8ccc9c(c8)c8ccccc8n9-c8ccccc8)ccc76)nc6ccccc56)cc4)ccc32)cc1]

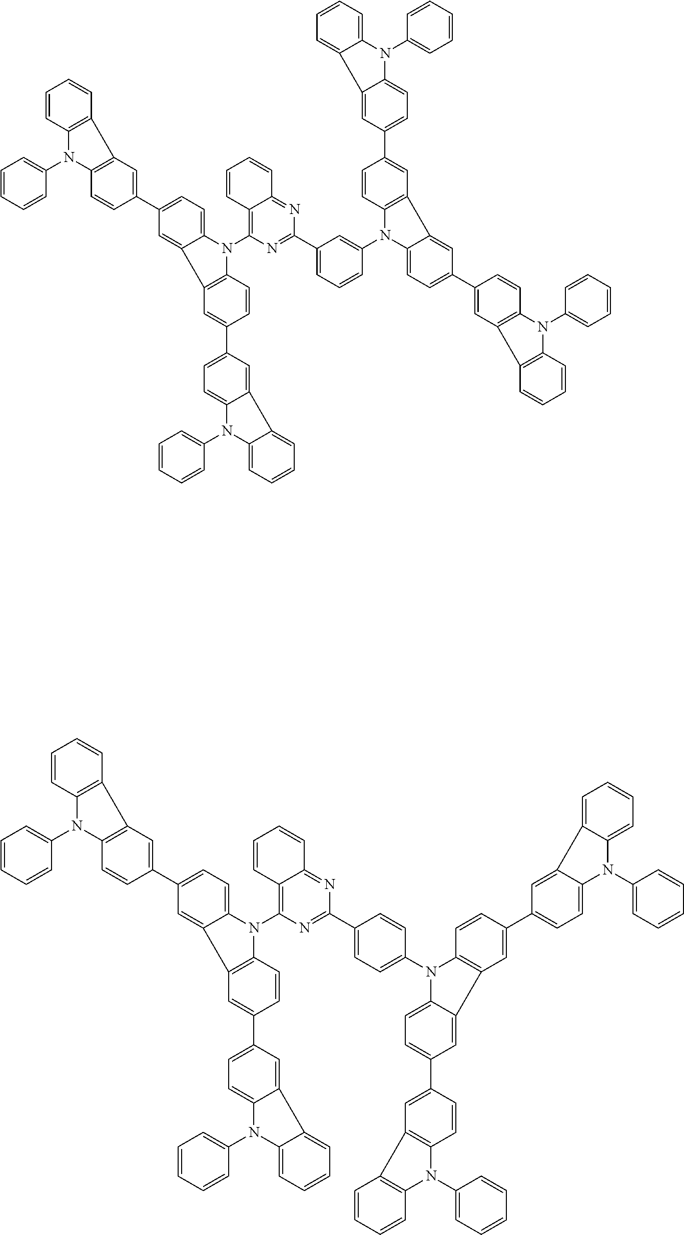 [SMILES: c1ccc(-n2c3ccccc3c3cc(-c4ccc5c(c4)c4cc(-c6ccc7c(c6)c6ccccc6n7-c6ccccc6)ccc4n5-c4cccc(-c5nc(-n6c7ccc(-c8ccc9c(c8)c8ccccc8n9-c8ccccc8)cc7c7cc(-c8ccc9c(c8)c8ccccc8n9-c8ccccc8)ccc76)c6ccccc6n5)c4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccc(-c5nc(-n6c7ccc(-c8ccc9c(c8)c8ccccc8n9-c8ccccc8)cc7c7cc(-c8ccc9c(c8)c8ccccc8n9-c8ccccc8)ccc76)c6ccccc6n5)cc4)ccc32)cc1]

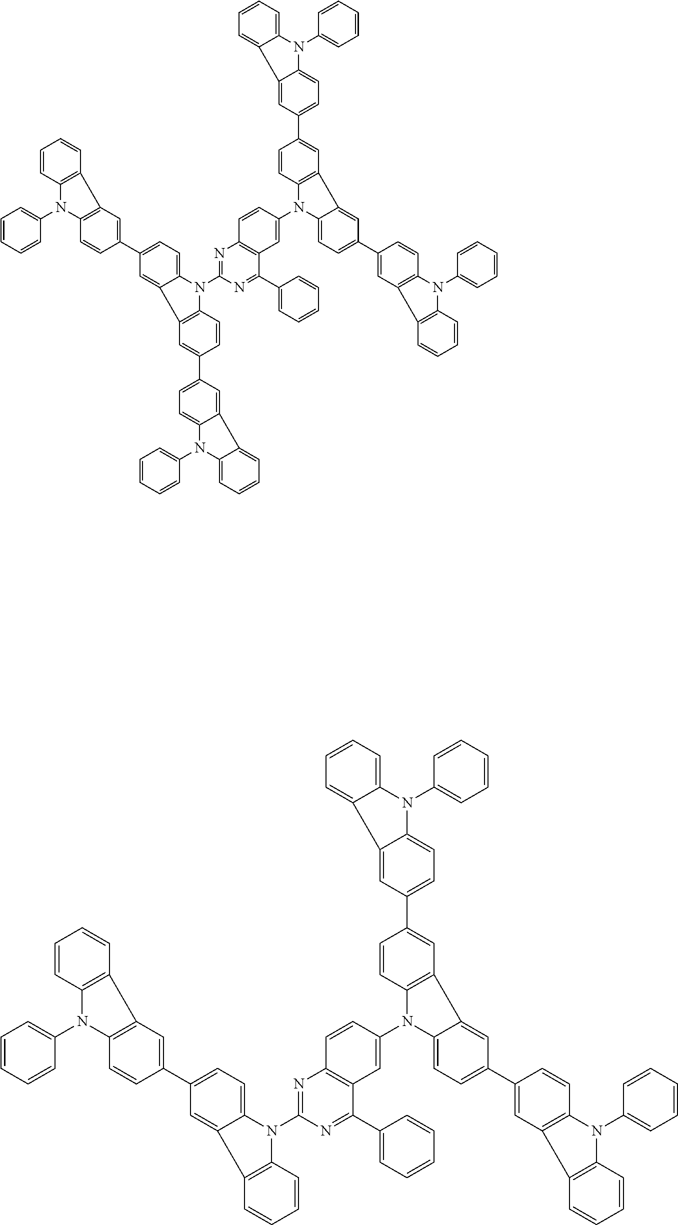 [SMILES: c1ccc(-c2nc(-n3c4ccc(-c5ccc6c(c5)c5ccccc5n6-c5ccccc5)cc4c4cc(-c5ccc6c(c5)c5ccccc5n6-c5ccccc5)ccc43)nc3ccc(-n4c5ccc(-c6ccc7c(c6)c6ccccc6n7-c6ccccc6)cc5c5cc(-c6ccc7c(c6)c6ccccc6n7-c6ccccc6)ccc54)cc23)cc1 ; c1ccc(-c2nc(-n3c4ccccc4c4cc(-c5ccc6c(c5)c5ccccc5n6-c5ccccc5)ccc43)nc3ccc(-n4c5ccc(-c6ccc7c(c6)c6ccccc6n7-c6ccccc6)cc5c5cc(-c6ccc7c(c6)c6ccccc6n7-c6ccccc6)ccc54)cc23)cc1]

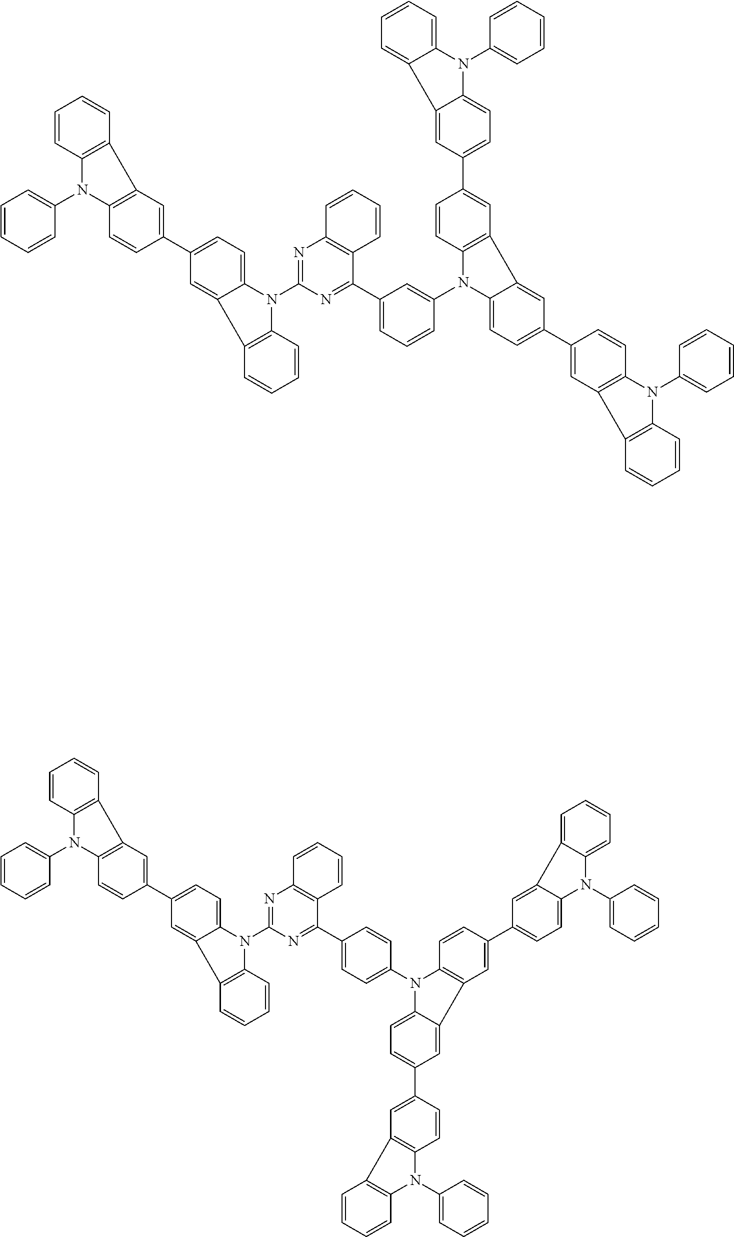 [SMILES: c1ccc(-n2c3ccccc3c3cc(-c4ccc5c(c4)c4cc(-c6ccc7c(c6)c6ccccc6n7-c6ccccc6)ccc4n5-c4cccc(-c5nc(-n6c7ccccc7c7cc(-c8ccc9c(c8)c8ccccc8n9-c8ccccc8)ccc76)nc6ccccc56)c4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccc(-c5nc(-n6c7ccccc7c7cc(-c8ccc9c(c8)c8ccccc8n9-c8ccccc8)ccc76)nc6ccccc56)cc4)ccc32)cc1]

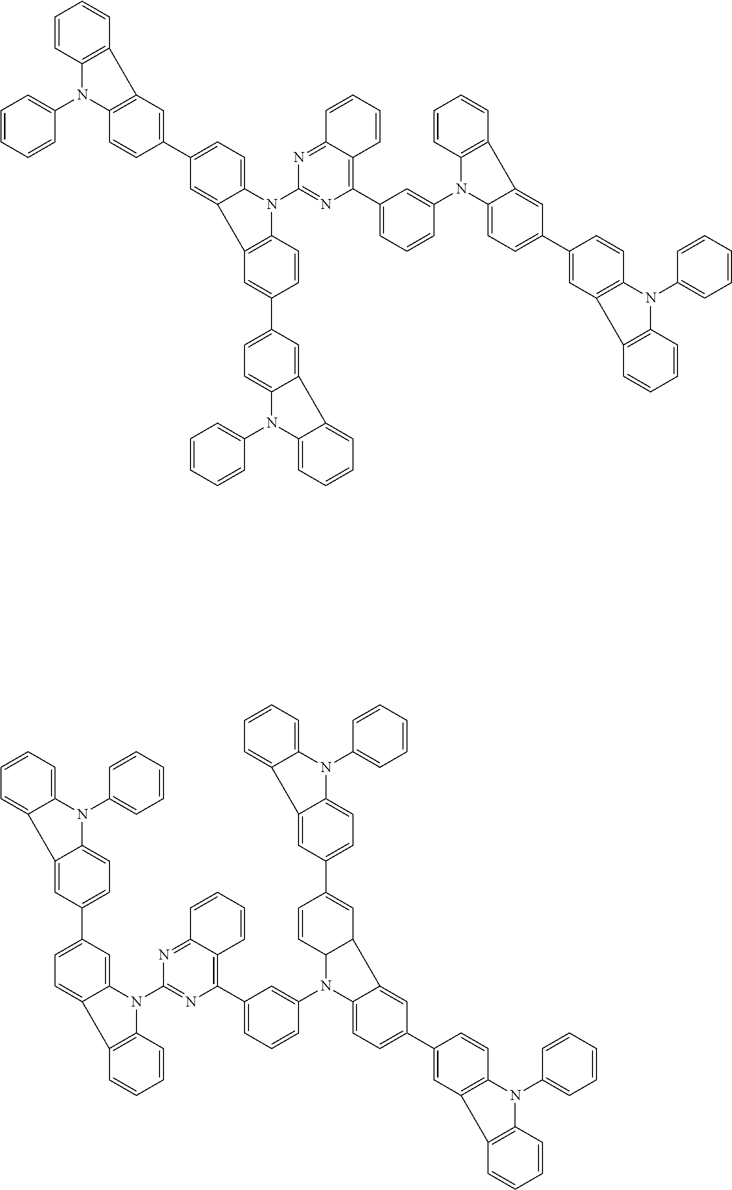 [SMILES: c1ccc(-n2c3ccccc3c3cc(-c4ccc5c(c4)c4ccccc4n5-c4cccc(-c5nc(-n6c7ccc(-c8ccc9c(c8)c8ccccc8n9-c8ccccc8)cc7c7cc(-c8ccc9c(c8)c8ccccc8n9-c8ccccc8)ccc76)nc6ccccc56)c4)ccc32)cc1 ; C1=CC2C(C=C1c1ccc3c(c1)c1ccccc1n3-c1ccccc1)c1cc(-c3ccc4c(c3)c3ccccc3n4-c3ccccc3)ccc1N2c1cccc(-c2nc(-n3c4ccccc4c4ccc(-c5ccc6c(c5)c5ccccc5n6-c5ccccc5)cc43)nc3ccccc23)c1]

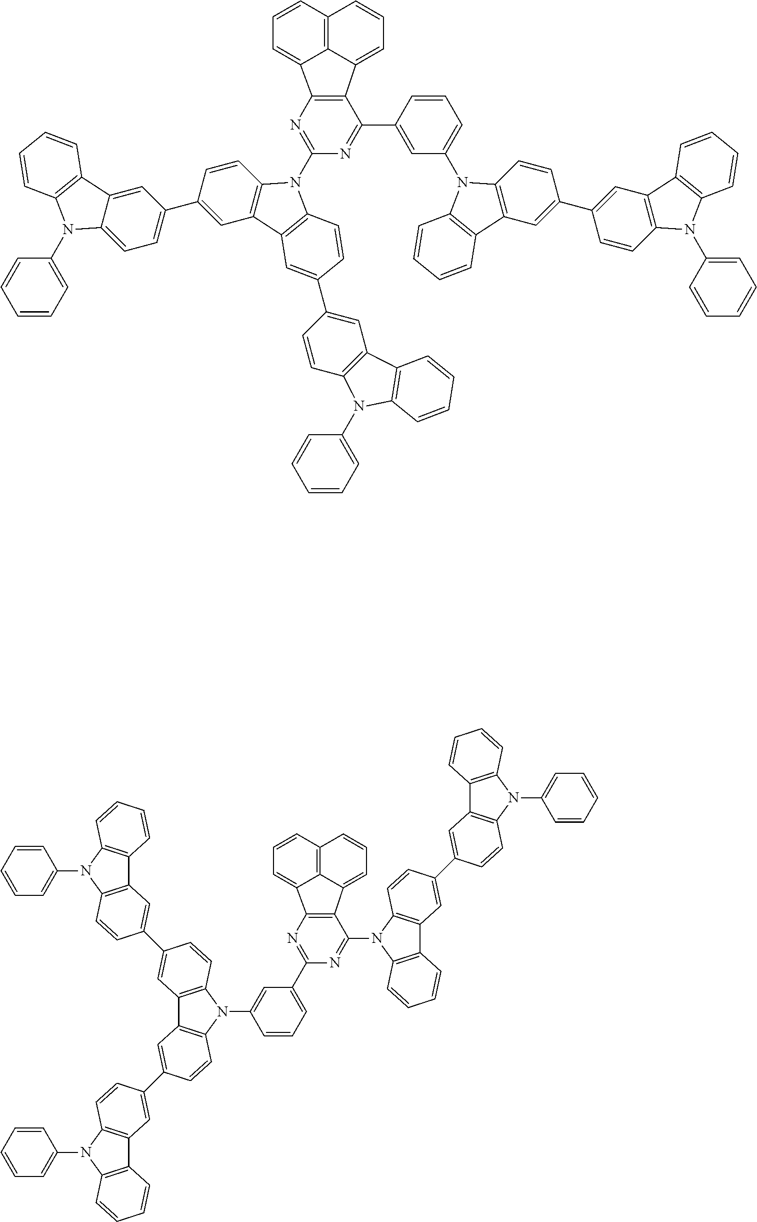 [SMILES: c1ccc(-n2c3ccccc3c3cc(-c4ccc5c(c4)c4ccccc4n5-c4cccc(-c5nc(-n6c7ccc(-c8ccc9c(c8)c8ccccc8n9-c8ccccc8)cc7c7cc(-c8ccc9c(c8)c8ccccc8n9-c8ccccc8)ccc76)nc6c5-c5cccc7cccc-6c57)c4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4cccc(-c5nc6c(c(-n7c8ccccc8c8cc(-c9ccc%10c(c9)c9ccccc9n%10-c9ccccc9)ccc87)n5)-c5cccc7cccc-6c57)c4)ccc32)cc1]

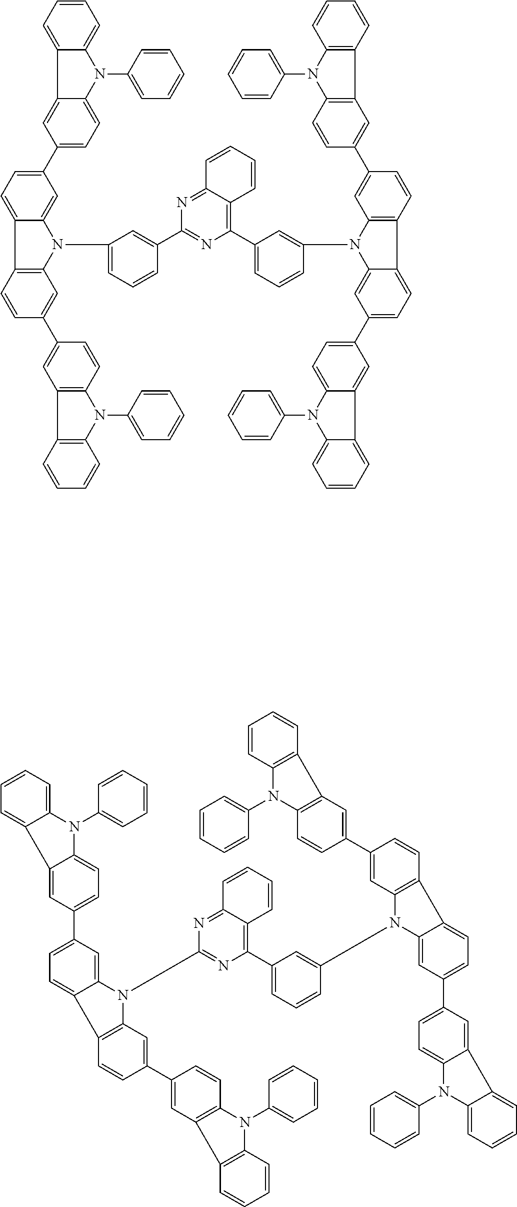 [SMILES: c1ccc(-n2c3ccccc3c3cc(-c4ccc5c6ccc(-c7ccc8c(c7)c7ccccc7n8-c7ccccc7)cc6n(-c6cccc(-c7nc(-c8cccc(-n9c%10cc(-c%11ccc%12c(c%11)c%11ccccc%11n%12-c%11ccccc%11)ccc%10c%10ccc(-c%11ccc%12c(c%11)c%11ccccc%11n%12-c%11ccccc%11)cc%109)c8)c8ccccc8n7)c6)c5c4)ccc32)cc1 ; c1ccc(-n2c3ccccc3c3cc(-c4ccc5c6ccc(-c7ccc8c(c7)c7ccccc7n8-c7ccccc7)cc6n(-c6cccc(-c7nc(-n8c9cc(-c%10ccc%11c(c%10)c%10ccccc%10n%11-c%10ccccc%10)ccc9c9ccc(-c%10ccc%11c(c%10)c%10ccccc%10n%11-c%10ccccc%10)cc98)nc8ccccc78)c6)c5c4)ccc32)cc1]

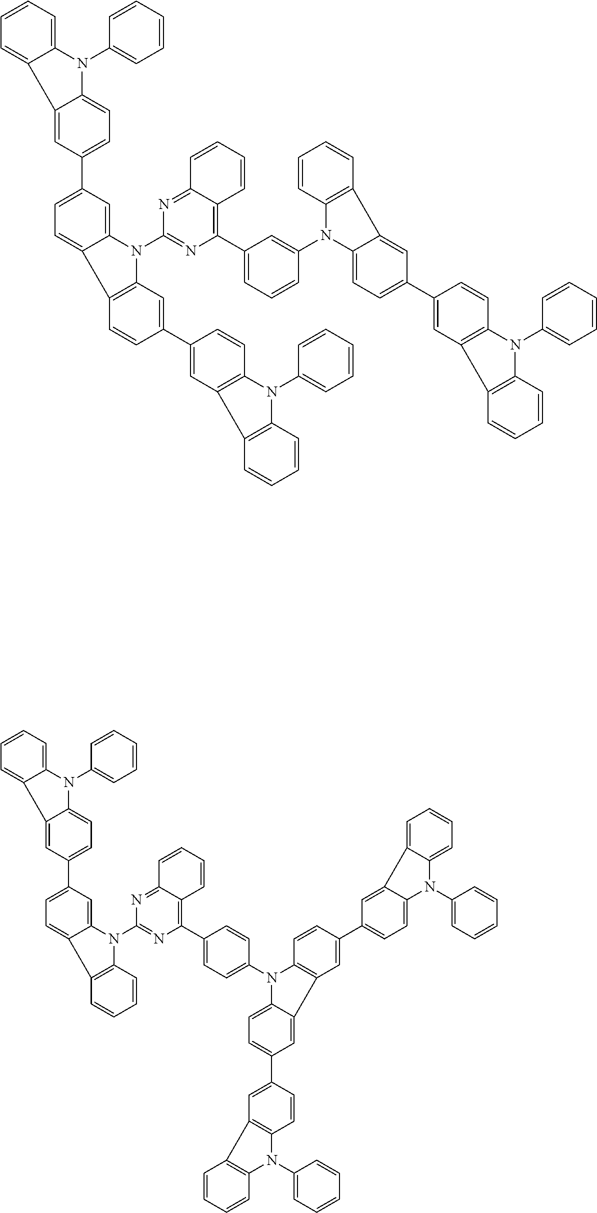 [SMILES: c1ccc(-n2c3ccccc3c3cc(-c4ccc5c(c4)c4ccccc4n5-c4cccc(-c5nc(-n6c7cc(-c8ccc9c(c8)c8ccccc8n9-c8ccccc8)ccc7c7ccc(-c8ccc9c(c8)c8ccccc8n9-c8ccccc8)cc76)nc6ccccc56)c4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccc(-c5nc(-n6c7ccccc7c7ccc(-c8ccc9c(c8)c8ccccc8n9-c8ccccc8)cc76)nc6ccccc56)cc4)ccc32)cc1]

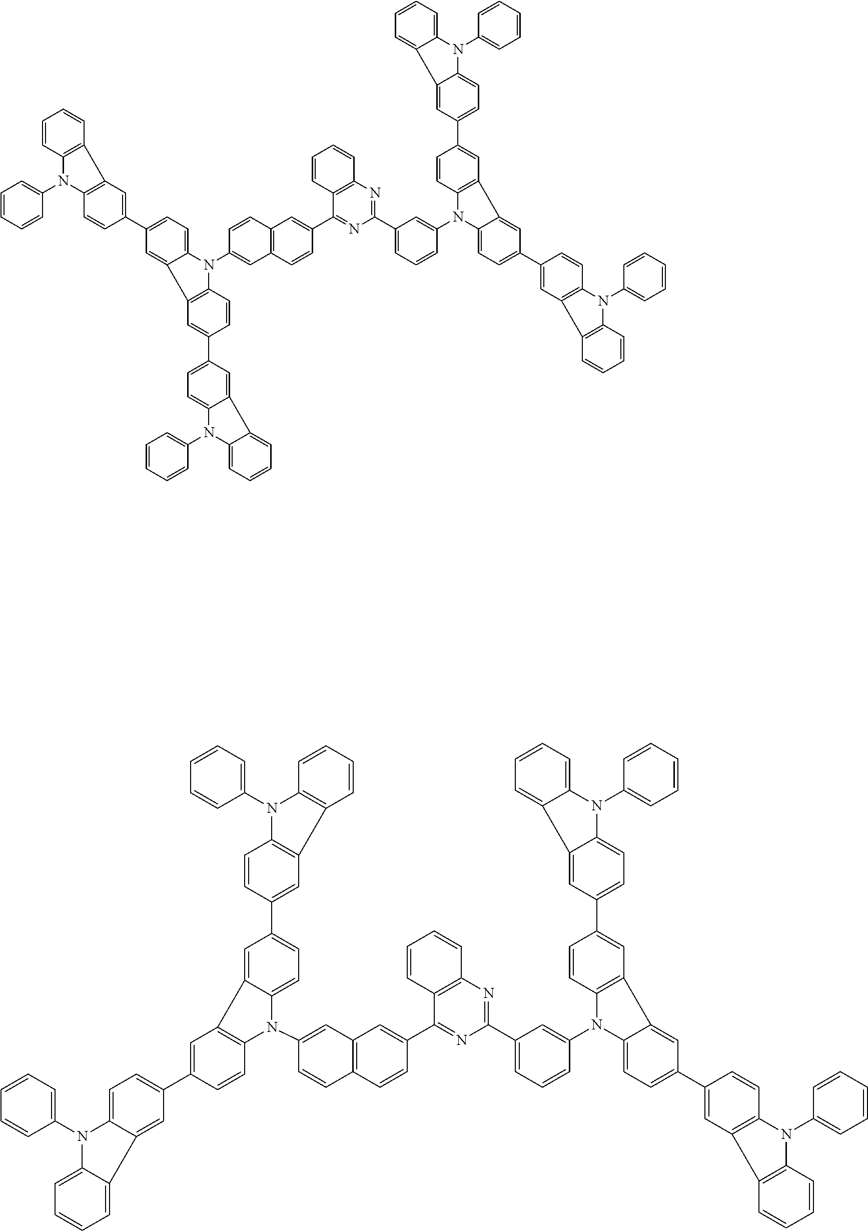

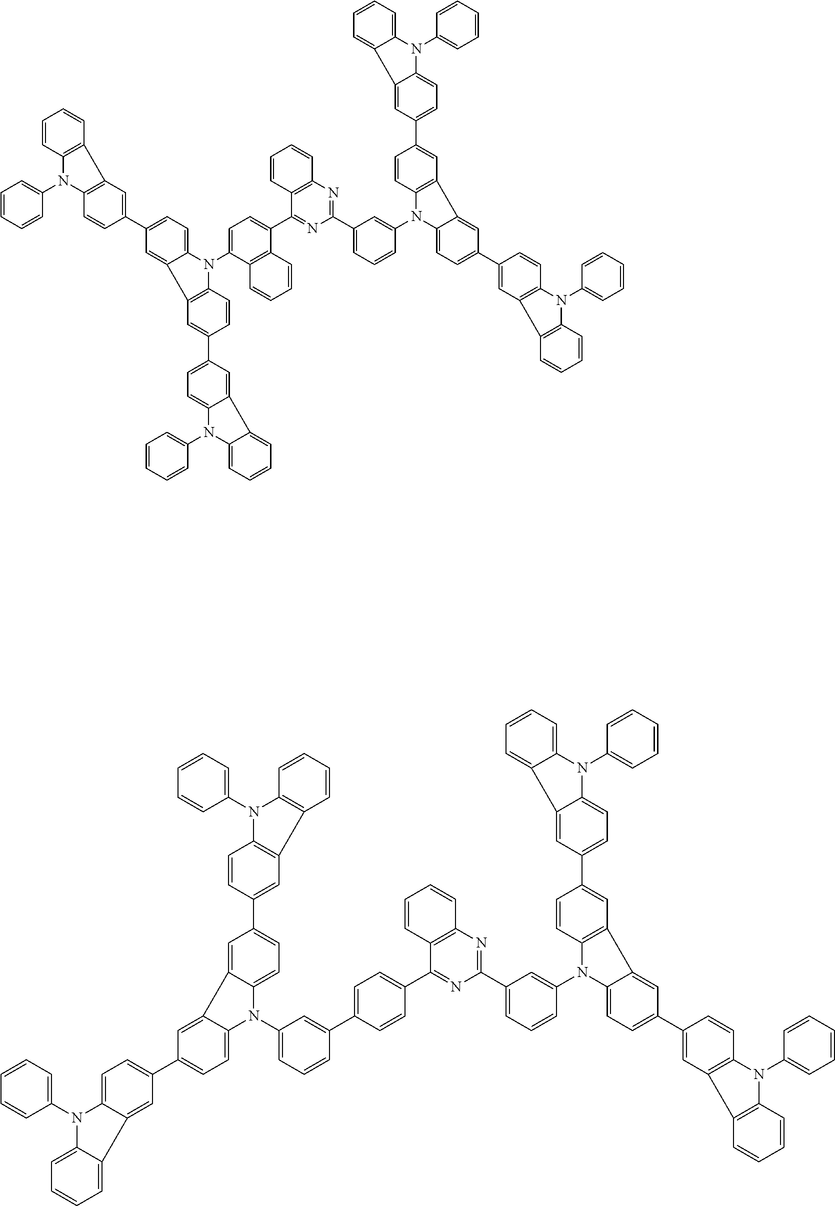 [SMILES: c1ccc(-n2c3ccccc3c3cc(-c4ccc5c(c4)c4cc(-c6ccc7c(c6)c6ccccc6n7-c6ccccc6)ccc4n5-c4cccc(-c5nc(-c6ccc(-n7c8ccc(-c9ccc%10c(c9)c9ccccc9n%10-c9ccccc9)cc8c8cc(-c9ccc%10c(c9)c9ccccc9n%10-c9ccccc9)ccc87)c7ccccc67)c6ccccc6n5)c4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4cccc(-c5ccc(-c6nc(-c7cccc(-n8c9ccc(-c%10ccc%11c(c%10)c%10ccccc%10n%11-c%10ccccc%10)cc9c9cc(-c%10ccc%11c(c%10)c%10ccccc%10n%11-c%10ccccc%10)ccc98)c7)nc7ccccc67)cc5)c4)ccc32)cc1]

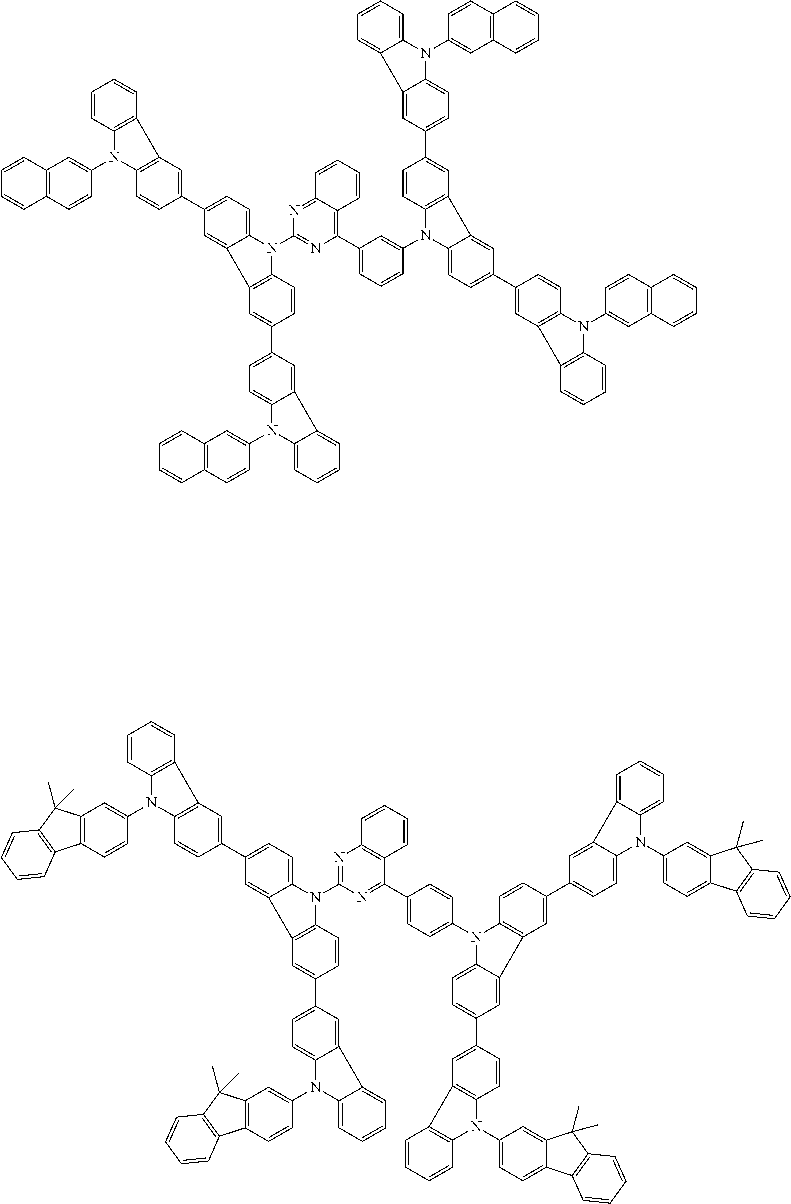

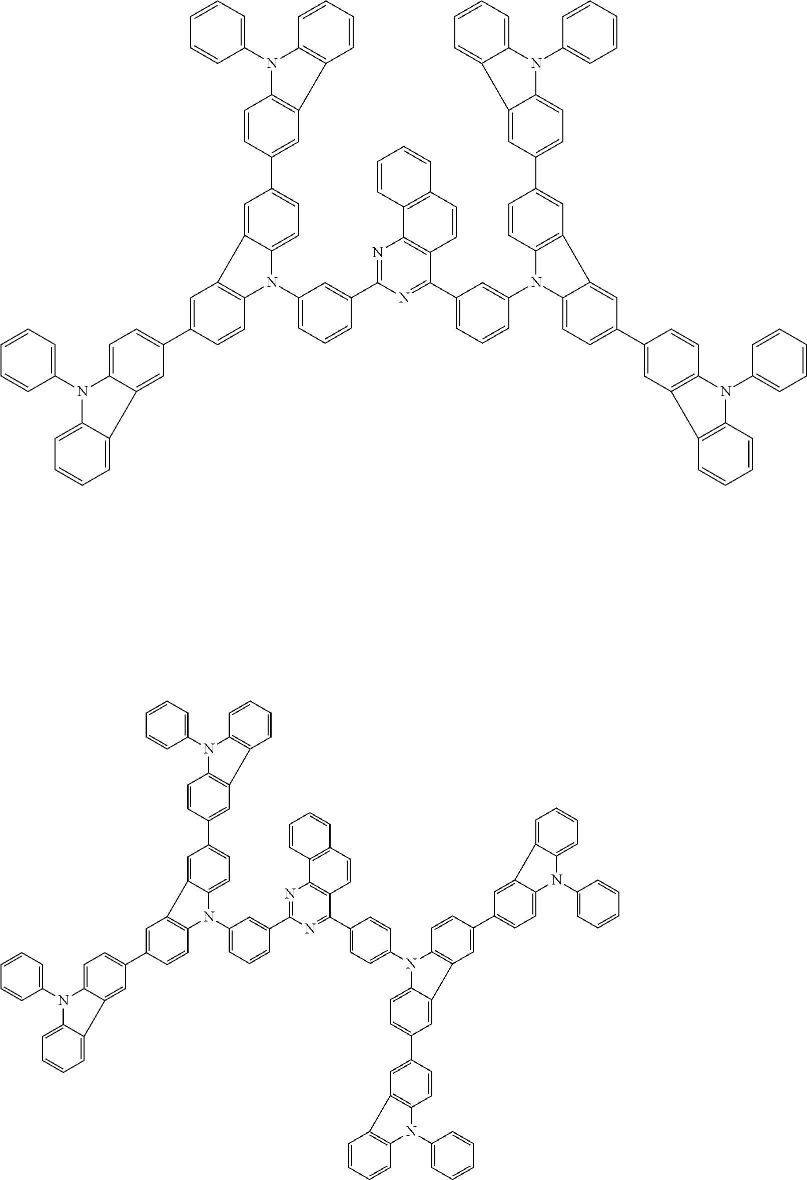 [SMILES: c1ccc(-n2c3ccccc3c3cc(-c4ccc5c(c4)c4cc(-c6ccc7c(c6)c6ccccc6n7-c6ccccc6)ccc4n5-c4cccc(-c5nc(-c6cccc(-n7c8ccc(-c9ccc%10c(c9)c9ccccc9n%10-c9ccccc9)cc8c8cc(-c9ccc%10c(c9)c9ccccc9n%10-c9ccccc9)ccc87)c6)c6ccc7ccccc7c6n5)c4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccc(-c5nc(-c6cccc(-n7c8ccc(-c9ccc%10c(c9)c9ccccc9n%10-c9ccccc9)cc8c8cc(-c9ccc%10c(c9)c9ccccc9n%10-c9ccccc9)ccc87)c6)nc6c5ccc5ccccc56)cc4)ccc32)cc1]

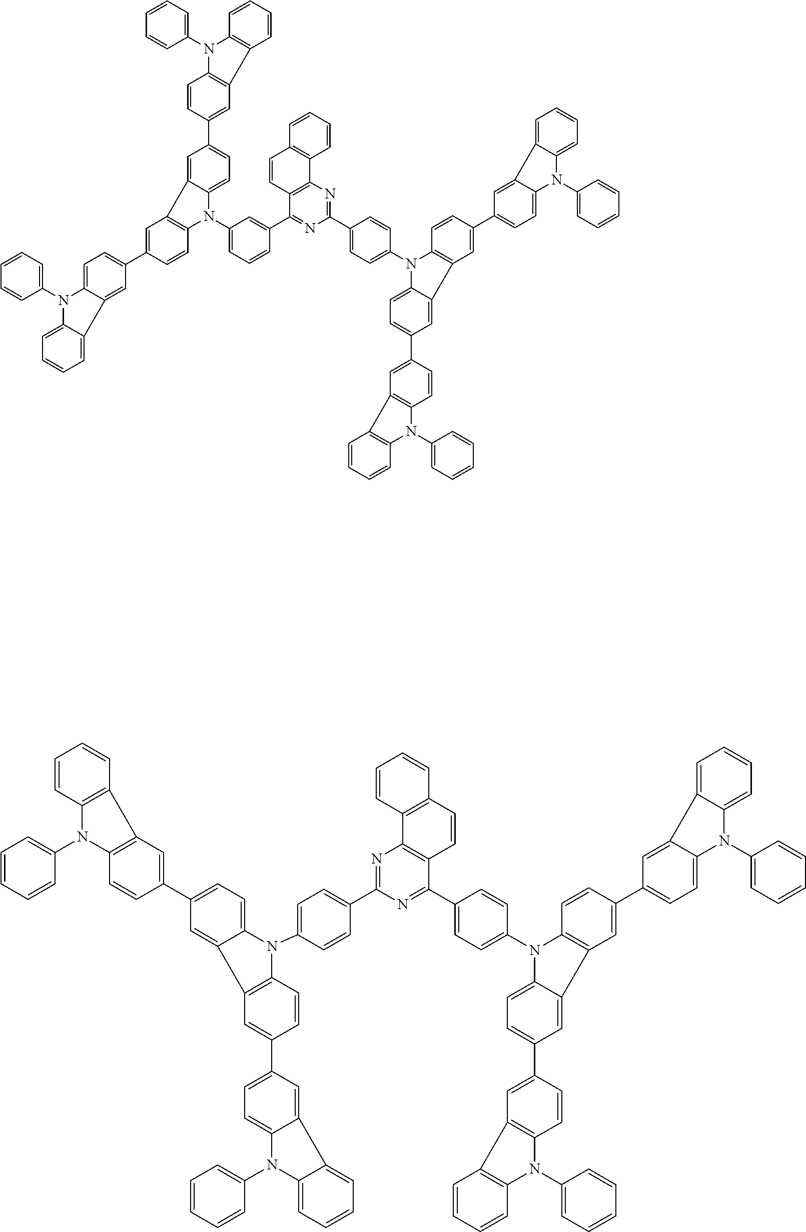 [SMILES: c1ccc(-n2c3ccccc3c3cc(-c4ccc5c(c4)c4cc(-c6ccc7c(c6)c6ccccc6n7-c6ccccc6)ccc4n5-c4ccc(-c5nc(-c6cccc(-n7c8ccc(-c9ccc%10c(c9)c9ccccc9n%10-c9ccccc9)cc8c8cc(-c9ccc%10c(c9)c9ccccc9n%10-c9ccccc9)ccc87)c6)c6ccc7ccccc7c6n5)cc4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccc(-c5nc(-c6ccc(-n7c8ccc(-c9ccc%10c(c9)c9ccccc9n%10-c9ccccc9)cc8c8cc(-c9ccc%10c(c9)c9ccccc9n%10-c9ccccc9)ccc87)cc6)c6ccc7ccccc7c6n5)cc4)ccc32)cc1]

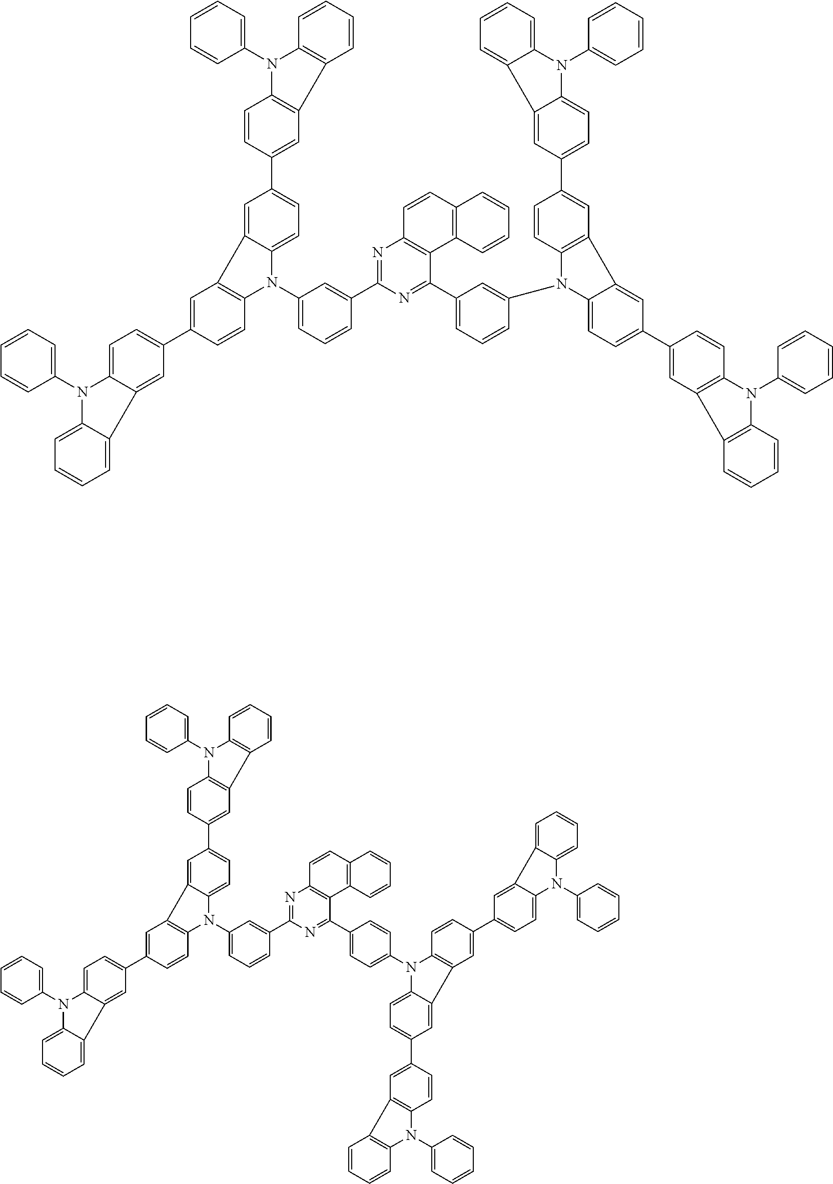 [SMILES: c1ccc(-n2c3ccccc3c3cc(-c4ccc5c(c4)c4cc(-c6ccc7c(c6)c6ccccc6n7-c6ccccc6)ccc4n5-c4cccc(-c5nc(-c6cccc(-n7c8ccc(-c9ccc%10c(c9)c9ccccc9n%10-c9ccccc9)cc8c8cc(-c9ccc%10c(c9)c9ccccc9n%10-c9ccccc9)ccc87)c6)c6c(ccc7ccccc76)n5)c4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccc(-c5nc(-c6cccc(-n7c8ccc(-c9ccc%10c(c9)c9ccccc9n%10-c9ccccc9)cc8c8cc(-c9ccc%10c(c9)c9ccccc9n%10-c9ccccc9)ccc87)c6)nc6ccc7ccccc7c56)cc4)ccc32)cc1]

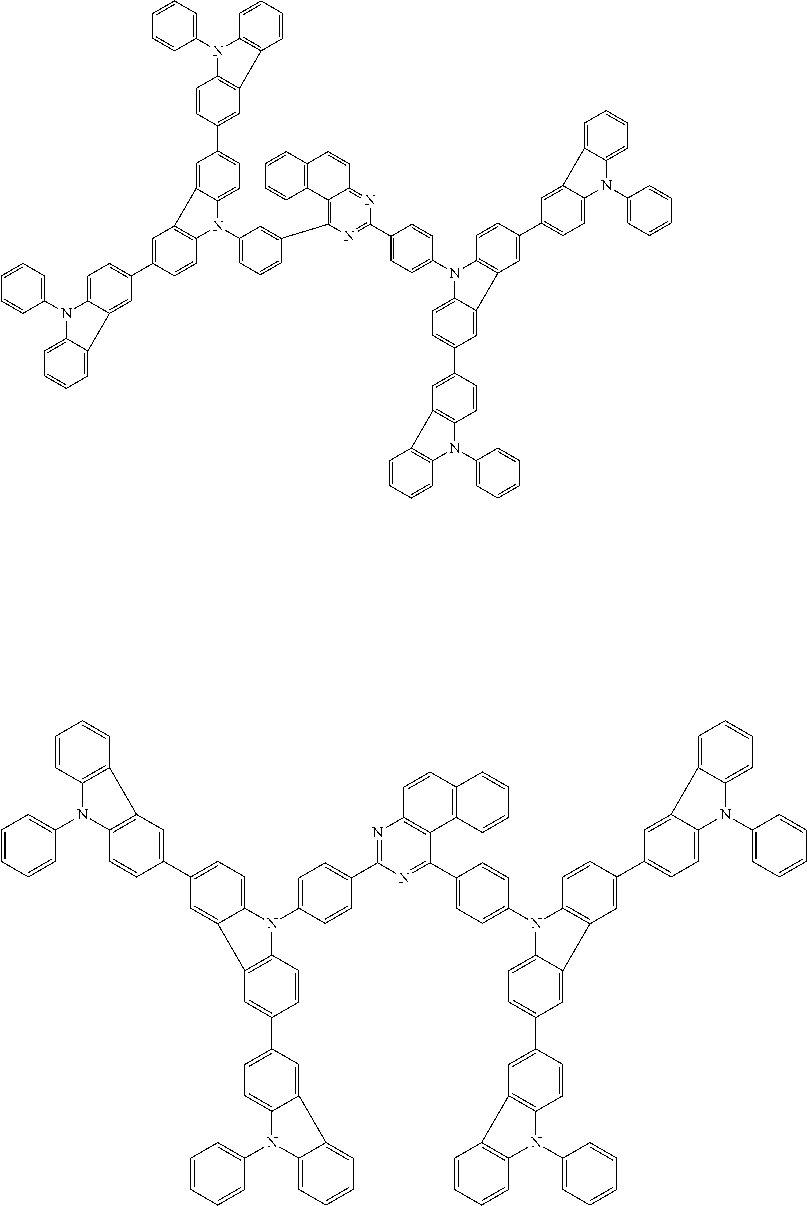 [SMILES: c1ccc(-n2c3ccccc3c3cc(-c4ccc5c(c4)c4cc(-c6ccc7c(c6)c6ccccc6n7-c6ccccc6)ccc4n5-c4ccc(-c5nc(-c6cccc(-n7c8ccc(-c9ccc%10c(c9)c9ccccc9n%10-c9ccccc9)cc8c8cc(-c9ccc%10c(c9)c9ccccc9n%10-c9ccccc9)ccc87)c6)c6c(ccc7ccccc76)n5)cc4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccc(-c5nc(-c6ccc(-n7c8ccc(-c9ccc%10c(c9)c9ccccc9n%10-c9ccccc9)cc8c8cc(-c9ccc%10c(c9)c9ccccc9n%10-c9ccccc9)ccc87)cc6)c6c(ccc7ccccc76)n5)cc4)ccc32)cc1]

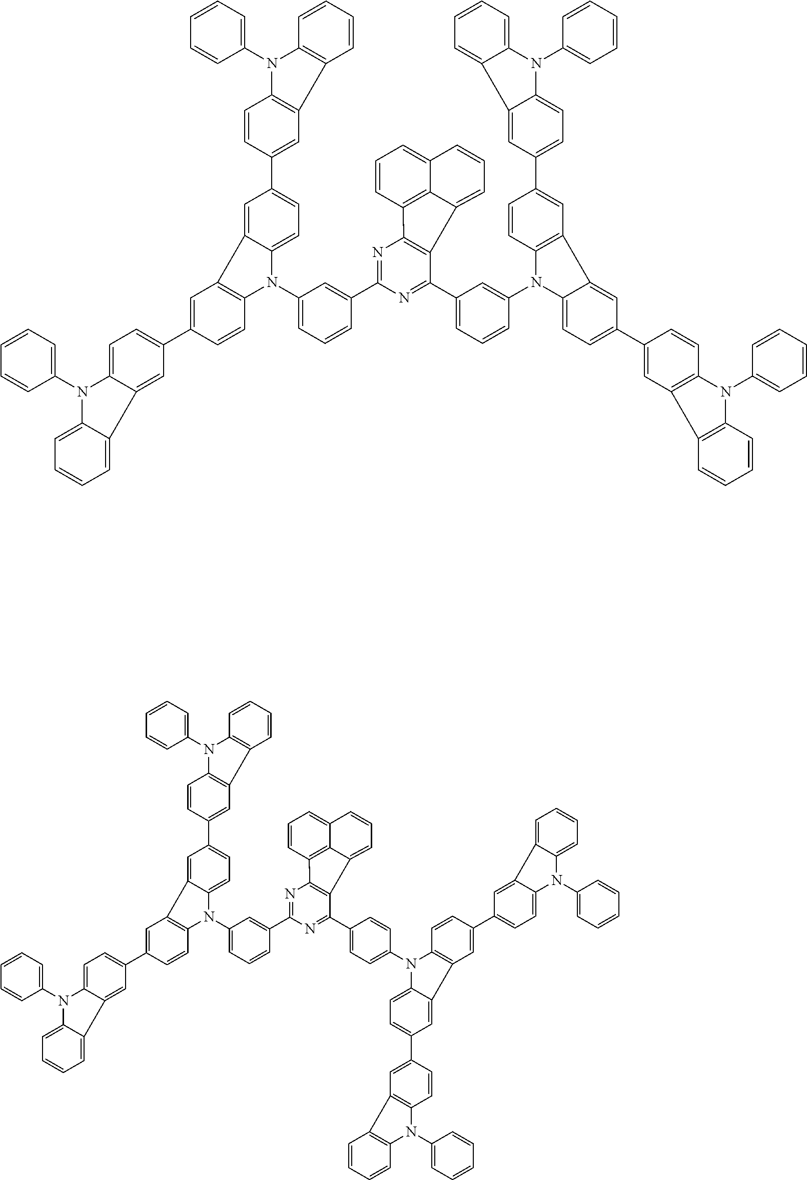 [SMILES: c1ccc(-n2c3ccccc3c3cc(-c4ccc5c(c4)c4cc(-c6ccc7c(c6)c6ccccc6n7-c6ccccc6)ccc4n5-c4cccc(-c5nc(-c6cccc(-n7c8ccc(-c9ccc%10c(c9)c9ccccc9n%10-c9ccccc9)cc8c8cc(-c9ccc%10c(c9)c9ccccc9n%10-c9ccccc9)ccc87)c6)c6c(n5)-c5cccc7cccc-6c57)c4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccc(-c5nc(-c6cccc(-n7c8ccc(-c9ccc%10c(c9)c9ccccc9n%10-c9ccccc9)cc8c8cc(-c9ccc%10c(c9)c9ccccc9n%10-c9ccccc9)ccc87)c6)nc6c5-c5cccc7cccc-6c57)cc4)ccc32)cc1]

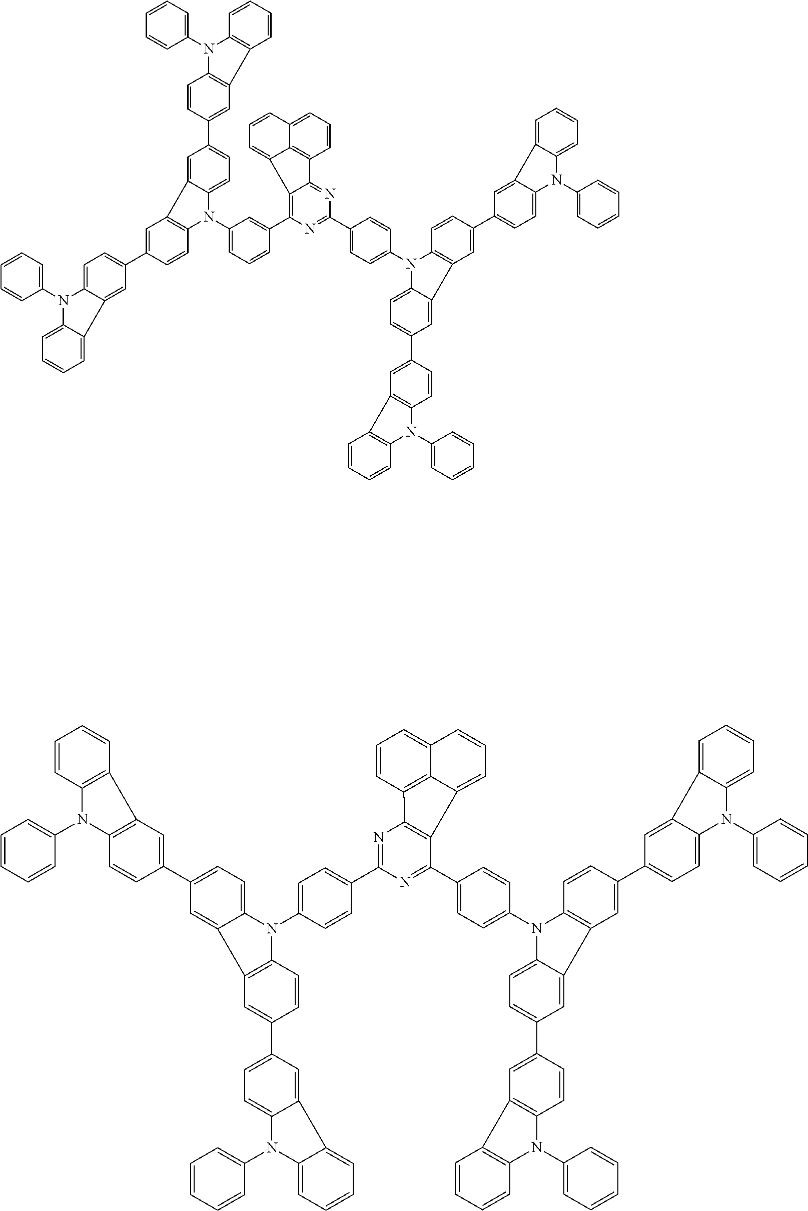 [SMILES: c1ccc(-n2c3ccccc3c3cc(-c4ccc5c(c4)c4cc(-c6ccc7c(c6)c6ccccc6n7-c6ccccc6)ccc4n5-c4ccc(-c5nc(-c6cccc(-n7c8ccc(-c9ccc%10c(c9)c9ccccc9n%10-c9ccccc9)cc8c8cc(-c9ccc%10c(c9)c9ccccc9n%10-c9ccccc9)ccc87)c6)c6c(n5)-c5cccc7cccc-6c57)cc4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccc(-c5nc(-c6ccc(-n7c8ccc(-c9ccc%10c(c9)c9ccccc9n%10-c9ccccc9)cc8c8cc(-c9ccc%10c(c9)c9ccccc9n%10-c9ccccc9)ccc87)cc6)c6c(n5)-c5cccc7cccc-6c57)cc4)ccc32)cc1]

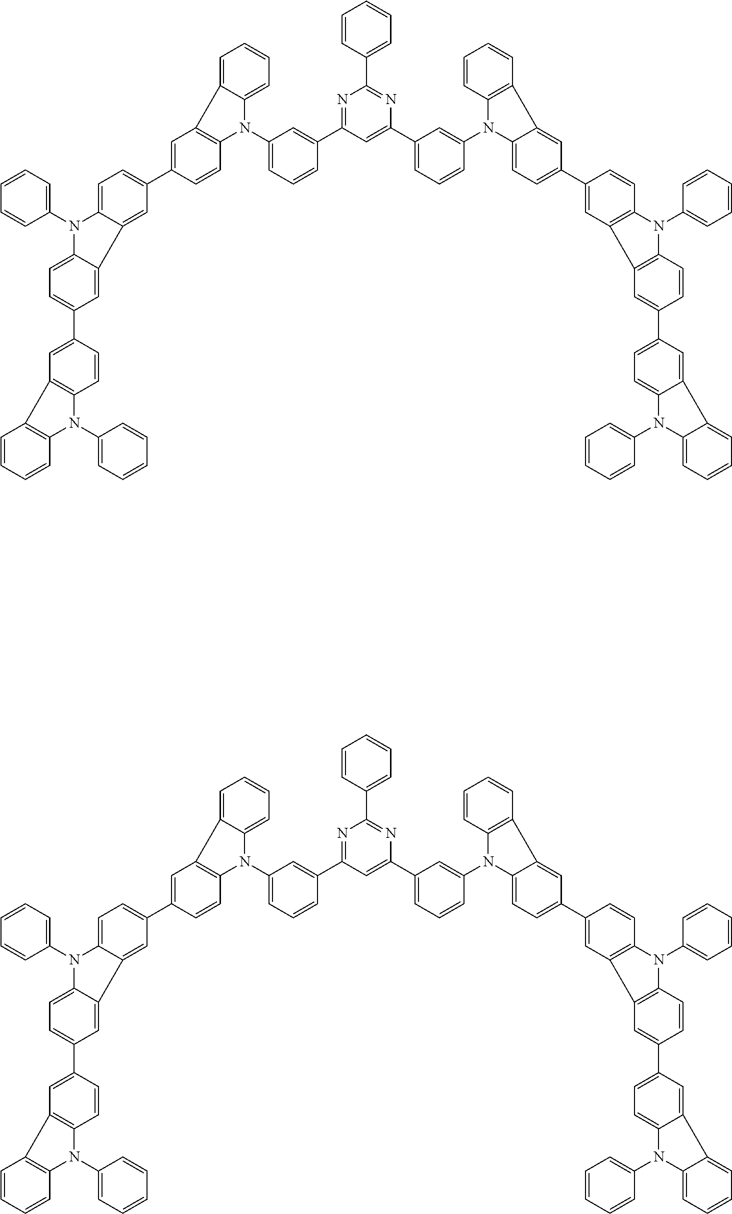 [SMILES: c1ccc(-c2nc(-c3cccc(-n4c5ccccc5c5cc(-c6ccc7c(c6)c6cc(-c8ccc9c(c8)c8ccccc8n9-c8ccccc8)ccc6n7-c6ccccc6)ccc54)c3)cc(-c3cccc(-n4c5ccccc5c5cc(-c6ccc7c(c6)c6cc(-c8ccc9c(c8)c8ccccc8n9-c8ccccc8)ccc6n7-c6ccccc6)ccc54)c3)n2)cc1 ; c1ccc(-c2nc(-c3cccc(-n4c5ccccc5c5cc(-c6ccc7c(c6)c6cc(-c8ccc9c(c8)c8ccccc8n9-c8ccccc8)ccc6n7-c6ccccc6)ccc54)c3)cc(-c3cccc(-n4c5ccccc5c5cc(-c6ccc7c(c6)c6cc(-c8ccc9c(c8)c8ccccc8n9-c8ccccc8)ccc6n7-c6ccccc6)ccc54)c3)n2)cc1]

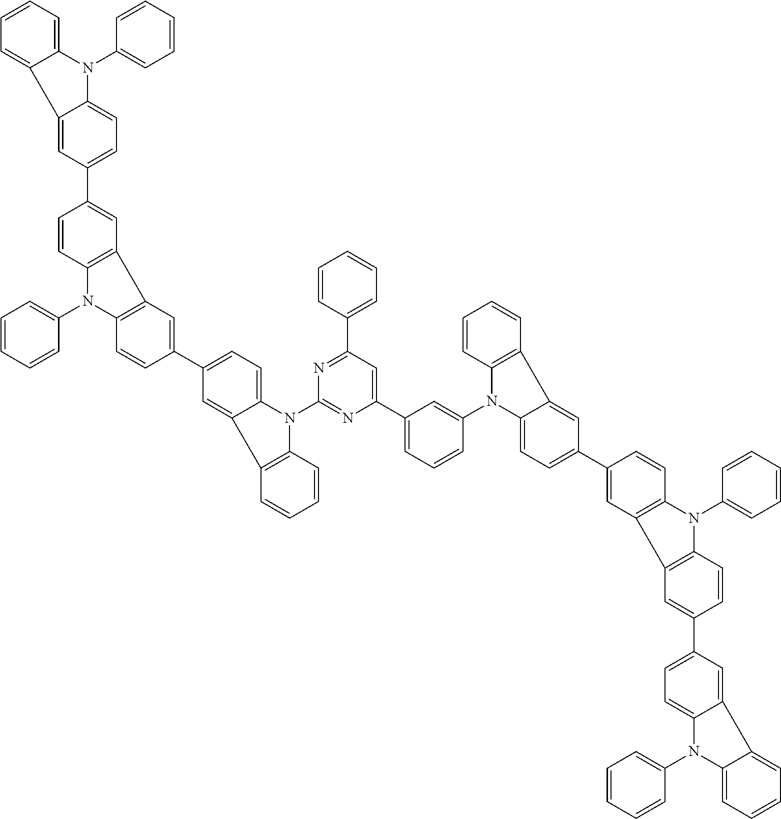 [SMILES: c1ccc(-c2cc(-c3cccc(-n4c5ccccc5c5cc(-c6ccc7c(c6)c6cc(-c8ccc9c(c8)c8ccccc8n9-c8ccccc8)ccc6n7-c6ccccc6)ccc54)c3)nc(-n3c4ccccc4c4cc(-c5ccc6c(c5)c5cc(-c7ccc8c(c7)c7ccccc7n8-c7ccccc7)ccc5n6-c5ccccc5)ccc43)n2)cc1]

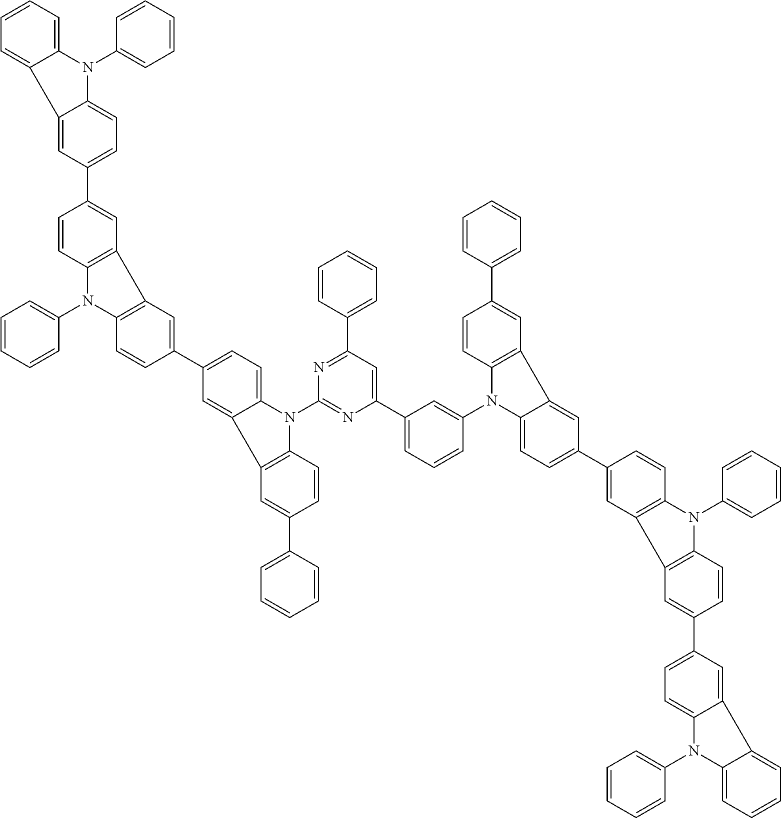 [SMILES: c1ccc(-c2ccc3c(c2)c2cc(-c4ccc5c(c4)c4cc(-c6ccc7c(c6)c6ccccc6n7-c6ccccc6)ccc4n5-c4ccccc4)ccc2n3-c2cccc(-c3cc(-c4ccccc4)nc(-n4c5ccc(-c6ccccc6)cc5c5cc(-c6ccc7c(c6)c6cc(-c8ccc9c(c8)c8ccccc8n9-c8ccccc8)ccc6n7-c6ccccc6)ccc54)n3)c2)cc1]

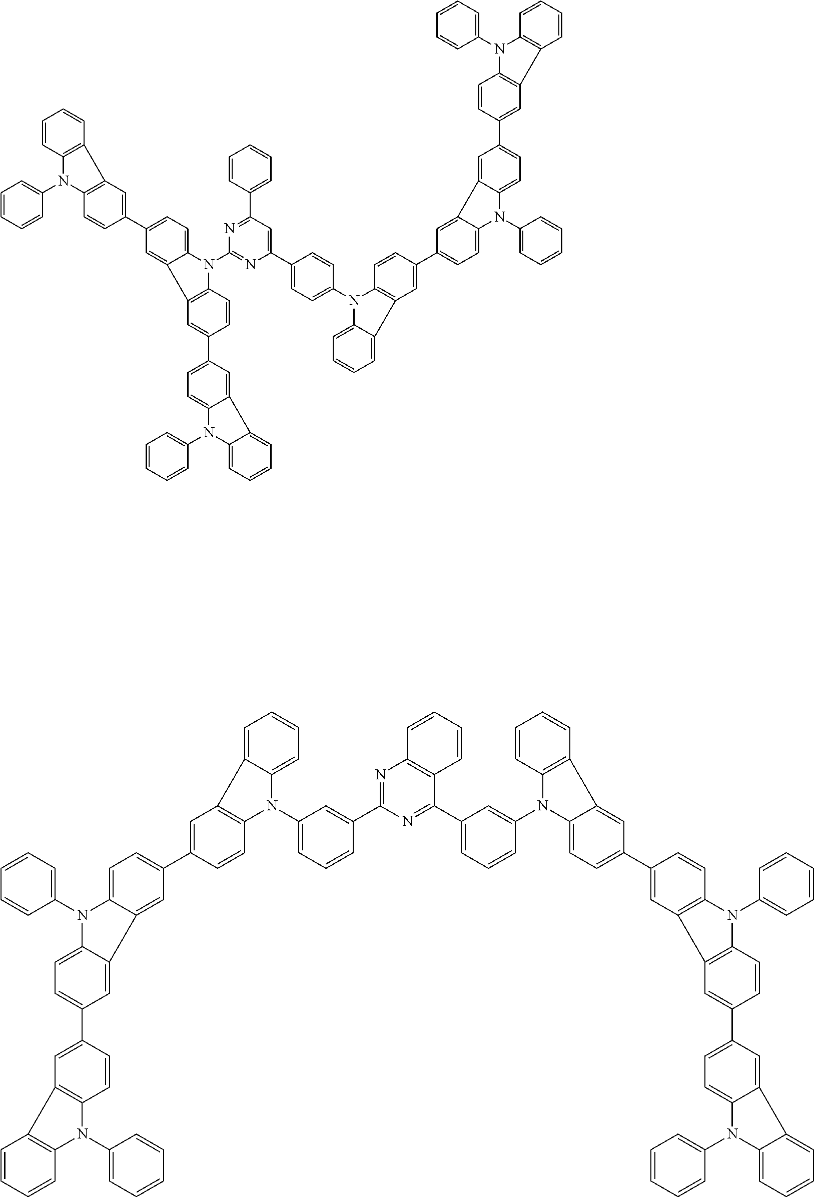 [SMILES: c1ccc(-c2cc(-c3ccc(-n4c5ccccc5c5cc(-c6ccc7c(c6)c6cc(-c8ccc9c(c8)c8ccccc8n9-c8ccccc8)ccc6n7-c6ccccc6)ccc54)cc3)nc(-n3c4ccc(-c5ccc6c(c5)c5ccccc5n6-c5ccccc5)cc4c4cc(-c5ccc6c(c5)c5ccccc5n6-c5ccccc5)ccc43)n2)cc1 ; c1ccc(-n2c3ccccc3c3cc(-c4ccc5c(c4)c4cc(-c6ccc7c(c6)c6ccccc6n7-c6cccc(-c7nc(-c8cccc(-n9c%10ccccc%10c%10cc(-c%11ccc%12c(c%11)c%11cc(-c%13ccc%14c(c%13)c%13ccccc%13n%14-c%13ccccc%13)ccc%11n%12-c%11ccccc%11)ccc%109)c8)c8ccccc8n7)c6)ccc4n5-c4ccccc4)ccc32)cc1]

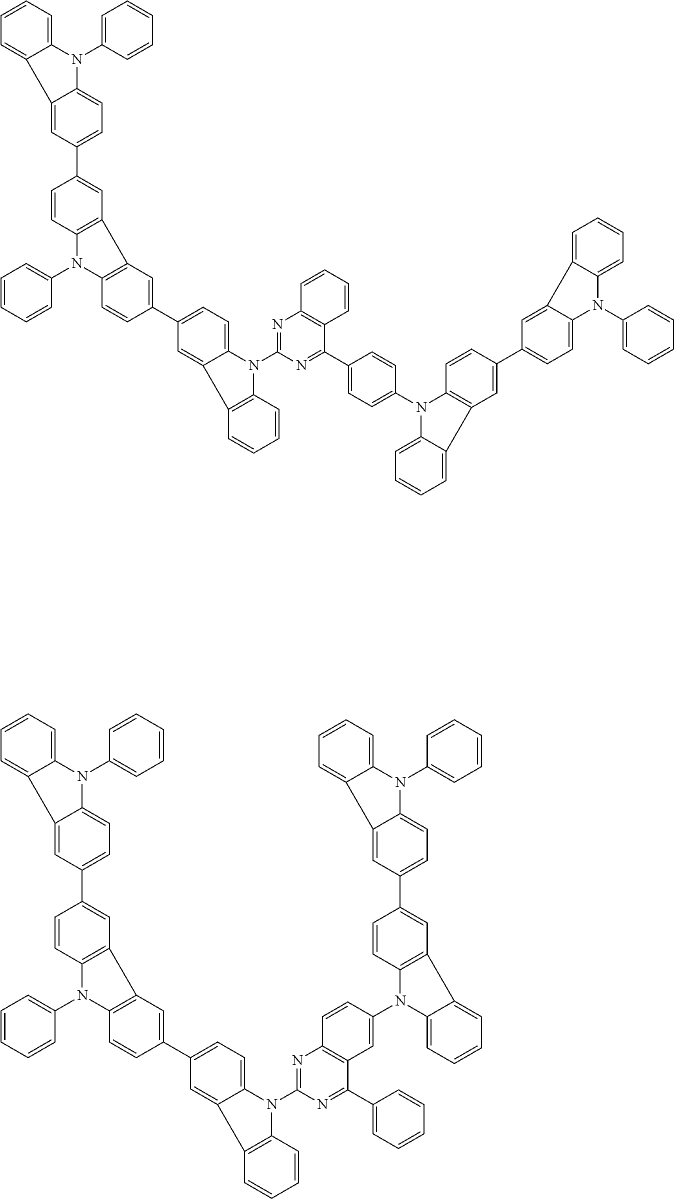 [SMILES: c1ccc(-n2c3ccccc3c3cc(-c4ccc5c(c4)c4ccccc4n5-c4ccc(-c5nc(-n6c7ccccc7c7cc(-c8ccc9c(c8)c8cc(-c%10ccc%11c(c%10)c%10ccccc%10n%11-c%10ccccc%10)ccc8n9-c8ccccc8)ccc76)nc6ccccc56)cc4)ccc32)cc1 ; c1ccc(-c2nc(-n3c4ccccc4c4cc(-c5ccc6c(c5)c5cc(-c7ccc8c(c7)c7ccccc7n8-c7ccccc7)ccc5n6-c5ccccc5)ccc43)nc3ccc(-n4c5ccccc5c5cc(-c6ccc7c(c6)c6ccccc6n7-c6ccccc6)ccc54)cc23)cc1]

### [Material for Organic Electroluminescence Devices]

The material for organic electroluminescence devices of one aspect of the present invention is described below. (Hereinafter the “material for organic electroluminescence devices” may be abbreviated as the “material for organic EL devices”.)

The material for organic EL devices of one aspect of the present invention includes the compound (1). The compound (1) is useful as a material in organic EL devices.

The content of the compound (1) in the material for organic EL devices may be 1% by mass or more, preferably 10% by mass or more, more preferably 50% by mass or more, even more preferably 80% by mass or more, and especially preferably 90% by mass or more.

The material for organic EL devices of one aspect of the present invention may be used, for example, as a host material or a dopant material in the light emitting layer in a fluorescent emitting unit, or may be used as a host material in the light emitting layer in a phosphorescent emitting unit. In this case, the light emitting layer contains the material for organic EL devices of one aspect of the present invention and a fluorescent light emitting material or a phosphorescent light emitting material. In any of the fluorescent emitting unit or the phosphorescent emitting unit, the material for organic EL devices of one aspect of the present invention is useful as a material for the organic thin film layers on the anode side to be arranged between the anode and the light emitting layer in an organic EL device or as a material for the organic thin film layers on the cathode side to be arranged between the cathode and the light emitting layer of an organic EL device, or that is, as a material for the hole transporting layer, the hole injection layer, the electron transporting layer, the electron injection layer, the hole blocking layer, the electron blocking layer, etc.

Here, “light emitting unit” means a minimum unit containing one or more organic layers, in which one layer is a light emitting layer and the injected holes and electrons are recombined for light emission.

### [Ink Composition]

The ink composition of one aspect of the present invention is described below.

The ink composition of one aspect of the present invention contains a solvent and the compound (1) dissolved in the solvent. The ink composition of one aspect of the present invention may be used for forming an organic thin layer to constitute an organic EL device.

The ink composition of one aspect of the present invention may contain additives such as a hole transporting material, an electron transporting material, a light emitting material, an acceptor material, a stabilizer and the like, in addition to the compound (1).

The ink composition of one aspect of the present invention ma contain additives for controlling viscosity and/or surface tension, for example, a viscosity improver (high-molecular-weight compound, etc.), a viscosity depressant (low-molecular-weight compound, etc.), a surfactant, etc. In addition, for improving the storage stability thereof, the ink composition may contain an antioxidant not having any influence on organic EL devices, such as a phenolic antioxidant, a phosphorus-containing antioxidant, etc.

The content of the compound (1) in the ink composition is preferably 0.1 to 15% by mass, more preferably 0.5 to 10% by mass.

The high-molecular-weight compound usable as a viscosity improver includes insulating resins such as polystyrene, polycarbonate, polyarylate, polyester, polyamide, polyurethane, polysulfone, polymethyl methacrylate, polymethyl acrylate, cellulose, and their copolymers, etc.; photoconductive resins such as poly-N-vinylcarbazole, polysilane, etc.; electroconductive resins such as polythiophene, polypyrrole, etc.

Examples of the solvent include chlorine-containing solvents such as chloroform, methylene chloride, 1,2-dichloroethane, 1, 1,2-trichloroethane, chlorobenzene, o-dichlorobenzene, etc.; ether solvents such as tetrahydrofuran, dioxane, dioxolane, anisole, etc.; aromatic hydrocarbon solvents such as toluene, xylene, etc.; aliphatic hydrocarbon solvents such as cyclohexane, methylcyclohexane, n-pentane, n-hexane, n-heptane, n-octane, n-nonane, n-decane, etc.; ketone solvents such as acetone, methyl ethyl ketone, cyclohexane, benzophenone, acetophenone, etc.; ester solvents such as ethyl acetate, butyl acetate, ethyl cellosolve acetate, methyl benzoate, phenyl acetate, etc.; polyalcohols and derivatives thereof such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, 12,-hexanediol, etc.; alcohol solvents such as methanol, ethanol, propanol, isopropanol, cyclohexanol, etc.; sulfoxide solvents such as dimethyl sulfoxide, etc.; amide solvents such as N-methyl-2-pyrrolidone, N,N-dimethylformamide, etc. One alone or two or more of these solvents may be used either singly or as combined.

Among these solvents, preferred are aromatic hydrocarbon solvents, ether solvents, aliphatic hydrocarbon solvents, ester solvents and ketone solvents, from the viewpoint of solubility, uniformity in film formation, viscosity characteristics, etc. More preferred are toluene, xylene, ethylbenzene, diethylbenzene, trimethylbenzene, n-propylbenzene, isopropylbenzene, n-butylbenzene, isobutylbenzene, 5-butylbenzene, n-hexylbenzene, cyclohexylbenzene, 1-methylnaphthalene, tetralin, 1,3-dioxane, 1,4-dioxane, 1,3-dioxolane, anisole, ethoxybenzene, cyclohexane, bicyclohexyl, cyclohexenylcyclohexanone, n-heptylcyclohexane, n-hexylcyclohexane, decalin, methyl benzoate, cyclohexanone, 2-propylcyclohexanone, 2-heptanone, 3-heptanone, 4-heptanone, 2-octanone, 2-nonanone, 2-decanone, dicyclohexyl ketone, acetophenone, benzophenone.

### [Organic Electroluminescence Device]

The organic electroluminescence device (hereinafter “organic electroluminescence device” may be abbreviated as “organic EL device”) of one aspect of the present invention is described.

The organic EL device of one aspect of the present invention includes a cathode, an anode, and one or more organic thin film layer including a light emitting layer between the cathode and the anode, wherein at least one of the one or more organic thin film layers contains the compound (1).

Examples of the organic thin film layers containing the compound (1) include, though not limited thereto, an anode-side organic thin film layers formed between an anode and a light emitting layer (i.e., a hole transporting layer, a hole injection layer, etc.), a light emitting layer, cathode-side organic thin film layers formed between a cathode and the light emitting layer (i.e., an electron transporting layer, an electron injection layer, etc.), a space layer, a blocking layer, etc. The compound (1) may be contained in any of the above-mentioned layers, and for example, may be used as a host material or a dopant material in the light emitting layer in a fluorescent emitting unit, or a host material in the light emitting layer in a phosphorescent emitting unit, or in a hole transporting layer, an electron transporting layer and the like in a light emitting unit, etc.

The organic EL device of one aspect of the present invention may be any of a single color emitting device of fluorescent or phosphorescent type, a white-emitting device of fluorescent-phosphorescent hybrid type, an emitting device of a simple type having a single light emitting unit, and an emitting device of a tandem type having two or more light emitting units, with the phosphorescent device being preferred. The “light emitting unit” referred to herein is the smallest unit for emitting light by the recombination of injected holes and injected electrons, which contains one or more organic layers wherein at least one layer is a light emitting layer.

Accordingly, representative device structures of the simple-type organic EL device are shown below.

### (1) Anode/Light Emitting Unit/Cathode

The light emitting unit may be a laminate containing two or more layers of phosphorescent light emitting layers and fluorescent light emitting layers. In the case, a space layer may be disposed between the light emitting layers to prevent the diffusion of excitons generated in the phosphorescent light emitting layer into the fluorescent light emitting layer. Representative layered structures of the light emitting unit are shown below.

(a) hole transporting layer/light emitting layer (/electron transporting layer);
(b) hole transporting layer/first phosphorescent light emitting layer/second phosphorescent light emitting layer (/electron transporting layer);
(c) hole transporting layer/phosphorescent light emitting layer/space layer/fluorescent light emitting layer (/electron transporting layer);
(d) hole transporting layer/first phosphorescent light emitting layer/second phosphorescent light emitting layer/space layer/fluorescent light emitting layer (/electron transporting layer);
(e) hole transporting layer/first phosphorescent light emitting layer/space layer/second phosphorescent light emitting layer/space layer/fluorescent light emitting layer (/electron transporting layer);
(f) hole transporting layer/phosphorescent light emitting layer/space layer/first fluorescent light emitting layer/second fluorescent light emitting layer (/electron transporting layer);
(g) hole transporting layer/electron blocking layer/light emitting layer (/electron transporting layer);
(h) hole transporting layer/light emitting layer/hole blocking layer (/electron transporting layer); and
(i) hole transporting layer/fluorescent light emitting layer/triplet blocking layer (/electron transporting layer).

The above-mentioned phosphorescent or fluorescent light emitting layers may emit different colors. For example, the layered structure of the laminated light emitting layer (d) may have a layer configuration of hole transporting layer/first phosphorescent light emitting layer (red emission)/second phosphorescent light emitting layer (green emission)/space layer/fluorescent light emitting layer (blue emission)/electron transporting layer, etc.

An electron blocking layer may be disposed between the light emitting layer and the hole transporting layer or between the light emitting layer and the space layer, if necessary. Also, a hole blocking layer may be disposed between the light emitting layer and the electron transporting layer, if necessary. With such an electron blocking layer or a hole blocking layer, electrons and holes are confined in the light emitting layer to increase the degree of charge recombination in the light emitting layer, thereby improving the emission efficiency.

A representative device structure of the tandem-type organic EL device is shown below.

### (2) Anode/First Light Emitting Unit/Intermediate Layer/Second Light Emitting Unit/Cathode

Here, the first light emitting unit and the second light emitting unit may be independently selected from those described above with respect to the light emitting unit.

Generally, the intermediate layer is also called an intermediate electrode, an intermediate conductive layer, a charge generation layer, an electron withdrawing layer, a connecting layer, or an intermediate insulating layer. The intermediate layer may be formed by known materials so as to supply electrons to the first light emitting unit and holes to the second light emitting unit.

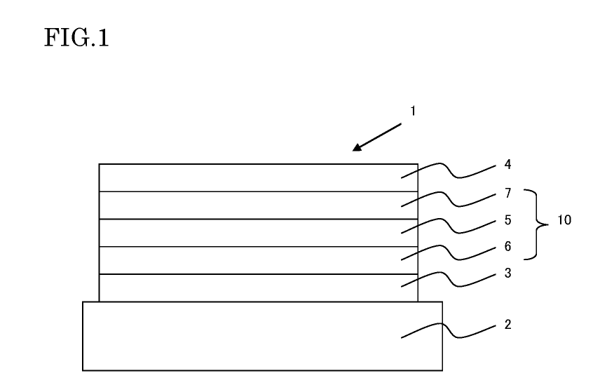

A schematic structure of an example of the organic EL device of one aspect of the invention is shown in FIG. 1, wherein the organic EL device 1 has a substrate 2, an anode 3, a cathode 4, and a light emitting unit 10 disposed between the anode 3 and the cathode 4. The light emitting unit 10 has, for example, a light emitting layer 5 which includes at least one phosphorescent emitting layer containing a phosphorescent host material and a phosphorescent dopant (phosphorescent light emitting material). A hole injecting/transporting layer (an anode-side organic thin film layer) 6 may be disposed between the light emitting layer 5 and the anode 3, and an electron injecting/transporting layer (a cathode-side organic thin film layer) 7 may be disposed between the light emitting layer 5 and the cathode 4. An electron blocking layer may be disposed on the anode 3 side of the light emitting layer 5, and a hole blocking layer may be disposed on the cathode 4 side of the light emitting layer 5. With these blocking layers, electrons and holes are confined in the light emitting layer 5 to increase the degree of exciton generation in the light emitting layer 5.

In this description, a host is referred to as a fluorescent host when combinedly used with a fluorescent dopant (fluorescent light emitting material) and referred to as a phosphorescent host when combinedly used with a phosphorescent dopant. Therefore, the fluorescent host and the phosphorescent host are not distinguished from each other merely by the difference in their molecular structures. Namely, in the present invention, the term phosphorescent host means a material for constituting a phosphorescent emitting layer containing a phosphorescent dopant and does not mean a material that cannot be used as a material for a fluorescent emitting layer. The same applies to the fluorescent host.

### (Substrate)

The organic EL device of one aspect of the present invention is formed on a light-transmissive substrate. The light-transmissive substrate serves as a support for the organic EL device and is preferably a flat substrate having a transmittance of 50% or more to 400 to 700 nm visible light. Examples of the substrate include a glass plate and a polymer plate. In particular, the glass plate may include a plate made of soda-lime glass, barium-strontium-containing glass, lead glass, aluminosilicate glass, borosilicate glass, barium borosilicate glass, or quartz. The polymer plate may include a plate made of polycarbonate, acryl, polyethylene terephthalate, polyether sulfide, or polysulfone.

### (Anode)

The anode of the organic EL devices plays a role of injecting holes into the hole transporting layer or the light emitting layer, and effectively has a work function of 4.5 eV or more. Examples of the material for anode include indium tin oxide alloy (ITO), tin oxide (NESA), indium zinc oxide alloy, gold, silver, platinum, and cupper. The anode is formed by making the electrode material into a thin film by a method, such as a vapor deposition method or a sputtering method. When getting the light emitted from the light emitting layer through the anode, the transmittance of the anode to visible light is preferably 10% or more. The sheet resistance of the anode is preferably several hundred Q/square or less. The film thickness of the anode depends upon the kind of the material thereof and is generally 10 nm to 1 μm, preferably 10 nm to 200 nm.

### (Cathode)

The cathode plays a role of injecting electrons into the electron injecting layer and the electron transporting layer or the light emitting layer, and is preferably formed of a material having a small work function. Examples of the cathode material include, but not limited to, indium, aluminum, magnesium, magnesium-indium alloy, magnesium-aluminum alloy, aluminum-lithium alloy, aluminum-scandium-lithium alloy, and magnesium-silver alloy. Like the anode, the cathode is also formed by making the material into a thin film by a method, such as a vapor deposition method and a sputtering method. If desired, an embodiment of taking out the emitted light through the cathode side may be employed.

### (Light Emitting Layer)

The light emitting layer is an organic layer having a light emitting function and contains a host material and a dopant material when a doping system is employed. In this, the major function of the host material is to promote the recombination of electrons and holes and confine excitons in the light emitting layer, and the dopant material has a function for efficient light emission from the recombined excitons.

In the case of a phosphorescent device, the function of the host material is to confine the excitons generated on the dopant in the light emitting layer.

Here, to control the carrier balance in the light emitting layer, the light emitting layer may employ a double host (host/co-host) layer, for example, by combinedly using an electron transporting host and a hole transporting host.

The light emitting layer may employ a double dopant layer, in which two or more kinds of dopant materials having high quantum yield are combinedly used and each dopant material emits light with its own color. For example, there is mentioned an embodiment of realizing yellow emission, in which a light emitting layer formed by co-depositing a host, a red-emitting dopant and a green-emitting dopant is used in common.

In the above-mentioned light emitting layer laminate, a laminate of two or more light emitting layers, electrons and holes are accumulated in the interface between the light emitting layers, and therefore, the recombination region is localized in the interface between the light emitting layers, to improve the quantum efficiency.

The easiness of hole injection to the light emitting layer and the easiness of electron injection to the light emitting layer may be different from each other. Also, the hole transporting ability and the electron transporting ability each being expressed by the mobility of holes and electrons in the light emitting layer may be different from each other.

The phosphorescent dopant (phosphorescent light emitting material) used in the light emitting layer is a compound capable of emitting light from the excited triplet state, and is preferably an organometallic complex containing at least one metal selected from Ir, Pt, Os, Au, Cu, Re, and Ru and a ligand, although not particularly limited thereto as long as capable of emitting light from the excited triplet state. The ligand is preferably ortho-metallated. In view of obtaining a high phosphorescent quantum yield and further improving the external quantum efficiency of light emitting devices, a metal complex containing a metal atom selected from Ir, Os, and Pt is preferred, with a metal complex, such as an iridium complex, an osmium complex and a platinum complex, particularly an ortho-metallated complex being more preferred, an iridium complex and a platinum complex being still more preferred, and an ortho-metallated iridium complex being particularly preferred.

The content of the phosphorescent dopant in the light emitting layer is not particularly limited and selected according to the use of the device, and is preferably 0.1 to 70% by mass, and more preferably 1 to 30% by mass. When the content of the phosphorescent dopant is 0.1% by mass or more, the amount of light emission is sufficient, and when 70% by mass or less, the concentration quenching can be avoided.

Preferred examples of the organometallic complex for the phosphorescent dopant are shown below.

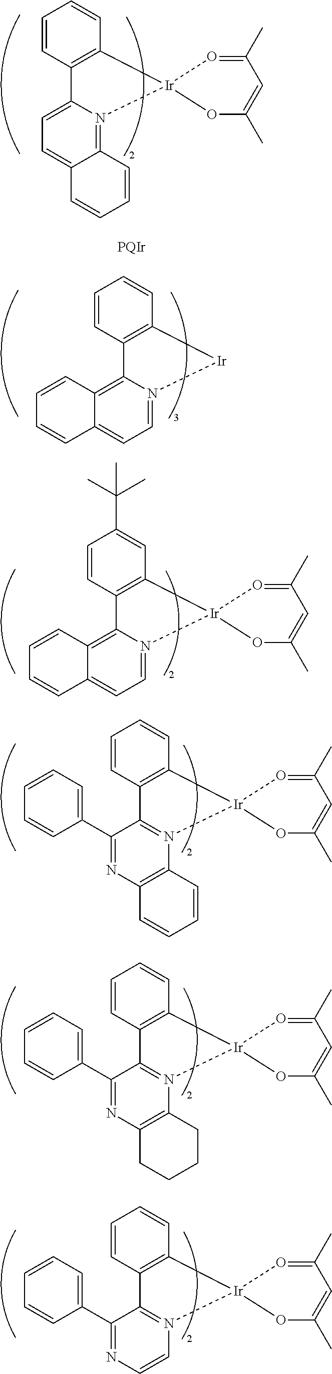

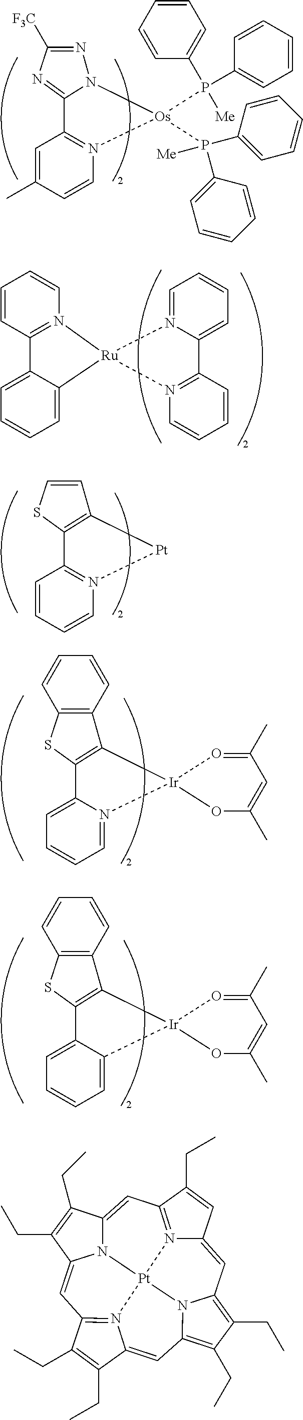

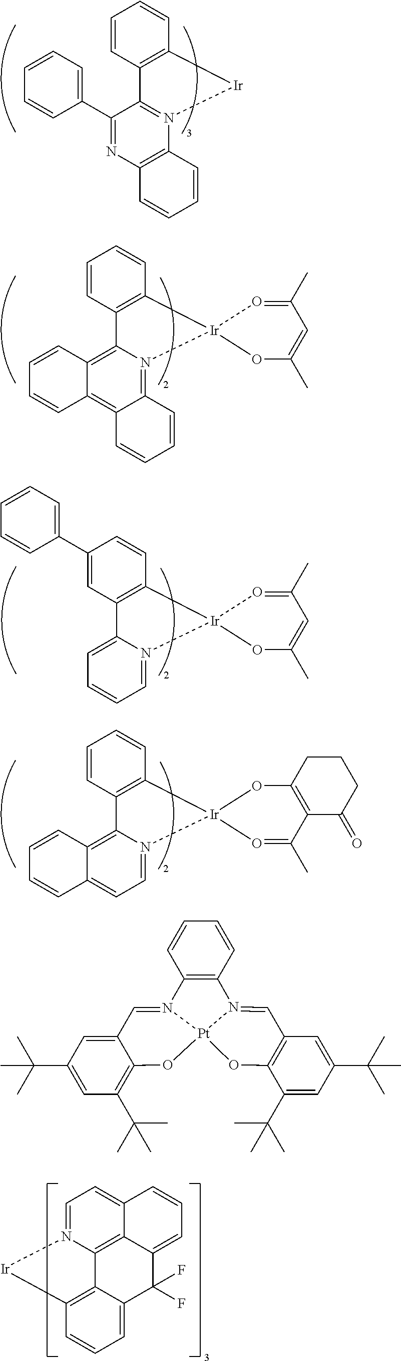

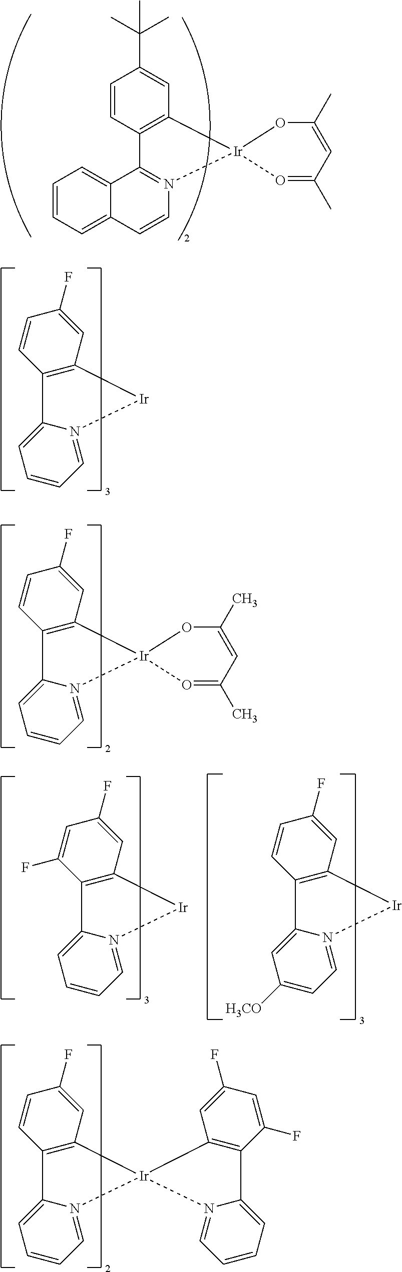

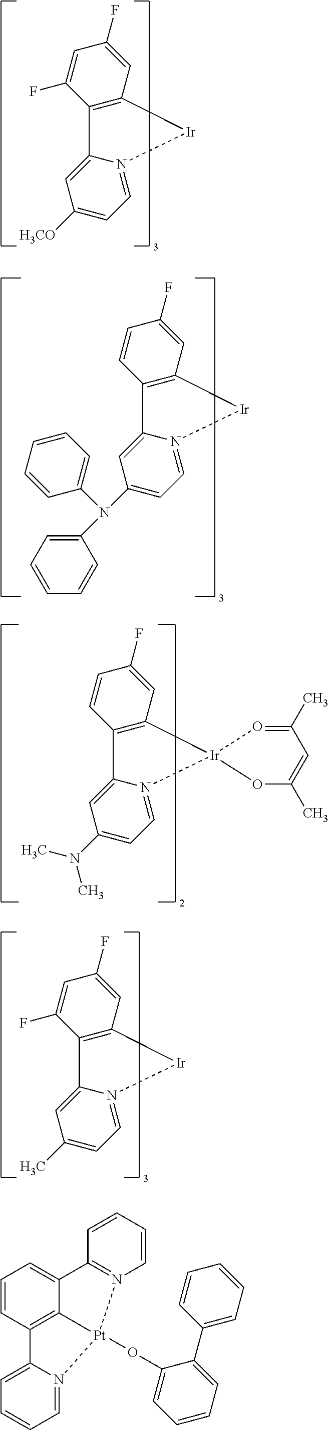

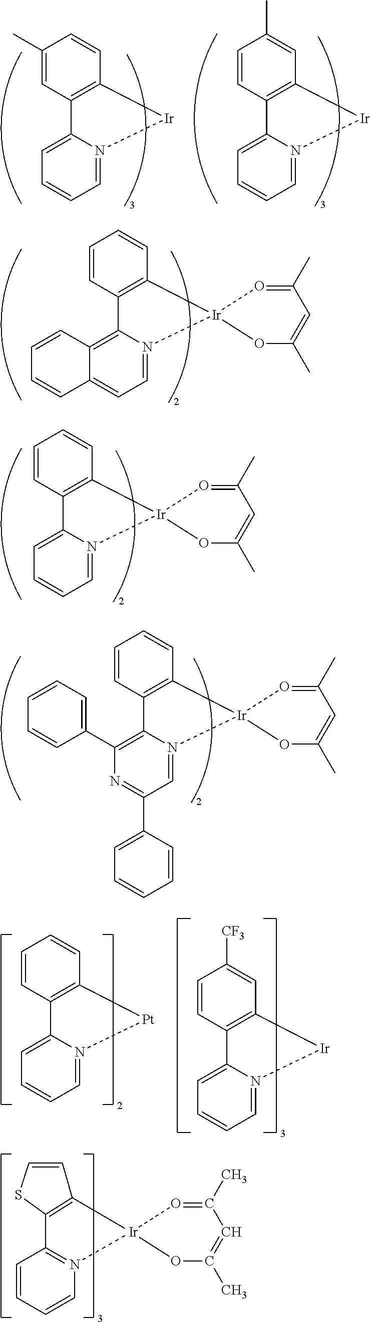

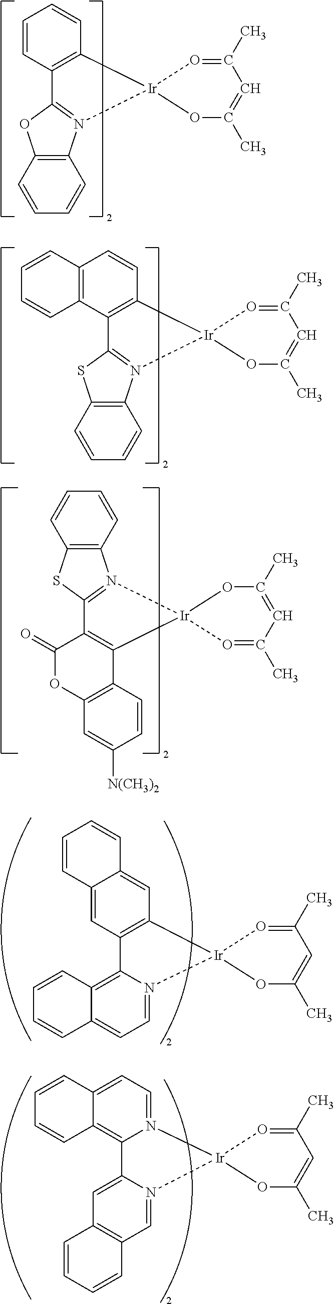

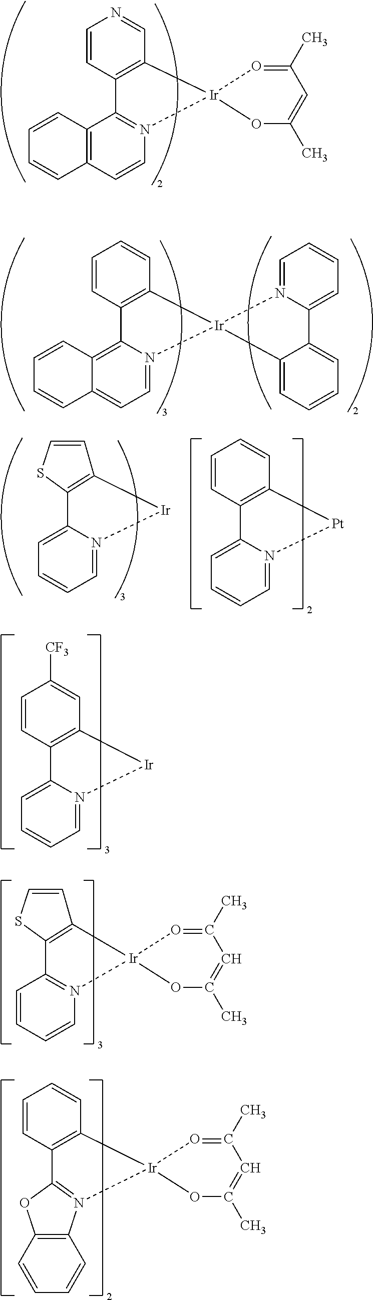

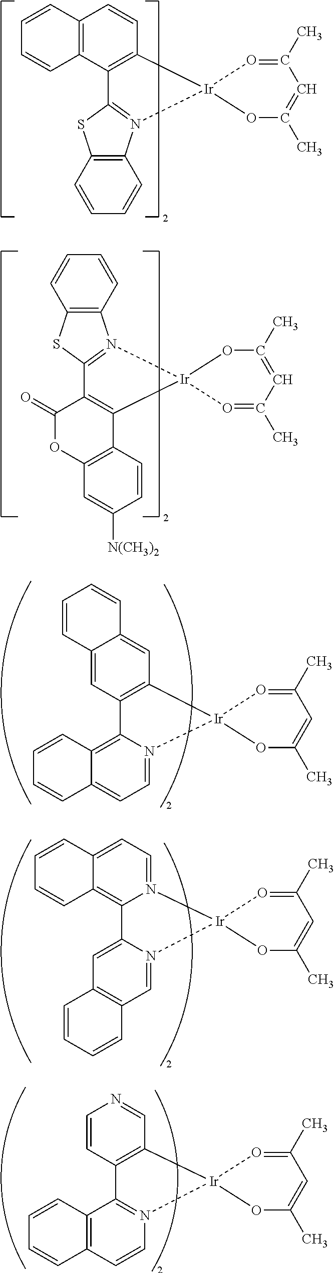

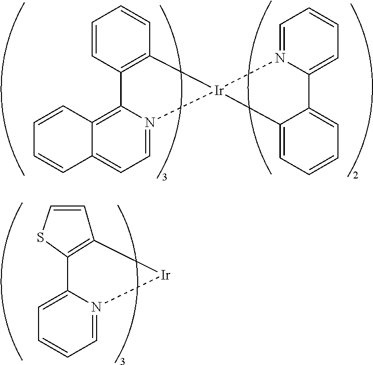

Further, in the organic EL device of one aspect of the present invention, a complex represented by the following formula (X) or (Y) is preferred as the phosphorescent light emitting material.

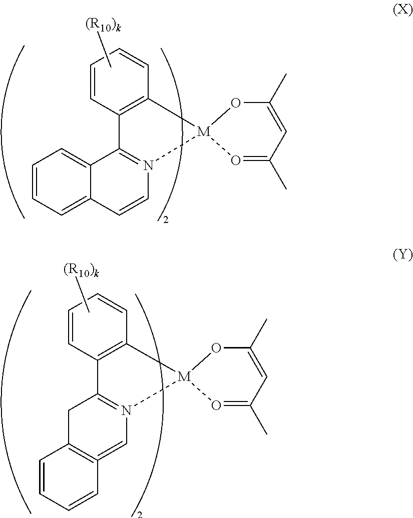

wherein R₁₀ represents a hydrogen atom or a substituent, k indicates an integer of 1 to 4. M represents Ir, Os or Pt.

The substituent that R₁₀ represents includes the same ones as those exemplified hereinabove for the substituents for R₀ to R₈ and others in the formula (1).

The phosphorescent host is a compound that has a function of confining the triplet energy of the phosphorescent dopant efficiently in the light emitting layer to cause the phosphorescent dopant to emit light efficiently. The compound (1) of one aspect of the present invention is useful as a phosphorescent host, but any other compound than the compound (1) may also be suitably selected as the phosphorescent host in accordance with the intended object thereof. The compound (1) is not limited to application to the above-mentioned phosphorescent host.

The compound (1) and any other compound may be combinedly used as a phosphorescent host material in one and the same light emitting layer, or in the case where the light emitting device has plural light emitting layers, the compound (1) may be used as the phosphorescent host material in one light emitting layers of those plural layers and any other compound than the compound (1) may be used as the phosphorescent host material in the other one light emitting layer. In addition, the compound (1) of one aspect of the present invention can be used in any other organic layer than the light emitting layer, and in the case, any other compound than the compound (1) may be used as the phosphorescent host in the light emitting layer.

Examples of the preferred phosphorescent host compounds other than the compound (1) include a carbazole derivative, a triazole derivative, a oxazole derivative, an oxadiazole derivative, an imidazole derivative, a polyarylalkane derivative, a pyrazoline derivative, a pyrazolone derivative, a phenylenediamine derivative, an arylamine derivative, an amino-substituted chalcone derivative, a styrylanthracene derivative, a fluorenone derivative, a hydrazone derivative, a stilbene derivative, a silazane derivative, an aromatic tertiary amine compound, a styrylamine compound, an aromatic dimethylidene compound, a porphyrin compound, an anthraquinodimethane derivative, an anthrone derivative, a diphenylquinone derivative, a thiopyran dioxide derivative, a carbodiimide derivative, a fluorenylidenemethane derivative, a distyrylpyrazine derivative, a heterocyclic tetracarboxylic anhydride such as naphthaleneperylene, a phthalocyanine derivative, various metal complexes typified by a metal complex of 8-quinolinol derivative, metal phthalocyanine, metal complexes having a ligand such as benzoxazole or benzothiazole, an electroconductive polymer oligomer, such as a polysilane compound, a poly(N-vinylcarbazole) derivative, an aniline copolymer, a thiophene oligomer, and a polythiophene, and a polymer compound such as a polythiophene derivative, a polyphenylene derivative, a polyphenylenevinylene derivative, and a polyfluorene derivative. These phosphorescent hosts may be used alone or in combination of two or more. Examples thereof are shown below.

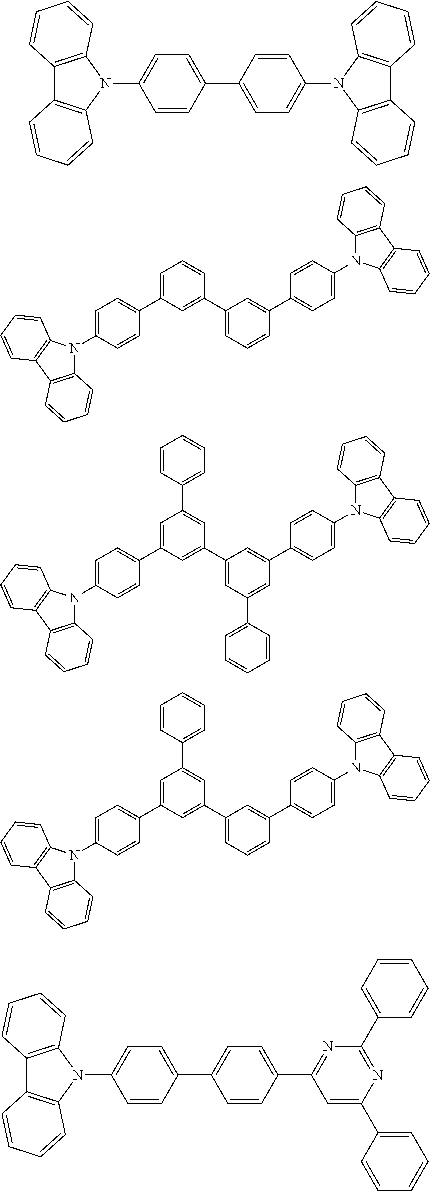

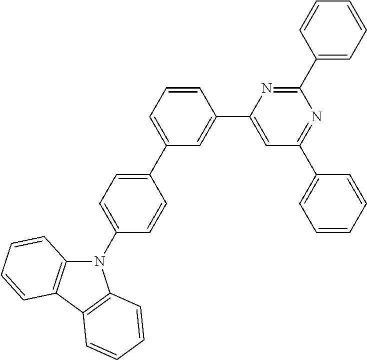 [SMILES: c1ccc(-c2cc(-c3cccc(-c4ccc(-n5c6ccccc6c6ccccc65)cc4)c3)nc(-c3ccccc3)n2)cc1]

The organic EL device of one aspect of the present invention may has a light emitting layer containing a fluorescent light emitting material, or that is, a fluorescent light emitting layer. For the fluorescent light emitting layer, any known fluorescent light emitting material may be used. The fluorescent light emitting material is preferably at least one selected from an anthracene derivative, a fluoranthene derivative, a styrylamine derivative and an arylamine derivative, more preferably from an anthracene derivative and an arylamine derivative. In particular, an anthracene derivative is preferred as the host material, and an arylamine derivative is preferred as the dopant. Specifically, the preferred materials described in WO2010/134350 and WO2010/134352 are selected. The organic EL device material of one aspect of the present invention may be used as the fluorescent light emitting material in the fluorescent light emitting layer, or as the host material in the fluorescent light emitting layer.

### (Electron Donating Dopant)

Preferably, the organic EL device of one aspect of the present invention has an electron donating dopant in the interface region between the cathode and the light emitting unit. This configuration improves the emission brightness of the organic EL device and prolongs the life thereof. Here, the electron donating dopant has a metal having a work function of 3.8 eV or less and examples thereof include at least one selected from alkali metals, alkali metal complexes, alkali metal compounds, alkaline earth metals, alkaline earth metal complexes, alkaline earth metal compounds, rare earth metals, rare earth metal complexes, and rare earth metal compounds.

Examples of the alkali metal include Na (work function: 2.36 eV), K (work function: 2.28 eV), Rb (work function: 2.16 eV), and Cs (work function: 1.95 eV), with those having a work function of 2.9 eV or less being particularly preferred. Of the above, preferred are K, Rb, and Cs, more preferred are Rb and Cs, and most preferred is Cs. Examples of the alkaline earth metal include Ca (work function: 2.9 eV), Sr (work function: 2.0 to 2.5 eV), and Ba (work function: 2.52 eV), with those having a work function of 2.9 eV or less being particularly preferred. Examples of the rare earth metal include Sc, Y, Ce, Tb, and Yb, with those having a work function of 2.9 eV or less being particularly preferred.

Examples of the alkali metal compound include alkali oxide, such as Li₂O, Cs₂O, K₂O, and alkali halide, such as LiF, NaF, CsF, and KF, with LiF, Li₂O, and NaF being preferred. Examples of the alkaline earth metal compound include BaO, SrO, CaO, and mixture thereof, such as BaₓSr₁₋ₓO (0<x<1) and BaₓCa₁₋ₓO (0<x<1), with BaO, SrO, and CaO being preferred. Examples of the rare earth metal compound include YbF₃, ScF₃, ScO₃, Y₂O₃, Ce₂O₃, GdF₃, and TbF₃, with YbF₃, ScF₃, and TbF₃ being preferred.

Examples of the alkali metal complex, alkaline earth metal complex, and rare earth metal are not particularly limited as long as containing at least one metal ion selected from alkali metal ions, alkaline earth metal ions, and rare earth metal ions, respectively. The ligand is preferably, but not limited to, quinolinol, benzoquinolinol, acridinol, phenanthridinol, hydroxyphenyloxazole, hydroxyphenylthiazole, hydroxydiaryloxadiazole, hydroxydiarylthiadiazole, hydroxyphenylpyridine, hydroxyphenylbenzimidazole, hydroxybenzotriazole, hydroxyfulborane, bipyridyl, phenanthroline, phthalocyanine, porphyrin, cyclopentadiene, β-diketones, azomethines, and derivative thereof.

The electron-donating dopant is added to the interfacial region preferably into a form of layer or island. The electron-donating dopant is added preferably by co-depositing the electron-donating dopant with the organic compound (light emitting material, electron injecting material) for forming the interfacial region by a resistance heating deposition method, thereby dispersing the electron-donating dopant into the organic compound. The disperse concentration expressed by the molar ratio of the organic compound to the electron-donating dopant is 100:1 to 1:100 and preferably 5:1 to 1:5.

When the electron-donating dopant is formed into a form of layer, a light emitting material or an electron injecting material is made into a layer which serves as an organic layer in the interface, and then, the reducing dopant alone is deposited by a resistance heating deposition method into a layer having a thickness preferably 0.1 to 15 nm. When the electron-donating dopant is formed into a form of island, a light emitting material or an electron injecting material is made into a form of island which serves as an organic layer in the interface, and then, the electron-donating dopant alone is deposited by a resistance heating deposition method into a form of island having a thickness preferably 0.05 to 1 nm.

Regarding the proportion of the main component and the electron-donating dopant in the organic EL device of one aspect of the present invention, the molar ratio of the main component to the electron-donating dopant is preferably 5:1 to 1:5 and more preferably 2:1 to 1:2.

### (Electron Transporting Layer)

The electron transporting layer is an organic layer disposed between the light emitting layer and the cathode and has a function of transporting electrons from the cathode to the light emitting layer. If two or more electron transporting layers are provided, the organic layer closer to the cathode may be called an electron injecting layer in some cases. The electron injecting layer has a function of injecting electrons from the cathode to the organic layer unit efficiently. The compound (1) of one aspect of the present invention may be used as an electron transporting material to be contained in the electron transporting layer (second charge transporting material).

An aromatic heterocyclic compound having one or more hetero atoms in the molecule thereof is preferably used as an electron transporting material used in the electron transporting layer, and a nitrogen-containing ring derivative is particularly preferred. In addition, the nitrogen-containing ring derivative is preferably an aromatic ring compound having a nitrogen-containing, 6- or 5-membered ring skeleton, or a condensed aromatic ring compound having a nitrogen-containing, 6- or 5-membered ring skeleton.

The nitrogen-containing ring derivative is preferably, for example, a metal chelate complex of a nitrogen-containing ring represented by the following formula (A).

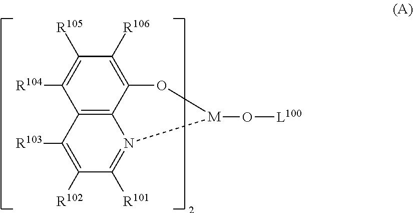

In the formula (A), each of R¹⁰ to R¹⁰⁶ each independently represent a hydrogen atom, a halogen atom, a hydroxyl group, an amino group, a hydrocarbon group having 1 to 40 (preferably 1 to 20, more preferably 1 to 10, even more preferably 1 to 5) carbon atoms, an alkoxy group having 1 to 40 (preferably 1 to 20, more preferably 1 to 10, even more preferably 1 to 5) carbon atoms, an aryloxy group having 6 to 50 (preferably 6 to 20, more preferably 6 to 12) ring carbon atoms, an alkoxycarbonyl group having 2 to 40 (preferably 2 to 20, more preferably 2 to 10, even more preferably 2 to 5) carbon atoms, or an aromatic heterocyclic group having 5 to 50 (preferably 5 to 30, more preferably 5 to 20) ring atoms, each being optionally substituted.

The halogen atom may include fluorine, chlorine, bromine, and iodine.

The substituted amino group may include an alkylamino group, an arylamino group, and an aralkylamino group.

The alkylamino group and the aralkylamino group are represented by —NQ¹Q². Each of Q¹ and Q² independently represents an alkyl group having 1 to 20 carbon atoms or an aralkyl group having 1 to 20 carbon atoms. One of Q¹ and Q² may be a hydrogen atom.

The arylamino group is represented by —NAr^{1′}Ar^{2′}, wherein each of Ar^{1′} and Ar^{2′} independently represents a non-condensed aromatic hydrocarbon group or a condensed aromatic hydrocarbon group, each having 6 to 50 carbon atoms. One of Ar^{1′} and Ar^{2′} may be a hydrogen atom.

Examples of the hydrocarbon group having 1 to 40 carbon atoms include an alkyl group, an alkenyl group, a cycloalkyl group, an aryl group, and an aralkyl group.

The alkoxycarbonyl group is represented by —COOY′, wherein Y′ is an alkyl group having 1 to 20 carbon atoms.

M is aluminum (Al), gallium (Ga), or indium (In), with In being preferred.

L¹⁰⁰ is a group represented by the following formula (A′) or (A″).

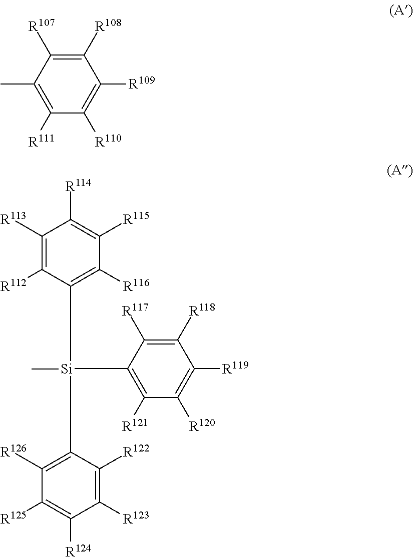

In the formula (A′), each of R¹⁰⁷ to R¹¹¹ independently represents a hydrogen atom, or a substituted or unsubstituted hydrocarbon group having 1 to 40 (preferably 1 to 20, more preferably 1 to 10, even more preferably 1 to 5) carbon atoms, and two or more of R¹⁰⁷ to R¹¹¹ may bond to form a cyclic structure. In the formula (A″), each of R¹¹² to R¹²⁶ independently represents a hydrogen atom, or a substituted or unsubstituted hydrocarbon group having 1 to 40 (preferably 1 to 20, more preferably 1 to 10, even more preferably 1 to 5) carbon atoms, and two or more of R¹¹² to R¹²⁶ may bond to form a cyclic structure.

The hydrocarbon group having 1 to 40 carbon atoms for R¹⁰⁷ to R¹²⁶ in the formulae (A′) and (A″) is the same as the hydrocarbon group for R¹⁰¹ to R¹⁰⁶ in the above-mentioned formula (A). The divalent group in the case where two or more of R¹⁰⁷ to R¹¹¹ bond to form a cyclic structure and in the case where two or more of R¹¹² to R¹²⁶ bond to form a cyclic structure includes a tetramethylene group, a pentamethylene group, a hexamethylene group, a diphenylmethane-2,2′-diyl group, a diphenylethane-3,3′-diyl group, and a diphenylpropane-4,4′-diyl group.

The electron transmitting compound for use in the electron transporting layer is preferably a metal complex including 8-hydroxyquinoline or its derivative, an oxadiazole derivative, or a nitrogen-containing heterocyclic derivative. Examples of the metal complex including 8-hydroxyquinoline or its derivative include a metal chelate oxinoid compound including a chelated oxine (generally, 8-quinolinol or 8-hydroxyquinoline), for example, tris(8-quinolinol)aluminum. Examples of the oxadiazole derivative are shown below.

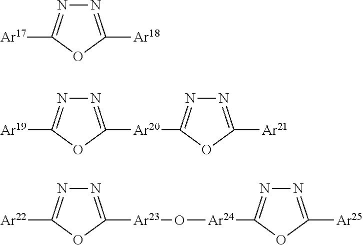

In the above formulae, each of Ar¹⁷, Ar¹⁸, Ar¹⁹, Ar²¹, Ar²², and Ar²⁵ is a substituted or unsubstituted aromatic hydrocarbon group or a substituted or unsubstituted condensed aromatic hydrocarbon group each having 6 to 50 carbon atoms, and Ar¹⁷ and Ar¹⁸, Ar¹⁹ and Ar²¹, and Ar²² and Ar²⁵ may be the same or different. Examples of the aromatic hydrocarbon group and the condensed aromatic hydrocarbon group include a phenyl group, a naphthyl group, a biphenyl group, an anthranyl group, a perylenyl group, and a pyrenyl group. The optional substituent may be an alkyl group having 1 to 10 carbon atoms, an alkoxyl group having 1 to 10 carbon atoms or a cyano group.

Each of Ar²⁰, Ar²³, and Ar²⁴ is a substituted or unsubstituted bivalent aromatic hydrocarbon group or a substituted or unsubstituted bivalent condensed aromatic hydrocarbon group each having 6 to 60 carbon atoms, and Ar²³ and Ar²⁴ may be the same or different. Examples of the bivalent aromatic hydrocarbon group or the bivalent condensed aromatic hydrocarbon group include a phenylene group, a naphthylene group, a biphenylene group, an anthranylene group, a perylenylene group, and a pyrenylene group. The optional substituent may be an alkyl group having 1 to 10 carbon atoms, an alkoxyl group having 1 to 10 carbon atoms or a cyano group.

Electron transmitting compounds which have a good thin film-forming property are preferably used. Examples of the electron transmitting compound are shown below.

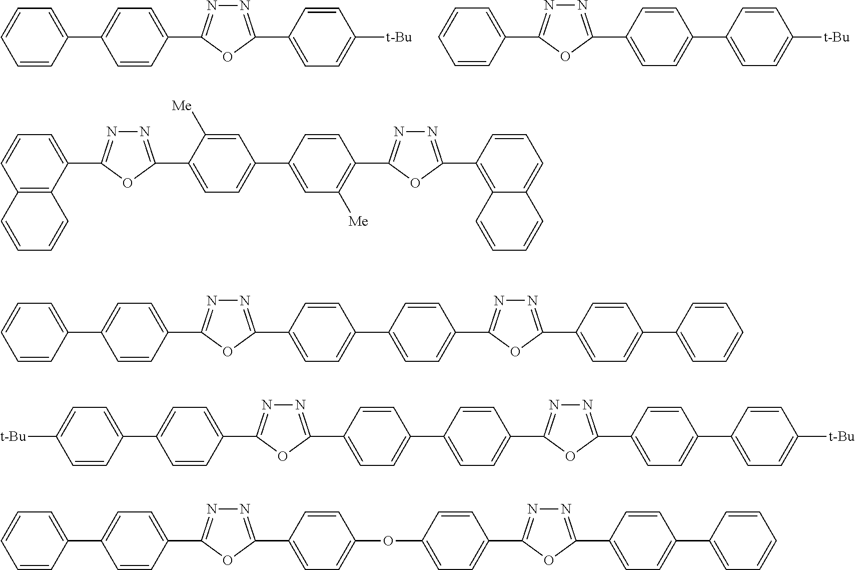

Examples of the nitrogen-containing heterocyclic derivative for use as the electron transmitting compound include a nitrogen-containing heterocyclic derivative of an organic compound having the following formulae but exclusive of metal complex, for example, a compound having a 5- or 6-membered ring which has the skeleton represented by the following formula (B) or having the structure represented by the following formula (C).

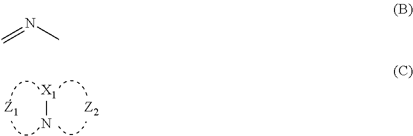

In the formula (C), X₁ is a carbon atom or a nitrogen atom and each of Z₁ and Z₂ independently represents a group of atoms for completing the nitrogen-containing heterocyclic ring.

The nitrogen-containing heterocyclic derivative is more preferably an organic compound which has a nitrogen-containing aromatic polycyclic ring of a 5-membered ring or a 6-membered ring. If two or more nitrogen atoms are included, the nitrogen-containing aromatic polycyclic compound preferably has a skeleton of a combination of the above formulae (B) and (C) or a combination of the above formula (B) and the following formula (D).

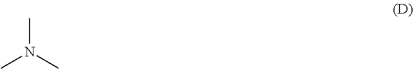

The nitrogen-containing group of the nitrogen-containing aromatic polycyclic compound is selected, for example, from the nitrogen-containing heterocyclic groups shown below.

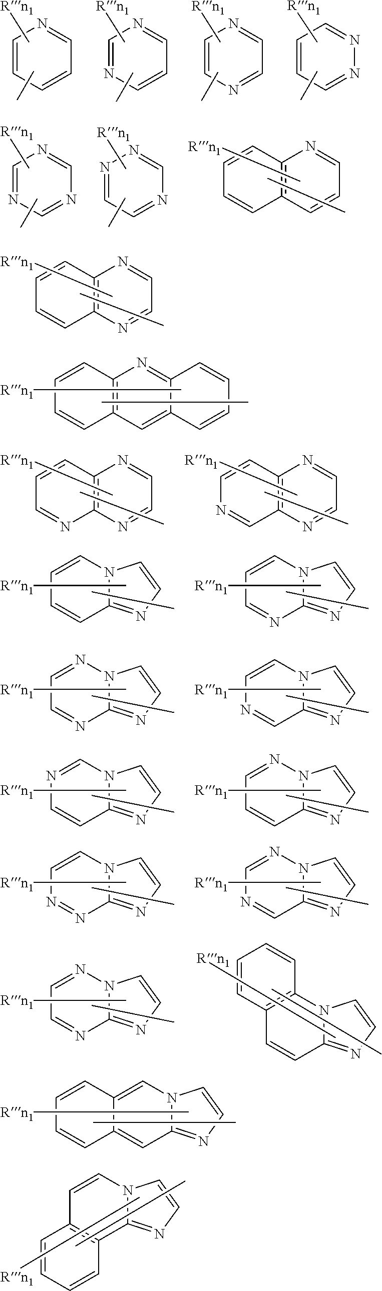

In the formulae, R′″ is an aromatic hydrocarbon group having 6 to 40 (preferably 6 to 30, more preferably 6 to 20, and still more preferably 6 to 12) ring carbon atoms, a condensed aromatic hydrocarbon group having 6 to 40 (preferably 6 to 30, more preferably 6 to 20, even more preferably 6 to 12) ring carbon atoms, an aromatic heterocyclic group having 5 to 40 (preferably 5 to 30, more preferably 5 to 20, even more preferably 5 to 12) ring atoms, a condensed aromatic heterocyclic group having 5 to 40 (preferably 5 to 30, more preferably 5 to 20, even more preferably 5 to 12) ring atoms, an alkyl group having 1 to 20 (preferably 1 to 10, and more preferably 1 to 5) carbon atoms, or an alkoxy group having 1 to 20 (preferably 1 to 10, and more preferably 1 to 5) carbon atoms.

n₁ is an integer of 0 to 5. When n₁ is an integer of 2 or more, (R′″)'s may be the same or different.

More preferred is a nitrogen-containing heterocyclic derivative represented by the following formula (D1).

HAr-L¹⁰¹-Ar¹⁰¹—Ar¹⁰² (D1)

wherein HAr is a substituted or unsubstituted nitrogen-containing heterocyclic group having 5 to 40 (preferably 5 to 30, more preferably 5 to 20, and still more preferably 5 to 12) ring atoms;

L¹⁰¹ is a single bond, a substituted or unsubstituted aromatic hydrocarbon group or condensed aromatic hydrocarbon group each having 6 to 40 (preferably 6 to 30, more preferably 6 to 20, and still more preferably 6 to 12) ring carbon atoms, a substituted or unsubstituted aromatic heterocyclic group having 5 to 40 (preferably 5 to 30, more preferably 5 to 20, even more preferably 5 to 12) ring carbon atom, a substituted or unsubstituted condensed aromatic heterocyclic group having 6 to 40 (preferably 6 to 30, more preferably 6 to 20, even more preferably 6 to 12) ring carbon atoms.

Ar¹⁰¹ is a substituted or unsubstituted divalent aromatic hydrocarbon group having 6 to 40 (preferably 6 to 30, more preferably 6 to 20, and still more preferably 6 to 12) ring carbon atoms; and Ar¹⁰² is a substituted or unsubstituted aromatic hydrocarbon group having 6 to 40 (preferably 6 to 30, more preferably 6 to 20, even more preferably 6 to 14) ring carbon atoms, a substituted or unsubstituted, condensed aromatic hydrocarbon group having 6 to 40 (preferably 6 to 30, more preferably 6 to 20, and still more preferably 6 to 12) ring carbon atoms, a substituted or unsubstituted aromatic heterocyclic group having 5 to 40 (preferably 5 to 30, more preferably 5 to 20, even more preferably 5 to 12) ring atoms or a substituted or unsubstituted, condensed aromatic heterocyclic group having 5 to 40 (preferably 5 to 30, more preferably 5 to 20, and still more preferably 5 to 12) ring atoms.

HAr is selected, for example, from the following groups.

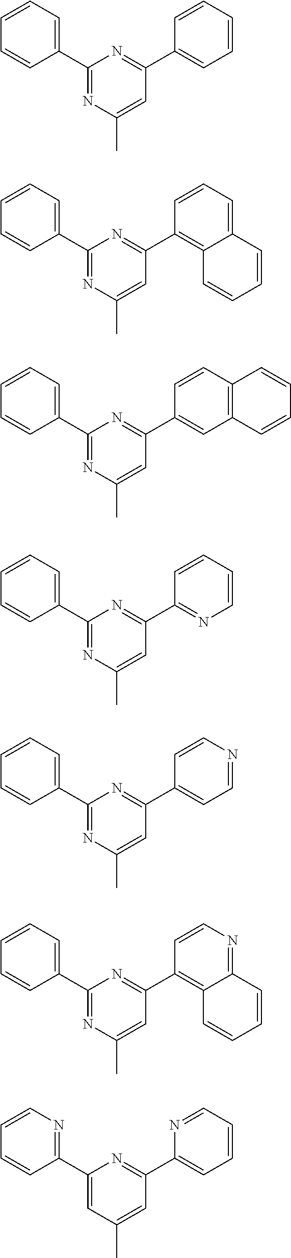

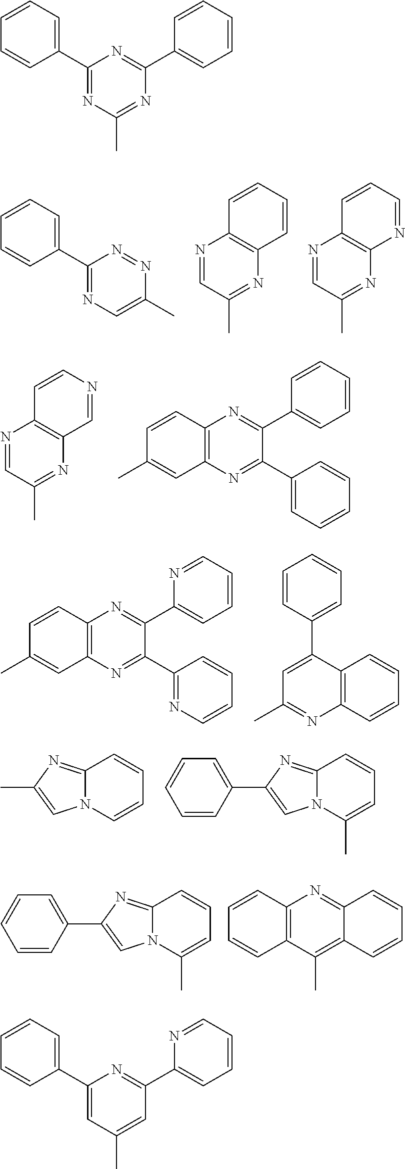

L¹⁰¹ is selected, for example, from the following groups.

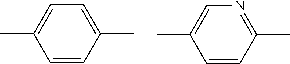

Ar¹⁰¹ is selected, for example, from the groups represented by the following formulae (D2) and (D3).

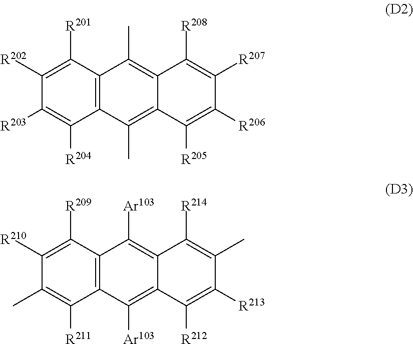

In the formulae (D2) and (D3), R²⁰¹ to R²¹⁴ are each independently a hydrogen atom, a halogen atom, a substituted or unsubstituted alkyl group having 1 to 20 (preferably 1 to 10, more preferably 1 to 5) carbon atoms, a substituted or unsubstituted alkoxy group having 1 to 20 (preferably 1 to 10, more preferably 1 to 5) carbon atoms, a substituted or unsubstituted aryloxy group having 6 to 40 (preferably 6 to 30, more preferably 6 to 20, even more preferably 6 to 12) ring carbon atoms, a substituted or unsubstituted aromatic hydrocarbon group having 6 to 40 (preferably 6 to 30, more preferably 6 to 20, even more preferably 6 to 12) ring carbon atoms, a substituted or unsubstituted condensed aromatic hydrocarbon group having 6 to 40 (preferably 6 to 30, more preferably 6 to 20, even more preferably 6 to 12) ring carbon atoms, a substituted or unsubstituted aromatic heterocyclic group having 5 to 40 (preferably 5 to 30, more preferably 5 to 20, even more preferably 5 to 12) ring carbon atoms, or a substituted or unsubstituted condensed aromatic heterocyclic group having 5 to 40 (preferably 5 top 30, more preferably 5 to 20, even more preferably 5 to 12) ring atoms.

Ar¹⁰³ is a substituted or unsubstituted aromatic hydrocarbon group having 6 to 40 (preferably 6 to 30, more preferably 6 to 20, even more preferably 6 to 12) ring carbon atoms, a substituted or unsubstituted condensed aromatic hydrocarbon group having 6 to 40 (preferably 6 to 30, more preferably 6 to 20, even more preferably 6 to 12) ring carbon atoms, a substituted or unsubstituted aromatic heterocyclic group having 5 to 40 (preferably 5 to 30, more preferably 5 to 20, even more preferably 5 to 12) ring carbon atoms, or a substituted or unsubstituted condensed aromatic heterocyclic group having 5 to 40 (preferably 5 to 30, more preferably 5 to 20, even more preferably 5 to 12) ring atoms.

Ar¹⁰² is, for example, selected from the following groups.

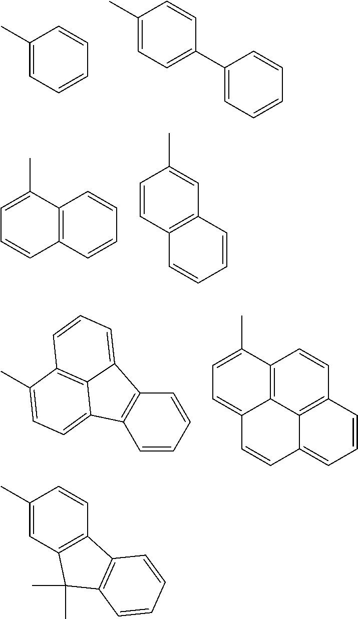

In addition, the following compound is preferably used as the nitrogen-containing aromatic polycyclic organic compound for use as the electron transmitting compound.

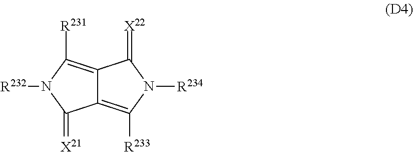

In the formula (D4), R²³¹ to R²³⁴ each independently represent a hydrogen atom, a substituted or unsubstituted aliphatic group having 1 to 20 carbon atoms, a substituted or unsubstituted alicyclic group having 3 to 20 carbon atoms, a substituted or unsubstituted aromatic ring group having 6 to 50 carbon atoms, or a substituted or unsubstituted heterocyclic group having 3 to 50 carbon atoms; and X²¹ and X²² each independently represent an oxygen atom, a sulfur atom, or a dicyanomethylene group.

Further, the following compound is also suitable as the electron transmitting compound.

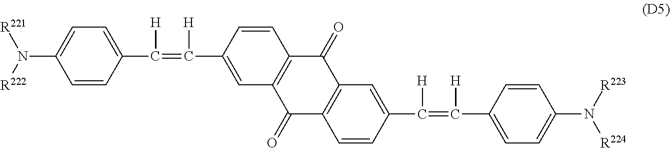

In the formula (D5), R²²¹, R²²², R²²³ and R²²⁴ may be the same or different and each represents an aromatic hydrocarbon group or a condensed aromatic hydrocarbon group represented by the following formula (D6).

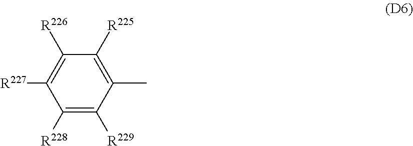

In the formula (D6), R²²⁵, R²²⁶, R²²⁷, R²²⁸ and R²²⁹ may be the same or different and each represents a hydrogen atom, a saturated or unsaturated alkoxyl group having 1 to 20 carbon atoms, a saturated or unsaturated alkyl group having 1 to 20 carbon atoms, an amino group, or an alkylamino group having 1 to 20 carbon atoms. At least one of R²²⁵, R²²⁶, R²²⁷, R²²⁸ and R²²⁹ represents a group other than a hydrogen atom.

Further, a polymer including the above nitrogen-containing heterocyclic group or the above nitrogen-containing heterocyclic derivative is also usable as the electron transmitting compound.

In a particularly preferred embodiment of the present invention, the electron transporting layer of the organic EL device of one aspect of the present invention contains at least one compound selected from the nitrogen-containing heterocyclic derivatives represented by the following formulae (E) to (G).

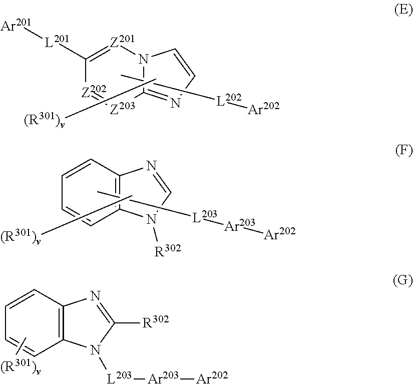

In the formulae (E) to (C), Z²⁰¹, Z²⁰², and Z²⁰³ each independently represent a nitrogen atom or a carbon atom.

R³⁰¹ and R³⁰² each independently represent a substituted or unsubstituted aryl group having 6 to 50 (preferably 6 to 30, more preferably 6 to 20, and still more preferably 6 to 12) ring carbon atoms, a substituted or unsubstituted heteroaryl group having 5 to 50 (preferably 5 to 30, more preferably 5 to 20, and still more preferably 5 to 12) ring atoms, a substituted or unsubstituted alkyl group having 1 to 20 carbon atoms, a substituted or unsubstituted haloalkyl group having 1 to 20 carbon atoms, or a substituted or unsubstituted alkoxyl group having 1 to 20 carbon atoms.

v is an integer of 0 to 5, when v is an integer of 2 or more, R³⁰¹'s may be the same or different, and two R³⁰¹'s may bond to each other to form a substituted or unsubstituted hydrocarbon ring.

Ar²⁰¹ represents a substituted or unsubstituted aryl group having 6 to 50 (preferably 6 to 30, more preferably 6 to 20, and still more preferably 6 to 12) ring carbon atoms, or a substituted or unsubstituted heteroaryl group having 5 to 50 (preferably 5 to 30, more preferably 5 to 20, even more preferably 5 to 12) ring atoms.

Ar²⁰² represents a hydrogen atom, a substituted or unsubstituted alkyl group having 1 to 20 (preferably 1 to 10, and more preferably 1 to 5) carbon atoms, a substituted or unsubstituted haloalkyl group having 1 to 20 (preferably 1 to 10, and more preferably 1 to 5) carbon atoms, a substituted or unsubstituted alkoxy group having 1 to 20 carbon atoms, a substituted or unsubstituted aryl group having 6 to 50 (preferably 6 to 30, more preferably 6 to 20, and still more preferably 6 to 12) ring carbon atoms, or a substituted or unsubstituted heteroaryl group having 5 to 50 (preferably 5 to 30, more preferably 5 to 20, and still more preferably 5 to 12) ring atoms.

One of Ar²⁰¹ and Ar²⁰² is a substituted or unsubstituted condensed aromatic hydrocarbon ring group having 10 to 50 (preferably 10 to 30, more preferably 10 to 20) ring carbon atoms or a substituted or unsubstituted condensed aromatic heterocyclic group having 9 to 50 (preferably 9 to 30, more preferably 9 to 20) ring atoms.

Ar²⁰³ represents a substituted or unsubstituted arylene group having 6 to 50 (preferably 6 to 30, more preferably 6 to 20, and still more preferably 6 to 12) ring carbon atoms or a substituted or unsubstituted heteroarylene group having 5 to 50 (preferably 5 to 30, more preferably 5 to 20, and still more preferably 5 to 12) ring atoms.

L²⁰¹, L²⁰², and L²⁰³ each independently represent a single bond, a substituted or unsubstituted arylene group having 6 to 50 (preferably 6 to 30, more preferably 6 to 20, and still more preferably 6 to 12) ring carbon atoms or a substituted or unsubstituted, divalent condensed aromatic heterocyclic group having 9 to 50 (preferably 9 to 30, more preferably 9 to 20) ring atoms.

Examples of the aryl group having 6 to 50 ring carbon atoms include a phenyl group, a naphthyl group, an anthryl group, a phenanthryl group, a naphthacenyl group, a chrysenyl group, pyrenyl group, a biphenyl group, a terphenyl group, a tolyl group, a fluoranthenyl group, and a fluorenyl group.

Examples of the heteroaryl group having 5 to 50 ring atoms include a pyrrolyl group, a furyl group, a thienyl group, a silolyl group, a pyridyl group, a quinolyl group, an isoquinolyl group, a benzofuryl group, an imidazolyl group, a pyrimidyl group, a carbazolyl group, a selenophenyl group, an oxadiazolyl group, a triazolyl group, a pyrazinyl group, a pyridazinyl group, a triazinyl group, a quinoxalinyl group, an acridinyl group, an imidazo[1,2-a]pyridinyl group, and an imidazo[1, 2-a]pyrimidinyl.

Examples of the alkyl group having 1 to 20 carbon atoms include a methyl group, an ethyl group, a propyl group, a butyl group, a pentyl group, and a hexyl group.

Examples of the haloalkyl group having 1 to 20 carbon atoms include the groups obtained by replacing one or more hydrogen atoms of the alkyl group mentioned above with at least one halogen atom selected from fluorine, chlorine, iodine, and bromine.

Examples of the alkyl moiety of the alkoxyl group having 1 to 20 carbon atoms include the alkyl group mentioned above.

Examples of the arylene group having 6 to 50 ring carbon atoms include the groups obtained by removing one hydrogen atom from the aryl group mentioned above.

Examples of the divalent condensed aromatic heterocyclic group having 9 to 50 ring atoms include the groups obtained by removing one hydrogen atom from the condensed aromatic heterocyclic group mentioned above as the heteroaryl group.

The thickness of the electron transporting layer is preferably, but not particularly limited to, 1 to 100 nm.

Preferred examples of the material for the electron injecting layer optionally formed adjacent to the electron transporting layer include, in addition to the nitrogen-containing ring derivative, an inorganic compound, such as an insulating material and a semiconductor. The electron injecting layer containing the insulating material or the semiconductor effectively prevents the leak of electric current to enhance the electron injecting properties.

The insulating material is preferably at least one metal compound selected from a group consisting of alkali metal chalcogenides, alkaline earth metal chalcogenides, alkali metal halides and alkaline earth metal halides. The alkali metal chalcogenide, etc. mentioned above are preferred because the electron injecting properties of the electron injecting layer are further enhanced. Examples of preferred alkali metal chalcogenide include Li₂O, K₂O, Na₂S, Na₂Se and Na₂O, and examples of preferred alkaline earth metal chalcogenide include CaO, BaO, SrO, BeO, BaS and CaSe. Examples of preferred alkali metal halide include LiF, NaF, KF, LiCl, KCl and NaCl. Examples of preferred alkaline earth metal halides include fluorides, such as CaF₂, BaF₂, SrF₂, MgF₂ and BeF₂, and halides other than fluorides.

Examples of the semiconductor include oxides, nitrides or oxynitrides of one element alone or two or more elements selected from a group consisting of Ba, Ca, Sr, Yb, Al, Ga, In, Li, Na, Cd, Mg, Si, Ta, Sb and Zn. The semiconductor may be used alone or in combination of two or more. The inorganic compound included in the electron injecting layer preferably forms a microcrystalline or amorphous insulating thin film. If the electron injecting layer is formed from such an insulating thin film, the pixel defects, such as dark spots, can be decreased because a more uniform thin film is formed. Examples of such inorganic compound include alkali metal chalcogenides, alkaline earth metal chalcogenides, alkali metal halides and alkaline earth metal halides.

When using the insulating material or the semiconductor, the thickness of its layer is preferably about 0.1 nm to 15 nm. The electron injecting layer in the organic EL device of one aspect of the present invention may contain the electron-donating dopant mentioned above.

### (Hole Transporting Layer)

The hole transporting layer is an organic layer formed between the light emitting layer and the anode and has a function of transporting holes from the anode to the light emitting layer. When the hole transporting layer is formed into two or more layers, the organic layer closer to the anode may be defined as a hole injecting layer in some cases. The hole injecting layer has a function of efficiently injecting holes from the anode to the organic layer unit. The compound (1) of one aspect of the present invention may be used as the hole transporting material to be contained in the hole transporting layer (first charge transporting layer).

Another preferred material for the hole transporting layer may include an aromatic amine compound, for example, an aromatic amine derivative represented by the following formula (H).

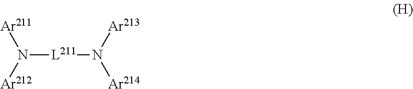

In the formula (H), each of Ar²¹¹ to Ar²¹⁴ independently represents a substituted or unsubstituted aromatic hydrocarbon group having 6 to 50 (preferably 6 to 30, more preferably 6 to 20, even more preferably 6 to 12) ring carbon atoms, a substituted or unsubstituted condensed aromatic hydrocarbon group having 6 to 50 (preferably 6 to 30, more preferably 6 to 20, and still more preferably 6 to 12) ring carbon atoms, a substituted or unsubstituted aromatic heterocyclic group having 5 to 50 (preferably 5 to 30, more preferably 5 to 20, even more preferably 5 to 12) ring atoms, a substituted or unsubstituted condensed aromatic heterocyclic group having 5 to 50 (preferably 5 to 30, more preferably 5 to 20, and still more preferably 5 to 12) ring atoms, or a group wherein the aromatic hydrocarbon group or the condensed aromatic hydrocarbon group is bonded to the aromatic heterocyclic group or the condensed aromatic heterocyclic group. Ar²¹¹ and Ar²¹², and Arm and Ar²¹⁴ may bond to each other to form a saturated or unsaturated cyclic structure.

In the formula (H), L²¹¹ represents a substituted or unsubstituted aromatic hydrocarbon group having 6 to 50 (preferably 6 to 30, more preferably 6 to 20, even more preferably 6 to 12) ring carbon atoms, a substituted or unsubstituted condensed aromatic hydrocarbon group having 6 to 50 (preferably 6 to 30, more preferably 6 to 20, and still more preferably 6 to 12) ring carbon atoms, a substituted or unsubstituted aromatic heterocyclic group having 5 to 50 (preferably 5 to 30, more preferably 5 to 20, even more preferably 5 to 12) ring atoms, or a substituted or unsubstituted condensed aromatic heterocyclic group 5 to 50 (preferably 5 to 30, more preferably 5 to 20, and still more preferably 5 to 12) ring atoms.

Examples of the compound represented by the formula (H) are shown below.

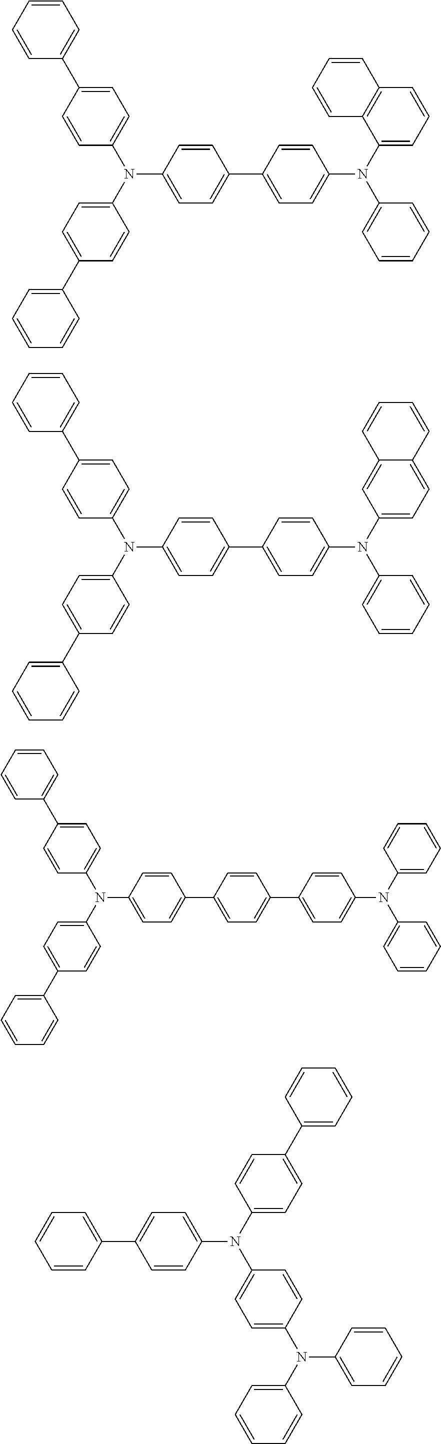

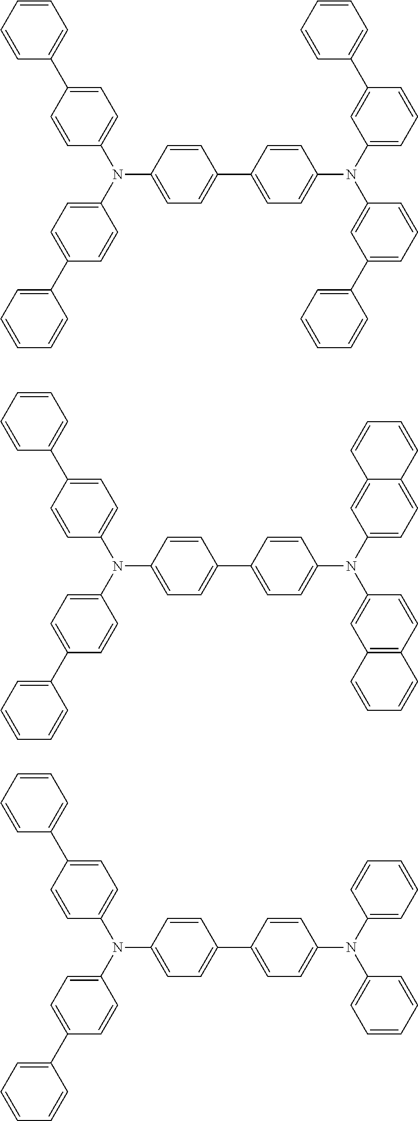

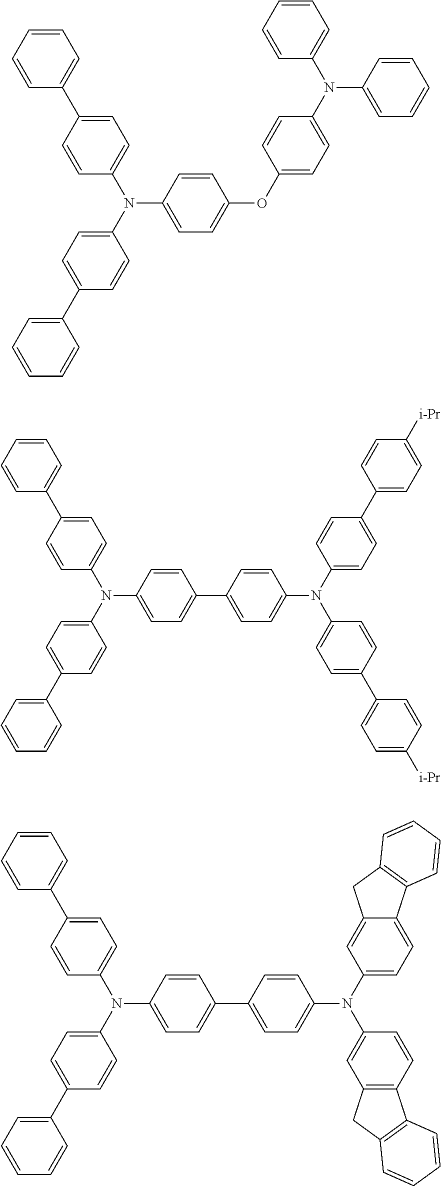

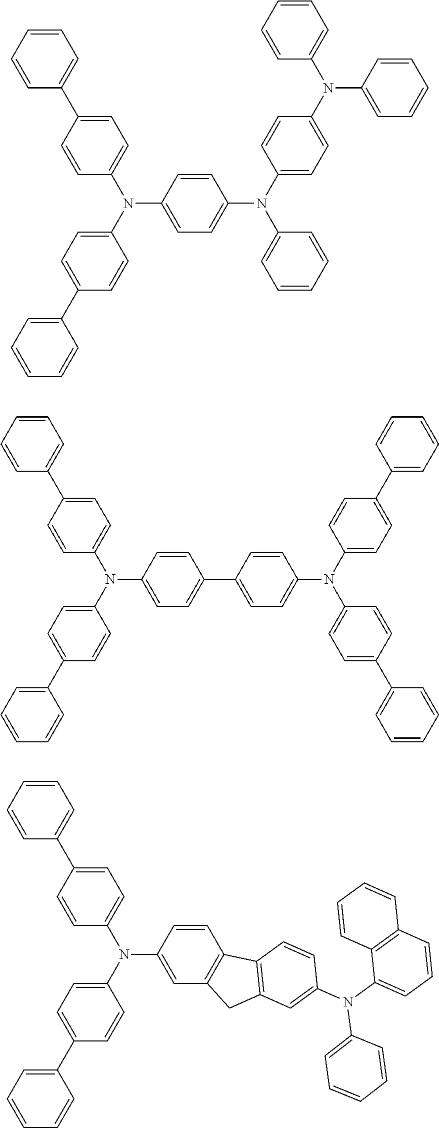

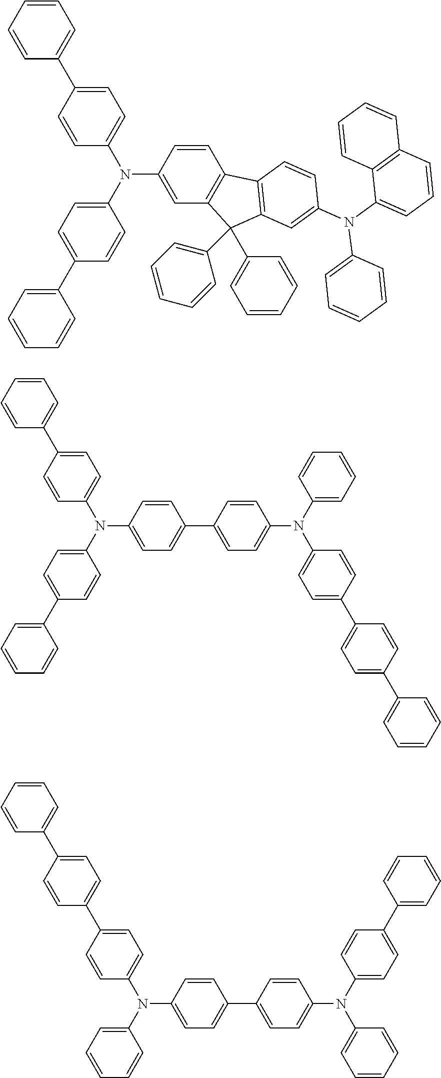

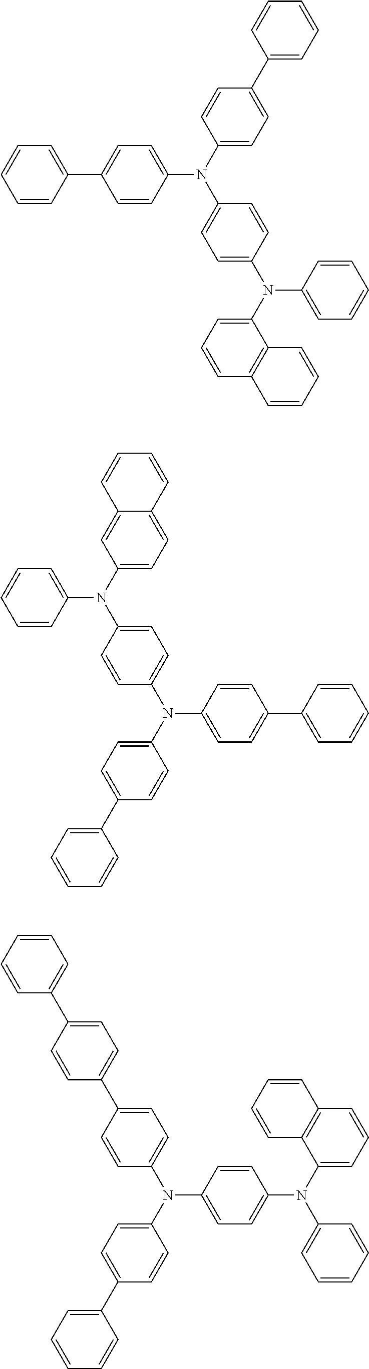 [SMILES: c1ccc(-c2ccc(N(c3ccc(-c4ccccc4)cc3)c3ccc(N(c4ccccc4)c4cccc5ccccc45)cc3)cc2)cc1 ; c1ccc(-c2ccc(N(c3ccc(-c4ccccc4)cc3)c3ccc(N(c4ccccc4)c4ccc5ccccc5c4)cc3)cc2)cc1 ; c1ccc(-c2ccc(-c3ccc(N(c4ccc(-c5ccccc5)cc4)c4ccc(N(c5ccccc5)c5cccc6ccccc56)cc4)cc3)cc2)cc1]

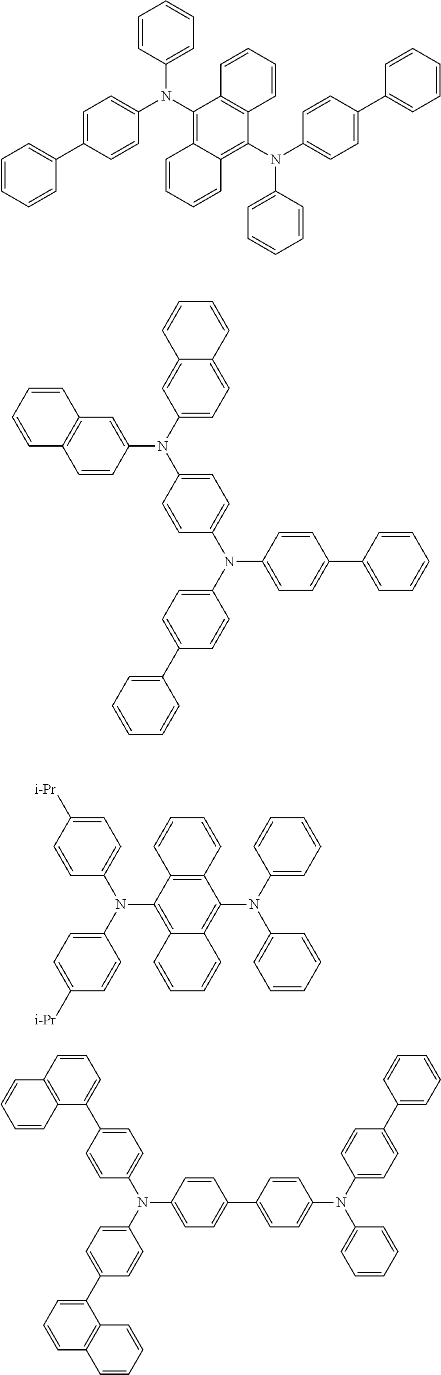

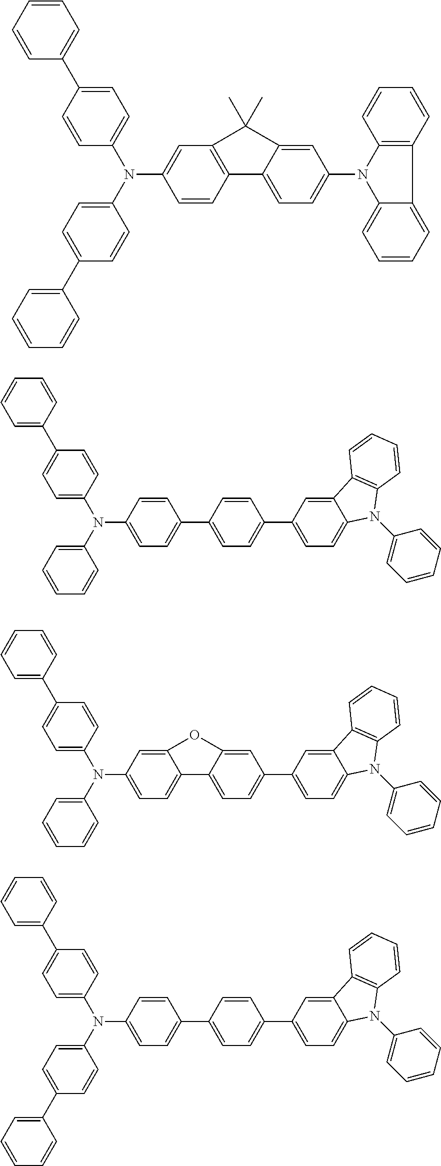

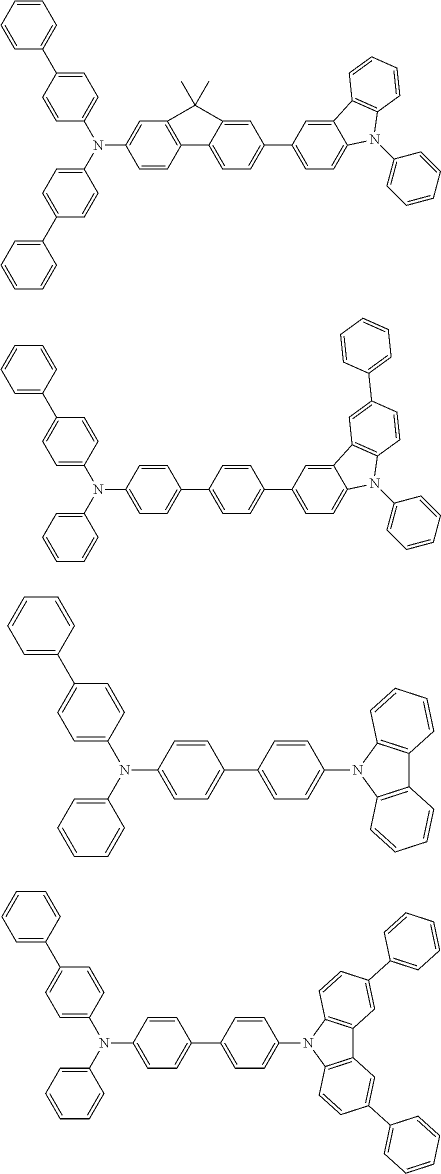

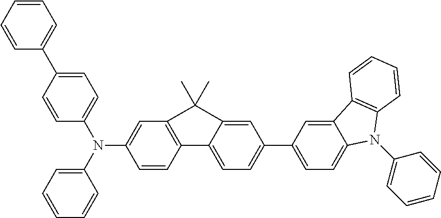 [SMILES: CC1(C)c2cc(-c3ccc4c(c3)c3ccccc3n4-c3ccccc3)ccc2-c2ccc(N(c3ccccc3)c3ccc(-c4ccccc4)cc3)cc21]

An aromatic amine represented by the following formula (J) is also preferably used to form the hole transporting layer.

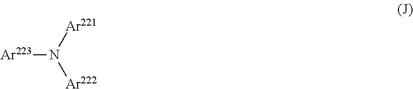

In the formula (J), Ar²²¹ to Ar²²³ and preferred examples thereof are as defined above with respect to Ar²¹¹ to Ar²¹⁴ of the above formula (H). Examples of the compound represented by the formula (J) are shown below, although not limited thereto.

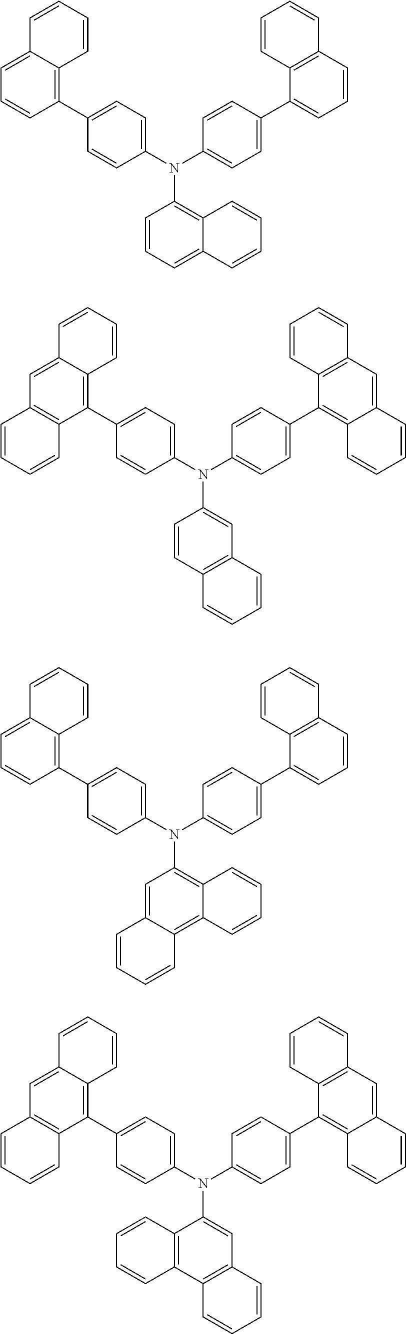 [SMILES: c1ccc2c(-c3ccc(N(c4ccc(-c5cccc6ccccc56)cc4)c4cccc5ccccc45)cc3)cccc2c1 ; c1ccc2cc(N(c3ccc(-c4c5ccccc5cc5ccccc45)cc3)c3ccc(-c4c5ccccc5cc5ccccc45)cc3)ccc2c1 ; c1ccc2c(-c3ccc(N(c4ccc(-c5cccc6ccccc56)cc4)c4cc5ccccc5c5ccccc45)cc3)cccc2c1 ; c1ccc2c(-c3ccc(N(c4ccc(-c5c6ccccc6cc6ccccc56)cc4)c4cc5ccccc5c5ccccc45)cc3)c3ccccc3cc2c1]

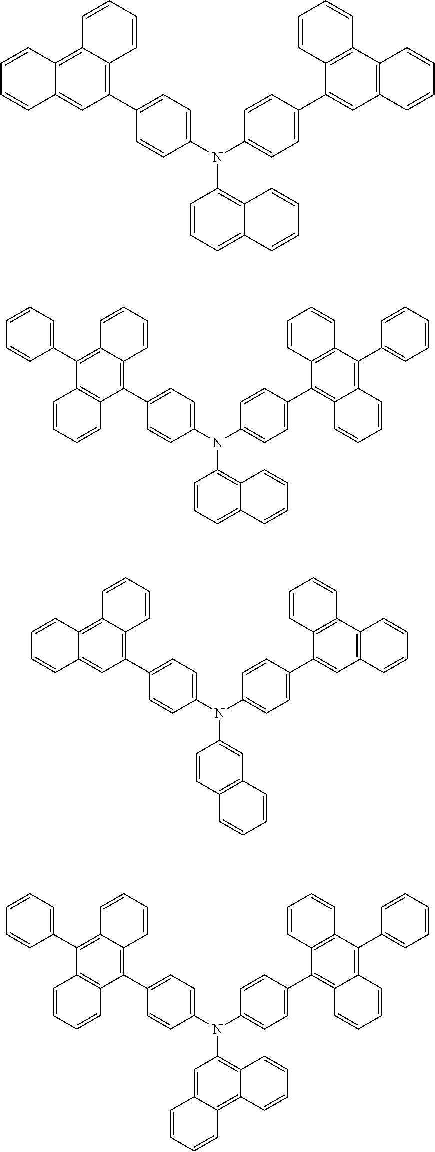 [SMILES: c1ccc2c(N(c3ccc(-c4cc5ccccc5c5ccccc45)cc3)c3ccc(-c4cc5ccccc5c5ccccc45)cc3)cccc2c1 ; c1ccc(-c2c3ccccc3c(-c3ccc(N(c4ccc(-c5c6ccccc6c(-c6ccccc6)c6ccccc56)cc4)c4cccc5ccccc45)cc3)c3ccccc23)cc1 ; c1ccc2cc(N(c3ccc(-c4cc5ccccc5c5ccccc45)cc3)c3ccc(-c4cc5ccccc5c5ccccc45)cc3)ccc2c1 ; c1ccc(-c2c3ccccc3c(-c3ccc(N(c4ccc(-c5c6ccccc6c(-c6ccccc6)c6ccccc56)cc4)c4cc5ccccc5c5ccccc45)cc3)c3ccccc23)cc1]

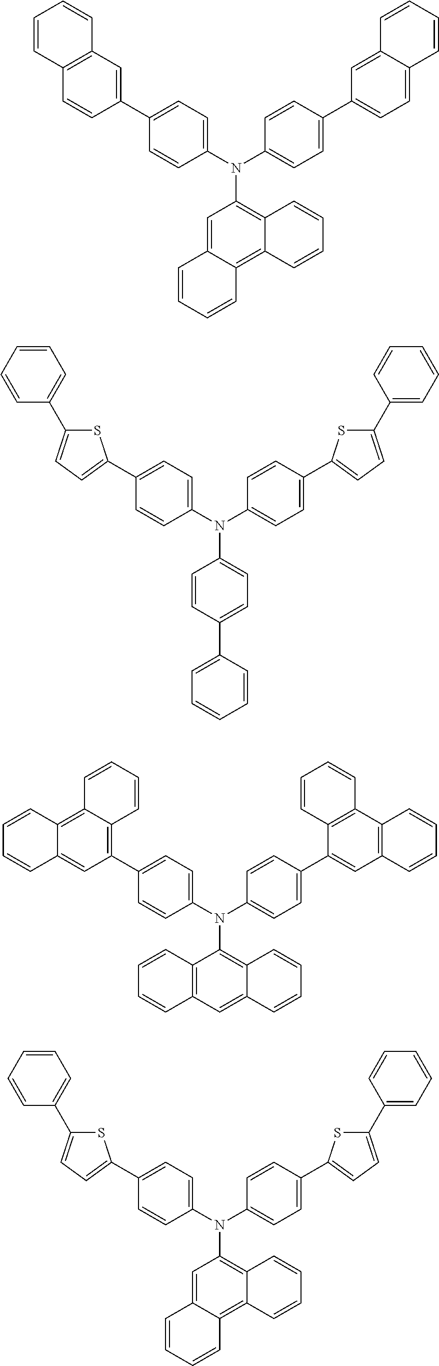 [SMILES: c1ccc2cc(-c3ccc(N(c4ccc(-c5ccc6ccccc6c5)cc4)c4cc5ccccc5c5ccccc45)cc3)ccc2c1 ; c1ccc(-c2ccc(N(c3ccc(-c4ccc(-c5ccccc5)s4)cc3)c3ccc(-c4ccc(-c5ccccc5)s4)cc3)cc2)cc1 ; c1ccc2c(N(c3ccc(-c4cc5ccccc5c5ccccc45)cc3)c3ccc(-c4cc5ccccc5c5ccccc45)cc3)c3ccccc3cc2c1 ; c1ccc(-c2ccc(-c3ccc(N(c4ccc(-c5ccc(-c6ccccc6)s5)cc4)c4cc5ccccc5c5ccccc45)cc3)s2)cc1]

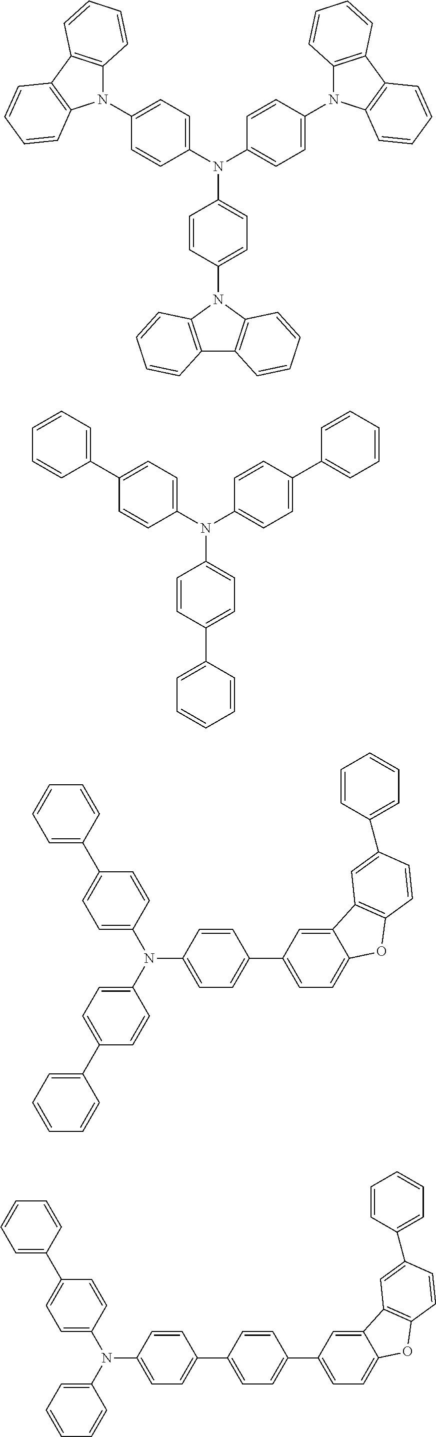 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1 ; c1ccc(-c2ccc(N(c3ccc(-c4ccccc4)cc3)c3ccc(-c4ccccc4)cc3)cc2)cc1 ; c1ccc(-c2ccc(N(c3ccc(-c4ccccc4)cc3)c3ccc(-c4ccc5oc6ccc(-c7ccccc7)cc6c5c4)cc3)cc2)cc1 ; c1ccc(-c2ccc(N(c3ccccc3)c3ccc(-c4ccc(-c5ccc6oc7ccc(-c8ccccc8)cc7c6c5)cc4)cc3)cc2)cc1]

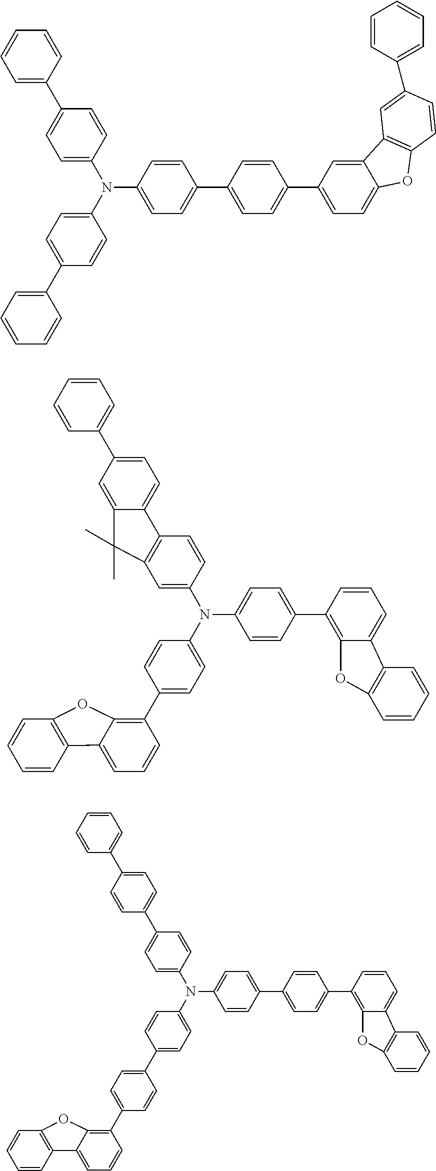

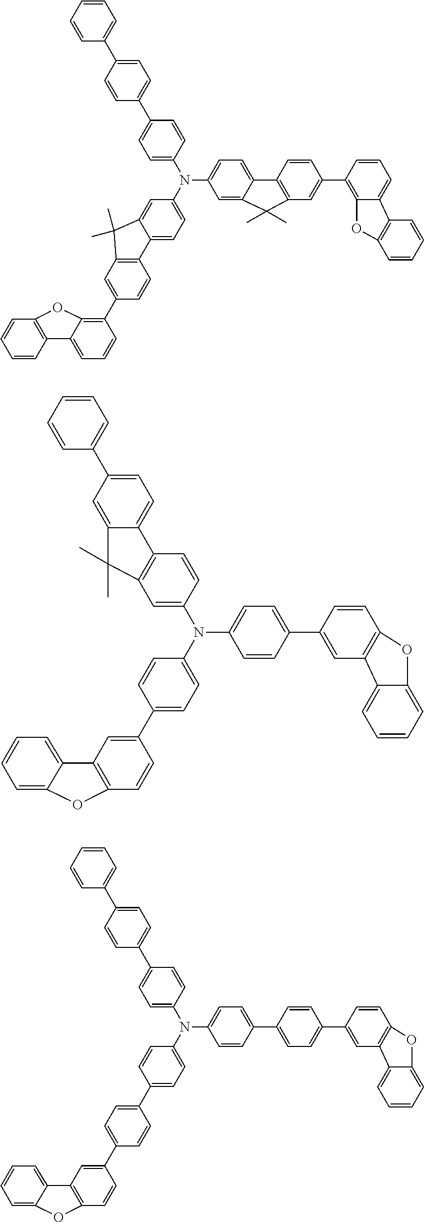

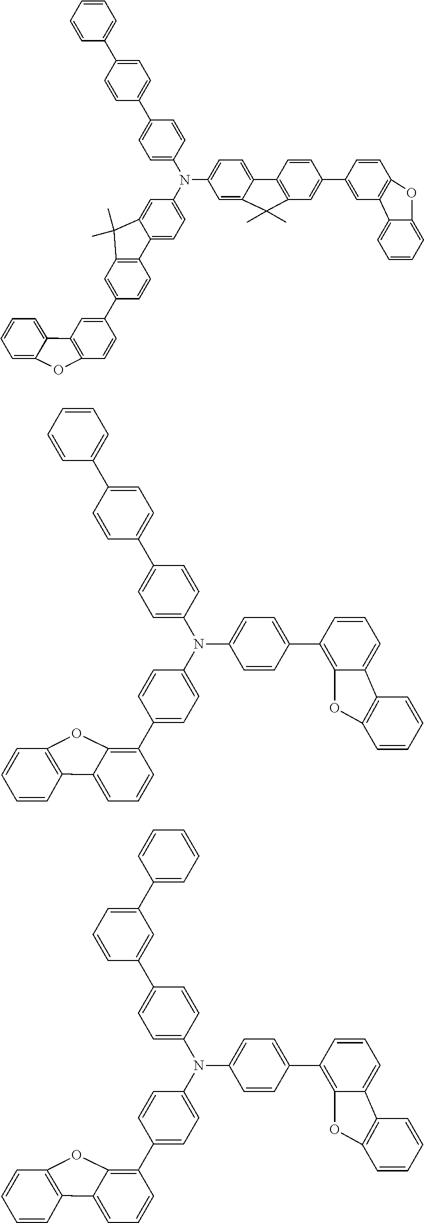

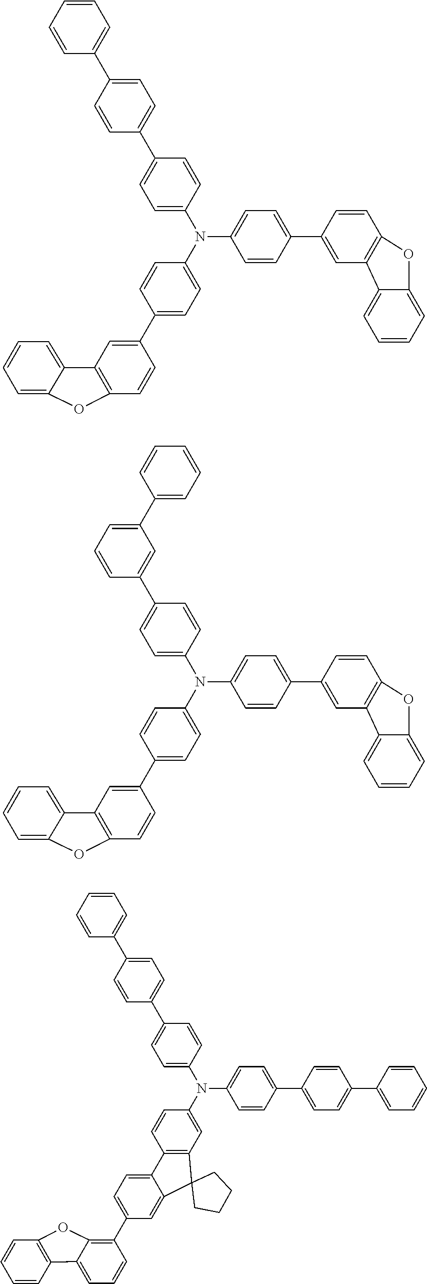

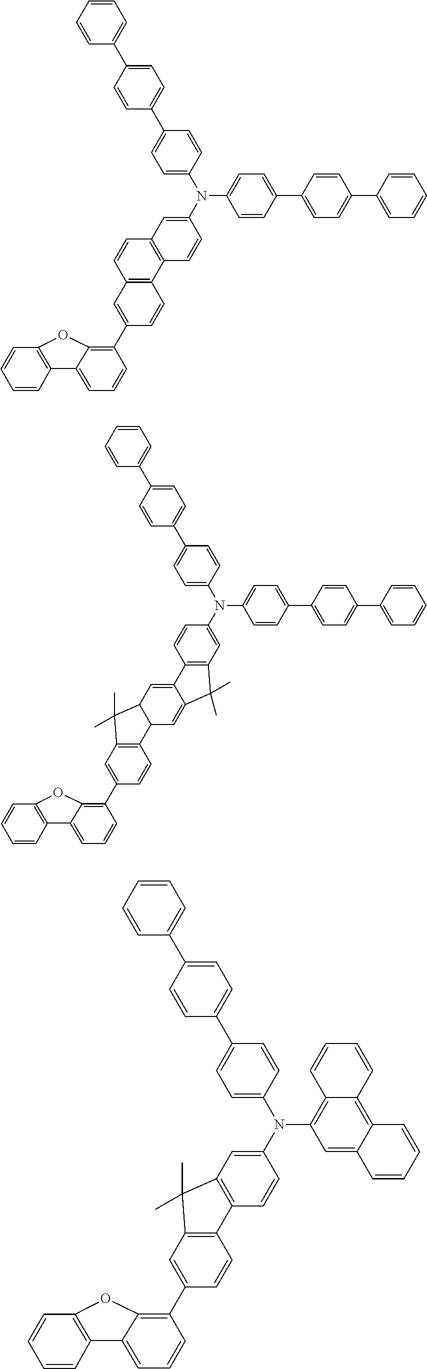 [SMILES: c1ccc(-c2ccc(-c3ccc(N(c4ccc(-c5ccc(-c6ccccc6)cc5)cc4)c4ccc5c(ccc6cc(-c7cccc8c7oc7ccccc78)ccc65)c4)cc3)cc2)cc1 ; CC1(C)C2=CC3c4ccc(-c5cccc6c5oc5ccccc56)cc4C(C)(C)C3C=C2c2ccc(N(c3ccc(-c4ccc(-c5ccccc5)cc4)cc3)c3ccc(-c4ccc(-c5ccccc5)cc4)cc3)cc21 ; CC1(C)c2cc(-c3cccc4c3oc3ccccc34)ccc2-c2ccc(N(c3ccc(-c4ccc(-c5ccccc5)cc4)cc3)c3cc4ccccc4c4ccccc34)cc21]

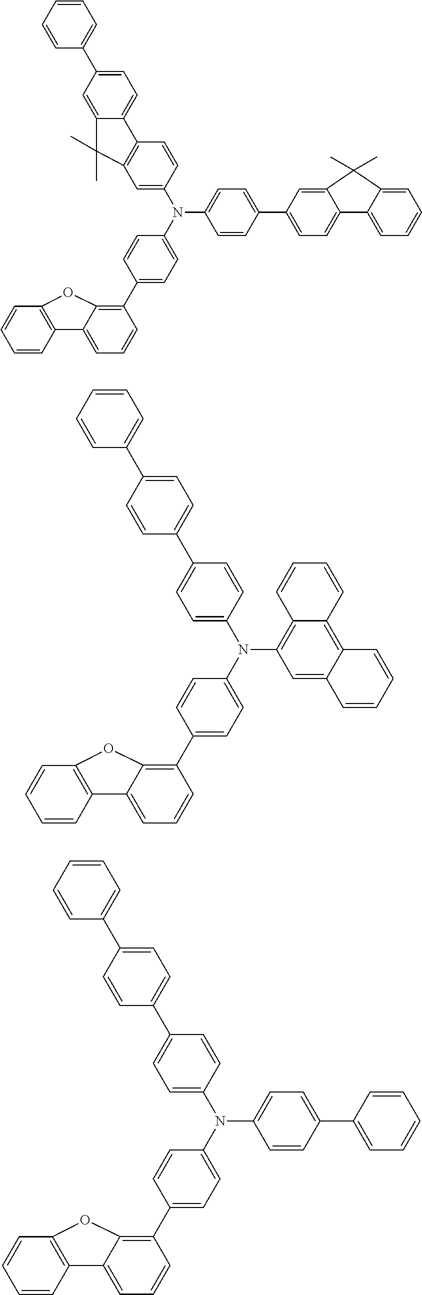

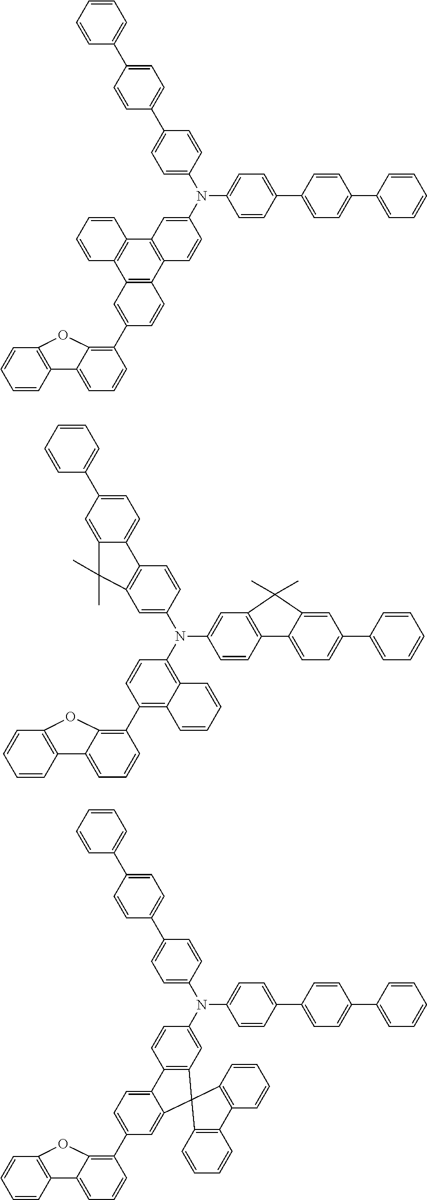

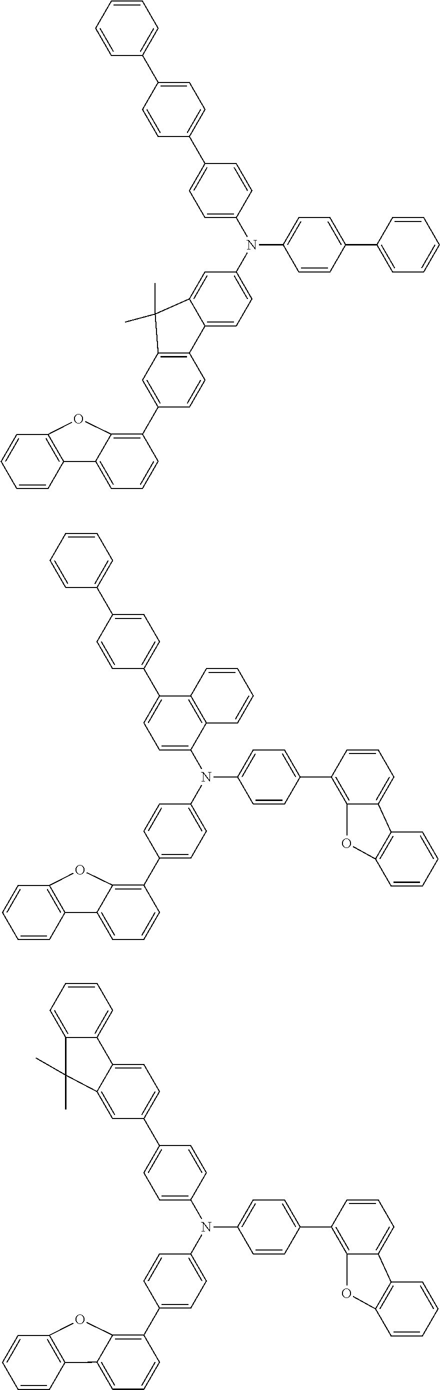 [SMILES: CC1(C)c2cc(-c3cccc4c3oc3ccccc34)ccc2-c2ccc(N(c3ccc(-c4ccccc4)cc3)c3ccc(-c4ccc(-c5ccccc5)cc4)cc3)cc21 ; c1ccc(-c2ccc(-c3ccc(N(c4ccc(-c5cccc6c5oc5ccccc56)cc4)c4ccc(-c5cccc6c5oc5ccccc56)cc4)c4ccccc34)cc2)cc1 ; CC1(C)c2ccccc2-c2ccc(-c3ccc(N(c4ccc(-c5cccc6c5oc5ccccc56)cc4)c4ccc(-c5cccc6c5oc5ccccc56)cc4)cc3)cc21]

In addition, an aromatic tertiary amine compound and a styrylamine compound selected from N, N,N′,N′-tetraphenyl-4, 4′-diaminophenyl; N, N′-diphenyl-N, N,N′-bis(3-methylphenyl)-[1,1′-biphenyl]-4,4′-diamine (TPD); 2,2-bis(4-di-p-tolylaminophenyl)propane; 1,1-bis(4-di-p-tolylaminophenyl)cyclohexane; N, N, N′,N′-tetra-p-tolyl-4, 4′-diaminobiphenyl; 1,1-bis(4-di-p-tolylaminophenyl)-4-phenylcyclohexane; bis(4-dimethylamino-2-methylphenyl)phenylmethane; bis(4-di-p-tolylaminophenyl)phenylmethane; N, N′-diphenyl-N, N′-di(4-methoxyphenyl)-4, 4′-diaminobiphenyl; N, N, N′,N′-tetraphenyl-4, 4′-diaminodiphenyl ether; 4, 4′-bis(diphenylamino)quardriphenyl; N,N,N-tri(p-tolyl)amine; 4-(di-p-tolylamino)-4′-[4-(di-p-tolylamino)styryl]stilbene; 4-N, N-diphenylamino-(2-diphenylvinyl)benzene; 3-methoxy-4′-N,N-diphenylaminostilbenzene; N-phenylcarbazole; those having two condensed aromatic rings in the molecule, for example, 4, 4′-bis(N-(1-naphthyl)-N-phenylamino)biphenyl (NPD); 4, 4′,4″-tris(N-(3-methylphenyl)-N-phenylamino)triphenylamine (MTDATA) in which the three triphenylamine units are connected like a starburst, may be used in the hole transporting layer.

In one aspect of the present invention, the hole transporting layer may be formed using a hole transporting layer composition containing a hole transporting material and a solvent.

The hole transporting material may be a high-molecular-weight compound such as a polymer, or may be a low-molecular-weight compound such as a monomer. From the viewpoint of charge injection obstacle avoidance, compounds having an ionization potential of 4.5 eV to 6.0 eV are preferred. Examples of the hole transporting material include aromatic amine derivatives. phthalocyanine derivatives, porphyrin derivatives, oligothiophene derivatives, polythiophene derivatives, benzylphenyl derivatives, compounds with tertiary amines bonding via a fluorene group, hydrazone derivatives, silazane derivatives, silanamine derivatives, phosphamine derivatives, quinacridone derivatives, polyaniline derivatives, polypyrrole derivatives, polyphenylenevinylene derivatives, polythienylenevinylene derivatives, polyquinoline derivatives, polyquinoxaline derivatives, carbon, etc.

Regarding the term derivative used herein, for example, when an aromatic amine derivative is referred to as one example, the derivative includes compounds containing an aromatic amine itself or having an aromatic amine as the main skeleton thereof, and may be a polymer or a monomer.

From the viewpoint of amorphousness and visible light transmittance, aromatic amine compounds of those exemplified above are preferred and aromatic tertiary amine compounds are especially preferred. Here, the aromatic tertiary amine compound is a compound having an aromatic tertiary amine structure, and includes compounds having an aromatic tertiary amine-derived group.

The type of the aromatic tertiary amine compound is not specifically limited, but from the viewpoint of uniform light emission owing to the surface smoothing effect thereof, polymer compounds having a weight-average molecular weight of 1,000 or more and 1,000,000 or less (polymer-type compounds with continuous repetitive units) are more preferred. Preferred examples of the aromatic tertiary amine polymer compound include polymer compounds with continuous repetitive units represented by the following formula (I).

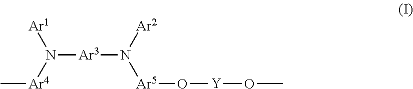

In the formula (I), Ar¹ and Ar² each independently represent a substituted or unsubstituted aromatic hydrocarbon group or a substituted or unsubstituted aromatic heterocyclic group. Ar³ to Ar⁵ each independently represent a substituted or unsubstituted aromatic hydrocarbon group or a substituted or unsubstituted aromatic heterocyclic group. Among Ar¹ to Ar⁵, two groups bonding to the same N atom may bond to each other to form a ring. Y represents a linking group selected from the following.

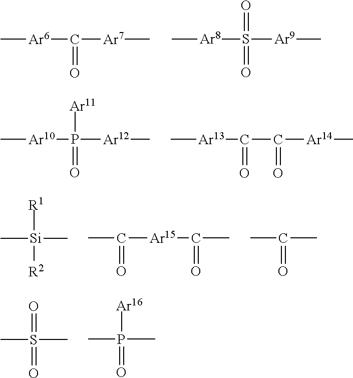

In the above formulae, Ar⁶ to Ar¹⁶ each independently represent a substituted or unsubstituted aromatic hydrocarbon group or a substituted or unsubstituted aromatic heterocyclic group. R¹ and R² each independently represent a hydrogen atom or a substituent.

The aromatic hydrocarbon group and the aromatic heterocyclic group for Ar¹ to Ar¹⁶ are, from the viewpoint of the solubility, the heat resistance, the hole injection/transportation capability of the polymer compound, preferably a group having a ring selected from a benzene ring, a naphthalene ring, a phenanthrene ring, a thiophene ring and a pyridine ring, and a group having a ring selected from a benzene ring and a naphthalene ring is more preferred.

The molecular weight of the optional substituent for the aromatic hydrocarbon group and the aromatic heterocyclic group for Ar¹ to Ar¹⁶ is generally 400 or less, but preferably around 250 or less. The substituent is preferably an alkyl group, an alkenyl group, an alkoxy group, an aromatic hydrocarbon group, an aromatic heterocyclic group, etc.

The substituent to be represented by R¹ and R² includes an alkyl group, an alkenyl group, an alkoxy group, a silyl group, a siloxy group, an aromatic hydrocarbon group, an aromatic heterocyclic group, etc.

As the hole transporting material, a polythiophene derivative of an electroconductive polymer prepared by polymerizing 3,4-ethylenedioxythiophene in a high-molecular-weight polystyrenesulfonic acid (PEDOT/PSS) is also preferred. The terminal of the polymer may be capped with a methacrylate, etc.

The concentration of the hole transporting material in the hole transporting layer composition may be any arbitrary one, but from the viewpoint of the uniformity of the thickness of the coating film, the concentration is generally 0.01% by mass or more, preferably 0.1% by mass or more, more preferably 0.5% by mass or more, and is generally 70% by mass or less, preferably 60% by mass or less, more preferably 50% by mass or less. When the concentration falls within the range, there may occur no film unevenness or may occur no defects in the hole transporting layer.

The hole transporting layer composition may contain an electron accepting compound.

The electron accepting compound is preferably a compound having an oxidation power and having an ability to accept one electron from the above-mentioned hole transporting material. Specifically, a compound having an electron affinity of 4 eV or more is preferred, and a compound having 5 eV or more is more preferred.

Examples of the electron accepting compound include one or more compounds selected from a group consisting of triarylboron compounds, metal halides, Lewis acids, organic acids, onium salts, salts of arylamines and metal halides, and salts of arylamines and Lewis acids. More specifically, there are mentioned onium salts having an organic group such as 4-isopropyl-4′-methyldiphenyliodonium tetrakis(pentafluorophenyl)borate, triphenylsulfonium tetrafluoroborate, etc.; high-valence inorganic compounds such as iron(III) chloride, ammonium peroxodisulfate, etc.; cyano compounds such as tetracyanoethylene, etc.; aromatic boron compounds such as tris(pentafluorophenyl)borane, etc.; fullerene derivatives; iodine; sulfonate ions such as polystyrenesulfonate ion, alkylbenzenesulfonate ion, camphorsulfonate ion, etc.

The electron accepting compound oxidizes the hole transporting material to thereby improve the electroconductivity of the hole transporting layer.

The content of the electron accepting compound to the hole transporting material in the hole transporting layer composition is generally 0.1 mol % or more, preferably 1 mol % or more. However, in general, the content is 100 mol % or less, preferably 40 mol % or less.

In addition to the above-mentioned hole transporting material and the electron accepting compound, further, any other component may be contained in the hole transporting layer composition. Examples of the other component include various light emitting materials, electron transporting compounds, binder resins, coating improver, etc. One alone or two or more such other components may be used in any combination and in any ratio.

In one aspect of the present invention, a hole transporting material suitable for a coating method is preferably used. Such a hole transporting material includes polyvinylcarbazole or its derivatives, polysilane or its derivatives, polysiloxane derivatives having an aromatic amine residue in the side chain or in the main chain, pyrazoline derivatives, arylamine derivatives, stilbene derivatives, triphenyldiamine derivatives, polyaniline or its derivatives, polythiophene or its derivatives, polypyrrole or its derivatives, polyarylamine or its derivatives, poly(p-phenylenevinylene) or its derivatives, polyfluorene derivatives, polymer compounds having an aromatic amine residue, and poly(2,5-thienylenevinylene) or its derivatives.

The hole transporting material is preferably a high-molecular-weight compound, for example, a polymer. The high-molecular-weight compound can improve film formability and can realize uniform light emission from organic EL devices. For example, the number-average molecular weight of the hole transporting material of the type, as calibrated with standard polystyrene, is 10,000 or more, preferably 3.0×10⁴ to 5.0×10⁵, more preferably 6.0×10⁴ to 1.2×10⁵. The weight-average molecular weight of the hole transporting material is 1.0×10⁴ or more, preferably 5.0×10⁴ to 1.0×10⁶, more preferably 1.0×10⁵ to 6.0×10⁵.

The hole transporting material is preferably a high-molecular compound of polyvinylcarbazole or its derivatives, polysilane or its derivatives, polysiloxane derivatives having an aromatic amine residue in the side chain or in the main chain, polyaniline or its derivatives, polythiophene or its derivatives, polyfluorene derivatives, polymer compounds having an aromatic amine residue, poly(p-phenylenevinylene) or its derivatives, and poly(2,5-thienylenevinylene) or its derivatives, etc., and is more preferably a high-molecular compound of polyvinylcarbazole or its derivatives, polysilane or its derivatives, polysiloxane derivatives having an aromatic amine residue in the side chain or in the main chain, polyfluorene derivatives, and high-molecular compounds having an aromatic amine residue. In the case where the hole transporting material is a low-molecular material, preferably, the material is dispersed in a polymer binder for use herein.

The polyvinylcarbazole or its derivatives may be produced, for example, by cationic polymerization or radical polymerization of a vinyl monomer.

Regarding polysiloxane or its derivatives, the siloxane skeleton structure does not almost have hole transporting capability, and therefore compounds having a residue of the above-mentioned low-molecular hole transporting material in the side chain or in the main chain are favorably used. In particular, there are mentioned compounds having a hole transporting aromatic amine residue in the side chain or in the main chain.

As the hole transporting material, a polymer having a fluorene-diyl unit represented by the following formula (Z) is preferred. This is because when the hole transporting material in an organic EL device is formed through contact with an organic compound having a condensed ring or plural aromatic rings, the hole injection efficiency is improved and the current density in operation can be enlarged.

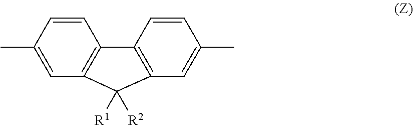

In the formula (Z), R¹ and R² may be the same or different and each independently represents a hydrogen atom, an alkyl group, an alkoxy group, an aryl group, or a monovalent heterocyclic group. The alkyl group includes a group having 1 to 10 carbon atoms. The alkoxy group includes a group having 1 to 10 carbon atoms. Examples of the aryl group include a phenyl group, a naphthyl group, etc. Examples of the monovalent heterocyclic group include a pyridyl group, etc. The aryl group and the monovalent heterocyclic group may have a substituent. Examples of the substituent include, from the viewpoint of improving the solubility of the polymer compound, an alkyl group having 1 to 10 carbon atoms, an alkoxy group having 1 to 10 carbon atoms, etc.

In the formula (Z), the aryl group and the monovalent heterocyclic group may have a crosslinking group. Examples of the crosslinking group include a vinyl group, an ethynyl group, a butenyl group, a group having an acrylic structure, a group having an acrylate structure, a group having an acrylamide structure, a group having a methacrylic structure, a group having a methacrylate structure, a group having a methacrylamide structure, a group having a vinyl ether structure, a vinylamino group, a group having a silanol structure, a group having a small ring (for example, cyclopropane, cyclobutane, epoxide, oxetane, diketone, episulfido, etc.), etc.

Preferred examples of the fluorene-diyl unit are shown below.

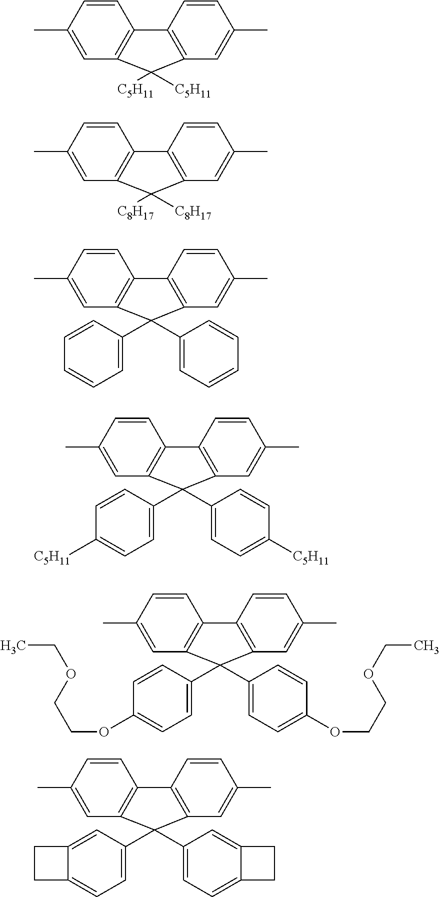

An especially preferred hole transporting material is a polymer containing the above-mentioned fluorene-diyl unit and an aromatic tertiary amine compound unit as repetitive units, for example, a polyarylamine polymer.

The aromatic tertiary amine compound unit includes a repetitive unit represented by the following formula (K).

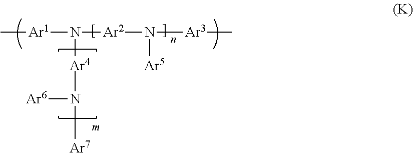

In the formula (K), Ar¹, Ar², Ar³ and Ar⁴ each independently represent an arylene group or a divalent heterocyclic group. Ar⁵, Ar⁶ and Ar⁷ each independently represent an aryl group or a monovalent heterocyclic group. Ar⁶ and Ar⁷ may form a ring along with the nitrogen atom to which Ar⁶ and Ar⁷ bond. m and n each independently indicate 0 or 1.

Examples of the arylene group include a phenylene group, etc. Examples of the divalent heterocyclic group include a pyridinediyl group, etc. These groups may have a substituent.

Examples of the aryl group include a phenyl group, a naphthyl group, etc. Examples of the monovalent heterocyclic group include a pyridyl group, etc. These groups may have a substituent.

Examples of the monovalent heterocyclic group include a thienyl group, a furyl group, a pyridyl group, etc.

The optional substituent for the arylene group, the aryl group, the divalent heterocyclic group and the monovalent heterocyclic group is, from the viewpoint of the solubility of the polymer compound, preferably an alkyl group, an alkoxy group and an aryl group, more preferably an alkyl group. The alkyl group includes a group having 1 to 10 carbon atoms. The alkoxy group includes a group having 1 to 10 carbon atoms. Examples of the aryl group include a phenyl group, a naphthyl group, etc.

The substituent may have a crosslinking group. Examples of the crosslinking group include a vinyl group, an ethynyl group, a butenyl group, a group having an acrylic structure, a group having an acrylate structure, a group having an acrylamide structure, a group having a methacrylic structure, a group having a methacrylate structure, a group having a methacrylamide structure, a group having a vinyl ether structure, a vinylamino group, a group having a silanol structure, a group having a small ring (for example, cyclopropane, cyclobutane, epoxide, oxetane, diketone, episulfido, etc.), etc.

In the formula (K), Ar¹, Ar², Ar³ and Ar⁴ each are preferably an arylene group, more preferably a phenylene group. Ar⁵, Ar⁶ and Ar⁷ each are preferably an aryl group, more preferably a phenyl group.

Further, the carbon atom in Are may directly bond to the carbon atom in Ar³, or may bond thereto via a divalent group such as —O—, —S—, etc.

From the viewpoint of easiness in monomer synthesis, m and n are preferably 0.

Specific examples of the repetitive unit represented by the formula (K) include repetitive units represented by the following formulae.

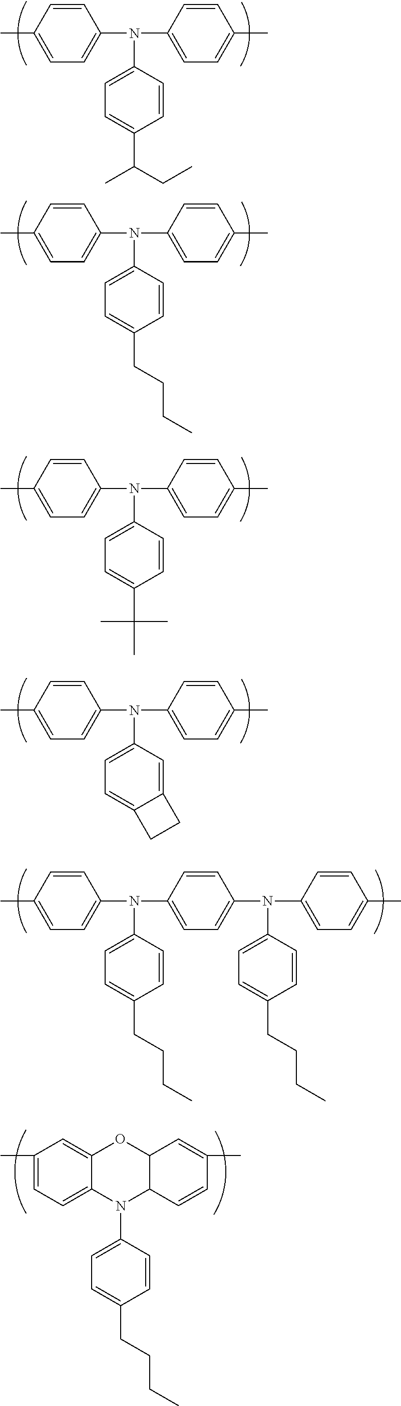

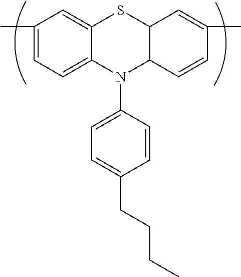

In the case where the hole transporting material does not have a crosslinking group, it is desirable that a crosslinking agent having a crosslinking group is used. Examples of the crosslinking agent include compounds having a polymerizable substituent selected from a group consisting of a vinyl group, an acetyl group, a butenyl group, an acryl group, an acrylamide group, a methacryl group, a methacrylamide group, a vinyl ether group, a vinylamino group, a silanol group, a cyclopropyl group, a cyclobutyl group, an epoxy group, an oxetane group, a diketene group, an episulfide group, a lactone group, and a lactam group. The crosslinking agent is, for example, preferably a polyfunctional acrylate, including dipentaerythritol hexaacrylate (DPHA), trispentaerythritol octaacrylate (TPEA), etc.

Using such a material having a crosslinking group or using such a crosslinking agent makes it possible to effectively prevent the lower layer from being dissolved by the solvent or the like for the upper layer formation, even though any additional functional layer (upper layer) is formed on the lower layer (hole transporting layer) according to a coating method.

In one aspect of the present invention, a hole transporting material having a hole transporting site and having a crosslinking group is preferably used. Examples of the hole transporting site include three-ring or more multi-ring aromatic ring structures such as triarylamine structure, fluorene ring, anthracene ring, pyrene ring, carbazole ring, dibenzofuran ring, dibenzothiophene ring, phenoxazine ring, phenanthroline ring, etc.; aromatic heterocyclic structures such as thiophene ring, silol ring. etc.; and metal complex structures.

Above all, from the viewpoint of improving electrochemical stability and hole transporting performance, triarylamine structures are preferred for the hole transporting site.

From the viewpoint that the crosslinking reaction may often make the resultant product insoluble in organic solvent, a polymer is preferred. In particular, from the viewpoint of improving electrochemical stability and hole transporting performance, a polymer having repetitive units represented by the following formula (L) is preferred.

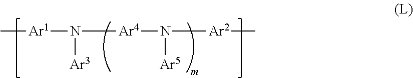

In the formula (L), m indicates an integer of 0 to 3, Ar¹ and Ar² each independently represent a single bond, a substituted or unsubstituted aromatic hydrocarbon group, or a substituted or unsubstituted aromatic heterocyclic group, Ar³ to Ar⁵ each independently represent a substituted or unsubstituted aromatic hydrocarbon group or a substituted or unsubstituted aromatic heterocyclic group. However, both Ar¹ and Ar² are not single bonds at the same time.

Examples of the aromatic hydrocarbon group include a monovalent group of a 6-membered single ring or a condensed ring containing two to five 6-membered rings, such as a benzene ring, a naphthalene ring, an anthracene ring, a phenanthrene ring, a perylene ring, a tetracene ring, a pyrene ring, a benzopyrene ring, a chrysene ring, a triphenylene ring, an acenaphthene ring, a fluoranthene ring, a fluorene ring, etc.

Examples of the aromatic heterocyclic group include a monovalent group of a 5- or 6-membered single ring or a condensed ring containing two to four 5- or 6-membered rings, such as a furan ring, a benzofuran ring, a thiophene ring, a benzothiophene ring, a pyrrole ring, a pyrazole ring, an imidazole ring, an oxadiazole ring, an indole ring, a carbazole ring, a pyrroloimidazole ring, a pyrrolopyrazole ring, a pyrrolopyrrole ring, a thienopyrrole ring, a thienothiophene ring, a furopyrrole ring, a furofuran ring, a thienofuran ring, a benzisoxazole ring, a benzisothiazole ring, a benzimidazole ring, a pyridine ring, a pyrazine ring, a pyridazine ring, a pyrimidine ring, a triazine ring, a quinoline ring, an isoquinoline ring, a cinnoline ring, a quinoxaline ring, a phenanthridine ring, a benzimidazole ring, a perimidine ring, a quinazoline ring, a quinazolinone ring, an azurene ring, etc.

From the viewpoint of solubility in solvent and heat resistance, Ar¹ to Ar⁵ each are preferably a monovalent group of a ring selected from a group consisting of a benzene ring, a naphthalene ring, an anthracene ring, a phenanthrene ring, a triphenylene ring, a pyrene ring, a thiophene ring, a pyridine ring, and a fluorene ring.

Also preferably, Ar¹ to Ar⁵ each are a group formed by bonding one or more rings selected from the above-mentioned group, via a single bond, and are more preferably any of a biphenyl group, a biphenylene group, a terphenyl group and a terphenylene group.

The optional substituent for the aromatic hydrocarbon group and the aromatic heterocyclic group includes a linear, branched or cyclic alkyl group having 1 to 24, preferably 1 to 12 carbon atoms, such as a methyl group, an ethyl group, an n-propyl group, an i-propyl group, an n-butyl group, an i-butyl group, a sec-butyl group, a tert-butyl group, an n-hexyl group, a cyclohexyl group, a dodecyl group, etc.; an alkenyl group having 2 to 24, preferably 2 to 12 carbon atoms, such as a vinyl group, etc.; an alkynyl group having 2 to 24, preferably 2 to 12 carbon atoms, such as an ethynyl group, etc.; an alkoxy group having 1 to 24, preferably 1 to 12 carbon atoms, such as a methoxy group, an ethoxy group, etc.; an aryloxy group having 4 or more, preferably 5 or more and 36 or less, preferably 24 or less carbon atoms, such as a phenoxy group, a naphthoxy group, a pyridyloxy group, etc.; an alkoxycarbonyl group having 2 to 24, preferably 2 to 12 carbon atoms, such as a methoxycarbonyl group, an ethoxycarbonyl group, etc.; a dialkylamino group having 2 to 24, preferably 2 to 12 carbon atoms, such as a dimethylamino group, a diethylamino group, etc.; a diarylamino group having 10 or more, preferably 12 or more and 36 or less, preferably 24 or less carbon atoms, such as a diphenylamino group, a ditolylamino group, an N-carbazolyl group, etc.; an arylalkylamino group having 7 to 36, preferably 7 to 24 carbon atoms, such as a phenylmethylamino group, etc.; an acyl group having 2 to 24, preferably 2 to 12 carbon atoms, such as an acetyl group, a benzoyl group, etc.; a halogen atom such as a fluorine atom, a chlorine atom, etc.; a haloalkyl group having 1 to 12, preferably 1 to 6 carbon atoms, such as a trifluoromethyl group, etc.; an alkylthio group having 1 to 24, preferably 1 to 12 carbon atoms such as a methylthio group, an ethylthio group, etc.; an arylthio group having 4 or more, preferably 5 or more and 36 or less, preferably 24 or less carbon atoms, such as a phenylthio group, a naphthylthio group, a pyridylthio group, etc.; a silyl group having 2 or more, preferably 3 or more and 36 or less, preferably 24 or less carbon atoms, such as a trimethylsilyl group, a triphenylsilyl group, etc.; a siloxy group having 2 or more, preferably 3 or more and 36 or less, preferably 24 or less carbon atoms, such as a trimethylsiloxy group, a triphenylsiloxy group, etc.; a cyano group; an aromatic hydrocarbon cyclic group having 6 to 36, preferably 6 to 24 carbon atoms such as a phenyl group, a naphthyl group, etc.; an aromatic heterocyclic group having 3 or more, preferably 4 or more and 36 or less, preferably 24 or less carbon atoms, such as a thienyl group, a pyridyl group, etc.

Among the above-mentioned optional substituents, an alkyl group having 1 to 12 carbon atoms and an alkoxy group having 1 to 12 carbon atoms are preferred from the viewpoint of solubility.

The above-mentioned optional substituents may further have a substituent, and examples of the substituent may be selected from the groups mentioned hereinabove as the optional substituents.

From the viewpoint of excellent hole transporting capability, the carbon number of Ar¹ to Ar⁵ may be, including the substituent thereof, 3 or more, preferably 5 or more, more preferably 6 or more, and may be 72 or less, preferably 48 or less, more preferably 25 or less.

m in the formula (L) indicates an integer of 0 to 3, and from the viewpoint of enhancing film formability, m is preferably 0. From the viewpoint of improving hole transporting capability, m is preferably 1 to 3.

When n is 2 or more, two or more Ar⁴'s and two or more Ar⁵'s may be the same as or different from each other. Further, Ar⁴'s and Ar⁵'s may bond to each other directly or via a linking group to form a cyclic structure.

When the hole transporting material has a crosslinking group, the solubility thereof in solvent may be greatly varied before and after the reaction (insolubilization reaction) to occur in heating and/or through irradiation with active energy rays.

The crosslinking group means a group that reacts with the same or a different group in the other molecule positioned closely in heating and/or through irradiation with active energy rays to form a novel chemical bond.

From the viewpoint of the ability to facilitate insolubilization, the crosslinking group includes, for example, the following crosslinking groups.

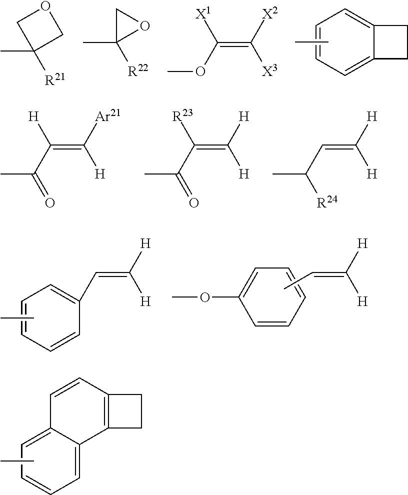

In the above formulae, R²¹ to R²³ each independently represent a hydrogen atom, or a substituted or unsubstituted alkyl group. Ar²¹ represents a substituted or unsubstituted aromatic group.

X¹, X² and X³ each independently represent a hydrogen atom or a halogen atom.

R²⁴ represents a hydrogen atom or a vinyl group.

The benzocyclobutene ring may have a substituents, and the substituents may bond to each other to form a ring.

The alkyl group for R²¹ to R²³ includes an alkyl group having 1 to 24, preferably 1 to 12 carbon atoms, for example, a methyl group, an ethyl group, etc.

The aromatic group for Ar²¹ includes the same aromatic groups that are represented by the above-mentioned Ar¹ to Ar⁵.

Examples of the optional substituent for R²¹ to R²³, and Ar²¹ include, though not limited thereto, the groups selected from the above-mentioned optional substituents.

Further, the crosslinking group is, from the viewpoint of high reactivity and easiness in insolubilization, preferably a group capable of being insolubilized through cationic polymerization, for example, a cyclic ether group such as an epoxy group, an oxetane group or the like, or a vinyl ether group, etc. Above all, from the viewpoint of facilitating cationic polymerization rate control, an oxetane group is especially preferred, and from the viewpoint that a hydroxyl group that may worsen devices is hardly formed during cationic polymerization, a vinyl ether group is preferred.

A group to undergo cyclization addition reaction, for example, an arylvinylcarbonyl group such as a cinnamoyl group or a group having a benzocyclobutene ring is preferred from the viewpoint of further enhancing electrochemical stability.

In addition, among the crosslinking group, a group having a benzocyclobutene ring is especially preferred from the viewpoint that the insolubilized structure is especially stable.

Specifically, a group represented by the following formula (M) is preferred.

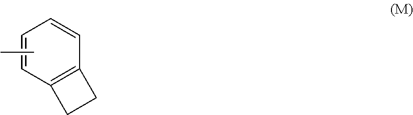

The benzocyclobutene ring in the formula (M) may have a substituent. The substituents may bond to each other to form a ring.

The crosslinking group may directly bond to the monovalent or divalent aromatic group in the molecule, but may bond thereto via a divalent group. The divalent group is preferably a divalent group to be formed by linking 1 to 30 groups selected from —O—, —C(═O)— and —CH²— optionally having a substituent, in any desired order. Specific examples of the crosslinking group that bonds via the divalent group are shown below, though not limited thereto.

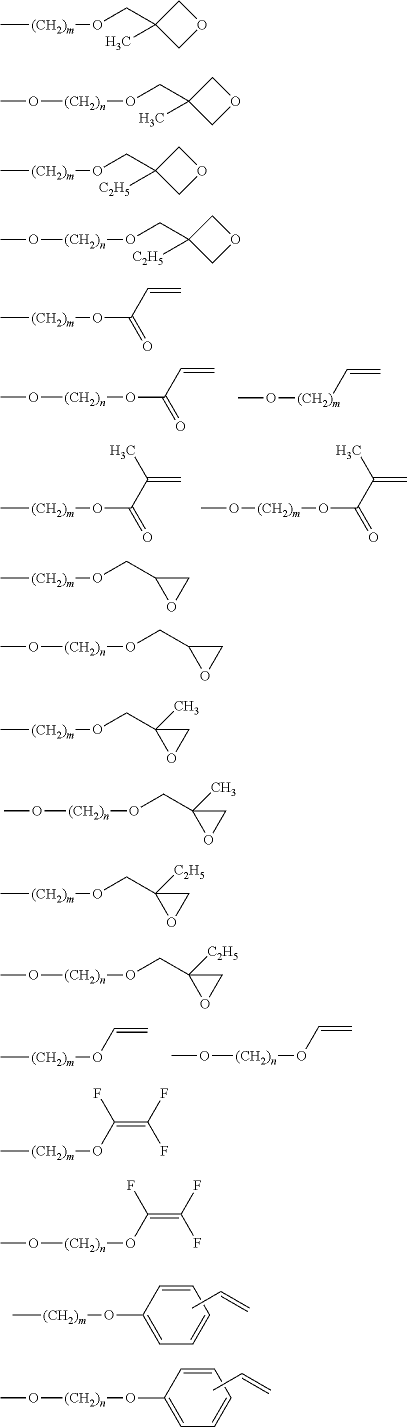

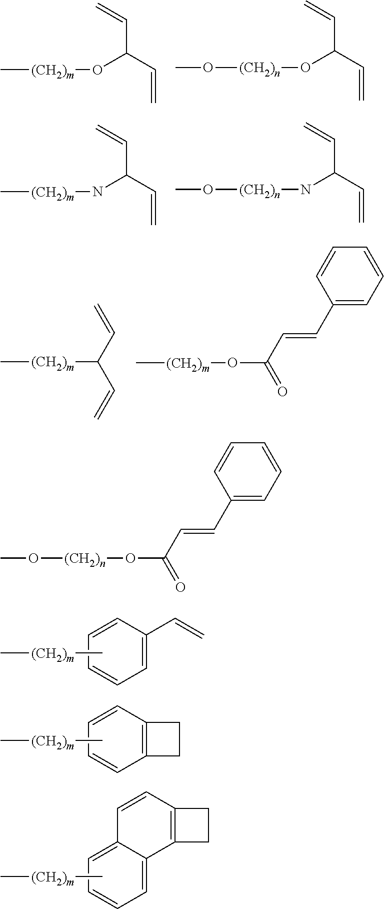

In the above formulae, m indicates an integer of 0 to 12, and n indicates an integer of 1 to 12.

Specific examples of a group containing any other crosslinking group are shown below.

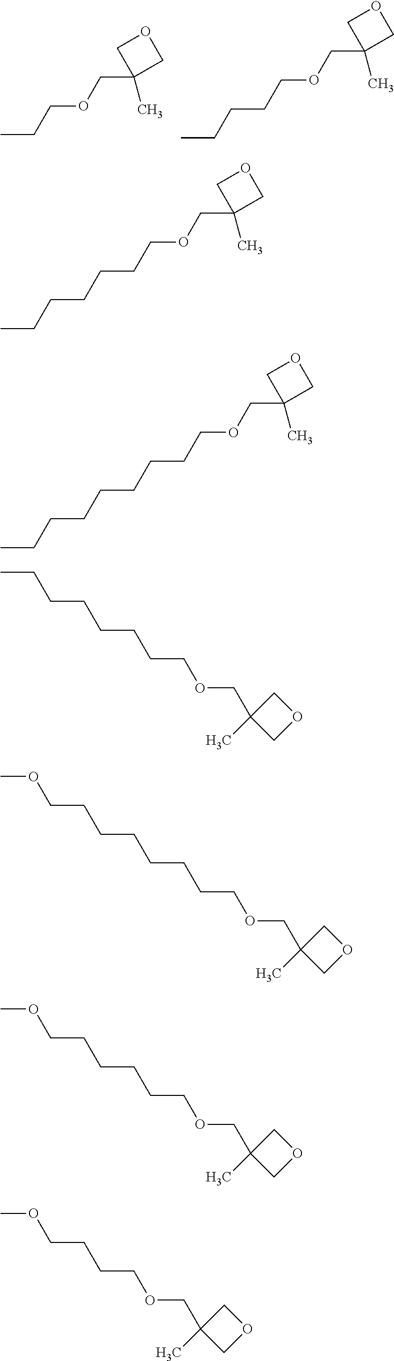

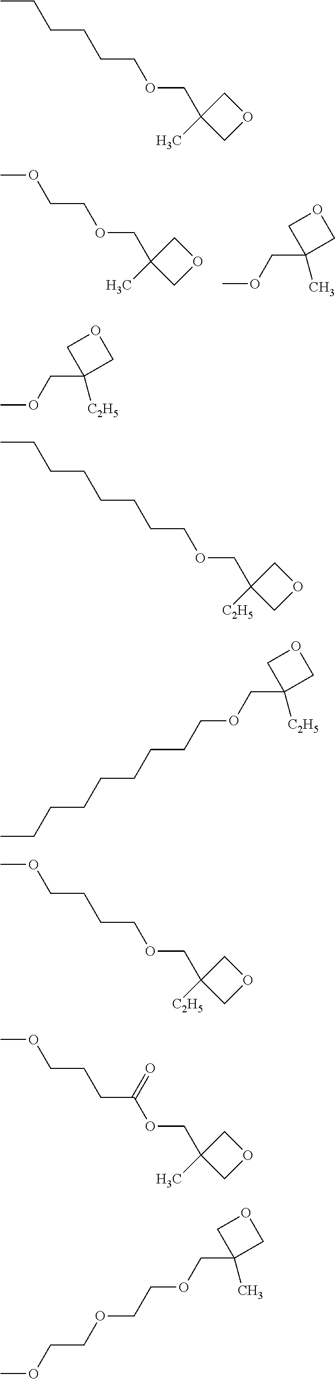

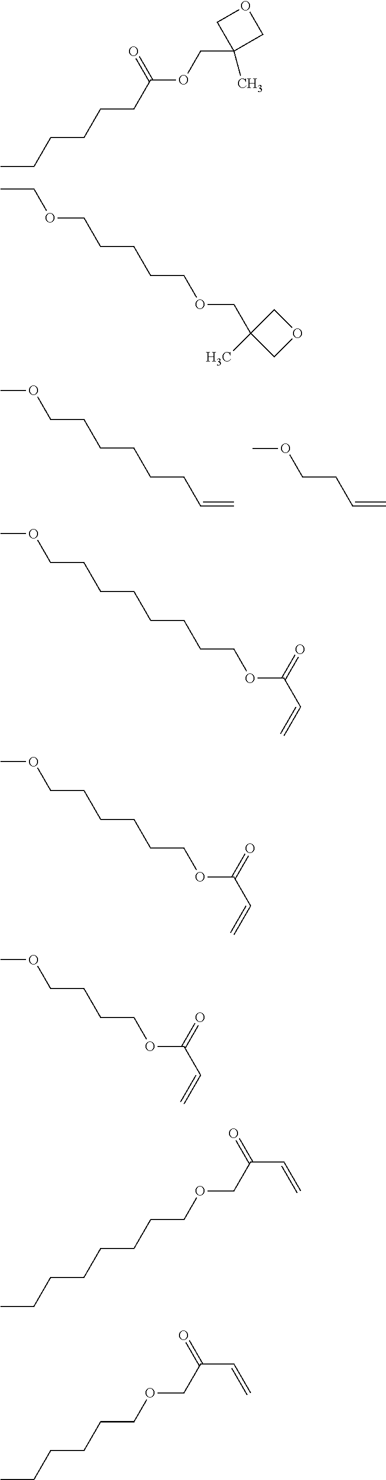

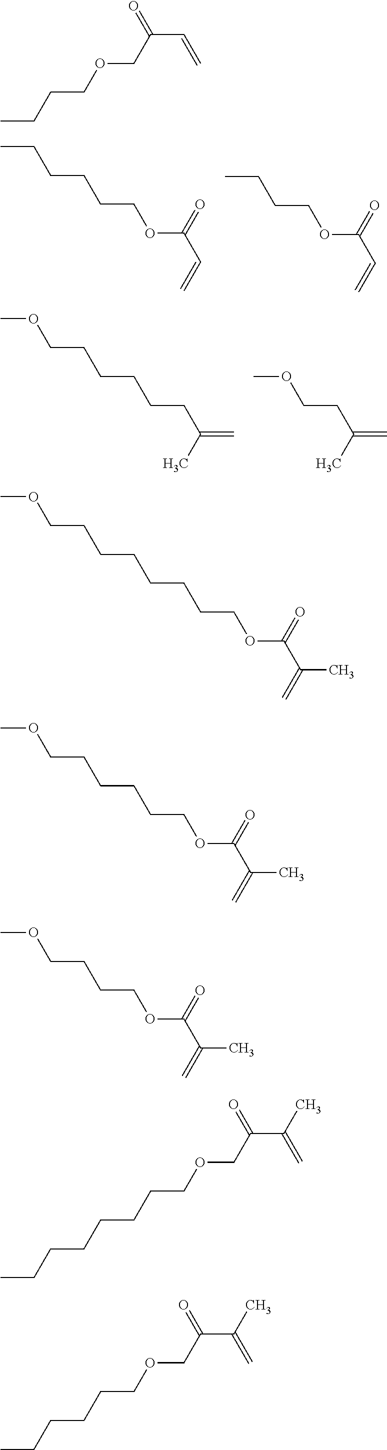

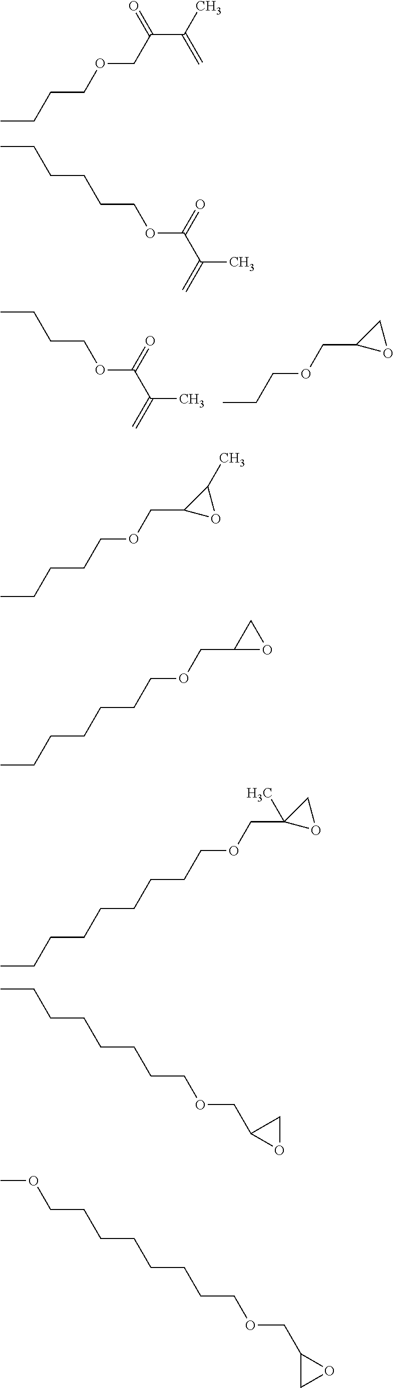

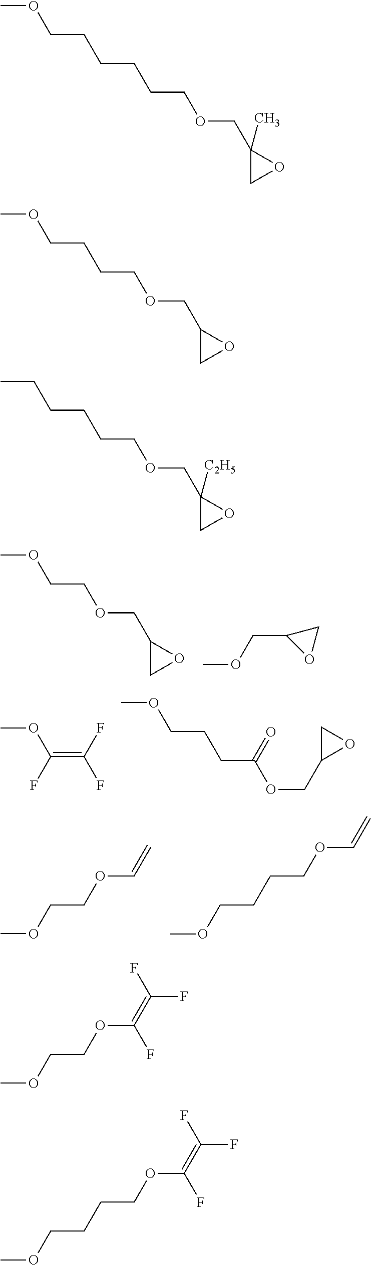

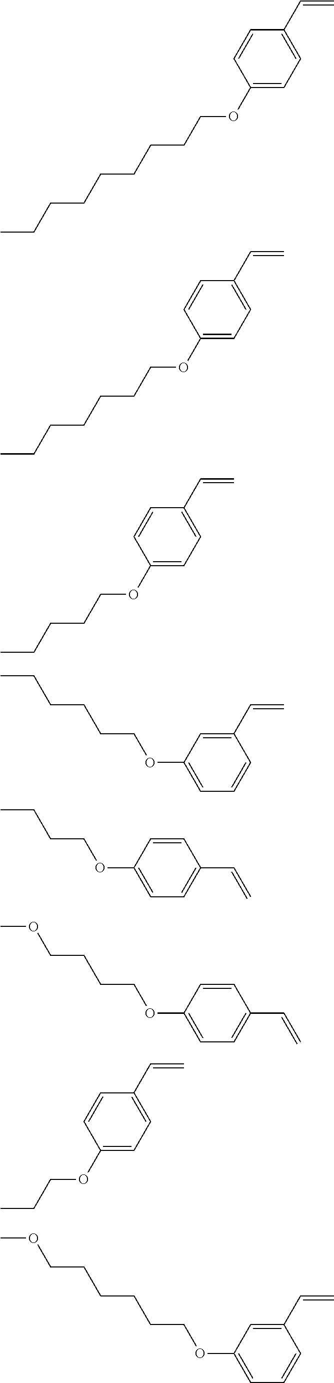

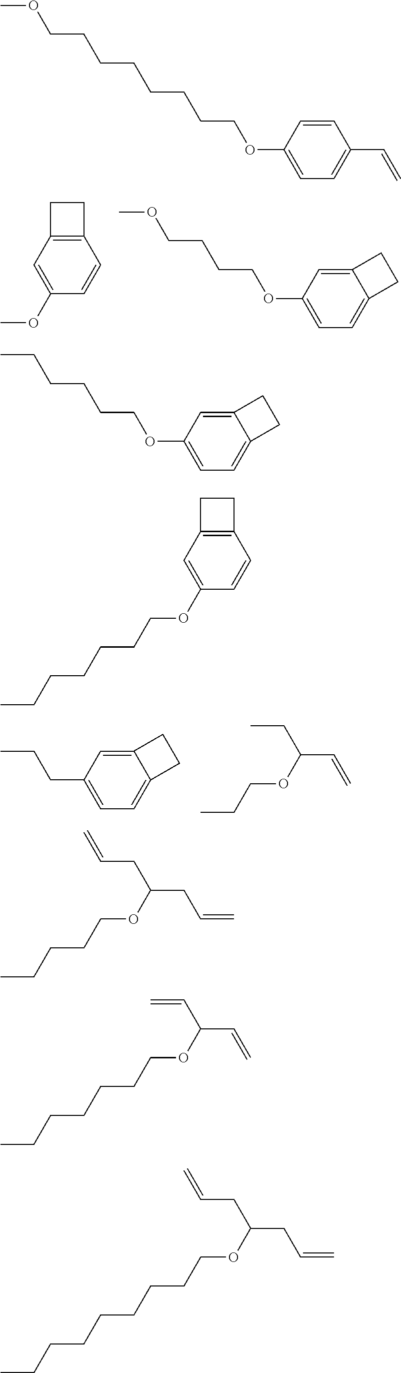

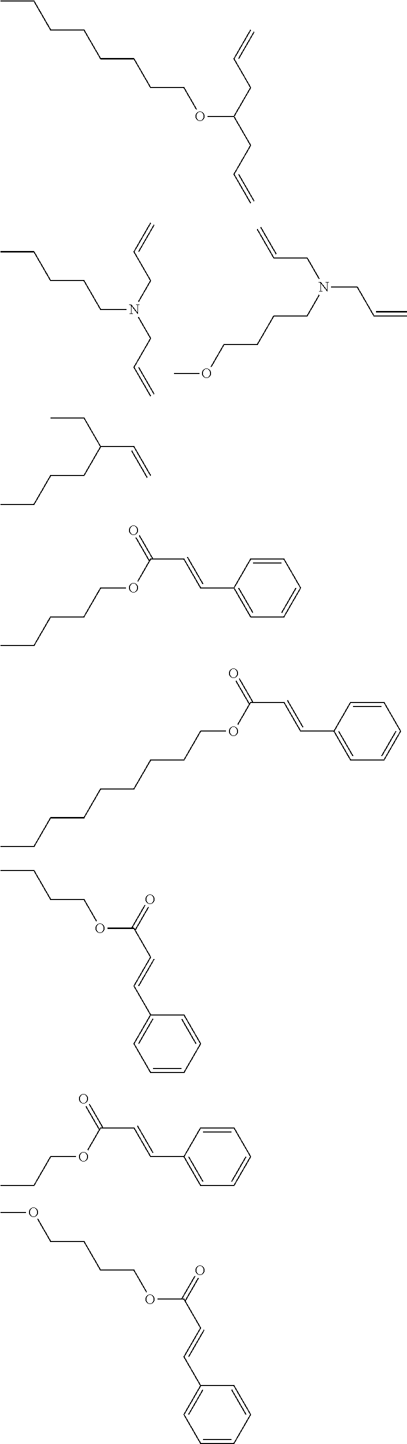

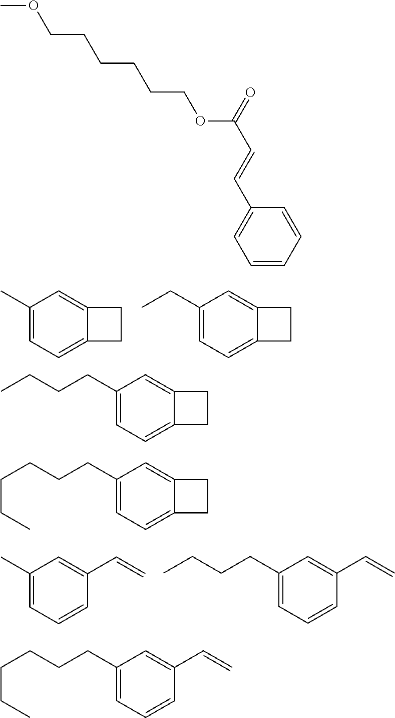

Further, in one aspect of the present invention, the hole transporting material preferably contains an electroconductive polymer or oligomer. The electroconductive polymer or oligomer is generally a mixture with an electron donating compound, an electron accepting compound, or an acidic compound. The mixture may be solid or liquid, but a solution, a dispersion, a colloid, an ink, a varnish or the like that is suitably used in a method of forming a solid film according to a coating method is preferred. In addition, for improving hole transporting capability and for improving film formability, an additive may be added to the mixture.

Examples of the electroconductive polymer or oligomer usable in one aspect of the present invention are shown below.

Typical examples of the electron donating compound include aromatic amine derivatives, phthalocyanine derivatives, porphyrin derivatives, thiophene derivatives, benzylphenyl derivatives, compounds with tertiary amines linked by a fluorene group, hydrazone derivatives, silazane derivatives, silanamine derivatives, phosphamine derivatives, quinacridone derivatives, aniline derivatives, pyrrole derivatives, phenylenevinylene derivatives, thienylenevinylene derivatives, quinoline derivatives, quinoxaline derivatives, carbon, etc. These derivatives may be any of low-molecular compounds having a molecular weight of less than 1,000, or oligomers or dendrimers having a molecular weight of 1,000 to 10,000, or polymers having a molecular weight of 10,000 or more. Above all, aromatic amine derivatives, polythiophene derivatives, polyaniline derivatives and oligoaniline derivatives are preferably used.

Typical examples of the electron accepting compound and the acidic compounds include one or more compounds selected from a group consisting of triarylboron compounds, metal halides, Lewis acids, organic acids, onium salts, salts of arylamines and metal halides, and salts of arylamines and Lewis acids. More specifically, there are mentioned onium salts having an organic group such as 4-isopropyl-4′-methyldiphenyliodonium tetrakis(pentafluorophenyl)borate, triphenylsulfonium tetrafluoroborate, etc.; high-valence inorganic compounds such as iron(III) chloride, ammonium peroxodisulfate, etc.; cyano compounds such as tetracyanoethylene, etc.; aromatic boron compounds such as tris(pentafluorophenyl)borane, etc.; fullerene derivatives; iodine; sulfonate ions such as polystyrenesulfonate ion, alkylbenzenesulfonate ion, camphorsulfonate ion, etc.

Like the electron donating compound, these derivatives may be any of low-molecular compounds having a molecular weight of less than 1,000, oligomers and dendrimers having a molecular weight of 1,000 to 10,000, and polymers having a molecular weight of 10,000 or more.

The electron accepting compound oxidizes the hole transporting material to increase the electroconductivity of the hole transporting layer. The content of the electron accepting compound relative to the hole transporting material in the hole transporting layer or in the hole transporting layer composition is generally 0.1 mol % or more, preferably 1 mol % or more and is generally 100 mol % or less, preferably 40 mol % or less.

Typical examples (i) to (x) of the hole transporting material usable in one aspect of the present invention are shown below. These may be used singly or as combined. Preferably, a relative electron donating one and a relatively electron accepting one are mixed. Further, additives for promoting charge transfer between the electron donating compound and the electron accepting compound and for improving coating film formability may be added as a third component. Plural third components may be used.

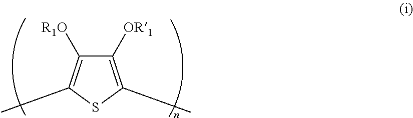

In the formula, R₁ and R₁′ each are independently selected from a hydrogen atom and an alkyl group having 1 to 4 carbon atoms. R₁ and R₁′ may bond to each other to form an alkylene chain having 1 to 4 carbon atoms. The alkylene chain may be optionally substituted with an alkyl group having 1 to 12 carbon atoms, an aromatic group having 6 to 12 carbon atoms, or a 1,2-cyclohexylene group. n indicates a number larger than about 6.

Polyaniline Having a Monomer Unit of (ii) and/or (iii):

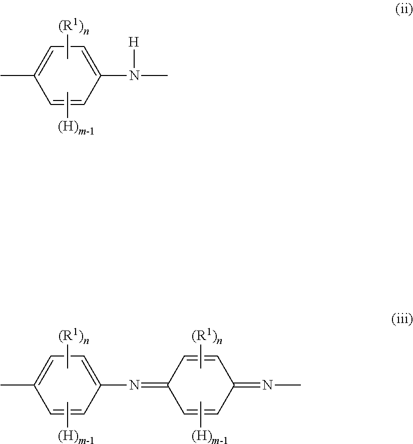

In the above formula, n indicates an integer of 0 to 4,

(m-1) indicates an integer of 1 to 5, and n+(m-1)=5,
R¹ may be the same or different and each is independently selected from an alkyl group, an alkenyl group, an alkoxy group, a cycloalkyl group, a cycloalkenyl group, an alkanoyl group, an alkylthio group, an aryloxy group, an alkylthioalkyl group, an alkylaryl group, ab arylalkyl group, an amino group, an alkylamino group, a dialkylamino group, an aryl group, an alkylsulfinyl group, an alkoxyalkyl group, an alkylsulfonyl group, an arylthio group, an arylsulfinyl group, an alkoxycarbonyl group, an arylsulfonyl group, a carboxyl group, a halogen atom, a cyano group, and an alkyl group substituted with one or more substituents of a sulfonic acid group, a carboxyl group, a halogen atom, a nitro group, a cyano group and an epoxy group. Neighboring two R¹'s may bond to each other to form an alkylene chain or an alkenylene chain to complete a 3-, 4-, 5-, 6- or 7-membered aromatic ring or alicyclic ring optionally containing one or more divalent nitrogen atoms, sulfur atoms or oxygen atoms.

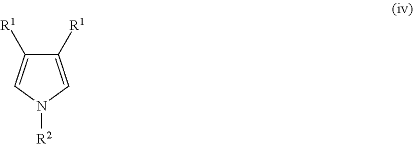

In the above formula, R¹ is independently selected from a hydrogen atom, an alkyl group, an alkenyl group, an alkoxy group, an alkanoyl group, an alkylthio group, an aryloxy group, an alkylthioalkyl group, an alkylaryl group, an arylalkyl group, an amino group, an alkylamino group, a dialkylamino group, an aryl group, an alkylsulfinyl group, an alkoxyalkyl group, an alkylsulfonyl group, an arylthio group, an arylsulfinyl group, an alkoxycarbonyl group, an arylsulfonyl group, an acrylic acid group, a phosphoric acid group, a phosphonic acid group, a halogen atom, a nitro group, a cyano group, a hydroxyl group, an epoxy group, a silyl group, a siloxane group, an alcohol group, a benzyl group, a carboxylate group, an ether group, an ether carboxylate group, an amide sulfonate group, an ether sulfonate group and an urethane group. Two R¹'s may bond to each other to form an alkylene chain or an alkenylene chain to complete a 3-, 4-, 5-, 6- or 7-membered aromatic ring or alicyclic ring, and the ring may contain one or more divalent nitrogen atoms, sulfur atoms or oxygen atoms.

R² is independently selected from a hydrogen atom, an alkyl group, an alkenyl group, an aryl group, an alkanoyl group, an alkylthioalkyl group, an alkylaryl group, an arylalkyl group, an amino group, an epoxy group, a silyl group, a siloxane group, an amide sulfonate group, an alcohol group, a benzyl group, a carboxylate group, an ether group, an ether carboxylate group, an amide sulfonate group, an ether sulfonate group and an urethane group.

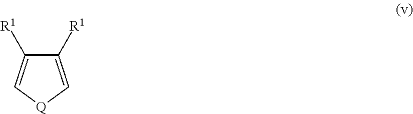

In the above formula, Q is selected from a group consisting of S, Se and Te, R¹ is each independently selected from a hydrogen atom, an alkyl group, an alkenyl group, an alkoxy group, an alkanoyl group, an alkylthio group, an aryloxy group, an alkylthioalkyl group, an alkylaryl group, an arylalkyl group, an amino group, an alkylamino group, a dialkylamino group, an aryl group, an alkylsulfinyl group, an alkoxyalkyl group, an alkylsulfonyl group, an arylthio group, an arylsulfinyl group, an alkoxycarbonyl group, an arylsulfonyl group, an acrylic acid group, a phosphoric acid group, a phosphonic acid group, a halogen atom, a nitro group, a cyano group, a hydroxyl group, an epoxy group, a silyl group, a siloxane group, an alcohol group, a benzyl group, a carboxylate group, an ether group, an ether carboxylate group, an amide sulfonate group, an ether sulfonate group, an ester sulfonate group and an urethane group. Two R¹'s may bond to each other to form an alkylene chain or alkenylene chain to complete a 3-, 4-, 5-, 6- or 7-membered aromatic ring or alicyclic ring. The ring may contain one or more divalent nitrogen atoms, selenium atoms, tellurium atoms, sulfur atoms or oxygen atoms.

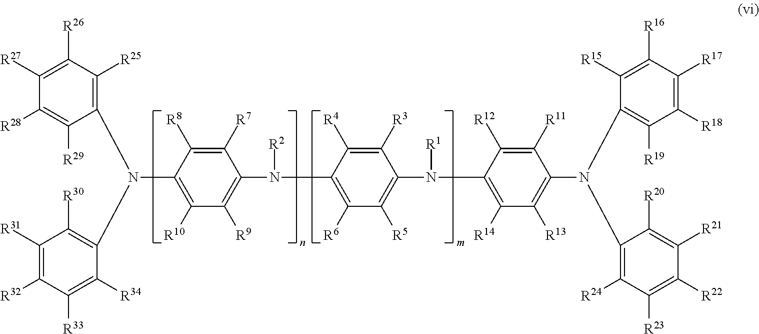

In the above formula, R¹ and R² each independently represent a hydrogen atom, a substituted or unsubstituted monovalent hydrocarbon group, a t-butoxycarbonyl group, or a benzyloxycarbonyl group, R³ to R³⁴ each independently represent a hydrogen atom, a hydroxyl group, a silanol group, a thiol group, a carboxyl group, a phosphoric acid group, a phosphate group, an ester group, a thioester group, an amide group, a nitro group, a substituted or unsubstituted monovalent hydrocarbon group, an organoxy group, an organoamino group, an organosilyl group, an organothio group, an acyl group, a sulfone group or a halogen atom, m and n each independently indicate an integer of 1 or more and satisfy m+n≦20.

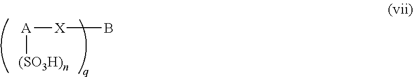

In the above formula, X represents O, S or NH, A represents a naphthalene ring or an anthracene ring optionally having any other substituent than X and n's (SO₃H)'s, B represents a substituted or unsubstituted hydrocarbon group, a 1,3,5-triazine group, or a substituted or unsubstituted group represented by the following formula (vii-1) or (vii-2) (wherein W¹ and W² each independently represent O, S, S(O), S(O₂), or a substituted or unsubstituted N, Si, P or P(O)), n is an integer that satisfies 1≦n≦4, and q is an integer that satisfies 1≦q.

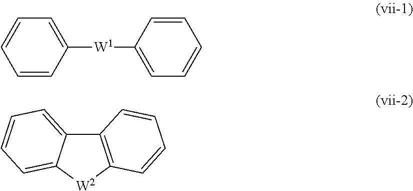

In consideration of the purpose of improving durability and improving charge transporting capability, B is preferably a divalent or more multivalent, substituted or unsubstituted hydrocarbon group containing at least one aromatic ring, a divalent or trivalent 1,3,5-triazine group, or a substituted or unsubstituted divalent diphenylsulfone group, and is especially preferably a divalent or trivalent, substituted or unsubstituted benzyl group, a divalent substituted or unsubstituted p-xylylene group, a divalent or trivalent substituted or unsubstituted naphthyl group, a divalent or trivalent 1,3,5-triazine group, a divalent substituted or unsubstituted diphenylsulfone group, a di- to tetravalent perfluorobiphenyl group, a divalent substituted or unsubstituted 2,2-bis((hydroxypropoxy)phenyl)propyl group, a substituted or unsubstituted polyvinylbenzyl group.

The compound represented by the formula (vii) is especially preferably represented by the formula (vii-3).

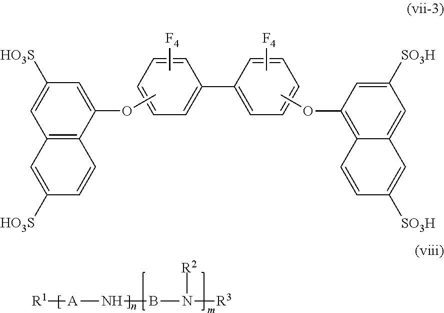

In the formula, R¹, R² and R³ each independently represent a hydrogen atom, a halogen atom, a hydroxyl group, an amino group, a silanol group, a thiol group, a carboxyl group, a phosphoric acid group, a phosphate group, an ester group, a thioester group, an amide group, a nitro group, a monovalent hydrocarbon group, an organoxy group, an organoamino group, an organosilyl group, an organothio group, an acyl group or a sulfonic acid group, A and B each independently represent a divalent group represented by the formula (viii-1) or (viii-2).

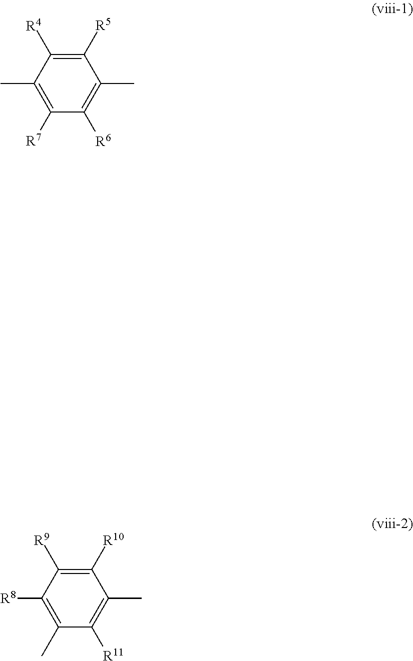

In the formula, R⁴ to R¹¹ each independently represent a hydrogen atom, a halogen atom, a hydroxyl group, an amino group, a silanol group, a thiol group, a carboxyl group, a phosphoric acid group, a phosphate group, an ester group, a thioester group, an amide group, a nitro group, a monovalent hydrocarbon group, an organoxy group, an organoamino group, an organosilyl group, an organothio group, an acyl group or a sulfonic acid group. m and n each independently indicate an integer of 1 or more, satisfying m+n≦20.

### Mixture of the Following Compounds (ix)

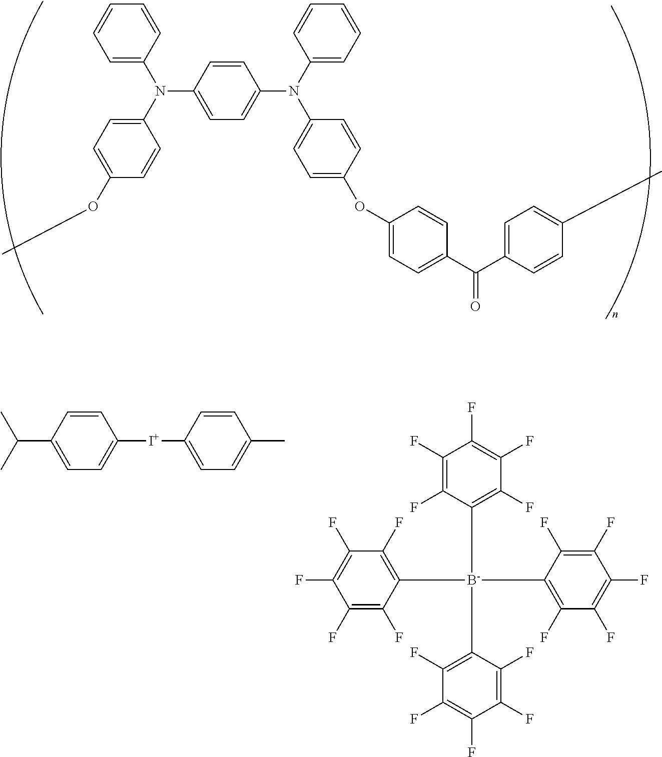

n indicates an integer of 3 or more.

### Mixture of the Following Compounds (x)

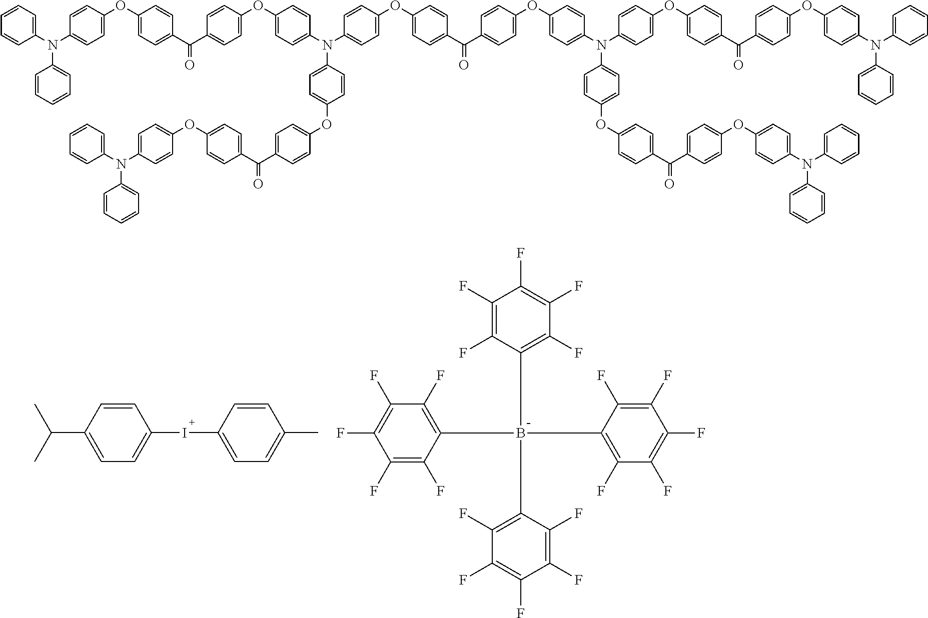

In one aspect of the present invention, a phenylamine polymer of the following formula (X) may be used as the hole transporting material:

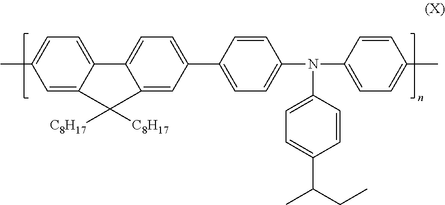

wherein n indicates an integer of 3 or more.

The hole transporting layer in the organic EL device of one aspect of the present invention may be made into a two-layered structure of a first hole transporting layer (anode side) and a second hole transporting layer (cathode side).

The thickness of the hole transporting layer is preferably 10 to 200 nm, although not particularly limited thereto.

The organic EL device of one aspect of the present invention may have a layer containing an acceptor material, which is disposed in contact with the anode side of the hole transporting layer or the first hole transporting layer. With such a layer, it is expected that the driving voltage is lowered and the production cost is reduced.

The acceptor material is preferably a compound represented by the following formula (Y).

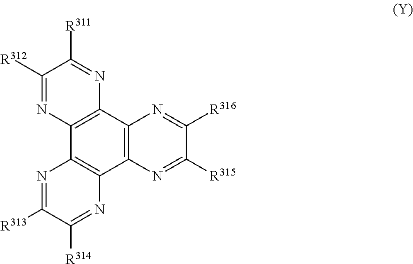

In the above formula (Y), R³¹¹ to R³¹⁶ may be the same as or different from each other, each independently representing a cyano group, —CONH₂, a carboxyl group, or —COOR³¹⁷ (R³¹⁷ represents an alkyl group having 1 to 20 carbon atoms or a cycloalkyl group having 3 to 20 carbon atoms). A pair or two or more pairs of R³¹¹ and R³¹², R³¹³ and R³¹⁴, and R³¹⁵ and R³¹⁶ may together form a group represented by —CO—O—CO—.

Examples of R³¹⁷ include a methyl group, an ethyl group, an n-propyl group, an isopropyl group, an n-butyl group, an isobutyl group, a t-butyl group, a cyclopentyl group, a cyclohexyl group, etc.

The thickness of the layer containing the acceptor material is preferably 5 to 20 nm, although not particularly limited thereto.

As the acceptor material, the following materials may be used.

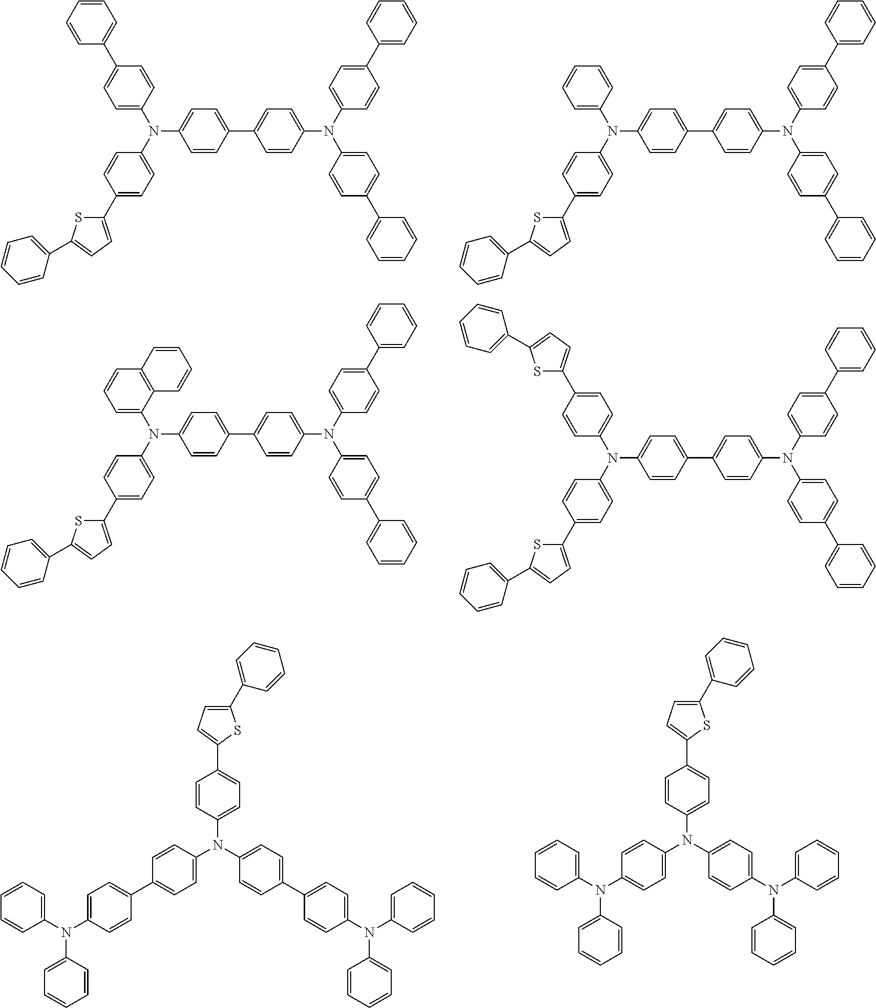

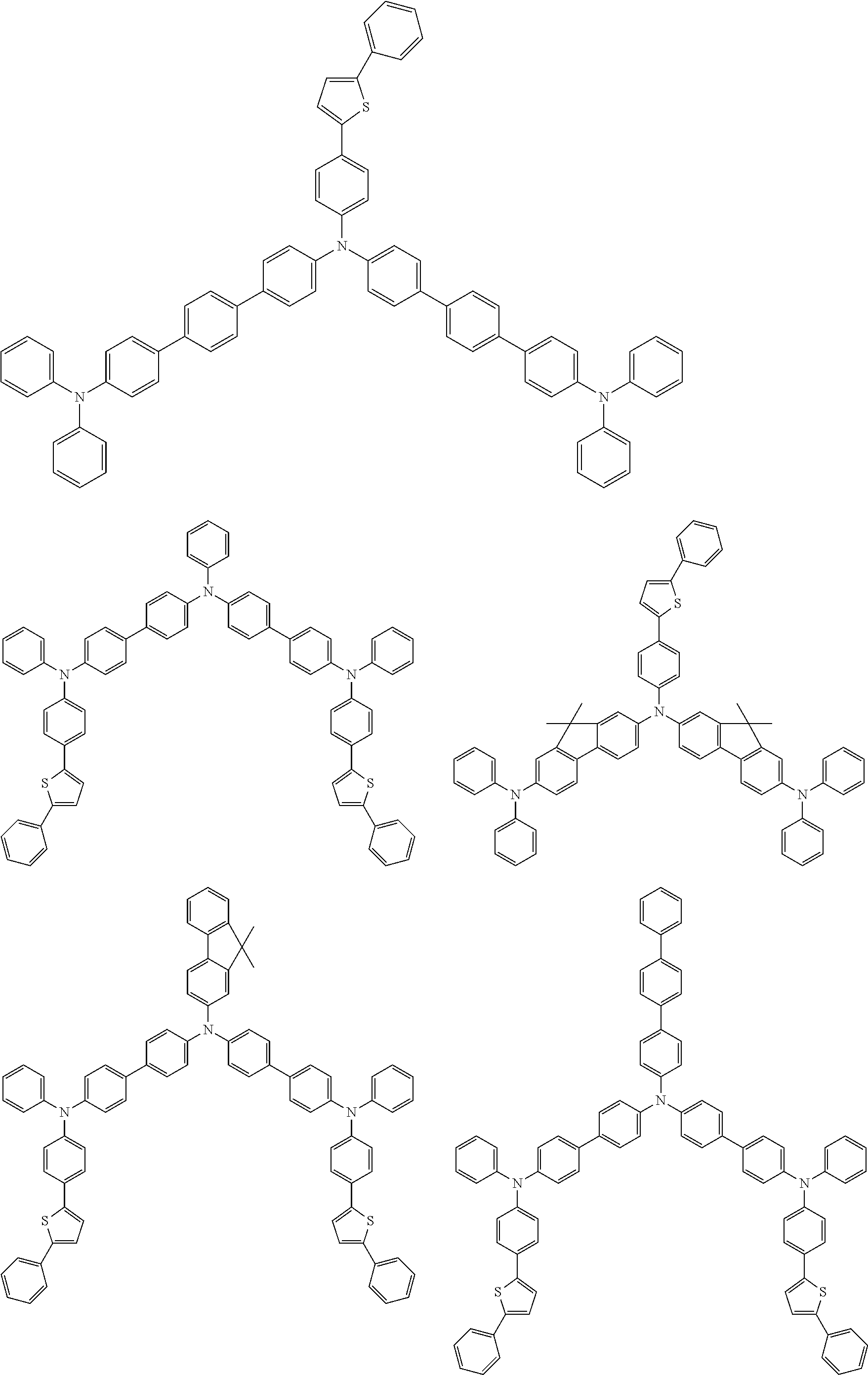

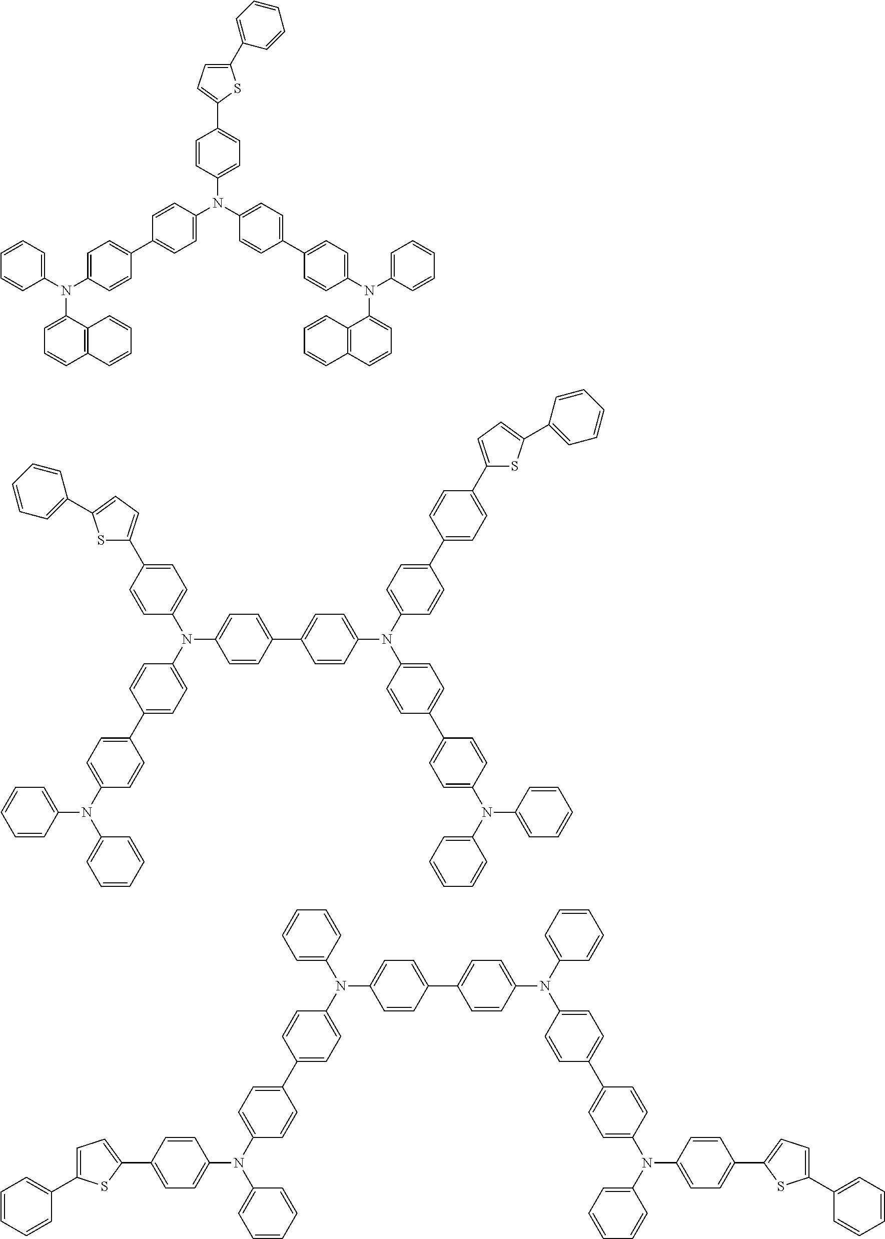 [SMILES: c1ccc(-c2ccc(-c3ccc(N(c4ccc(-c5ccc(N(c6ccccc6)c6cccc7ccccc67)cc5)cc4)c4ccc(-c5ccc(N(c6ccccc6)c6cccc7ccccc67)cc5)cc4)cc3)s2)cc1 ; c1ccc(-c2ccc(-c3ccc(-c4ccc(N(c5ccc(-c6ccc(N(c7ccccc7)c7ccccc7)cc6)cc5)c5ccc(-c6ccc(N(c7ccc(-c8ccc(N(c9ccccc9)c9ccccc9)cc8)cc7)c7ccc(-c8ccc(-c9ccccc9)s8)cc7)cc6)cc5)cc4)cc3)s2)cc1 ; c1ccc(-c2ccc(-c3ccc(N(c4ccccc4)c4ccc(-c5ccc(N(c6ccccc6)c6ccc(-c7ccc(N(c8ccccc8)c8ccc(-c9ccc(N(c%10ccccc%10)c%10ccc(-c%11ccc(-c%12ccccc%12)s%11)cc%10)cc9)cc8)cc7)cc6)cc5)cc4)cc3)s2)cc1]

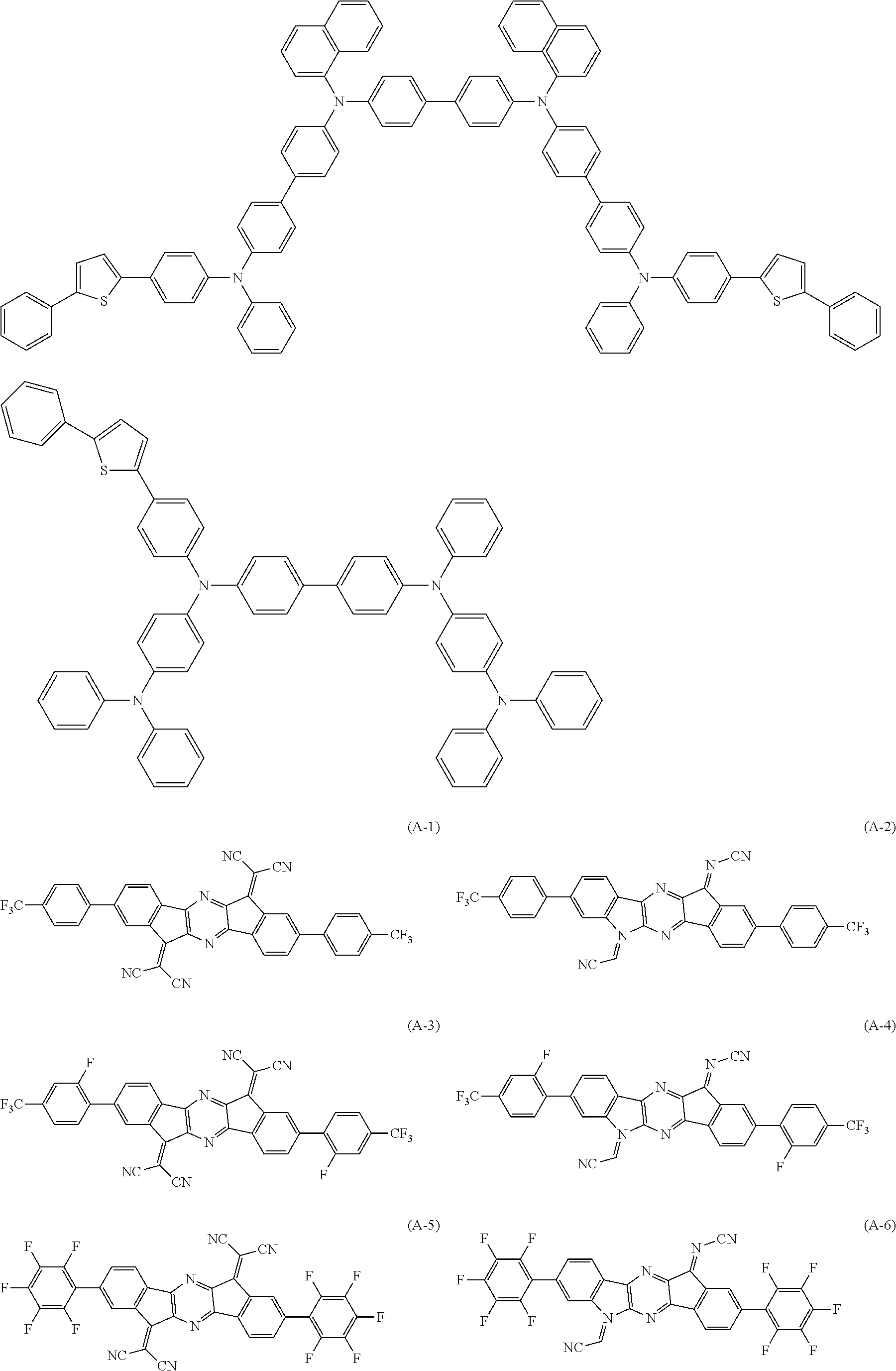

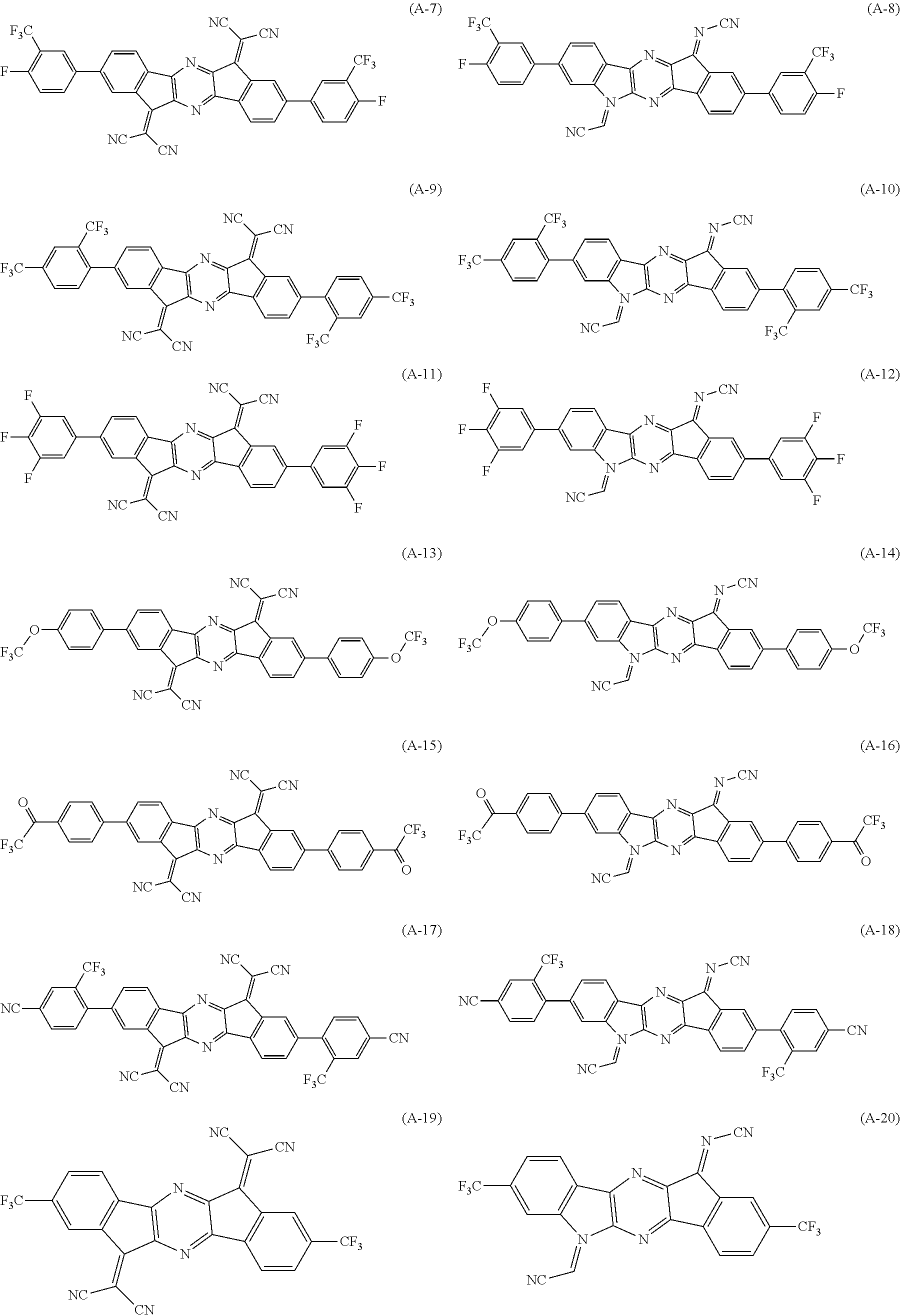

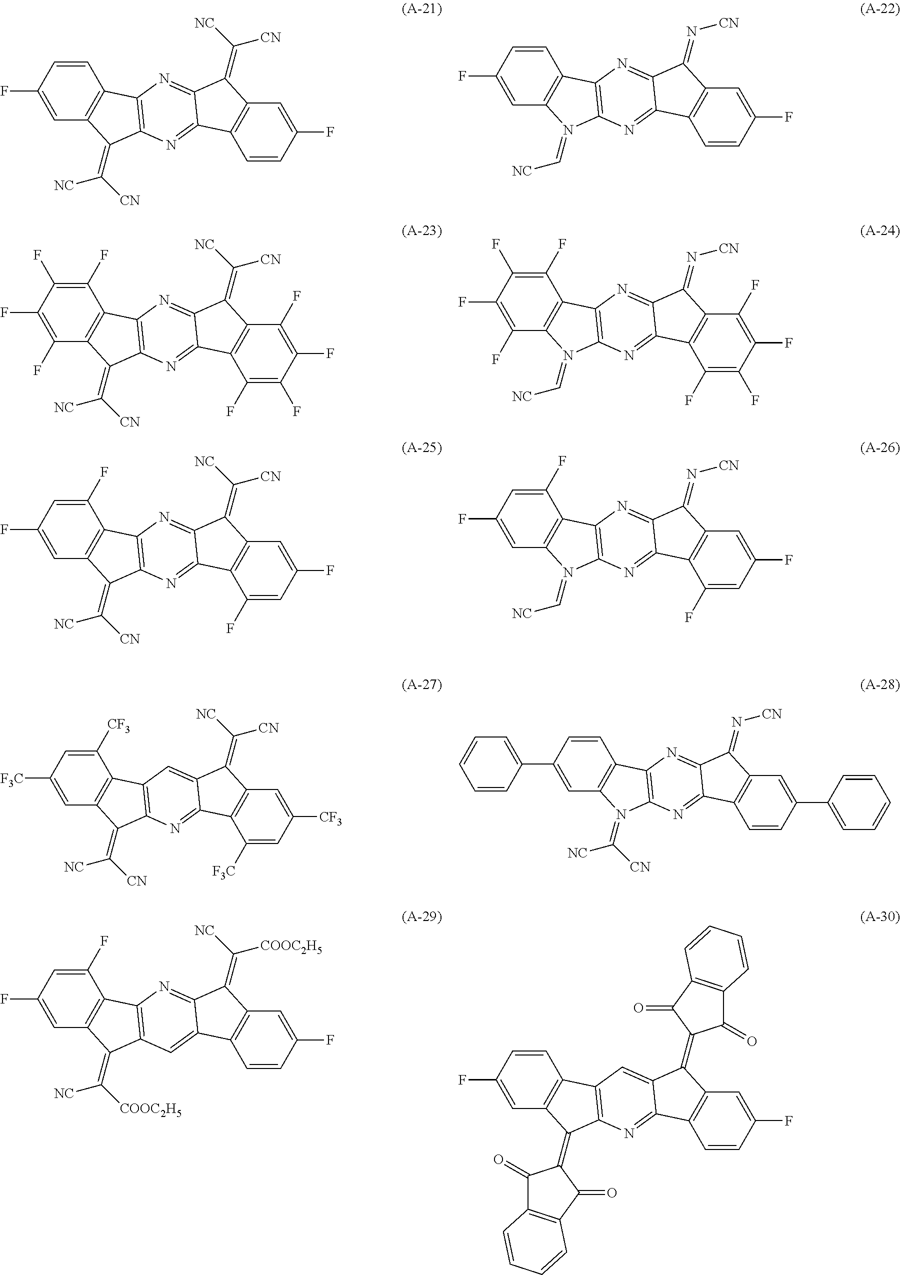

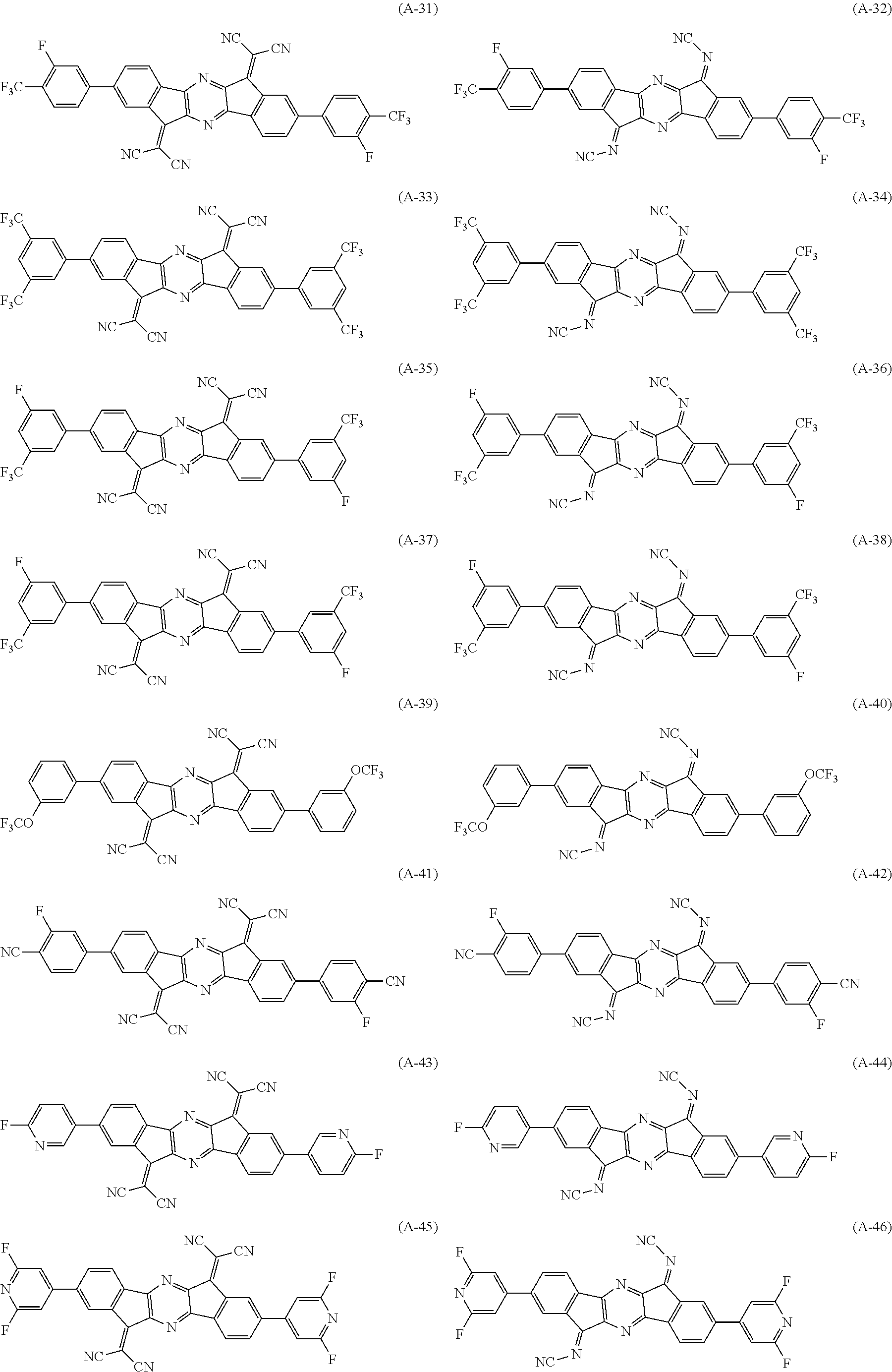

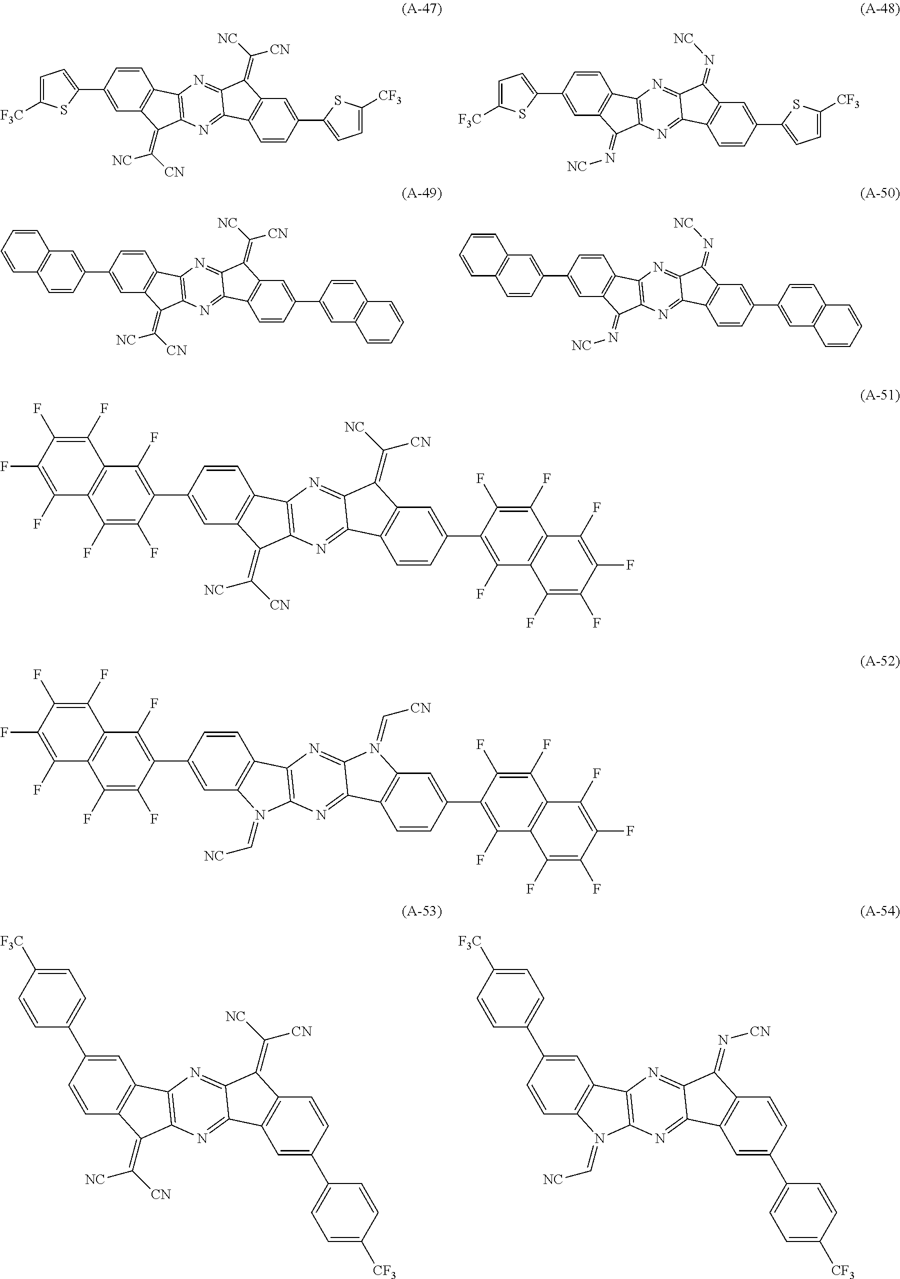

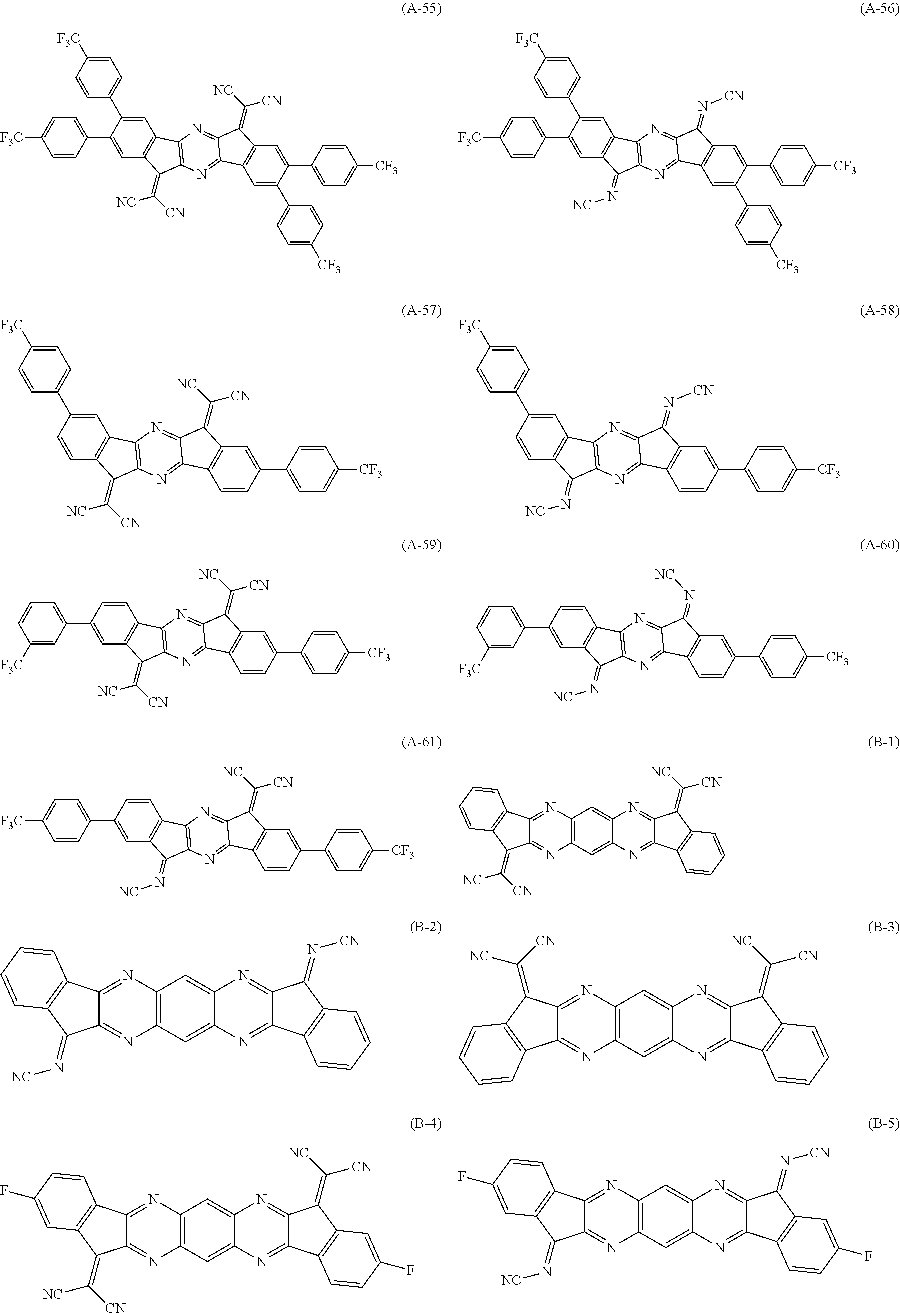

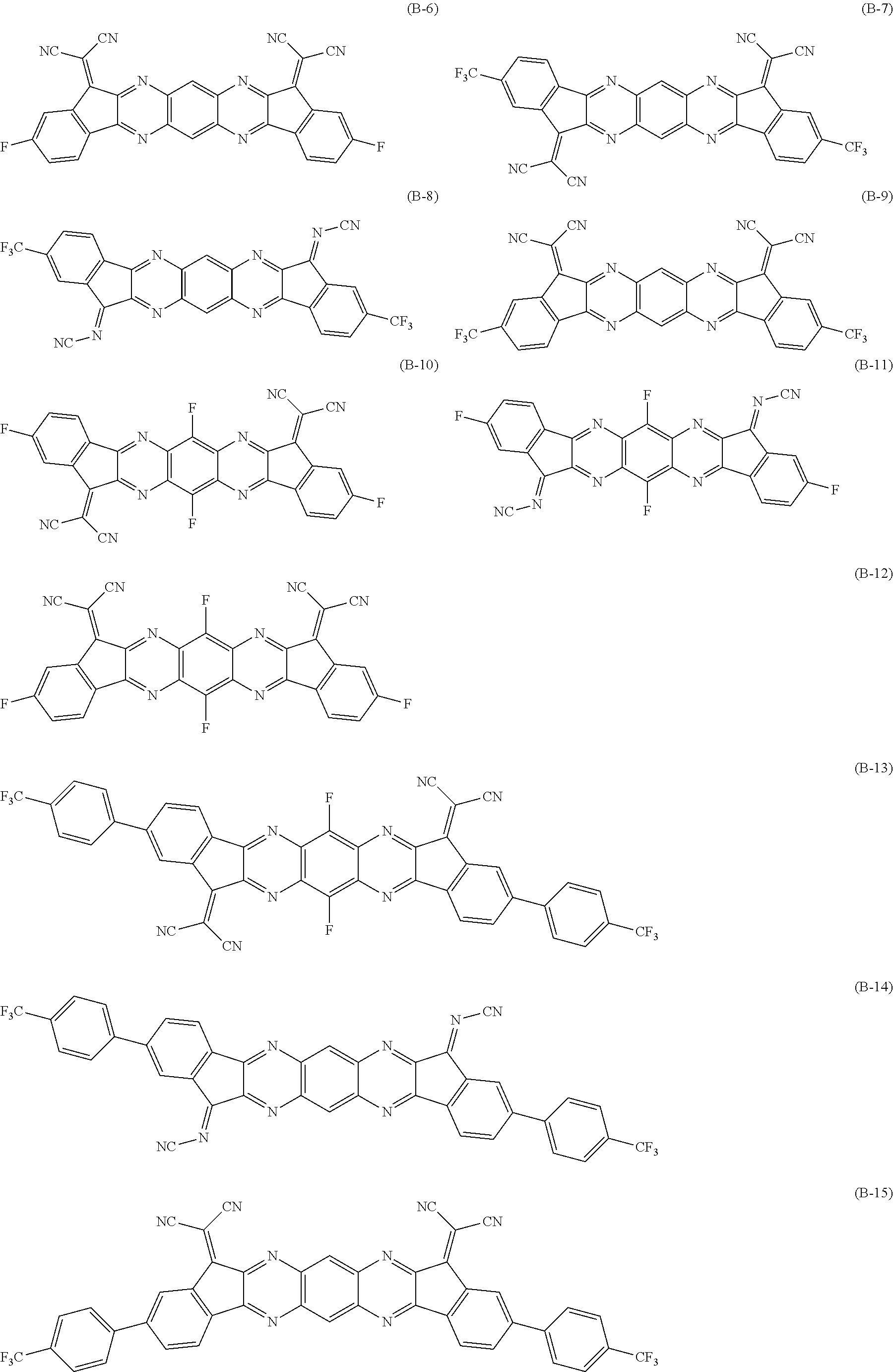

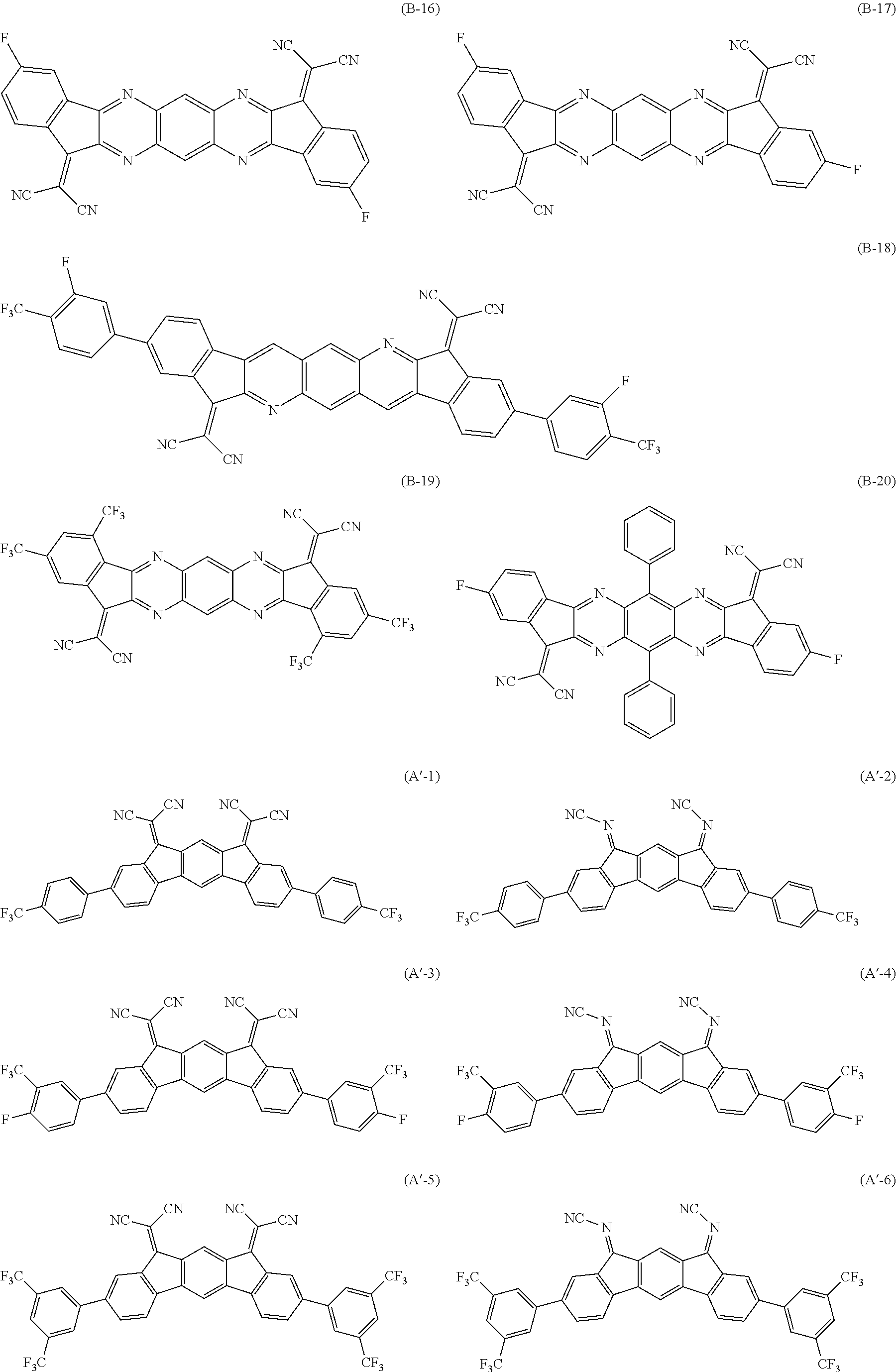

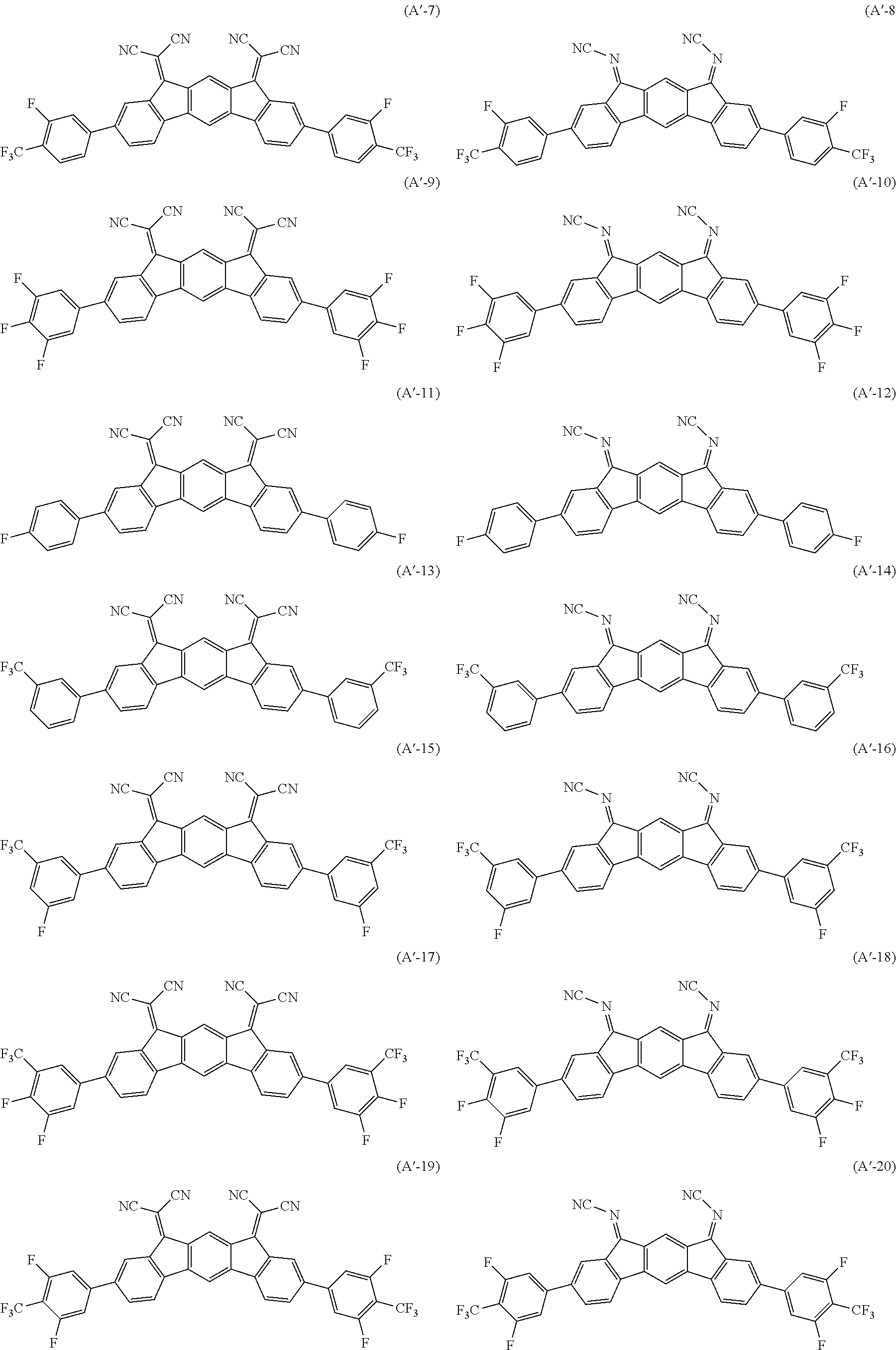

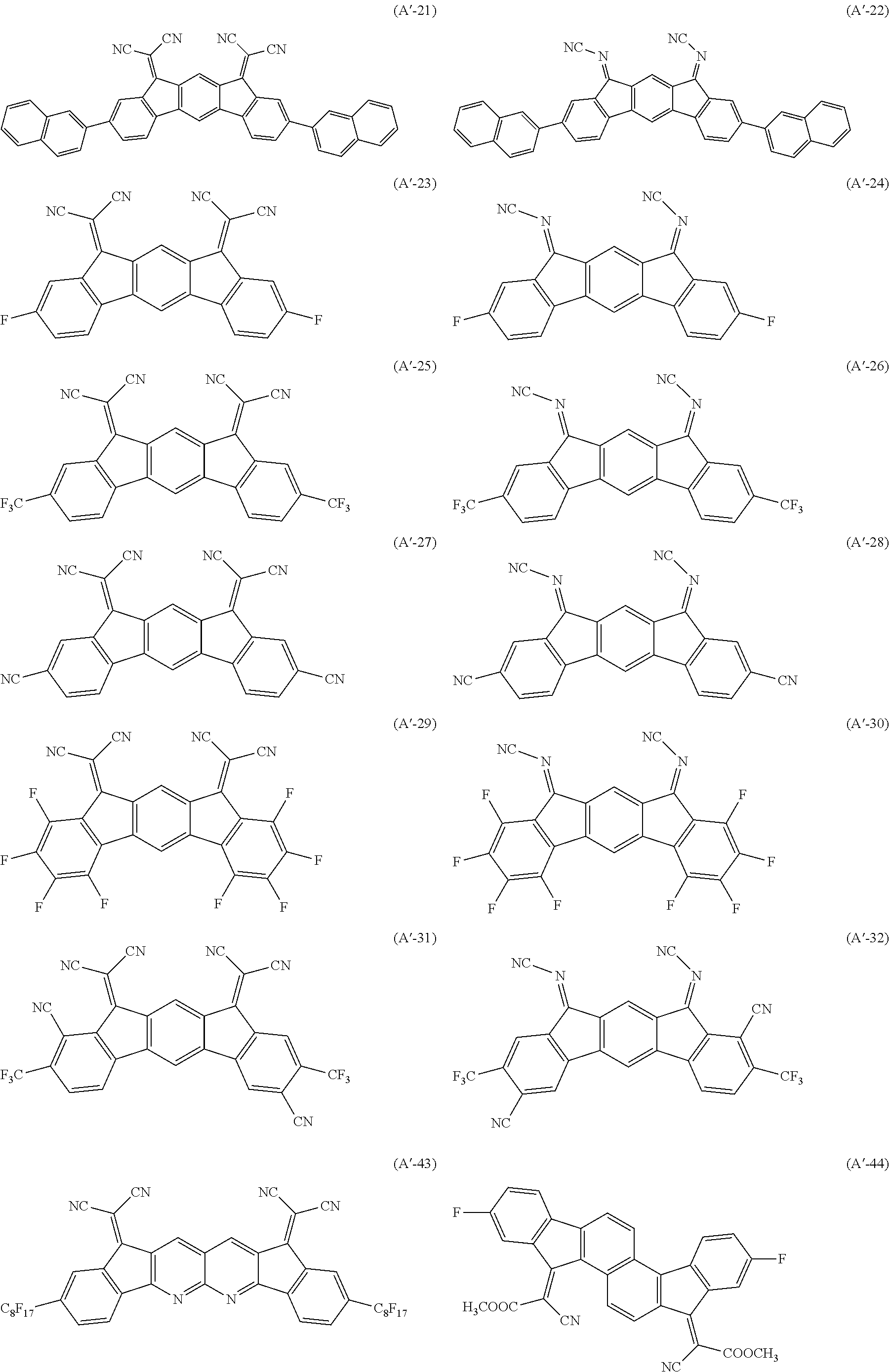

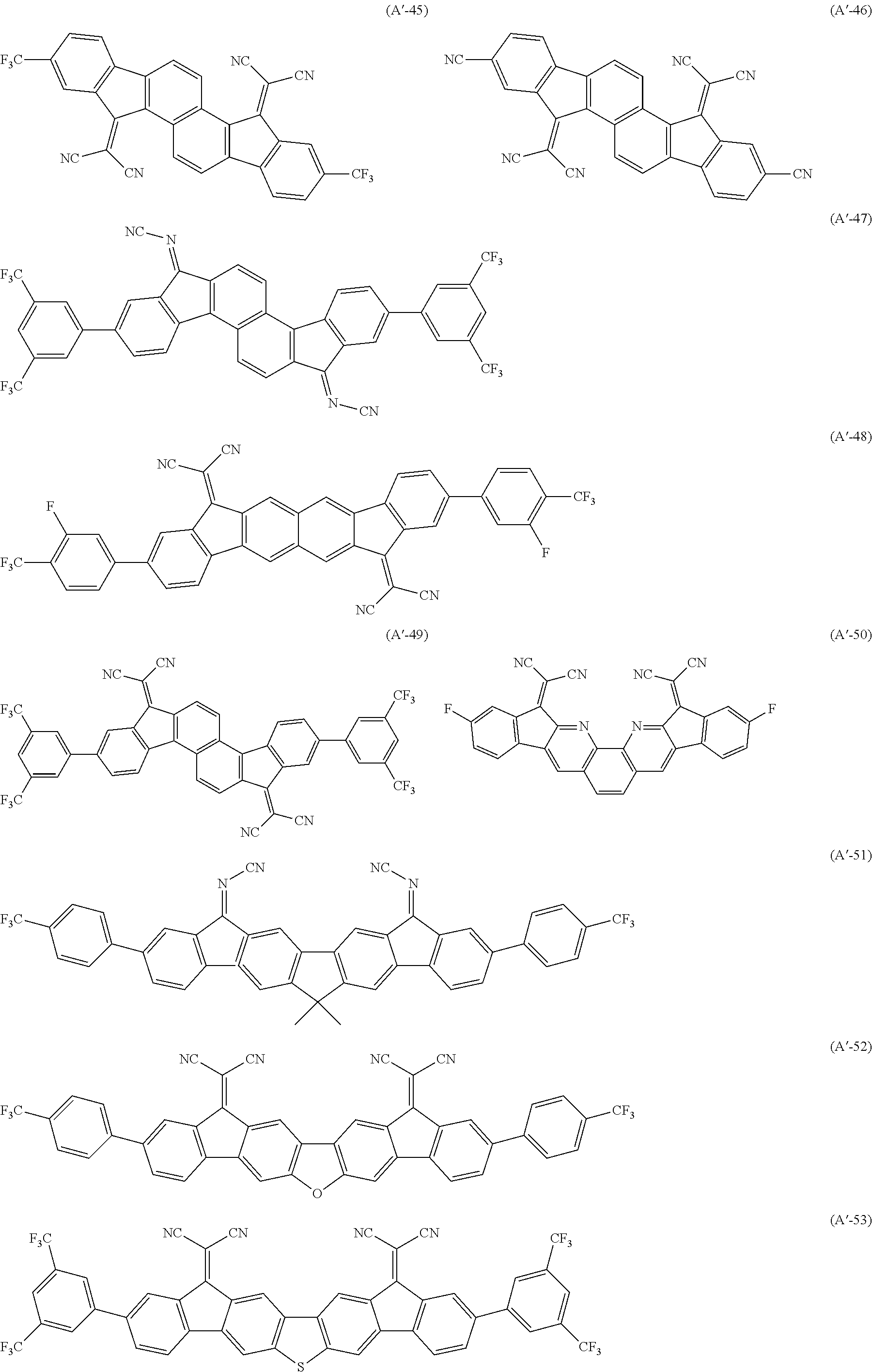

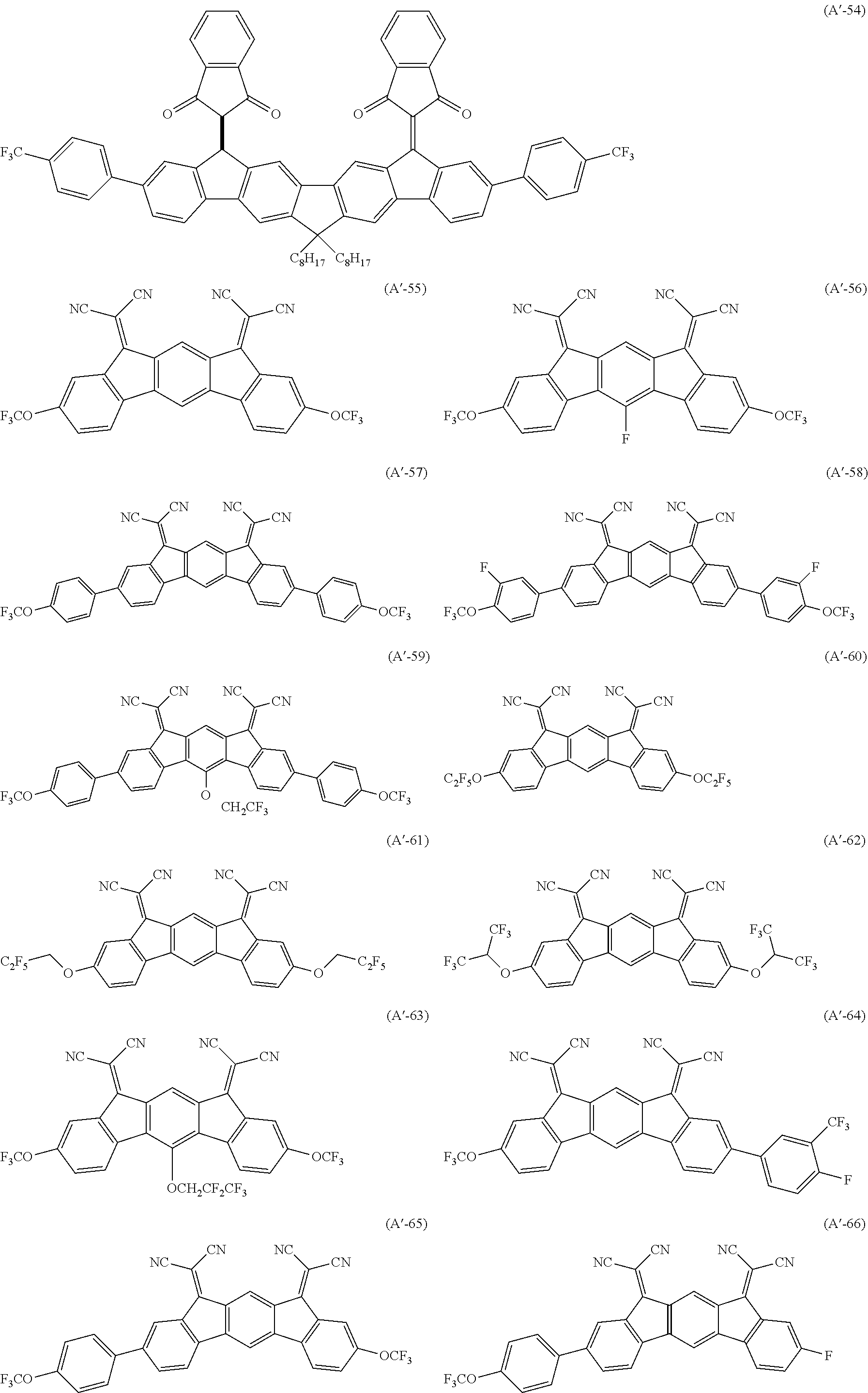

### (n/p Doping)

The carrier injecting properties of the hole transporting layer and the electron transporting layer can be controlled by doping (n) with a donor material or doping (p) with an acceptor material.

A typical example of the n-doping is an electron transporting material doped with a metal, such as Li and Cs, and a typical example of the p-doping is a hole transporting material doped with an acceptor material, such as F₄TCNQ (2, 3,5, 6-tetrafluoro-7, 7, 8, 8-tetracyanoquinodimethane).

### (Space Layer)

For example, when a fluorescent light emitting layer and a phosphorescent light emitting layer are laminated, a space layer is disposed between the fluorescent light emitting layer and the phosphorescent light emitting layer to prevent the diffusion of excitons generated in the phosphorescent light emitting layer to the fluorescent light emitting layer or to control the carrier balance. The space layer may be disposed between two or more phosphorescent light emitting layers.

Since the space layer is disposed between the light emitting layers, a material combining the electron transporting ability and the hole transporting ability is preferably used for forming the space layer. To prevent the diffusion of triplet energy in the adjacent phosphorescent light emitting layer, the triplet energy of the material for the space layer is preferably 2.6 eV or more. The materials described above with respect to the hole transporting layer are usable as the material for the space layer. The material for organic EL devices of one aspect of the present invention may also be used as the material for the space layer.

### (Blocking Layer)

The organic EL device of one aspect of the present invention preferably has a blocking layer, such as an electron blocking layer, a hole blocking layer or a triplet blocking layer, which is disposed adjacent to the light emitting layer. The electron blocking layer is a layer which prevents the diffusion of electrons from the light emitting layer to the hole transporting layer. The hole blocking layer is a layer which prevents the diffusion of holes from the light emitting layer to the electron transporting layer. The material for organic EL devices of one aspect of the present invention may be used as the material for the hole blocking layer.

The triplet blocking layer prevents the diffusion of triplet excitons generated in the light emitting layer to the adjacent layers and has a function of confining the triplet excitons in the light emitting layer, thereby preventing the deactivation of energy on any other molecules in the electron transporting layer than the light emitting dopant of triplet excitons.

If a phosphorescent device having a triplet blocking layer satisfies the following energy relationship: E^{T}_{d}<E^{T}_{TB} wherein E^{T}_{d} is the triplet energy of the phosphorescent dopant in the light emitting layer and E^{T}_{TB} is the triplet energy of the compound forming the triplet blocking layer, the triplet excitons of the phosphorescent dopant are confined (not diffusing to other molecules). Therefore, the energy deactivation process other than the emission on the phosphorescent dopant may be prevented to cause the emission with high efficiency. However, even in case of satisfying the relationship of E^{T}_{d}<E^{T}_{TB}, the triplet excitons may move into any other molecules if the energy difference ΔE^{T}=E^{T}_{TB}−E^{T}_{d} is small, because the energy difference ΔE^{T} may be overcome by the absorption of the ambient heat energy when driving the device at around room temperature as generally employed in practical drive of devices. As compared with the fluorescent emission, the phosphorescent emission is relatively likely to be affected by the diffusion of excitons due to the heat absorption because the lifetime of triplet excitons is longer. Therefore, as for the energy difference ΔE^{T}, the larger as compared with the heat energy of room temperature, the better. The energy difference ΔE^{T} is more preferably 0.1 eV or more and particularly preferably 0.2 eV or more. On the other hand, in fluorescent devices, the material for organic EL devices in one aspect of the present invention is usable as the material for triplet blocking layer of the TTF device configuration described in WO2010/134350 A1.

The electron mobility of the material for the triplet blocking layer is preferably 10⁻⁶ cm²/Vs or more at an electric field strength of 0.04 to 0.5 MV/cm. There are several methods for measuring the electron mobility of organic materials, for example, Time of Flight method. In the present invention, the electron mobility is determined by impedance spectroscopy.

The electron mobility of the electron injecting layer is preferably 10⁻⁶ cm²/Vs or more at an electric field strength of 0.04 to 0.5 MV/cm. Within the above range, the injection of electrons from the cathode to the electron transporting layer is promoted and the injection of electrons to the adjacent blocking layer and light emitting layer is also promoted, thereby enabling to drive devices at a lower voltage.

The method for forming each layer of the organic EL device of one aspect of the present invention is not specifically limited. Any forming method of conventionally-known vacuum evaporation method, spin coating method and the like may be employed. The organic thin film layer containing the compound (1) for use in the organic EL device of one aspect of the present invention may be formed according to a known method such as a vapor deposition method, a molecular beam evaporation method (MBE method), or a coating method of a dipping method, a spin coating method, a casting method, a bar coating method, a roll coating method or the like using a solution of the compound dissolved in a solvent.

The thickness of each organic layer of the organic EL device of one aspect of the present invention is not specifically limited, but in general, when the thickness is too small, there may readily form defects such as pin holes and the like, but when too large, a high application voltage is needed and the efficiency worsens. In general, therefore, the thickness is preferably within a range of a few nm to 1 μm. As the method for forming the layer (especially the light emitting layer) containing the compound (1) of one aspect of the present invention, for example, a method of using the above-mentioned ink composition of one aspect of the present invention for film formation is preferred.

For the film formation method, any known coating method may be effectively used. For example, there are mentioned a spin coating method, a casting method, a microgravure coating method, a gravure coating method, a bar coating method, a roll coating method, a slit coating method, a wire bar coating method, a dip coating method, a spray coating method, a screen printing method, a flexographic printing method, an offset printing method, an ink jet method, a nozzle printing method, etc. For pattern formation, a screen printing method, a flexographic printing method, an offset printing method and an ink jet printing method are preferred. The film formation according to these methods may be carried out under the condition well known by anyone skilled in the art.

After the film formation, the solvent may be removed by drying under heat (upper limit 250° C.) under vacuum, and any polymerization through exposure to light or by high-temperature heating at higher than 250° C. is unnecessary. Accordingly, it is possible to prevent the devices from being degraded through exposure to light or by high-temperature heating at higher than 250° C.

### [Electronic Equipment]

The electronic equipment of one aspect of the present invention is described.

The electronic equipment of one aspect of the present invention contains the organic electroluminescence device of one aspect of the present invention. The organic electroluminescence device of one aspect of the present invention can be used in electronic equipments, for example, as display parts, such as organic EL panel modules, display devices of television sets, mobile phones, personal computer, etc., and light emitting sources of lighting equipment and vehicle lighting equipment.

### EXAMPLES

The present invention will be described below in more detail with reference to Examples. However, the present invention is not limited by these Examples.

### Synthesis Example 1

### Synthesis of Compound H-1

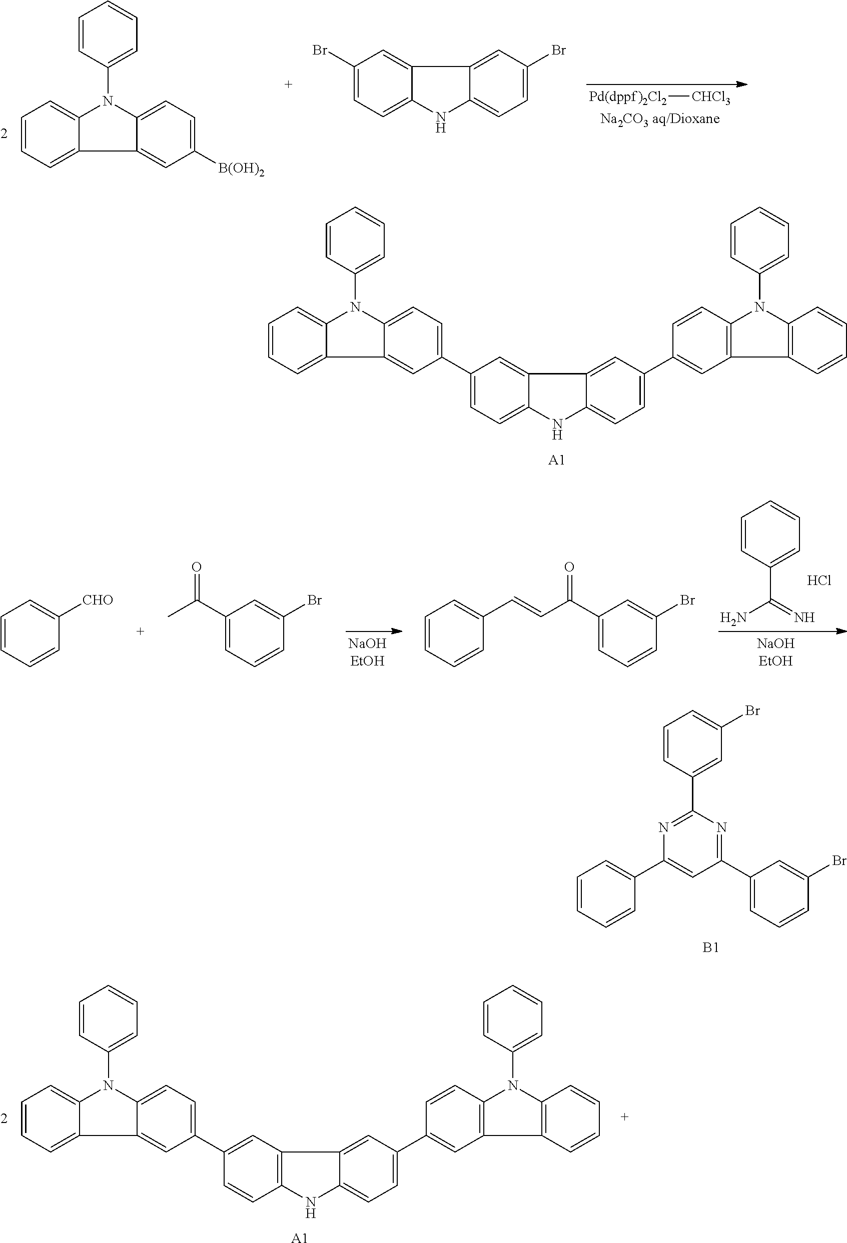

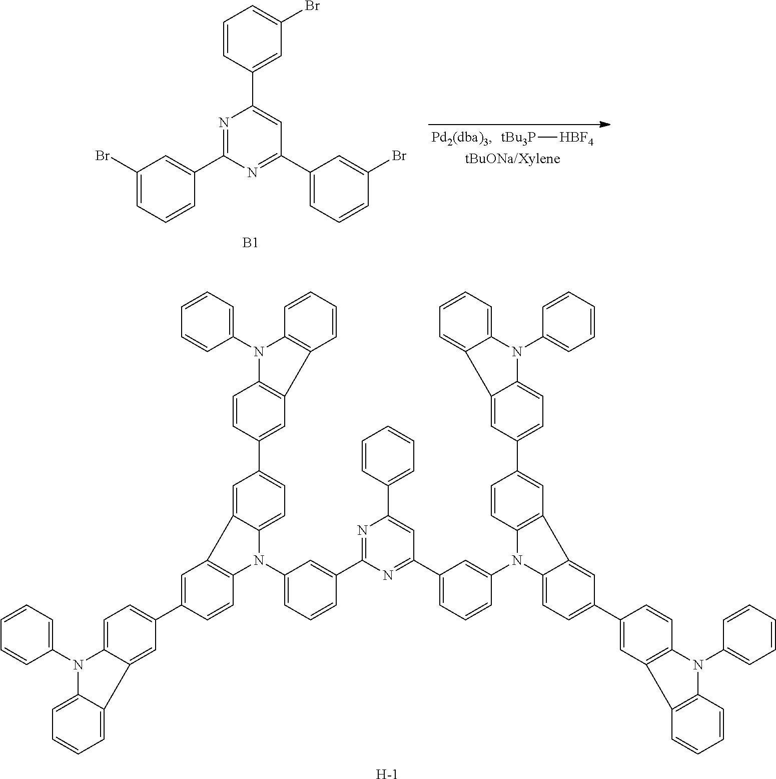

In an argon atmosphere, 9-phenylcarbazole-3-boronic acid (12.06 g, 42 mmol), 3,6-dibromocarbazole (5.60 g, 20 mmol), dichloro(diphenylphosphinoferrocene)palladium-methylene chloride complex (0.32 g, 0.4 mmol), 1,4-dioxane (60 mL) and aqueous 2 M sodium carbonate solution (60 mL) were sequentially added and heated under reflux for 7 hours.

The reaction liquid was cooled to room temperature, then the precipitated solid was taken out through filtration, washed with 1,4-dioxane and water, and dried under reduced pressure. The resultant residue was purified through silica gel column chromatography, and recrystallized from 1,4-dioxane to give the tricarbazolyl intermediate A1 (11.05 g, yield 85%).

Benzaldehyde (4.24 g, 40 mmol) and 3′-bromoacetophenone (7.96, 40 mmol) were dissolved in ethanol (80 mL), and sodium hydroxide (0.16 g, 4 mmol) was added thereto and stirred at room temperature for 8 hours. Subsequently, 4-bromobenzamidine hydrochloride (4.71 g, 20 mmol) and sodium hydroxide (1.60 g, 40 mmol) were added, ethanol (40 mL) was added, and reacted under heat with reflux for 8 hours. The formed white powder was collected through filtration, washed with ethanol until the wash could be colorless, and further this was washed with water and ethanol and dried in vacuum to give the intended pyrimidine intermediate B1 (8.58 g, yield 92%).

In an argon atmosphere, the tricarbazolyl intermediate A1 (4.09 g, 6.3 mmol), the pyrimidine intermediate B1 (1.40 g, 3.0 mmol), tris(dibenzylideneacetone)dipalladium (0.055 g, 0.06 mmol), tri-t-butylphosphonium tetrafluoroborate (0.070 g, 0.24 mmol), t-butoxy sodium (0.87 g, 9.0 mmol) and anhydrous xylene (60 mL) were sequentially added and heated under reflux for 8 hours.

The reaction liquid was cooled to room temperature, then the insoluble matter was removed through filtration and the organic solvent was evaporated away under reduced pressure. The resultant residue was purified through silica gel column chromatography to give the compound H-1 (3.27 g, yield 68%).

The results of analysis through HPLC (high performance liquid chromatography) and FD-MS (field desorption ionization-mass spectrometry) of the compound H-1 are shown below.

HPLC: purity 99.3%

FD-MS: calcd for C118H74N8=1603.

found m/z=1603 (M+, 100).

### Synthesis Example 2

### Synthesis of Compound H-2

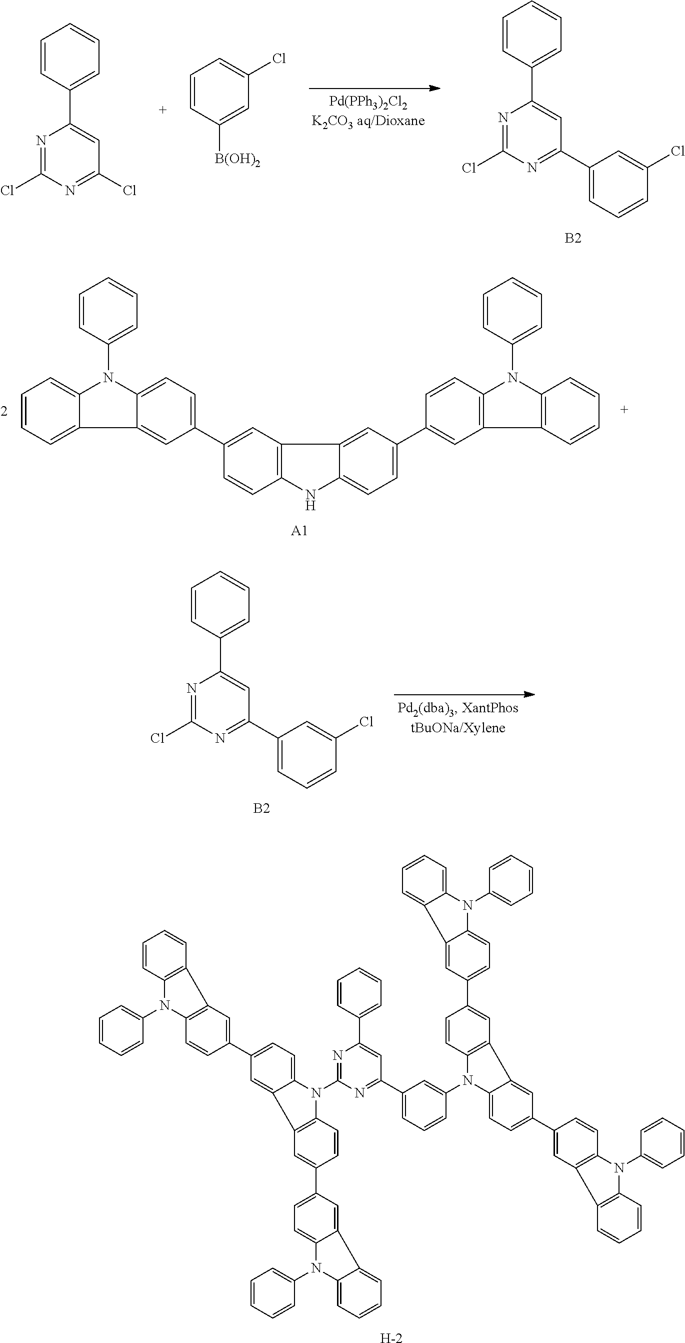

In an argon atmosphere, 4-phenyl-2,6-dichloropyrimidine (4.50 g, 20 mmol), 3-chlorophenylboronic acid (3.13 g, 20 mmol), dichloro(bistriphenylphosphine)palladium complex (0.351 g, 0.5 mmol), 1,4-dioxane (80 mL), and aqueous 2M potassium carbonate solution (40 mL) were sequentially added and heated under reflux for 8 hours. The reaction liquid was cooled to room temperature, then diluted with toluene, washed with water, and dried under reduced pressure. The resultant residue was purified through silica gel column chromatography to give the pyrimidine intermediate B2 (4.70 g, yield 78%).

In an argon atmosphere, the tricarbazolyl intermediate A1 (4.09 g, 6.3 mmol), the pyrimidine intermediate B2 (0.90 g, 3.0 mmol), tris(dibenzylideneacetone)dipalladium (0.055 g, 0.06 mmol), Xantphos (4,5-bis(diphenylphosphino)-9,9-dimethylxanthene) (0.069 g, 0.12 mmol), t-butoxy sodium (0.87 g, 9.0 mmol) and anhydrous xylene (60 mL) were sequentially added and heated under reflux for 8 hours.

The reaction liquid was cooled to room temperature, then the insoluble matter was removed through filtration and the organic solvent was evaporated away under reduced pressure. The resultant residue was purified through silica gel column chromatography to give the compound H-2 (2.34 g, yield 73%).

The results of analysis through HPLC and FD-MS of the compound H-2 are shown below.

HPLC: purity 99.0%

FD-MS: calcd for C112H70N8=1527.

found m/z=1527 (M+, 100).

### Synthesis Example 3

### Synthesis of Compound H-3

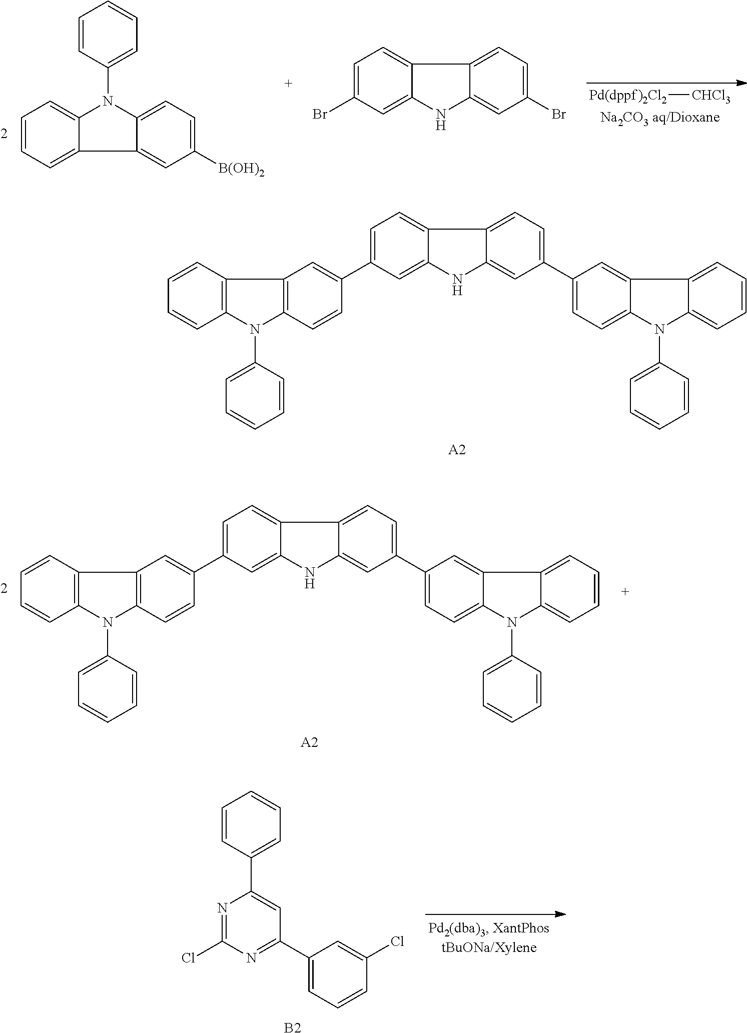

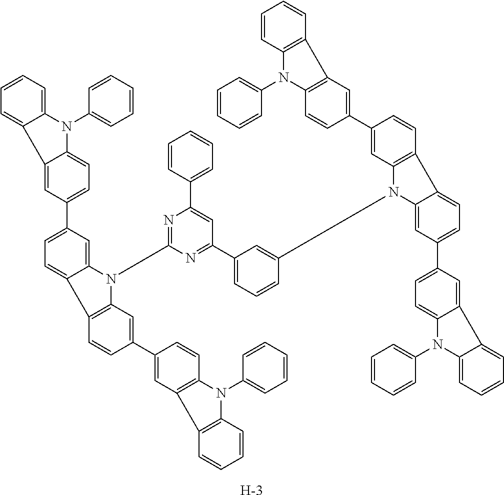

In an argon atmosphere, 9-phenylcarbazole-3-boronic acid (12.06 g, 42 mmol), 2, 7-dibromocarbazole (5.60 g, 20 mmol), dichloro(diphenylphosphinferrocene)palladium-methylene chloride complex (0.32 g, 0.4 mmol), 1,4-dioxane (60 mL), and aqueous 2M sodium carbonate solution (60 mL) were sequentially added and heated under reflux for 7 hours.

The reaction liquid was cooled to room temperature, then the precipitated solid was collected through filtration, washed with 1,4-dioxane and water, and dried under reduced pressure. The resultant residue was purified through silica gel column chromatography and then recrystallized from 1,4-dioxane to give the tricarbazolyl intermediate A2 (11.05 g, yield 90%).

In an argon atmosphere, the tricarbazolyl intermediate A2 (4.09 g, 6.3 mmol), the pyrimidine intermediate B2 (0.90 g, 3.0 mmol), tris(dibenzylideneacetone)dipalladium (0.055 g, 0.06 mmol), Xantphos (0.069 g, 0.12 mmol), t-butoxy sodium (0.87 g, 9.0 mmol) and anhydrous xylene (60 mL) were sequentially added and heated under reflux for 8 hours.

The reaction liquid was cooled to room temperature, then the insoluble matter was removed through filtration and the organic solvent was evaporated away under reduced pressure. The resultant residue was purified through silica gel column chromatography to give the compound H-3 (2.43 g, yield 53%).

The results of analysis through HPLC and FD-MS of the compound H-3 are shown below.

HPLC: purity 99.2%

FD-MS: calcd for C112H70N8=1527.

found m/z=1527 (M+, 100).

### Synthesis Example 4

### Synthesis of Compound H-4

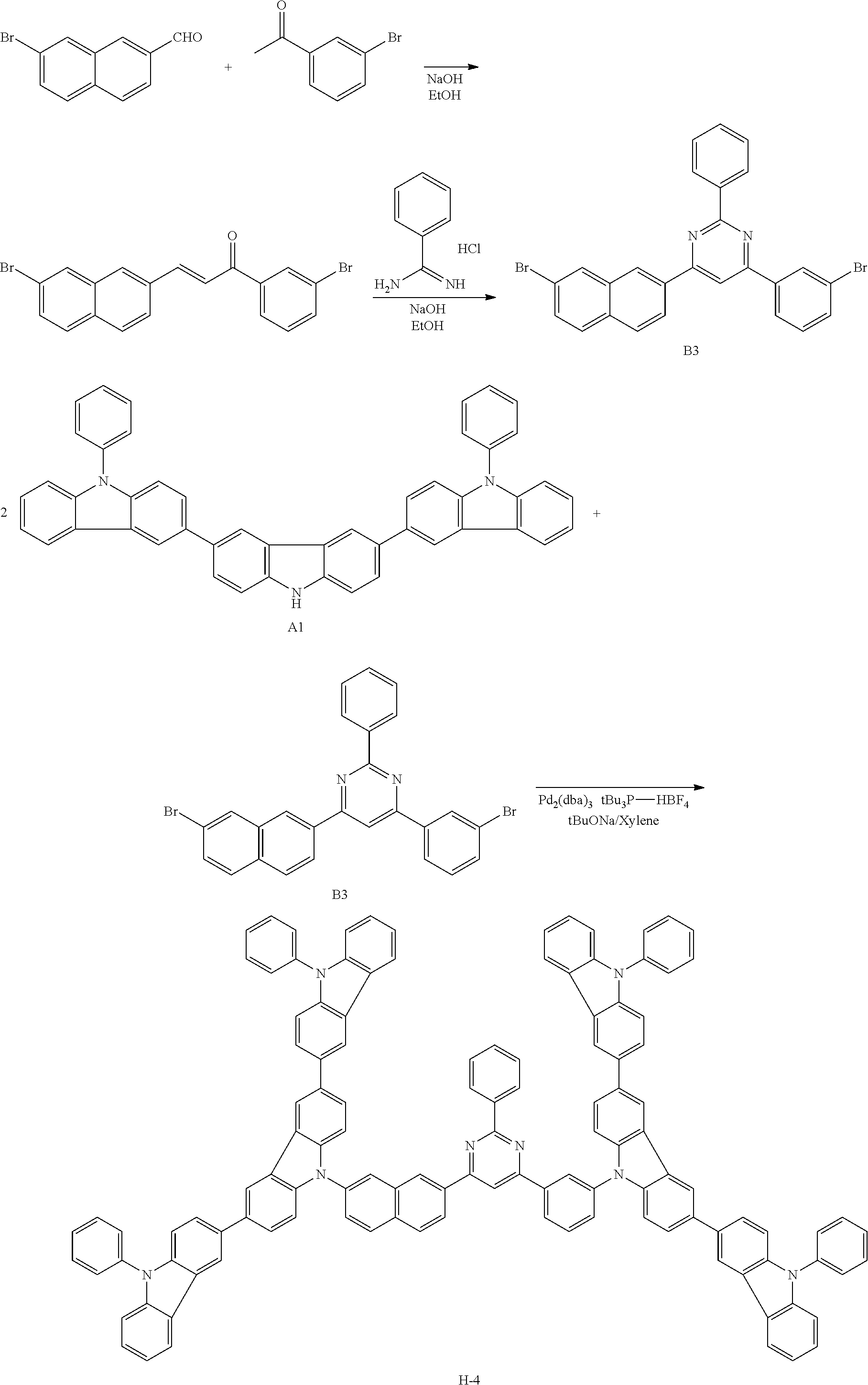

7-Bromonaphthaldehyde (9.40 g, 40 mmol) and 3′-bromoacetophenone (7.96, 40 mmol) were dissolved in ethanol (80 mL), and sodium hydroxide (0.16 g, 4 mmol) was added thereto and stirred at room temperature for 8 hours. Subsequently, benzamidine hydrochloride (3.13 g, 20 mmol) and sodium hydroxide (1.60 g, 40 mmol) were added, ethanol (40 mL) was added, and reacted under heat with reflux for 8 hours. The formed white powder was collected through filtration, washed with ethanol until the wash could be colorless, and further this was washed with water and ethanol and dried in vacuum to give the intended pyrimidine intermediate B3 (9.29 g, yield 90%).

In an argon atmosphere, the tricarbazolyl intermediate A1 (4.09 g, 6.3 mmol), the pyrimidine intermediate B3 (1.55 g, 3.0 mmol), tris(dibenzylideneacetone)dipalladium (0.055 g, 0.06 mmol), tri-t-butylphosphonium tetrafluoroborate (0.070 g, 0.24 mmol), t-butoxy sodium (0.87 g, 9.0 mmol) and anhydrous xylene (60 mL) were sequentially added and heated under reflux for 8 hours.

The reaction liquid was cooled to room temperature, then the insoluble matter was removed through filtration and the organic solvent was evaporated away under reduced pressure. The resultant residue was purified through silica gel column chromatography to give the compound H-4 (3.47 g, yield 70%).

The results of analysis through HPLC and FD-MS of the compound H-4 are shown below.

HPLC: purity 99.1%

FD-MS: calcd for C122H76N8=1654.

found m/z=1654 (M+, 100).

### Synthesis Example 5

### Synthesis of Compound H-5

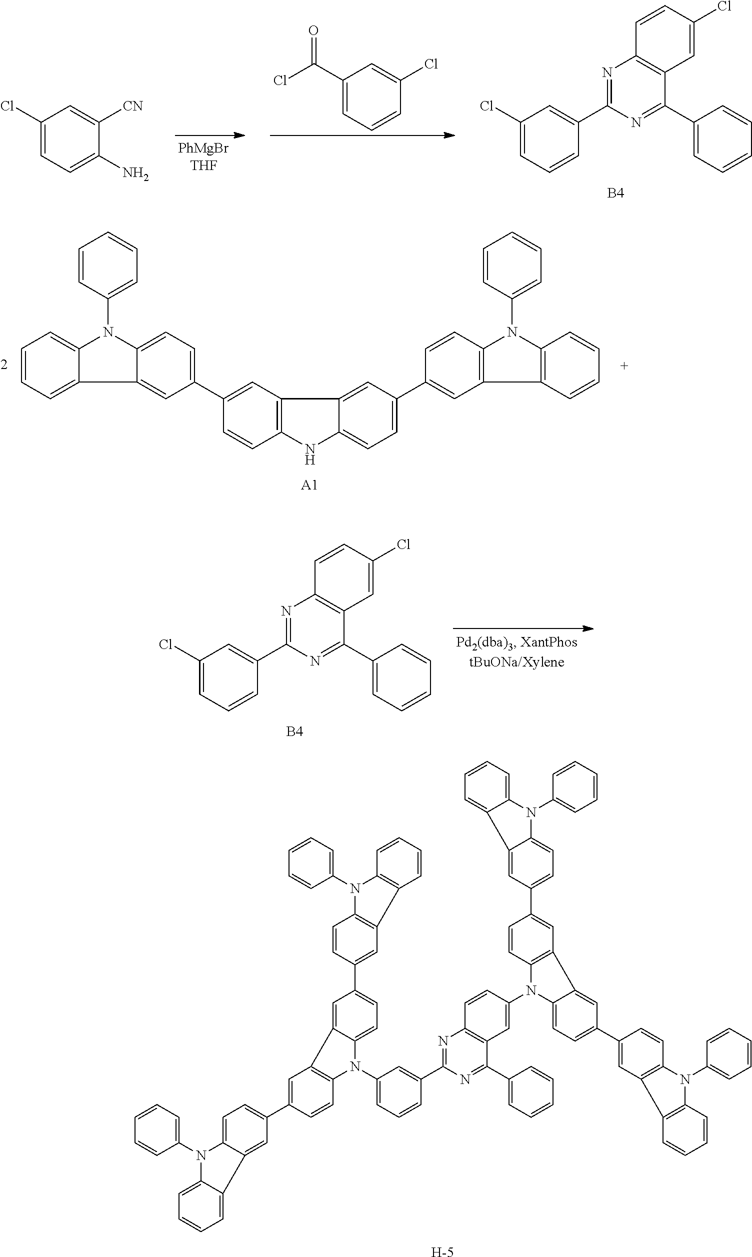

In an argon atmosphere, 2-amino-5-chlorobenzonitrile (3.81 g, 25 mmol) was dissolved in anhydrous THF (100 mL), cooled to 0° C., and then 2M phenyl-Grignard/THF solution (25 mL, 50 mmol) was dropwise added thereto, taking 30 minutes. Subsequently, 3-chlorobenzoyl chloride (4.37 g, 25 mmol) was dropwise added taking 30 minutes. Subsequently, this was heated up to room temperature, saturated saline water was added thereto, and extracted with ether. The organic layer was dried with magnesium sulfate, and the organic solvent was evaporated away under reduced pressure. The resultant residue was purified through silica gel column chromatography to give the quinazoline intermediate B4 (8.17 g, yield 93%).

In an argon atmosphere, the tricarbazolyl intermediate A1 (4.09 g, 6.3 mmol), the quinazoline intermediate B4 (1.05 g, 3.0 mmol), tris(dibenzylideneacetone)dipalladium (0.055 g, 0.06 mmol), Xantphos (0.069 g, 0.12 mmol), t-butoxy sodium (0.87 g, 9.0 mmol) and anhydrous xylene (60 mL) were sequentially added and heated under reflux for 8 hours.

The reaction liquid was cooled to room temperature, then the insoluble matter was removed through filtration and the organic solvent was evaporated away under reduced pressure. The resultant residue was purified through silica gel column chromatography to give the compound H-5 (2.32 g, yield 49%).

The results of analysis through HPLC and FD-MS of the compound H-5 are shown below.

HPLC: purity 98.9%

FD-MS: calcd for C116H72N8=1577.

found m/z=1577 (M+, 100).

### Synthesis Example 6

### Synthesis of Compound H-6

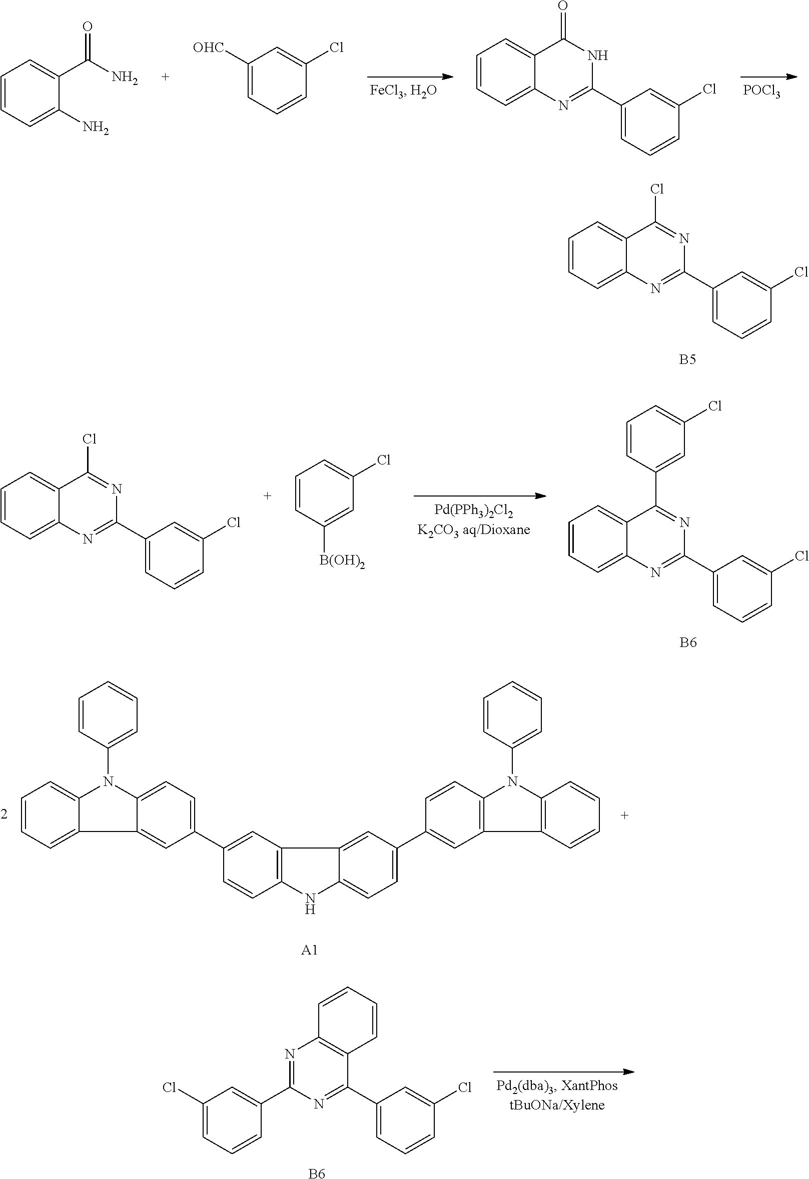

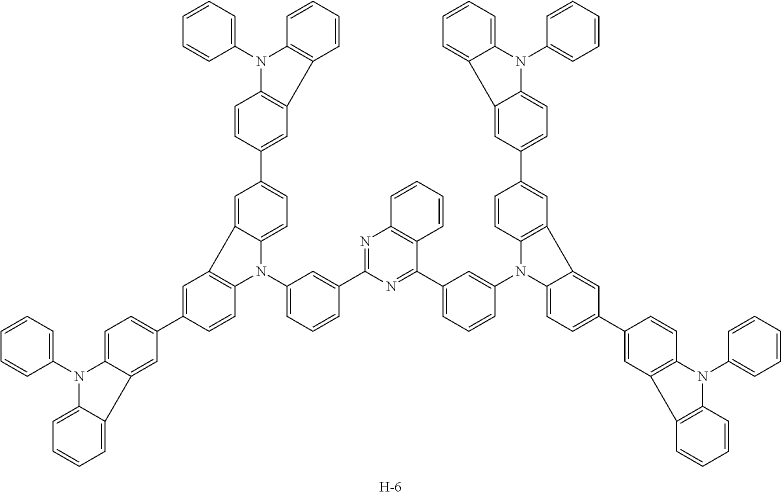

2-Aminobenzamide (2.72 g, 20 mmol) and 3-chlorobenzaldehyde (2.81 g, 20 mmol) were sequentially added to a solution of iron(III) chloride (6.45 g, 40 mmol) dissolved in water (200 mL), and heated under reflux for 3 hours. The reaction liquid was cooled to room temperature, and the precipitated solid was collected through filtration, washed with water and acetone, and dried under reduced pressure. Phosphoryl chloride (50 mL) was added thereto and heated under reflux for 3 hours. The reaction liquid was cooled to room temperature, and then the reaction liquid was poured into iced water, and extracted with methylene chloride. The organic layer was washed with water, dried with magnesium sulfate, and the organic solvent was evaporated away under reduced pressure. The resulting residue was purified through silica gel column chromatography to give the quinazoline intermediate B5 (4.40 g, yield 80%).

In an argon atmosphere, the quinazoline intermediate B5 (2.75 g, 10 mmol), 3-chlorophenylboronic acid (1.56 g, 10 mmol), dichloro(bistriphenylphosphine)palladium complex (0.17 g, 0.25 mmol), toluene (40 mL) and aqueous 2M potassium carbonate solution (20 mL) were sequentially added and heated under reflux for 8 hours. The reaction liquid was cooled to room temperature, then diluted with toluene, washed with water, and dried under reduced pressure. The resultant residue was purified through silica gel column chromatography to give the quinazoline intermediate B6 (2.49 g, yield 71%).

In an argon atmosphere, the tricarbazolyl intermediate A1 (4.09 g, 6.3 mmol), the quinazoline intermediate B6 (1.05 g, 3.0 mmol), tris(dibenzylideneacetone)dipalladium (0.055 g, 0.06 mmol), Xantphos (0.069 g, 0.12 mmol), t-butoxy sodium (0.87 g, 9.0 mmol) and anhydrous xylene (60 mL) were sequentially added and heated under reflux for 8 hours.

The reaction liquid was cooled to room temperature, then the insoluble matter was removed through filtration and the organic solvent was evaporated away under reduced pressure. The resultant residue was purified through silica gel column chromatography to give the compound H-6 (3.41 g, yield 72%).

The results of analysis through HPLC and FD-MS of the compound H-6 are shown below.

HPLC: purity 98.7%

FD-MS: calcd for C116H72N8=1577.

found m/z=1577 (M+, 100).

Other compounds within the claimed scope can be synthesized according to the reactions mentioned above, while using a known reaction and a known starting materials according to the target compound.

### Example 1

### Washing of Substrate

A glass substrate of 25 mm×25 mm×1.1 mm thickness having an ITO transparent electrode (product of Geomatec Company) was cleaned by ultrasonic cleaning in isopropyl alcohol for 5 minutes and then UV ozone cleaning for 5 minutes.

### (Formation of Underlayer)

As a hole transporting material, HERAEUS' CLEVIOUS A14083 (trade name) was formed into a film having a thickness of 30 nm on the above-mentioned ITO substrate according to a spin coating method. After the film formation, the unnecessary part was removed away with acetone, and then the coated substrate was fired on a hot plate at 200° C. in an air for 10 minutes to prepare a ground substrate.

### (Formation of Light Emitting Layer)

Using the compound H-1 obtained in Synthesis Example 1 as a host material, and using the following compound D-1 as a dopant material, these were mixed in a ratio by mass of compound H-1/compound D-1=95/5 to prepare a 1.6 mass % toluene solution. The toluene solution was applied onto the above-mentioned ground substrate to form a coating film having a thickness of 50 nm according to a spin coating method. After the film formation, the unnecessary part was removed away with toluene, and dried and heated on a hot plate at 150° C. to give a layer-coated substrate with a light emitting layer formed thereon. All the operation to form the light emitting layer was carried out in a nitrogen glove box.

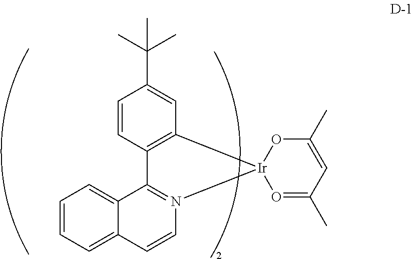

### (Vapor Deposition, Sealing)

The layer-coated substrate was conveyed into a vapor deposition chamber, and the following compound ET-1 was vapor-deposited thereon in a thickness of 50 nm as an electron transporting layer. Further, 1 nm of lithium fluoride and 80 nm of aluminium were layered through vapor deposition. After all the vapor deposition steps, this was sealed up with facing glass in a nitrogen glove box to produce an organic EL device.

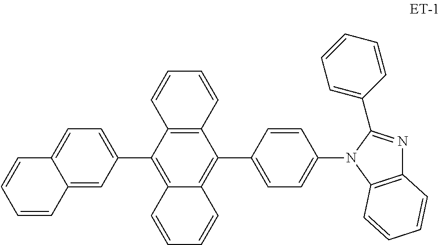 [SMILES: c1ccc(-c2nc3ccccc3n2-c2ccc(-c3c4ccccc4c(-c4ccc5ccccc5c4)c4ccccc34)cc2)cc1]

The resultant organic EL device was driven with a direct current for light emission, and the external quantum efficiency (EQE) at a current density of 10 mA/cm² was measured. The measurement result is shown in Table 1.

### Examples 2 to 5

Organic EL devices were produced in the same manner as in Example 1 except that the compounds H-2 to H-5 obtained in Synthesis Examples 2 to 5 were used as the host material.

The resultant organic EL devices were evaluated in the same manner as in Example 1, and the results are shown in Table 1.

### Comparative Example 1

An organic EL device was produced in the same manner as in Example except that the following comparative compound C-1 was used as the host material.

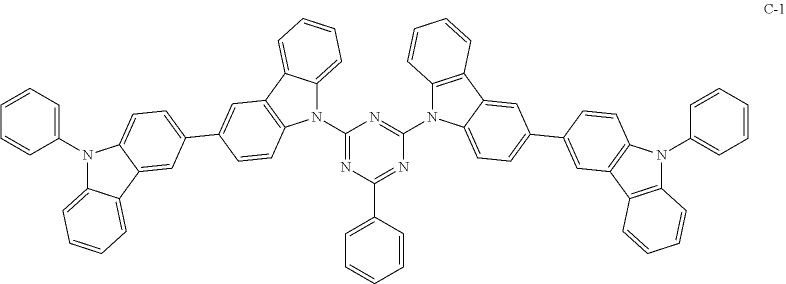 [SMILES: c1ccc(-c2nc(-n3c4ccccc4c4cc(-c5ccc6c(c5)c5ccccc5n6-c5ccccc5)ccc43)nc(-n3c4ccccc4c4cc(-c5ccc6c(c5)c5ccccc5n6-c5ccccc5)ccc43)n2)cc1]

TABLE 1

| | Host Material in Light | External Quantum |
|---|---|---|
| | Emitting Layer | Efficiency (EQE) % |
| Example 1 | H-1 | 5.3 |
| Example 2 | H-2 | 5.1 |
| Example 3 | H-3 | 4.9 |
| Example 4 | H-4 | 5.0 |
| Example 5 | H-5 | 4.8 |
| Comparative Example 1 | C-1 | 2.2 |

Using the compound (1) that is one aspect of the present invention promotes hole injection to the neighboring light emitting layer or hole injection from the transporting material and improves the efficiency in hole transfer inside the light emitting layer. As a result, it is known that the efficiency is further improved in the organic EL device using the compound. These are findings that have been obtained by applying the compound (1) of one aspect of the present invention to an organic EL device.

### REFERENCE SIGNS LISTS

1: Organic electroluminescence device
2: Substrate
3: Anode
4: Cathode
5: Light emitting layer
6: Anode-side organic thin film layer
7: Cathode-side organic thin film layer
10: Light emitting unit

1. An organic electroluminescence device comprising a cathode, an anode and one or more organic thin film layers including a light emitting layer between the cathode and the anode, wherein:
at least one layer of the one or more organic thin film layers comprises a compound represented by the formula (1): 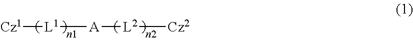
wherein Cz¹ represents a group represented by the formula (Cz-1),
Cz² represents a group represented by the formula (Cz-2),
A represents a residue of a substituted or unsubstituted nitrogen-containing aromatic hetero ring having 6 to 30 ring atoms,
L¹ and L² each independently represent a substituted or unsubstituted arylene group having 6 to 60 ring carbon atoms,
n1 and n2 each independently indicate an integer of 0 to 4,
when n1=0, A and Cz¹ bond via a single bond,
when n2=0, A and Cz² bond via a single bond,
when n1 is an integer of 2 to 4, L¹'s may be the same as or different from each other, and L¹'s may form a ring,
when n2 is an integer of 2 to 4, L²'s may be the same as or different from each other, and L²'s may form a ring; 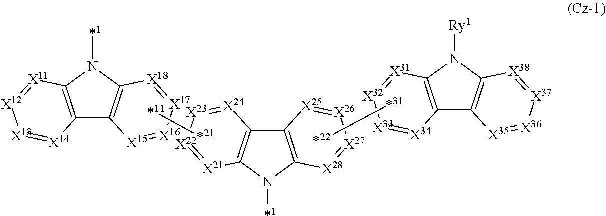
wherein X¹¹ to X¹⁴ each independently represent N or C-*²,
one of X¹⁵ to X¹⁸ is a carbon atom bonding to *¹¹, and the other three each are independently N or C-*²,
one of X²¹ to X²⁴ is a carbon atom bonding to *²¹, and the other three each are independently N or C-*²,
one of X²⁵ to X²⁸ is a carbon atom bonding to *²², and the other three each are independently N or C-*²,
one of X³¹ to X³⁴ is a carbon atom bonding to *³¹, and the other three are independently N or CRx¹,
X³⁵ to X³⁸ each independently represent N or CRx¹,
one of *¹'s or one of *²'s bonds to L¹ in the formula (1),
*¹ not bonding to L¹ bonds to Ry¹,
*² not bonding to L¹ bonds to Rx¹,
Ry¹ each independently represents a hydrogen atom or a substituent,
Rx¹ each independently represents a hydrogen atom or a substituent, and
Rx¹'s may form a ring; 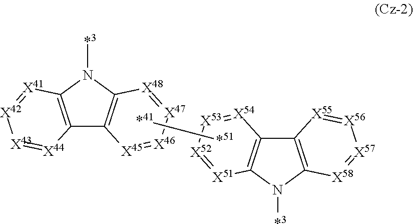
wherein X⁴¹ to X⁴⁴ each independently represent N or C-*⁴,
one of X⁴⁵ to X⁴⁸ is a carbon atom bonding to *⁴¹, and the other three each are independently N or C-*⁴,
one of X⁵¹ to X⁵⁴ is a carbon atom bonding to *⁵¹, and the other three each are independently N or C-*⁴,
X⁵⁵ to X⁵⁸ each independently represent N or C-*⁴,
one of *³'s or one of *⁴'s bonds to L² in the formula (1),
*³ not bonding to L² bonds to Ry²,
*⁴ not bonding to L² bonds to Rx²,
Ry² each independently represents a hydrogen atom or a substituent,
Rx² each independently represents a hydrogen atom or a substituent, and
Rx²'s may form a ring.

2. The organic electroluminescence device according to claim 1, wherein the compound represented by the formula (1) is a compound represented by the formula (1-2): 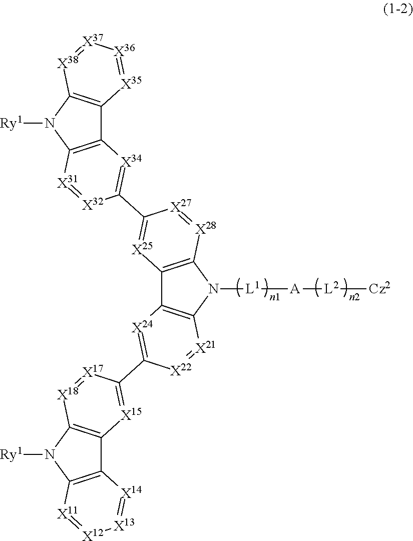
wherein X¹¹ to X¹⁵, X¹⁷ to X¹⁸, X²¹ to X²², X²⁴ to X²⁵, X²⁷ to X²⁸, X³¹ to X³² and X³⁴ to X³⁸ each independently represent N or CRx¹,
Ry¹ each independently represents a hydrogen atom or a substituent,
Rx¹ each independently represents a hydrogen atom or a substituent,
Rx¹'s may form a ring, and
in the formula (1-2), Cz², A, L¹, L², n1 and n2 are the same as Cz², A, L¹, L², n1 and n2 in the formula (1).

3. The organic electroluminescence device according to claim 1, wherein the compound represented by the formula (1) is a compound represented by the formula (1-4): 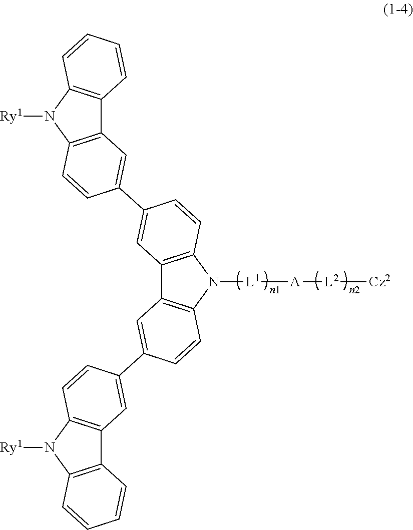
wherein Ry¹ each independently represents a hydrogen atom or a substituent, and Cz², A, L¹, L², n1 and n2 are the same as Cz², A, L¹, L², n1 and n2 in the formula (1).

4. The organic electroluminescence device according to claim 1, wherein Cz² is a group represented by the formula (Cz-2a): 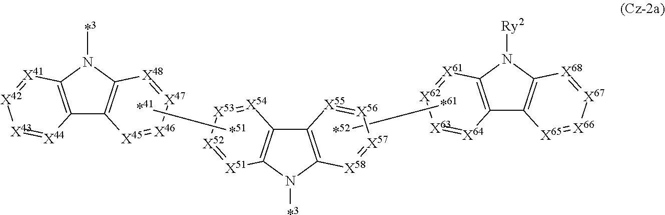
wherein X⁴¹ to X⁴⁴ each independently represent N or C-*⁴,
one of X⁴⁵ to X⁴⁸ is a carbon atom bonding to *⁴¹, and the other three each are independently N or C-*⁴,
one of X⁵¹ to X⁵⁴ is a carbon atom bonding to *⁵¹, and the other three each are independently N or C-*⁴,
one of X⁵⁵ to X⁵⁸ is a carbon atom bonding to *⁵², and the other three each are independently N or C-*⁴,
one of X⁶′ to X⁶⁴ is a carbon atom bonding to *⁶¹, and the other three each are independently N or CRx²,
X⁶⁵ to X⁶⁸ each independently represent N or CRx²,
one of *³'s or one of *⁴'s bonds to L² in the formula (1),
*³ not bonding to L² bonds to Ry²,
*⁴ not bonding to L² bonds to Rx²,
Ry² each independently represents a hydrogen atom or a substituent,
Rx² each independently represents a hydrogen atom or a substituent, and
Rx²'s may form a ring.

5. The organic electroluminescence device according to claim 1, wherein the compound represented by the formula (1) is a compound represented by the formula (1-2a): 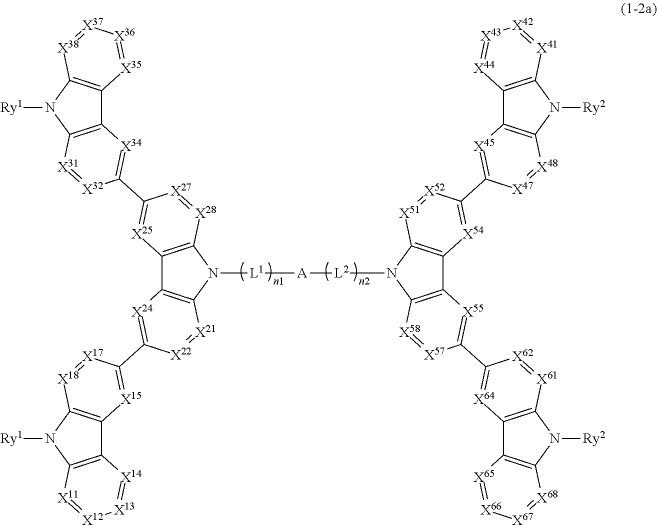
wherein X¹¹ to X¹⁵, X¹⁷ to X¹⁸, X²¹ to X²², X²⁴ to X²⁵, X²⁷ to X²⁸, X³¹ to X³² and X³⁴ to X³⁸ each independently N or CRx¹,
Ry¹ each independently represents a hydrogen atom or a substituent,
Rx¹ each independently represents a hydrogen atom or a substituent,
X⁴¹ to X⁴⁵, X⁴⁷ to X⁴⁸, X⁵¹ to X⁵², X⁵⁴ to X⁵⁵, X⁵⁷ to X⁵⁸, X⁶¹ to X⁶² and X⁶⁴ to X⁶⁸ each independently represent N or CRx²,
Ry² each independently represents a hydrogen atom or a substituent,
Rx² each independently represents a hydrogen atom or a substituent,
Rx¹'s may form a ring,
Rx²'s may form a ring, and
A, L¹, L², n1 and n2 are the same as A, L¹, L², n1 and n2 in the formula (1).

6. The organic electroluminescence device according to claim 1, wherein the compound represented by the formula (1) is a compound represented by the formula (1-4a): 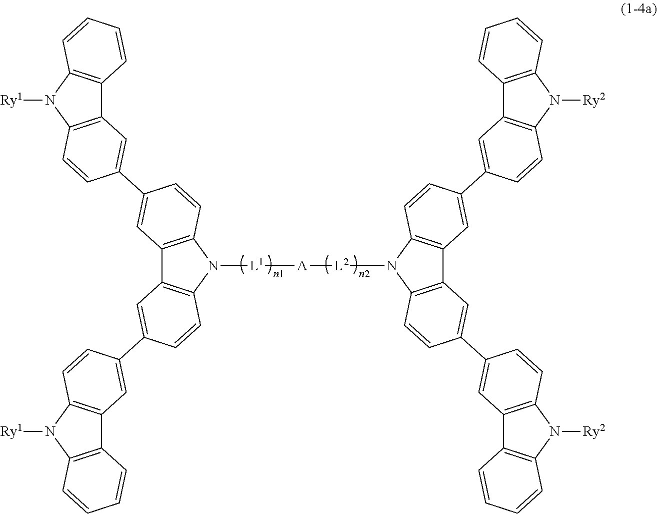
wherein Ry¹ each independently represents a hydrogen atom or a substituent,
Ry² each independently represents a hydrogen atom or a substituent, and
A, L¹, L², n1 and n2 are the same as A, L¹, L², n1 and n2 in the formula (1).

7. The organic electroluminescence device according to claim 1, wherein the compound represented by the formula (1) is a compound represented by the formula (1-4b): 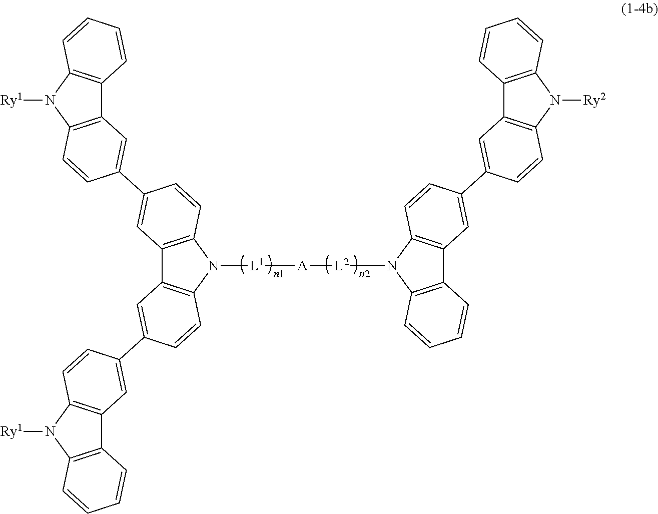
wherein Ry¹ each independently represents a hydrogen atom or a substituent,
Ry² represents a hydrogen atom or a substituent, and
A, L¹, L², n1 and n2 are the same as A, L¹, L², n1 and n2 in the formula (1).

8. The organic electroluminescence device according to claim 1, wherein the light emitting layer comprises the compound represented by the formula (1) as a hot material.

9. The organic electroluminescence device according to claim 1, wherein the light emitting layer comprises a phosphorescent light emitting material.

10. The organic electroluminescence device according to claim 1, which comprises an electron transporting layer between the cathode and the light emitting layer and wherein the electron transporting layer comprises a compound represented by the formula (1).

11. The organic electroluminescence device according to claim 1, which comprises a hole transporting layer between the cathode and the light emitting layer and wherein the hole transporting layer comprises a compound represented by the formula (1).

12. An electronic equipment comprising the organic electroluminescence device of claim 1.

13. A compound represented by the formula (1): 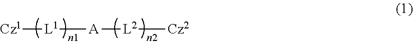
wherein Cz¹ represents a group represented by the formula (Cz-1),
Cz² represents a group represented by the formula (Cz-2),
A represents a residue of a substituted or unsubstituted nitrogen-containing aromatic hetero ring having 6 to 30 ring atoms,
L¹ and L² each independently represent a substituted or unsubstituted arylene group having 6 to 60 ring carbon atoms,
n1 and n2 each independently indicate an integer of 0 to 4,
provided that the structure represented by -(L¹)ₙ₁-Cz¹ differs from the structure represented by -(L²)ₙ₂-Cz²,
when n1=0, A and Cz¹ bond via a single bond,
when n2=0, A and Cz² bond via a single bond,
when n1 is an integer of 2 to 4, L¹'s may be the same as or different from each other, and L¹'s may form a ring,
when n2 is an integer of 2 to 4, L²'s may be the same as or different from each other, and L²'s may form a ring; 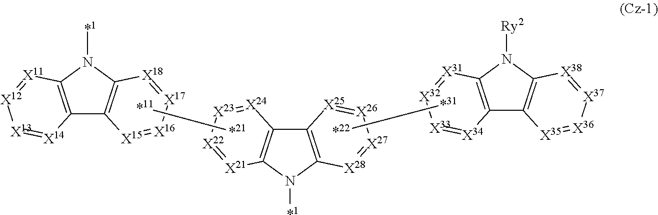
wherein X¹¹ to X¹⁴ each independently represent N or C-*²,
one of X¹⁵ to X¹⁸ is a carbon atom bonding to *¹¹, and the other three each are independently N or C-*²,
one of X²¹ to X²⁴ is a carbon atom bonding to *²¹, and the other three each are independently N or C-*²,
one of X²⁵ to X²⁸ is a carbon atom bonding to *²², and the other three each are independently N or C-*²,
one of X³¹ to X³⁴ is a carbon atom bonding to *³¹, and the other three are independently N or CRx¹,
X³⁵ to X³⁸ each independently represent N or CRx¹,
one of *¹'s or one of *²'s bonds to L¹ in the formula (1),
*¹ not bonding to L¹ bonds to Ry¹,
*² not bonding to L¹ bonds to Rx¹,
Ry¹ each independently represents a hydrogen atom or a substituent,
Rx¹ each independently represents a hydrogen atom or a substituent, and
Rx¹'s may form a ring; 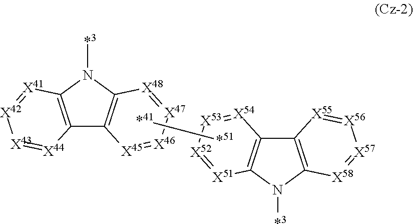
wherein X⁴¹ to X⁴⁴ each independently represent N or C-*⁴,
one of X⁴⁵ to X⁴⁸ is a carbon atom bonding to *⁴¹, and the other three each are independently N or C-*⁴,
one of X⁵¹ to X⁵⁴ is a carbon atom bonding to *⁵¹, and the other three each are independently N or C-*⁴,
X⁵⁵ to X⁵⁸ each independently represent N or C-*⁴,
one of *³'s or one of *⁴'s bonds to L² in the formula (1),
*³ not bonding to L² bonds to Ry²,
*⁴ not bonding to L² bonds to Rx²,
Ry² each independently represents a hydrogen atom or a substituent,
Rx² each independently represents a hydrogen atom or a substituent, and
Rx²'s may form a ring.

14. The compound according to claim 13, wherein Cz¹ is a group represented by the formula (Cz-11): 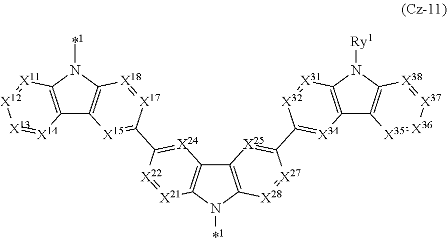
wherein X¹¹ to X¹⁵, X¹⁷ to X¹⁸, X²¹ to X²², X²⁴ to X²⁵ and X²⁷ to X²⁸ each independently represent N or C-*²,
X³¹ to X³² and X³⁴ to X³⁸ each independently represent N or CRx¹,
one of *¹'s or one of *²'s bonds to L¹ in the formula (1),
*¹ not bonding to L¹ bonds to Ry¹,
*² not bonding to L¹ bonds to Rx¹,
Ry¹ each independently represents a hydrogen atom or a substituent,
Rx¹ each independently represents a hydrogen atom or a substituent, and
Rx¹'s may form a ring.

15. The compound according to claim 13, which is represented by the formula (1-2): 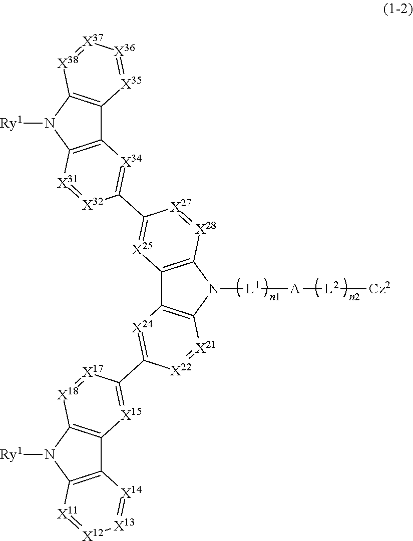
wherein X¹¹ to X¹⁵, X¹⁷ to X¹⁸, X²¹ to X²², X²⁴ to X²⁵, X²⁷ to X²⁸, X³¹ to X³² and X³⁴ to X³⁸ each independently represent N or CRx¹,
Ry¹ each independently represents a hydrogen atom or a substituent,
Rx¹ each independently represents a hydrogen atom or a substituent,
Rx¹'s may form a ring, and
Cz², A, L¹, L², n1 and n2 are the same as Cz², A, L¹, L², n1 and n2 in the formula (1),
provided that the structure represented by the formula (1-2-L) differs from the structure represented by -(L²)ₙ₂-Cz²:

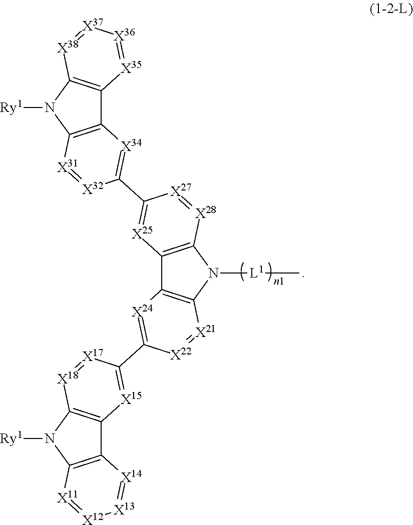

16. The compound according to claim 13, which is represented by the formula (1-3): 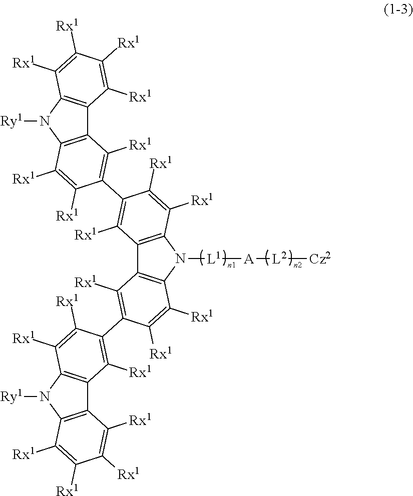
wherein Ry¹ each independently represents a hydrogen atom or a substituent,
Rx¹ each independently represents a hydrogen atom or a substituent,
Rx¹'s may form a ring, and
Cz², A, L¹, L², n1 and n2 are the same as Cz², A, L¹, L², n1 and n2 in the formula (1),
provided that the structure represented by the formula (1-3-L) differs from the structure represented by -(L²)ₙ₂-Cz².

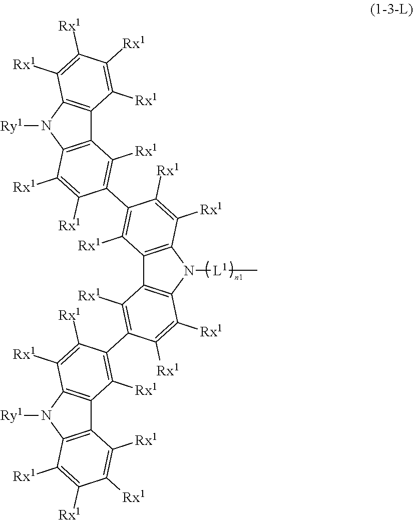

17. The compound according to claim 13, which is represented by the formula (1-4): 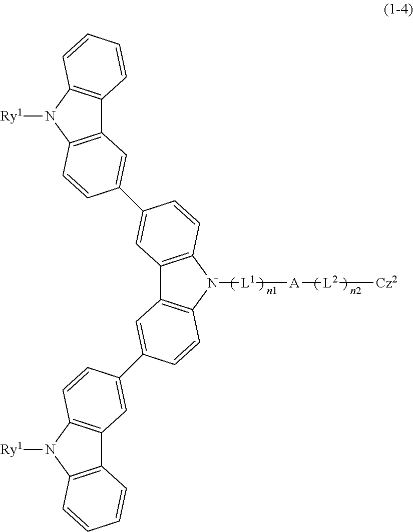
wherein Ry¹ each independently represents a hydrogen atom or a substituent, and
Cz², A, L¹, L², n1 and n2 are the same as Cz², A, L¹, L², n1 and n2 in the formula (1),
provided that the structure represented by the formula (1-4-L) differs from the structure represented by -(L²)ₙ₂-Cz²:

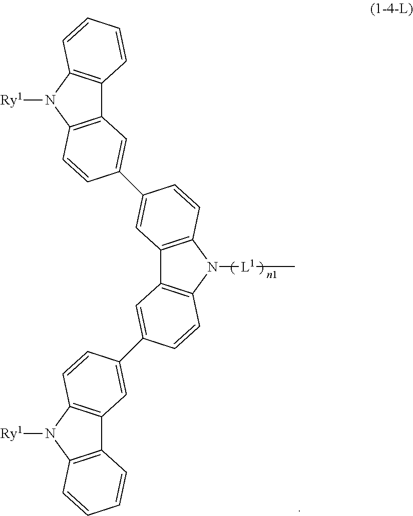

18. The compound according to claim 13, wherein Cz² is a group represented by the formula (Cz-2a): 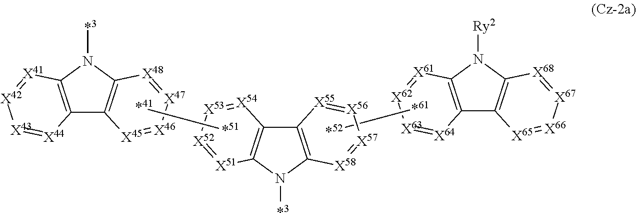
wherein X⁴¹ to X⁴⁴ each independently represent N or C-*⁴,
one of X⁴⁵ to X⁴⁸ is a carbon atom bonding to *⁴¹, and the other three each are independently N or C-*⁴,
one of X⁵¹ to X⁵⁴ is a carbon atom bonding to *⁵¹, and the other three each are independently N or C-*⁴,
one of X⁵⁵ to X⁵⁸ is a carbon atom bonding to *⁵², and the other three each are independently N or C-*⁴,
one of X⁶¹ to X⁶⁴ is a carbon atom bonding to *⁶¹, and the other three each are independently N or CRx²,
X⁶⁵ to X⁶⁸ each independently represent N or CRx²,
one of *³'s or one of *⁴'s bonds to L² in the formula (1),
*³ not bonding to L² bonds to Ry²,
*⁴ not bonding to L² bonds to Rx²,
Ry² each independently represents a hydrogen atom or a substituent,
Rx² each independently represents a hydrogen atom or a substituent, and
Rx²'s may form a ring.

19. The compound according to claim 13, wherein Cz² is a group represented by the formula (Cz-21a): 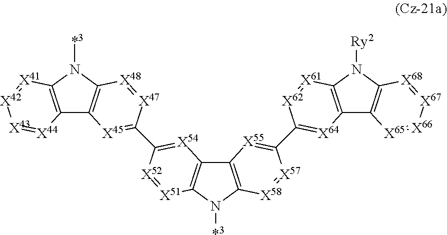
wherein X⁴¹ to X⁴⁵, X⁴⁷ to X⁴⁸, X⁵¹ to X⁵², X⁵⁴ to X⁵⁵ and X⁵⁷ to X⁵⁸ each independently represent N or C-*⁴,
X⁶¹ to X⁶² and X⁶⁴ to X⁶⁸ each independently represent N or CRx²,
one of *³'s or one of *⁴'s bonds to L² in the formula (1),
*³ not bonding to L² bonds to Ry²,
*⁴ not bonding to L² bonds to Rx²,
Ry² each independently represents a hydrogen atom or a substituent,
Rx² each independently represents a hydrogen atom or a substituent, and
Rx²'s may form a ring.

20. The compound according to claim 13, which is represented by the general formula (1-2a): 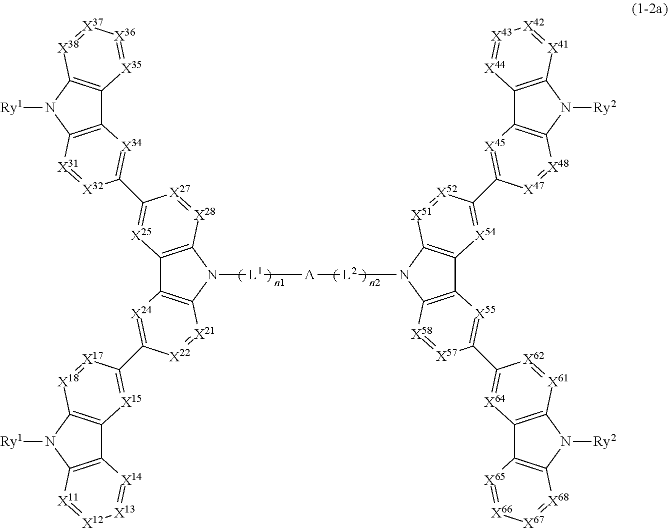
wherein X¹¹ to X¹⁵, X¹⁷ to X¹⁸, X²¹ to X²², X²⁴ to X²⁵, X²⁷ to X²⁸, X³¹ to X³² and X³⁴ to X³⁸ each independently N or CRx¹,
Ry¹ each independently represents a hydrogen atom or a substituent,
Rx¹ each independently represents a hydrogen atom or a substituent,
X⁴¹ to X⁴⁵, X⁴⁷ to X⁴⁸, X⁵¹ to X⁵², X⁵⁴ to X⁵⁵, X⁵⁷ to X⁵⁸, X⁶¹ to X⁶² and X⁶⁴ to X⁶⁸ each independently represent N or CRx²,
Ry² each independently represents a hydrogen atom or a substituent,
Rx² each independently represents a hydrogen atom or a substituent,
Rx¹'s may form a ring,
Rx²'s may form a ring, and
A, L¹, L², n1 and n2 are the same as A, L¹, L², n1 and n2 in the formula (1), provided that the structure represented by the formula (1-2a-L) differs from the structure represented by the formula (1-2a-R):

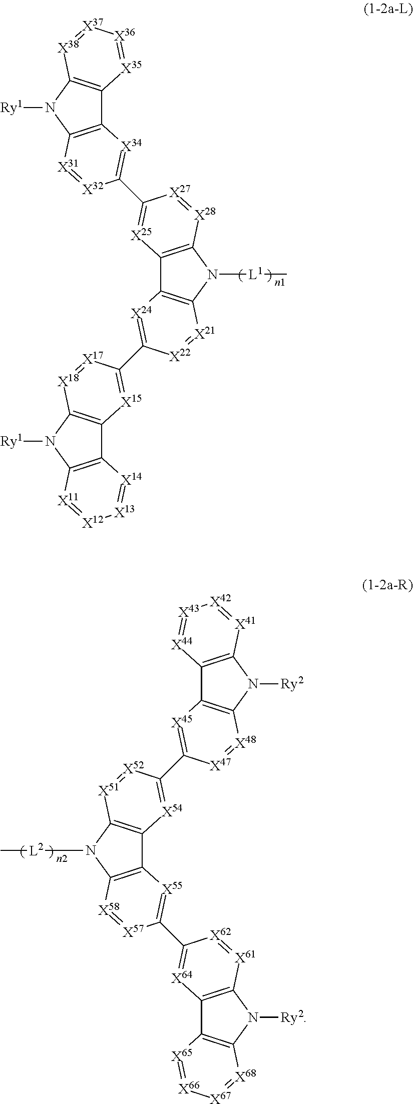

21. The compound according to claim 13, which is represented by the formula (1-4a): 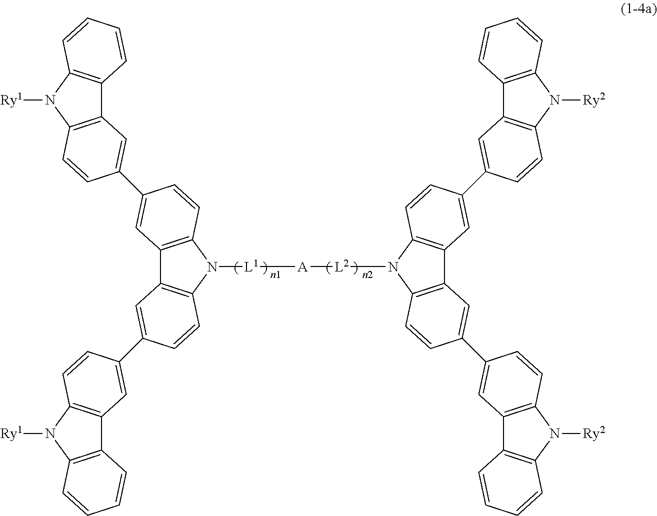
wherein Ry¹ each independently represents a hydrogen atom or a substituent,
Ry² each independently represents a hydrogen atom or a substituent, and
A, L¹, L², n1 and n2 are the same as A, L¹, L², n1 and n2 in the formula (1),
provided that the structure represented by the formula (1-4a-L) differs from the structure represented by the formula (1-4a-R):

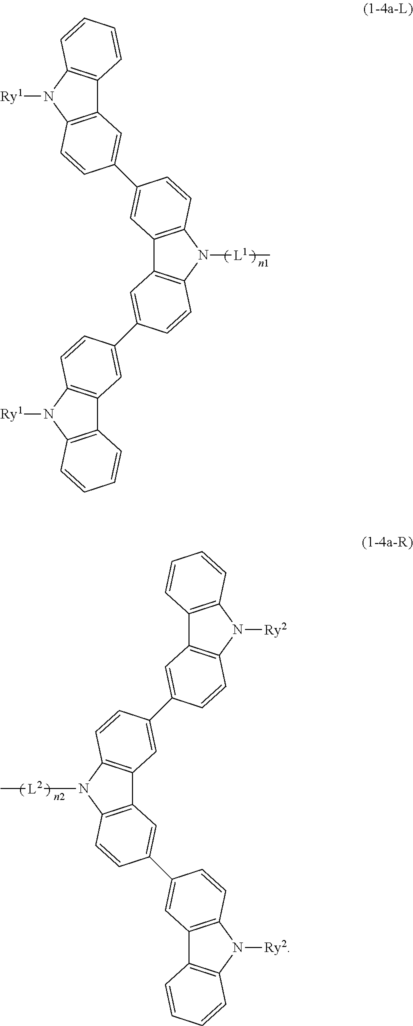

22. The compound according to claim 13, wherein Cz² is a group represented by the formula (Cz-21b): 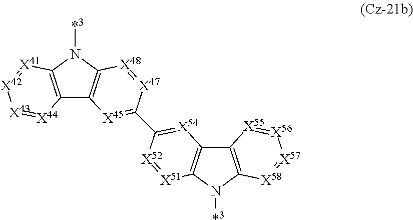
wherein X⁴¹ to X⁴⁵, X⁴⁷ to X⁴⁸, X⁵¹ to X⁵² and X⁵⁴ to X⁵⁸ each independently represent N or C-*⁴,
one of *³'s or one of *⁴'s bonds to L² in the formula (1),
*³ not bonding to L² bonds to Ry²,
*⁴ not bonding to L² bonds to Rx²,
Ry² each independently represents a hydrogen atom or a substituent,
Rx² each independently represents a hydrogen atom or a substituent, and
Rx²'s may form a ring.

23. The compound according to claim 13, which is represented by the formula (1-2b): 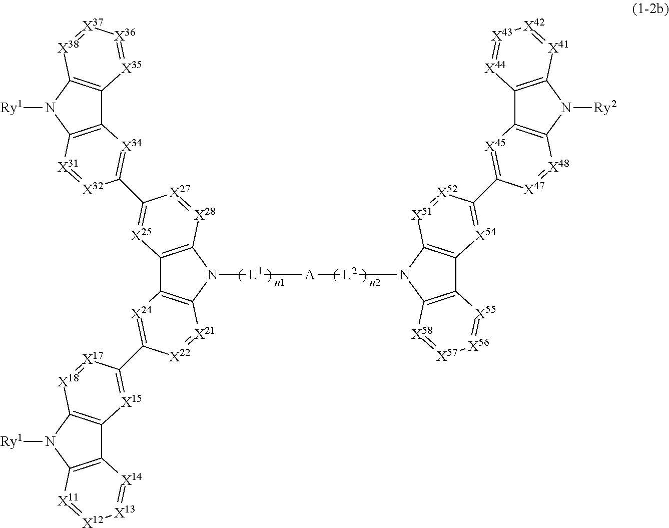
wherein X¹¹ to X¹⁵, X¹⁷ to X¹⁸, X²¹ to X²², X²⁴ to X²⁵, X²⁷ to X²⁸, X³¹ to X³² and X³⁴ to X³⁸ each independently represent N or CRx¹,
Ry¹ each independently represents a hydrogen atom or a substituent,
Rx¹ each independently represents a hydrogen atom or a substituent,
X⁴¹ to X⁴⁵, X⁴⁷ to X⁴⁸, X⁵¹ to X⁵² and X⁵⁴ to X⁵⁸ each independently represent N or CRx²,
Ry² represents a hydrogen atom or a substituent,
Rx² each independently represents a hydrogen atom or a substituent,
Rx¹'s may form a ring,
Rx²'s may form a ring, and
A, L¹, L², n1 and n2 are the same as A, L¹, L², n1 and n2 in the formula (1),
provided that the structure represented by the formula (1-2b-L) differs from the structure represented by the formula (1-2b-R):

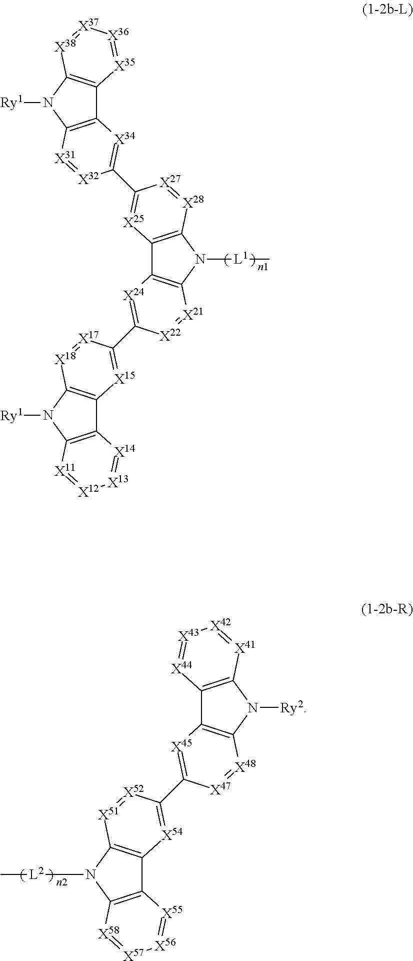

24. The compound according to claim 13, which is represented by the formula (1-4b): 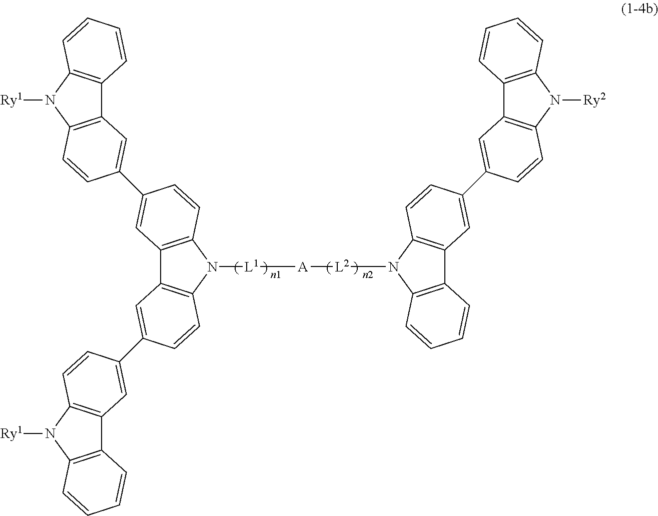
wherein Ry¹ each independently represents a hydrogen atom or a substituent,
Ry² represents a hydrogen atom or a substituent, and
A, L¹, L², n1 and n2 are the same as A, L¹, L², n1 and n2 in the formula (1).

25. The compound according to claim 13, wherein Rx¹ and Rx² each independently represent a hydrogen atom or a substituent selected from a group consisting of a substituted or unsubstituted alkyl group having 1 to 50 carbon atoms, a substituted or unsubstituted cycloalkyl group having 3 to 50 ring carbon atoms, a substituted or unsubstituted aryl group having 6 to 60 ring carbon atoms, a substituted or unsubstituted aralkyl group having 7 to 61 carbon atoms, an amino group, a mon-substituted or di-substituted amino group having a substituent selected from a substituted or unsubstituted alkyl group with 1 to 50 carbon atoms and a substituted or unsubstituted aryl group with 6 to 60 ring carbon atoms, a substituted or unsubstituted alkoxy group having 1 to 50 carbon atoms, a substituted or unsubstituted cycloalkoxy group having 3 to 50 ring carbon atoms, a substituted or unsubstituted aryloxy group having 6 to 60 ring carbon atoms, a substituted or unsubstituted alkylthio group having 1 to 50 carbon atoms, a substituted or unsubstituted arylthio group having 6 to 60 ring carbon atoms, a mono-substituted, di-substituted or tri-substituted silyl group having a substituent selected from a substituted or unsubstituted alkyl group with 1 to 50 carbon atoms and a substituted or unsubstituted aryl group with 6 to 60 ring carbon atoms, a substituted or unsubstituted heteroaryl group having 5 to 60 ring atoms, a substituted or unsubstituted haloalkyl group having 1 to 50 carbon atoms, a halogen atom, a cyano group, a nitro group, a sulfonyl group having a substituent selected from a substituted or unsubstituted alkyl group with 1 to 50 carbon atoms and a substituted or unsubstituted aryl group with 6 to 60 ring carbon atoms, a di-substituted phosphoryl group having a substituent selected from a substituted or unsubstituted alkyl group with 1 to 50 carbon atoms and a substituted or unsubstituted aryl group with 6 to 60 ring carbon atoms, an alkylsulfonyloxy group, an arylsulfonyloxy group, an alkylcarbonyloxy group, an arylcarbonyloxy group, a boron-containing group, a zinc-containing group, a tin-containing group, a silicon-containing group, a magnesium-containing group, a lithium-containing group, a hydroxy group, an alkyl-substituted or aryl-substituted carbonyl group, a carboxyl group, a vinyl group, a (meth)acryloyl group, an epoxy group, and an oxetanyl group.

26. The compound according to claim 13, wherein Ry¹ and Ry² each independently represent a hydrogen atom or a substituent selected from a group consisting of a substituted or unsubstituted alkyl group having 1 to 50 carbon atoms, a substituted or unsubstituted cycloalkyl group having 3 to 50 ring carbon atoms, a substituted or unsubstituted aryl group having 6 to 60 ring carbon atoms, a substituted or unsubstituted aralkyl group having 7 to 61 carbon atoms, an amino group, a mon-substituted or di-substituted amino group having a substituent selected from a substituted or unsubstituted alkyl group with 1 to 50 carbon atoms and a substituted or unsubstituted aryl group with 6 to 60 ring carbon atoms, a substituted or unsubstituted alkoxy group having 1 to 50 carbon atoms, a substituted or unsubstituted cycloalkoxy group having 3 to 50 ring carbon atoms, a substituted or unsubstituted aryloxy group having 6 to 60 ring carbon atoms, a substituted or unsubstituted alkylthio group having 1 to 50 carbon atoms, a substituted or unsubstituted arylthio group having 6 to 60 ring carbon atoms, a mono-substituted, di-substituted or tri-substituted silyl group having a substituent selected from a substituted or unsubstituted alkyl group with 1 to 50 carbon atoms and a substituted or unsubstituted aryl group with 6 to 60 ring carbon atoms, a substituted or unsubstituted heteroaryl group having 5 to 60 ring atoms, a substituted or unsubstituted haloalkyl group having 1 to 50 carbon atoms, a halogen atom, a cyano group, a nitro group, a sulfonyl group having a substituent selected from a substituted or unsubstituted alkyl group with 1 to 50 carbon atoms and a substituted or unsubstituted aryl group with 6 to 60 ring carbon atoms, a di-substituted phosphoryl group having a substituent selected from a substituted or unsubstituted alkyl group with 1 to 50 carbon atoms and a substituted or unsubstituted aryl group with 6 to 60 ring carbon atoms, an alkylsulfonyloxy group, an arylsulfonyloxy group, an alkylcarbonyloxy group, an arylcarbonyloxy group, a boron-containing group, a zinc-containing group, a tin-containing group, a silicon-containing group, a magnesium-containing group, a lithium-containing group, a hydroxy group, an alkyl-substituted or aryl-substituted carbonyl group, a carboxyl group, a vinyl group, a (meth)acryloyl group, an epoxy group, and an oxetanyl group.

27. The compound according to claim 13, wherein n1≠n2.

28. The compound according to claim 13, wherein any one of n1 and n2 is 0 and the other is an integer of 1 to 4.

29. The compound according to claim 13, wherein n1=n2 and L¹ differs from L².

30. The compound according to claim 13, wherein the compound represented by the formula (1) is a compound represented by the formula (1-x) or (1-y): 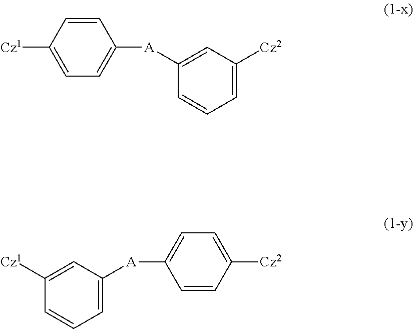
wherein Cz¹, Cz² and A are the same as Cz¹, Cz² and A in the formula (1).

31. The compound according to claim 13, wherein A is a residue of a nitrogen-containing aromatic hetero ring selected from a group consisting of a substituted or unsubstituted pyridine ring, a substituted or unsubstituted pyrazine ring, a substituted or unsubstituted pyrimidine ring, a substituted or unsubstituted pyridazine ring, a substituted or unsubstituted triazine ring, a substituted or unsubstituted quinoline ring, a substituted or unsubstituted isoquinoline ring, a substituted or unsubstituted quinoxaline ring, a substituted or unsubstituted quinazoline ring, a substituted or unsubstituted cinnoline ring, a substituted or unsubstituted benzoquinazoline ring, and a substituted or unsubstituted azafluoranthene ring.

32. The compound according to claim 13, wherein A is a residue of a nitrogen-containing aromatic hetero ring selected from a group consisting of a substituted or unsubstituted pyrimidine ring, a substituted or unsubstituted triazine ring, a substituted or unsubstituted quinazoline ring, a substituted or unsubstituted benzoquinazoline ring, and a substituted or unsubstituted azafluoranthene ring.

33. The compound according to claim 13, wherein A is a residue of a nitrogen-containing aromatic hetero ring selected from a group consisting of a substituted or unsubstituted pyrimidine ring, and a substituted or unsubstituted quinazoline ring.

34. The compound according to claim 13, wherein A is a group represented by the formula (A-1) or (A-2): 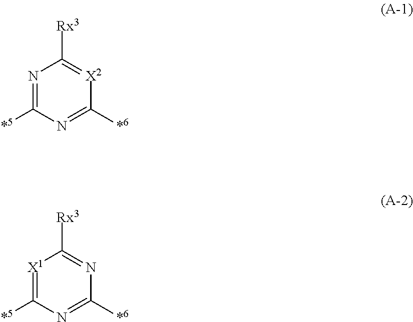
wherein X¹ and X² each independently represent N or CRx³,
Rx³ each independently represents a hydrogen atom or a substituent,
*⁵ bonds to L¹ in the formula (1),
*⁶ bonds to L² in the formula (1), and
Rx³'s may form a ring.

35. The compound according to claim 13, wherein A is a group represented by the formula (A-3) or (A-4): 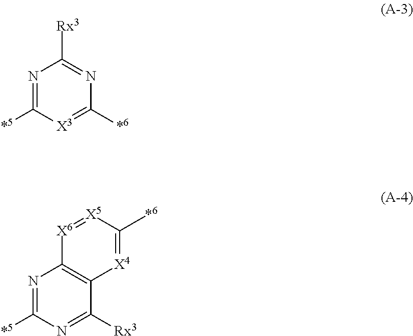
wherein X³ to X⁶ each independently represent N or CRx³,
Rx³ each independently represents a hydrogen atom or a substituent,
*⁵ bonds to L¹ in the formula (1),
*⁶ bonds to L² in the formula (1), and
Rx³'s may form a ring.

36. The compound according to claim 35, wherein Rx³ each independently represents a hydrogen atom or a substituent selected from a group consisting of a substituted or unsubstituted alkyl group having 1 to 50 carbon atoms, a substituted or unsubstituted cycloalkyl group having 3 to 50 ring carbon atoms, a substituted or unsubstituted aryl group having 6 to 60 ring carbon atoms, a substituted or unsubstituted aralkyl group having 7 to 61 carbon atoms, an amino group, a mon-substituted or di-substituted amino group having a substituent selected from a substituted or unsubstituted alkyl group with 1 to 50 carbon atoms and a substituted or unsubstituted aryl group with 6 to 60 ring carbon atoms, a substituted or unsubstituted alkoxy group having 1 to 50 carbon atoms, a substituted or unsubstituted cycloalkoxy group having 3 to 50 ring carbon atoms, a substituted or unsubstituted aryloxy group having 6 to 60 ring carbon atoms, a substituted or unsubstituted alkylthio group having 1 to 50 carbon atoms, a substituted or unsubstituted arylthio group having 6 to 60 ring carbon atoms, a mono-substituted, di-substituted or tri-substituted silyl group having a substituent selected from a substituted or unsubstituted alkyl group with 1 to 50 carbon atoms and a substituted or unsubstituted aryl group with 6 to 60 ring carbon atoms, a substituted or unsubstituted heteroaryl group having 5 to 60 ring atoms, a substituted or unsubstituted haloalkyl group having 1 to 50 carbon atoms, a halogen atom, a cyano group, a nitro group, a sulfonyl group having a substituent selected from a substituted or unsubstituted alkyl group with 1 to 50 carbon atoms and a substituted or unsubstituted aryl group with 6 to 60 ring carbon atoms, a di-substituted phosphoryl group having a substituent selected from a substituted or unsubstituted alkyl group with 1 to 50 carbon atoms and a substituted or unsubstituted aryl group with 6 to 60 ring carbon atoms, an alkylsulfonyloxy group, an arylsulfonyloxy group, an alkylcarbonyloxy group, an arylcarbonyloxy group, a boron-containing group, a zinc-containing group, a tin-containing group, a silicon-containing group, a magnesium-containing group, a lithium-containing group, a hydroxy group, an alkyl-substituted or aryl-substituted carbonyl group, a carboxyl group, a vinyl group, a (meth)acryloyl group, an epoxy group, and an oxetanyl group.

37. A material for organic electroluminescence devices comprising the compound of claim 13.

38. An ink composition comprising a solvent and the compound of claim 13 dissolved in the solvent.
